(19) United States
(12) Patent Application Publication
(10) Pub. No.: US 2026/0174778 A1
(43) Pub. Date: Jun. 25, 2026

(54) SULFONAMIDE COMPOUND, AND PREPARATION METHOD THEREFOR AND USE THEREOF

(71) Applicants: SHANGHAI HENLIUS BIOTECH, INC., Shanghai (CN); SHANGHAI HENLIUS BIOLOGICS CO., LTD., Shanghai (CN); SHANGHAI HENLIUS BIOSCIENCE CO., LTD., Shanghai (CN)

(72) Inventors: Yushi CHI, Shanghai (CN); Rui LIU, Shanghai (CN); Xiayan ZHANG, Shanghai (CN); Yongqiang SHAN, Shanghai (CN); Xuemei TIAN, Shanghai (CN)

(21) Appl. No.: 19/539,580

(22) Filed: Feb. 13, 2026

Related U.S. Application Data
(63) Continuation of application No. PCT/CN2024/111949, filed on Aug. 14, 2024.

(30) Foreign Application Priority Data
Aug. 14, 2023 (CN) 202311021822.3
Sep. 27, 2023 (CN) 202311258886.5
Dec. 29, 2023 (CN) 202311862643.2
Apr. 19, 2024 (CN) 202410481362.0

(51) Int. Cl. A61K 31/553; A61K 31/423; A61K 31/428; A61K 31/437; A61K 31/538; A61P 35/00; C07B 59/00; C07D 413/06; C07D 413/14; C07D 417/06; C07D 417/14; C07D 471/04; C07D 495/10; C07D 498/04; C07D 519/00

(52) U.S. Cl. CPC A61K 31/553; A61K 31/423; A61K 31/428; A61K 31/437; A61K 31/538; A61P 35/00; C07B 59/002; C07D 413/06; C07D 413/14; C07D 417/06; C07D 417/14; C07D 471/04; C07D 495/10; C07D 498/04; C07D 519/00

(57) ABSTRACT

The present disclosure relates to a substituted sulfonamide derivative of formula (I), a preparation method therefor, a pharmaceutical composition containing the compound, and the use thereof in the treatment and/or prevention of cancers in which lysine acetyltransferase 6A/B (KAT6A/B) plays a role.

### TECHNICAL FIELD

The present disclosure pertains to the field of pharmaceutics, and relates to a preparation method for a substituted sulfonamide compound and use thereof. Specifically, the present disclosure relates to a substituted sulfonamide compound of formula (I), a preparation method therefor, a pharmaceutical composition comprising the compound, and use thereof in the treatment and/or prevention of a cancer in which lysine acetyltransferase 6A/B (KAT6A/B) plays a role.

### BACKGROUND

Lysine acetyltransferases (KATs) use acetyl coenzyme A as an acetyl donor, catalyzing the acetylation of F-amino groups of lysine residues on histones and non-histone proteins. Acetylation, as a reversible post-translational modification, affects protein function and gene expression through various mechanisms, including modulation of protein stability, enzymatic activity, subcellular localization, interactions with other types of post-translational modifications, and protein-DNA interactions.

Based on the homology of amino acid sequences, KATs can be divided into 4 subfamilies, namely the MYST family, the p300/CBP family, the SRC/p160 family, and the GNAT family. Among them, the largest subfamily is the MYST family, whose name is composed of the first letters of four early discovered members: MOZ, Ybf2, Sas2, and Tip60. The MYST family in mammals has a total of five members: KAT5 (TIP60), KAT6A (MOZ; MYST3), KAT6B (MORF; MYST4), KAT7 (HBO; MYST2), and KAT8 (MOF; MYST1), all of which have homologous genes in humans.

The members of the MYST family of proteins all contain a highly conserved MYST domain. This domain functions as an adapter protein that can interact with other proteins to form protein complexes. It has also been reported that the MYST domain may possess DNA-binding ability. In addition, zinc finger structures and chromatin-binding domains are also characteristic domains of this family of KATs. The MYST family of proteins regulates gene transcription activity by modulating the acetylation levels of lysine residues on histones (H2A, H3, H4). The downstream biological functions involved include the initiation of DNA replication, the regulation of gene expression, the repair of DNA damage, the modulation of Treg cells, the maintenance of cell stemness, the development and differentiation of the central nervous system and hematopoietic system, etc. Abnormal activation of KATs of the MYST family or related complexes thereof can easily lead to uncontrolled cell growth and apoptosis, thereby resulting in the occurrence of malignant tumors.

KAT6A (lysine acetyltransferase 6A, also known as MOZ) and KAT6B (lysine acetyltransferase 6B, also known as MORF), as important members of the MYST family, are primarily responsible for the acetylation modification of lysine 23 on histone H3 (H3K23). In addition, KAT6A is also capable of acetylating H3K9 and H3K14. The physiological and pathological functions regulated by KAT6A and KAT6B are quite extensive. They are involved in the development of the nervous system, the development of the cardiac septum, the proliferation and differentiation of hematopoietic progenitor cells, the maintenance of cell stemness, and the occurrence, development, and drug resistance generation of various tumors.

Abnormal alterations in the KAT6A gene are closely related to the occurrence and development of human tumors. In hematological tumors (e.g., acute myeloid leukemia), chromosomal translocations of KAT6A have been reported, forming fusion genes, such as KAT6A-CBP, KAT6A-TIF2, KAT6A-NcoA3, KAT6A-LEUTX, and KAT6A-EP300. Meanwhile, in a variety of solid tumors (e.g., breast cancer, ovarian cancer, cervical cancer, lung adenocarcinoma, colon cancer, and rectal cancer), KAT6A gene amplification has been discovered. Particularly in breast cancer, it has been found that 10%-15% of patients have a duplicated amplification region at 8p11-12, with KAT6A precisely located in this amplified region. Additionally, Chromosomal rearrangements of the KAT6B gene have been reported in acute myeloid leukemia (e.g., KAT6B-CBP), and up-regulated expression of KAT6B has also been observed in breast cancer. In tumor cells with KAT6A/B amplification, the gene expression level is closely related to the gene copy number. Studies have shown that in breast cancer cells with 8p11 amplification, the KAT6A protein localizes to the promoter region of the estrogen receptor ERα, promoting the expression of ERα. Conversely, shRNA-mediated knockdown of KAT6A reduces the mRNA and protein levels of ERα. These data indicate that KAT6A positively regulates the gene expression of ERα, and the growth of ER-positive breast cancer cells is dependent on the expression of this gene. Molecular genetic studies have shown that knockdown of KAT6A can induce down-regulation of ERα expression and inhibit the growth of T47D tumors in vivo. CTx-648, a small molecule inhibitor of KAT6A/B, has also shown excellent in vivo and in vitro anti-tumor activity against ER-positive breast cancer in preclinical studies. In addition to ER-positive breast cancer, KAT6A/B inhibitors have also demonstrated potential prospects for indication expansion in a variety of other solid tumors. For example, in glioma, KAT6A exerts a cancer-promoting function. It binds to TRIM24 to promote PI3K/AKT signal transduction activity, thereby promoting glioma cell proliferation and tumor growth.

Given the important role of KAT6A/B in human diseases such as cancer, inhibitors against KAT6A/B have broad application prospects.

### SUMMARY

The present application relates to a compound of formula (I) as defined herein, which can act as a lysine acetyltransferase 6A/B (KAT6A/B) inhibitor. The present application is characterized by a method for treating and/or preventing a cancer in which lysine acetyltransferase 6A/B (KAT6A/B) plays a role, comprising administering to a subject in need thereof a therapeutically effective amount of the compound of formula (I) (including formulas II-1 to VII-3) as defined herein or a pharmaceutically acceptable salt, an ester, a stereoisomer, a tautomer, a polymorph, a hydrate, a solvate, an N-oxide, an isotopically labeled compound, a metabolite, or a prodrug thereof. The method of the present application can be used for treating a cancer in which lysine acetyltransferase 6A/B (KAT6A/B) plays a role by inhibiting the activity of lysine acetyltransferase 6A/B (KAT6A/B).

A first aspect of the present application relates to a compound of formula (I):

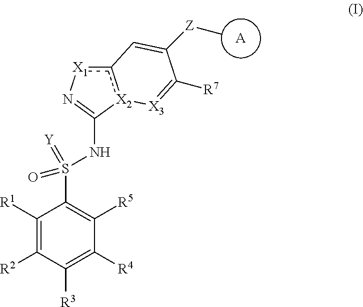

or a pharmaceutically acceptable salt, an ester, a stereoisomer, a tautomer, a polymorph, a hydrate, a solvate, an N-oxide, an isotopically labeled compound, a metabolite, or a prodrug thereof, wherein R¹, R², R³, R⁴, R⁵, R⁷, X₁, X₂, X₃, Y, ring A, and Z are as detailed herein below. Another aspect of the present application relates to a pharmaceutical composition, comprising the compound of formula (I) or

the pharmaceutically acceptable salt, the ester, the stereoisomer, the tautomer, the polymorph, the hydrate, the solvate, the N-oxide, the isotopically labeled compound, the metabolite, or the prodrug thereof, and one or more pharmaceutically acceptable carriers, diluents, or excipients. Another aspect of the present application relates to a method for inhibiting lysine acetyltransferase 6A/B (KAT6A/B). The method comprises administering to a subject in need thereof the compound of formula (I) or the pharmaceutically acceptable salt, the ester, the stereoisomer, the tautomer, the polymorph, the hydrate, the solvate, the N-oxide, the isotopically labeled compound, the metabolite, or the prodrug thereof, or the pharmaceutical composition comprising the same.

Another aspect of the present application relates to a method for treating a cancer in which lysine acetyltransferase 6A/B (KAT6A/B) plays a role. The method comprises administering to a subject in need thereof the compound of formula (I) or the pharmaceutically acceptable salt, the ester, the stereoisomer, the tautomer, the polymorph, the hydrate, the solvate, the N-oxide, the isotopically labeled compound, the metabolite, or the prodrug thereof, or the pharmaceutical composition comprising the same. The cancer is preferably selected from lung cancer, breast cancer, rectal cancer, colon cancer, esophageal cancer, gastric cancer, liver cancer, gallbladder cancer, cholangiocarcinoma, kidney cancer, bladder cancer, urothelial cancer, head and neck cancer, nasopharyngeal cancer, prostate cancer, cervical cancer/endometrial cancer, ovarian cancer, pancreatic cancer, melanoma, bone cancer, mesothelioma, gastrointestinal stromal tumor, sarcoma, brain glioma, thyroid cancer, salivary gland tumor, glioblastoma, neuroblastoma, gastric myxoma, lymphoma, leukemia, plasmacytoma, sinoatrial node tumor, and tenosynovial giant cell tumor, and more preferably selected from breast cancer, prostate cancer, lung cancer, pancreatic cancer, ovarian cancer, cervical cancer/endometrial cancer, bladder cancer, brain glioma, malignant lymphoma, liver cancer, and leukemia, wherein the breast cancer is preferably ER⁺ breast cancer or ER⁺/HER2⁻ breast cancer, the lung cancer is preferably non-small cell lung cancer, and the prostate cancer is preferably castration-resistant prostate cancer.

The present application further provides compounds and compositions having significantly improved inhibitory activity against lysine acetyltransferase 6A/B (KAT6A/B), significantly improved pharmacodynamics and pharmacokinetics, and/or a significantly improved safety profile compared to known KAT6A/B inhibitors.

The details of the present application are set forth in the accompanying description below. Although methods and materials similar or equivalent to those described herein can be used in the practice or testing of the present application, the illustrative methods and materials are now described. Other features, objectives, and advantages of the present application will be apparent from the specification and the claims. In the specification and the appended claims, the singular forms also include their plural forms, unless the context clearly indicates otherwise. Unless otherwise defined, all technical and scientific terms used herein have the same meanings as commonly understood by those of ordinary skill in the art to which the present application belongs. All patents and publications cited in this specification are incorporated herein by reference in their entirety.

The content of all references (including literature references, issued patents, published patent applications, and co-pending patent applications) cited throughout the present application is hereby expressly incorporated by reference in its entirety. Unless otherwise defined, all technical and scientific terms used herein have the meanings commonly known to those of ordinary skill in the art.

### BRIEF DESCRIPTION OF THE DRAWINGS

FIG. 1 shows the color development results of the effects of compounds on histone acetylation in estrogen receptor-positive breast cancer cells.

FIG. 2 shows the tumor volume change results from an anti-tumor experiment for compounds conducted in an ER⁺KAT6^{high} breast cancer cell-derived xenograft tumor model in Test Example 5.

FIG. 3 shows the mouse body weight change results from the anti-tumor experiment for the compounds conducted in the ER⁺KAT6^{high} breast cancer cell-derived xenograft tumor model in Test Example 5.

FIG. 4 shows the tumor volume change results from an anti-tumor experiment for compounds conducted in an ER⁺KAT6^{high} breast cancer cell-derived xenograft tumor model in Test Example 6.

FIG. 5 shows the mouse body weight change results from the anti-tumor experiment for the compounds conducted in the ER⁺KAT6^{high} breast cancer cell-derived xenograft tumor model in Test Example 6.

### DETAILED DESCRIPTION

### Definitions

Unless otherwise stated, the terms used in the specification and claims have the following meanings.

The term “alkyl” refers to a saturated aliphatic hydrocarbon group, which is a linear or branched group containing 1 to 20 (e.g., 1, 2, 3, 4, 5, 6, 7, 8, 9, 10, 11, 12, 13, 14, 15, 16, 17, 18, 19, or 20) carbon atoms (i.e., C₁₋₂₀ alkyl), preferably alkyl containing 1 to 12 (e.g., 1, 2, 3, 4, 5, 6, 7, 8, 9, 10, 11, and 12) carbon atoms (i.e., C₁₋₁₂ alkyl), and more preferably alkyl containing 1 to 6 carbon atoms (i.e., C₁₋₆ alkyl). Non-limiting examples include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and various branched isomers thereof, etc. Alkyl may be substituted or unsubstituted, and when it is substituted, the substitution may occur at any available point of attachment, and the substituent is preferably selected from one or more of a D atom, halogen, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “alkylene” refers to a saturated linear or branched aliphatic hydrocarbon group, which is a residue derived from the parent alkane by removal of two hydrogen atoms from the same carbon atom or two different carbon atoms, having 1 to 20 (e.g., 1, 2, 3, 4, 5, 6, 7, 8, 9, 10, 11, 12, 13, 14, 15, 16, 17, 18, 19, or 20) carbon atoms (i.e., C₁₋₂₀ alkylene), preferably 1 to 12 carbon atoms (i.e., C₁₋₁₂ alkylene), and more preferably 1 to 6 carbon atoms (i.e., C₁₋₆ alkylene). Non-limiting examples include: methylene (—CH₂—), 1,1-ethylene (—CH(CH₃)—), 1,2-ethylene (—CH₂CH₂—), 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), etc. Alkylene may be substituted or unsubstituted, and when it is substituted, the substitution may occur at any available point of attachment, and the substituent is preferably selected from one or more of a D atom, halogen, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “heteroalkylene” refers to alkylene in which one or more —CH₂— are replaced by one or more groups selected from N, O, S, S(O), and S(O)₂, wherein the alkyl is as defined above. Heteroalkylene may be substituted or unsubstituted, and when it is substituted, the substitution with a substituent may occur at any available point of attachment, and the substituent is preferably selected from one or more of a D atom, halogen, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “alkenyl” refers to an alkyl compound containing at least one carbon-carbon double bond in the molecule, wherein the alkyl is as defined above; the alkenyl has 2 to 12 (e.g., 2, 3, 4, 5, 6, 7, 8, 9, 10, 11, or 12) carbon atoms (i.e., C₂₋₁₂ alkenyl). The alkenyl is preferably alkenyl having 2 to 6 carbon atoms (i.e., C₂₋₆ alkenyl). Alkenyl may be substituted or unsubstituted, and when it is substituted, the substituent is preferably selected from one or more of alkoxy, halogen, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “alkynyl” refers to an alkyl compound containing at least one carbon-carbon triple bond in the molecule, wherein the alkyl is as defined above; the alkynyl has 2 to 12 (e.g., 2, 3, 4, 5, 6, 7, 8, 9, 10, 11, or 12) carbon atoms (i.e., C₂₋₁₂ alkynyl). The alkynyl is preferably alkynyl having 2 to 6 carbon atoms (i.e., C₂₋₆ alkynyl). Alkynyl may be substituted or unsubstituted, and when it is substituted, the substituent is preferably selected from one or more of alkoxy, halogen, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “cycloalkyl” refers to a saturated or partially unsaturated monocyclic or polycyclic hydrocarbon substituent; the cycloalkyl ring contains 3 to 20 (e.g., 3, 4, 5, 6, 7, 8, 9, 10, 11, 12, 13, 14, 15, 16, 17, 18, 19, or 20) carbon atoms (i.e., 3- to 20-membered cycloalkyl), preferably 3 to 12 (e.g., 3, 4, 5, 6, 7, 8, 9, 10, 11, or 12) carbon atoms (i.e., 3- to 12-membered cycloalkyl), preferably 3 to 8 (e.g., 3, 4, 5, 6, 7, and 8) carbon atoms (i.e., 3- to 8-membered cycloalkyl), further preferably 4 to 7 (e.g., 4, 5, 6, and 7) carbon atoms (i.e., 4- to 7-membered cycloalkyl), and more preferably 3 to 6 (e.g., 3, 4, 5, and 6) carbon atoms (i.e., 3- to 6-membered cycloalkyl). Non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes spirocycloalkyl, fused cycloalkyl, and bridged cycloalkyl.

The term “spirocycloalkyl” refers to a 5- to 20-membered (e.g., 5, 6, 7, 8, 9, 10, 11, 12, 13, 14, 15, 16, 17, 18, 19 or 20 ring atoms, i.e., 5- to 20-membered spirocycloalkyl) polycyclic group in which the monocyclic rings share one carbon atom (referred to as a spiro atom), and it may contain one or more double bonds. It is preferably 6- to 14-membered (i.e., 6- to 14-membered spirocycloalkyl), and is more preferably 7- to 10-membered (e.g., 7-, 8-, 9-, or 10-membered, i.e., 7- to 10-membered spirocycloalkyl). According to the number of spiro atoms shared between the rings, spirocycloalkyl may be monospirocycloalkyl, bispirocycloalkyl, or polyspirocycloalkyl, preferably monospirocycloalkyl and bispirocycloalkyl, and more preferably 3-membered/5-membered, 3-membered/6-membered, 4-membered/4-membered, 4-membered/5-membered, 4-membered/6-membered, 5-membered/5-membered, or 5-membered/6-membered monospirocycloalkyl. Non-limiting examples of spirocycloalkyl include:

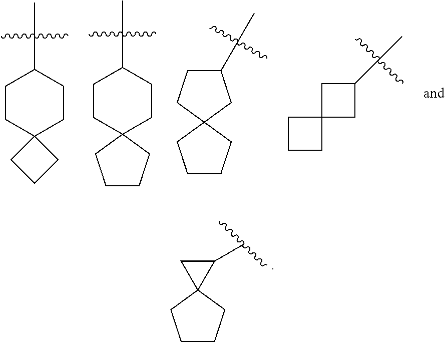

The term “fused cycloalkyl” refers to a 5- to 20-membered (e.g., 5, 6, 7, 8, 9, 10, 11, 12, 13, 14, 15, 16, 17, 18, 19, or 20 ring atoms, i.e., 5- to 20-membered fused cycloalkyl) all-carbon polycyclic group in which each of the rings in the system shares a pair of adjacent carbon atoms with the other rings in the system, wherein one or more of the rings may contain one or more double bonds. It is preferably 6- to 14-membered (i.e., 6- to 14-membered fused cycloalkyl), and is more preferably 7- to 10-membered (e.g., 7-, 8-, 9-, or 10-membered, i.e., 7- to 10-membered fused cycloalkyl). According to the number of constituent rings, it may be bicyclic, tricyclic, tetracyclic, or polycyclic fused cycloalkyl, preferably bicyclic or tricyclic fused cycloalkyl, and more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered, and 6-membered/6-membered bicyclic fused cycloalkyl. Non-limiting examples of fused cycloalkyl include:

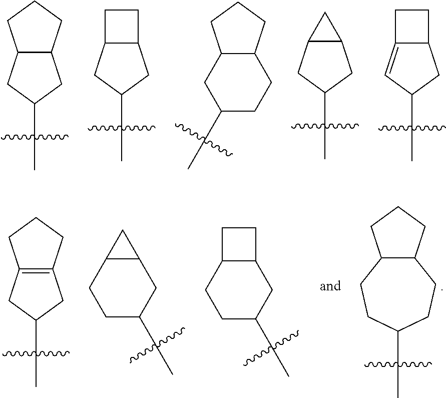

The term “bridged cycloalkyl” refers to a 5- to 20-membered (e.g., 5, 6, 7, 8, 9, 10, 11, 12, 13, 14, 15, 16, 17, 18, 19, or 20 carbon atoms, i.e., 5- to 20-membered bridged cycloalkyl) all-carbon polycyclic group in which any two of the rings share two carbon atoms that are not directly connected, and it may contain one or more double bonds. It is preferably 6- to 14-membered (i.e., 6- to 14-membered bridged cycloalkyl), and is more preferably 7- to 10-membered (e.g., 7-, 8-, 9-, or 10-membered, i.e., 7- to 10-membered bridged cycloalkyl). According to the number of constituent rings, it may be bicyclic, tricyclic, tetracyclic, or polycyclic bridged cycloalkyl, preferably bicyclic, tricyclic, or tetracyclic bridged cycloalkyl, and more preferably bicyclic or tricyclic bridged cycloalkyl. Non-limiting examples of bridged cycloalkyl include:

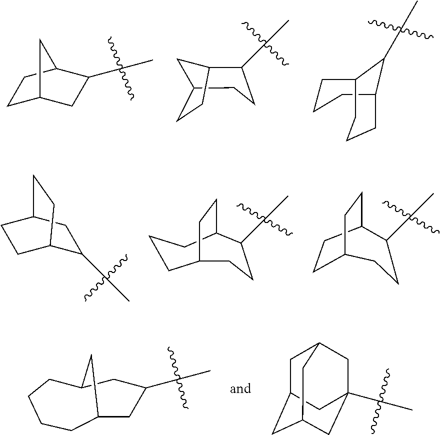

The cycloalkyl ring includes those in which the cycloalkyl described above (including monocyclic cycloalkyl, spirocycloalkyl, fused cycloalkyl, and bridged cycloalkyl) is fused to an aryl, heteroaryl, or heterocycloalkyl ring, wherein the ring attached to the parent structure is cycloalkyl. Non-limiting examples include

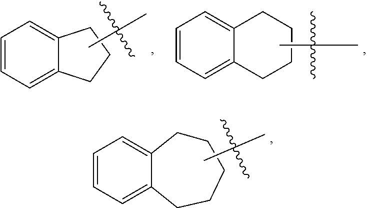

etc.;

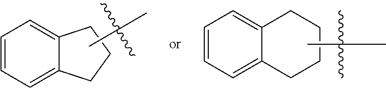

is preferred.

Cycloalkyl may be substituted or unsubstituted, and when it is substituted, the substitution may occur at any available point of attachment, and the substituent is preferably selected from one or more of halogen, alkyl, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “alkoxy” refers to —O-(alkyl), wherein the alkyl is as defined above. Non-limiting examples of alkoxy include: methoxy, ethoxy, propoxy, and butoxy. Alkoxy may be optionally substituted or unsubstituted, and when it is substituted, the substituent is preferably one or more groups independently selected from a D atom, halogen, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “heterocyclyl” refers to a saturated or partially unsaturated monocyclic or polycyclic substituent containing 3 to 20 ring atoms (e.g., 3, 4, 5, 6, 7, 8, 9, 10, 11, 12, 13, 14, 15, 16, 17, 18, 19, or 20 ring atoms, i.e. 3-to 20-membered heterocyclyl), wherein one or more of the ring atoms are heteroatoms selected from nitrogen, oxygen, and sulfur (the sulfur may be optionally substituted with oxo (i.e., forming sulfoxide or sulfone)), but excluding a ring moiety of —O—O—, —O—S—, or —S—S—, and the other ring atoms are carbon. Preferably, it contains 3 to 12 (e.g., 3, 4, 5, 6, 7, 8, 9, 10, 11, and 12) ring atoms (i.e., 3- to 12-membered heterocyclyl), 1-4 (e.g., 1, 2, 3, and 4) of which are heteroatoms; more preferably, it contains 3 to 8 (e.g., 3, 4, 5, 6, 7, and 8) ring atoms (i.e., 3- to 8-membered heterocyclyl), 1-3 (e.g., 1, 2, and 3) of which are heteroatoms; further preferably, it contains 4 to 7 (e.g., 4, 5, 6, and 7) ring atoms (i.e., 4- to 7-membered heterocyclyl), 1-3 (e.g., 1, 2, and 3) of which are heteroatoms; more preferably, it contains 3 to 6 (e.g., 3, 4, 5, and 6) ring atoms (i.e., 3- to 6-membered heterocyclyl), 1-3 (e.g., 1, 2, and 3) of which are heteroatoms; most preferably, it contains 5 or 6 ring atoms (i.e., 5- or 6-membered heterocyclyl), 1-2 (e.g., 1 or 2) of which are heteroatoms. Non-limiting examples of monocyclic heterocyclyl include pyrrolidinyl, tetrahydropyranyl, 1,2,3,6-tetrahydropyridinyl, piperidinyl, piperazinyl, morpholinyl, thiomorpholinyl, homopiperazinyl, etc. Polycyclic heterocyclyl includes spiroheterocyclyl, fused heterocyclyl, and bridged heterocyclyl.

The term “spiroheterocyclyl” refers to a 5- to 20-membered (e.g., 5, 6, 7, 8, 9, 10, 11, 12, 13, 14, 15, 16, 17, 18, 19, or 20 ring atoms, i.e., 5- to 20-membered spiroheterocyclyl) polycyclic heterocyclyl group in which the monocyclic rings share one atom (referred to as a spiro atom), wherein one or more of the ring atoms are heteroatoms selected from nitrogen, oxygen, and sulfur (the sulfur may be optionally substituted with oxo (i.e., forming sulfoxide or sulfone)), and the other ring atoms are carbon. It may contain one or more double bonds. It is preferably 6- to 14-membered (i.e., 6- to 14-membered spiroheterocyclyl), and is more preferably 7- to 10-membered (e.g., 7-, 8-, 9-, or 10-membered, i.e., 7- to 10-membered spiroheterocyclyl). According to the number of spiro atoms shared between the rings, spiroheterocyclyl may be monospiroheterocyclyl, bispiroheterocyclyl, or polyspiroheterocyclyl, preferably monospiroheterocyclyl and bispiroheterocyclyl, and more preferably 3-membered/5-membered, 3-membered/6-membered, 4-membered/4-membered, 4-membered/5-membered, 4-membered/6-membered, 5-membered/5-membered, or 5-membered/6-membered monospiroheterocyclyl. Non-limiting examples of spiroheterocyclyl include:

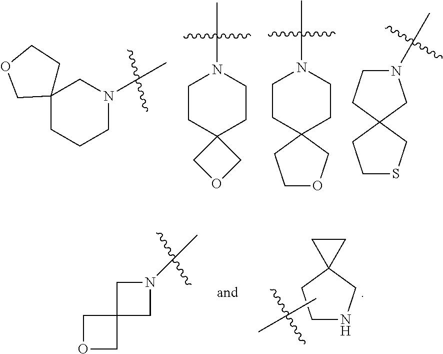

The term “fused heterocyclyl” refers to a 5- to 20-membered (e.g., 5, 6, 7, 8, 9, 10, 11, 12, 13, 14, 15, 16, 17, 18, 19, or 20 ring atoms, i.e., 5- to 20-membered fused heterocyclyl) polycyclic heterocyclyl group in which each of the rings in the system shares a pair of adjacent atoms with the other rings in the system, wherein one or more of the rings may contain one or more double bonds; one or more of the ring atoms are heteroatoms selected from nitrogen, oxygen, and sulfur (the sulfur may be optionally substituted with oxo (i.e., forming sulfoxide or sulfone)), and the other ring atoms are carbon. It is preferably 6- to 14-membered (i.e., 6- to 14-membered fused heterocyclyl), and is more preferably 7- to 10-membered (e.g., 7-, 8-, 9-, or 10-membered, i.e., 7-to 10-membered fused heterocyclyl). According to the number of constituent rings, it may be bicyclic, tricyclic, tetracyclic, or polycyclic fused heterocyclyl, preferably bicyclic or tricyclic fused heterocyclyl, and more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered, and 6-membered/6-membered bicyclic fused heterocyclyl. Non-limiting examples of fused heterocyclyl include.

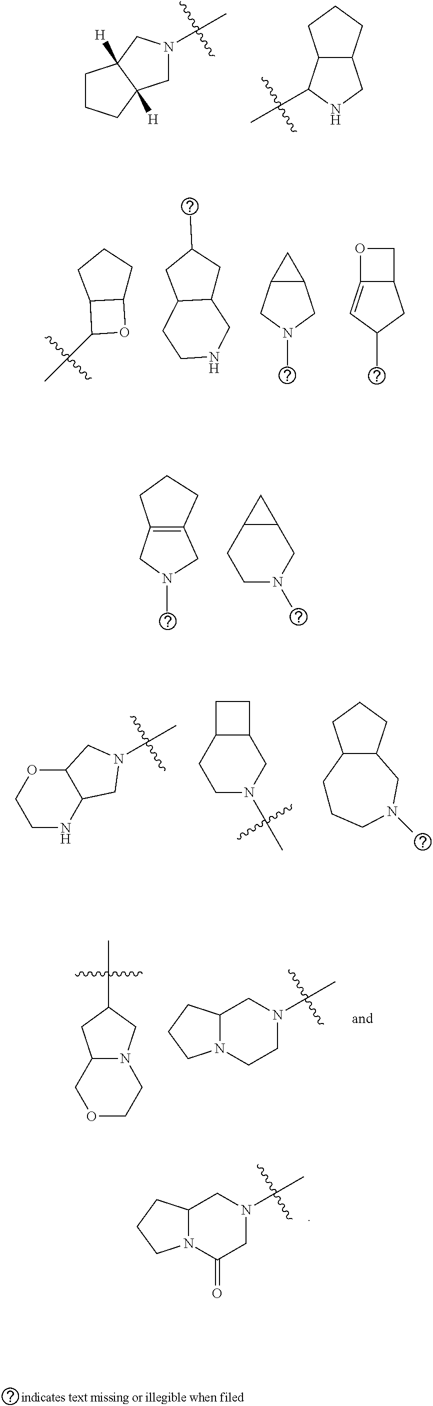

The term “bridged heterocyclyl” refers to a 5- to 14-membered (e.g., 5, 6, 7, 8, 9, 10, 11, 12, 13, or 14 ring atoms, i.e., 5- to 14-membered bridged heterocyclyl) polycyclic heterocyclyl group in which any two of the rings share two atoms that are not directly connected, and it may contain one or more double bonds, wherein one or more of the ring atoms are heteroatoms selected from nitrogen, oxygen, and sulfur (the sulfur may be optionally substituted with oxo (i.e., forming sulfoxide or sulfone)), and the other ring atoms are carbon. It is preferably 6- to 14-membered (i.e., 6- to 14-membered bridged heterocyclyl), and is more preferably 7- to 10-membered (e.g., 7-, 8-, 9-, or 10-membered, i.e., 7- to 10-membered bridged heterocyclyl). According to the number of constituent rings, it may be bicyclic, tricyclic, tetracyclic, or polycyclic bridged heterocyclyl, preferably bicyclic, tricyclic, or tetracyclic bridged heterocyclyl, and more preferably bicyclic or tricyclic bridged heterocyclyl. Non-limiting examples of bridged heterocyclyl include:

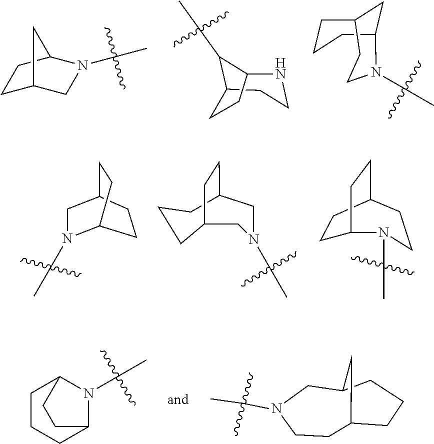

The heterocyclyl ring includes those in which the heterocyclyl described above (including monocyclic heterocyclyl, spiroheterocyclyl, fused heterocyclyl, and bridged heterocyclyl) is fused to an aryl, heteroaryl, or cycloalkyl ring, wherein the ring attached to the parent structure is heterocyclyl; its non-limiting examples include:

and etc.

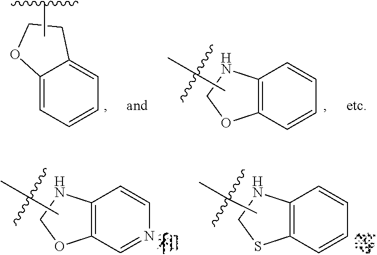

Heterocyclyl may be substituted or unsubstituted, and when it is substituted, the substitution may occur at any available point of attachment, and the substituent is preferably selected from one or more of halogen, alkyl, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “aryl” refers to a 6- to 14-membered (e.g., 6, 7, 8, 9, 10, 11, 12, 13 or 14 ring atoms, i.e., 6- to 14-membered aryl) all-carbon monocyclic or fused polycyclic (fused polycyclic describes the rings sharing a pair of adjacent carbon atoms) group having a conjugated π-electron system, preferably 6- to 10-membered (i.e., 6- to 10-membered aryl), such as phenyl and naphthyl. The aryl ring includes those in which the aryl ring described above is fused to a heteroaryl, heterocyclyl, or cycloalkyl ring, wherein the ring attached to the parent structure is the aryl ring; its non-limiting examples include:

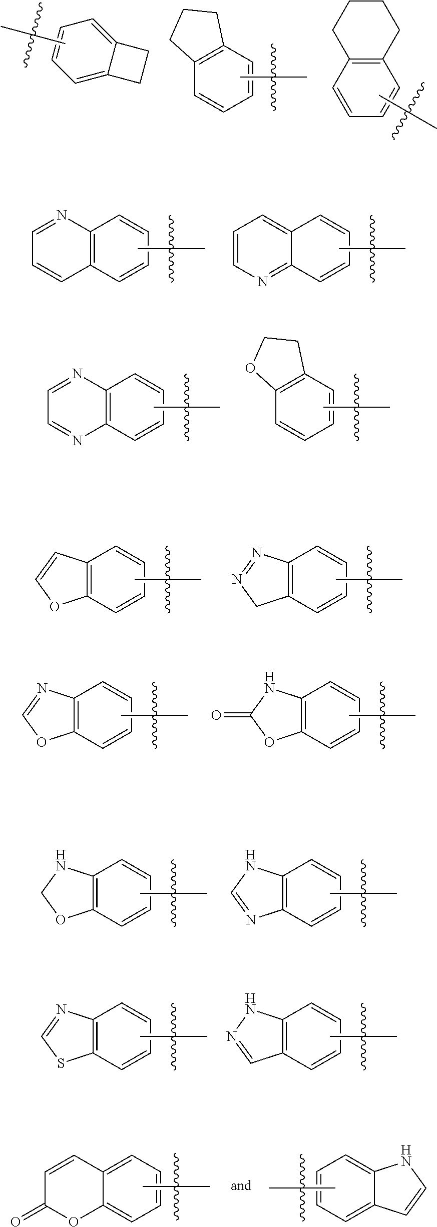

Aryl may be substituted or unsubstituted, and when it is substituted, the substitution may occur at any available point of attachment, and the substituent is preferably selected from one or more of halogen, alkyl, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The term “heteroaryl” refers to a heteroaromatic system containing 1 to 4 (e.g., 1, 2, 3, and 4) heteroatoms and 5 to 14 ring atoms (e.g., 5, 6, 7, 8, 9, 10, 11, 12, 13, or 14 ring atoms, i.e., 5- to 14-membered heteroaryl), wherein the heteroatoms are selected from oxygen, sulfur, and nitrogen. The heteroaryl is preferably 5- to 10-membered (e.g., 5-, 6-, 7-, 8-, 9- or 10-membered, i.e., 5- to 10-membered heteroaryl), and is more preferably 5- or 6-membered (i.e., 5- or 6-membered heteroaryl), such as furanyl, thienyl, pyridinyl, pyrrolyl, N-alkylpyrrolyl, pyrimidinyl, pyrazinyl, pyridazinyl, imidazolyl, pyrazolyl, triazolyl, tetrazolyl, etc. The heteroaryl ring includes those in which the heteroaryl ring described above is fused to an aryl, heterocyclyl, or cycloalkyl ring, wherein the ring attached to the parent structure is the heteroaryl ring; its non-limiting examples include.

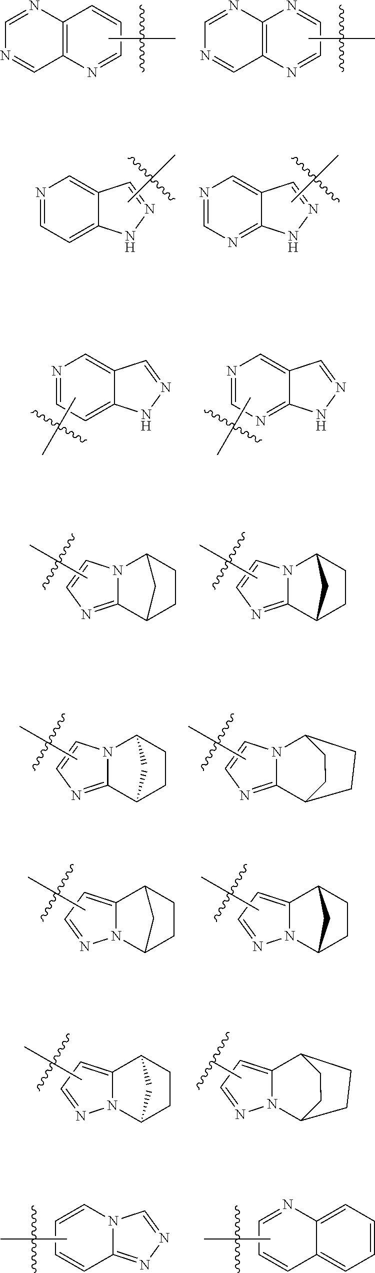

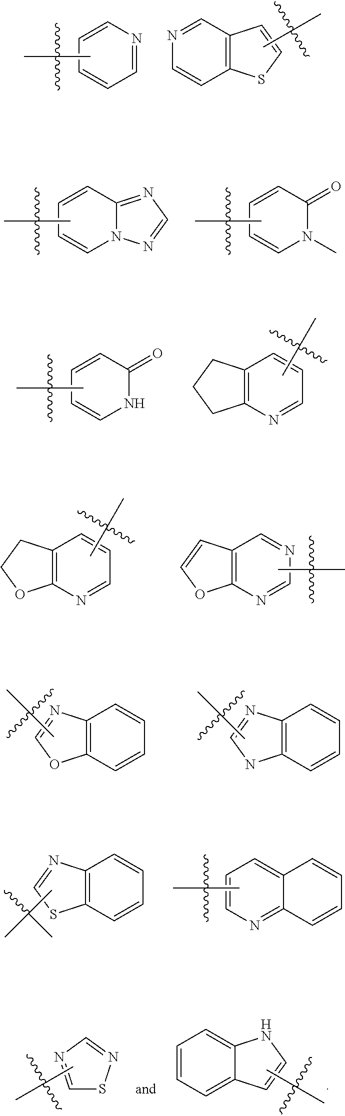

Heteroaryl may be substituted or unsubstituted, and when it is substituted, the substitution may occur at any available point of attachment, and the substituent is preferably selected from one or more of halogen, alkyl, alkoxy, haloalkyl, haloalkoxy, cycloalkyloxy, heterocyclyloxy, hydroxyl, hydroxyalkyl, cyano, amino, nitro, cycloalkyl, heterocyclyl, aryl, and heteroaryl.

The cycloalkyl, heterocyclyl, aryl, and heteroaryl described above include residues derived from the parent ring by removal of one hydrogen atom from a ring atom, or residues derived from the parent ring by removal of two hydrogen atoms from the same ring atom or two different ring atoms, i.e., “divalent cycloalkyl”, “divalent heterocyclyl”

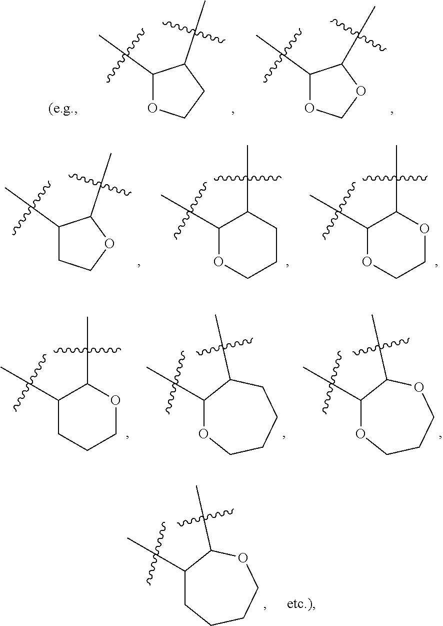

arylene and heteroaryl”.

The term “cycloalkylalkyl” refers to alkyl substituted with one or more cycloalkyl groups, wherein the cycloalkyl and alkyl are as defined above.

The term “heterocyclylalkyl” refers to alkyl substituted with one or more heterocyclyl groups, wherein the heterocyclyl and alkyl are as defined above.

The term “heteroarylalkyl” refers to alkyl substituted with one or more heteroaryl groups, wherein the heteroaryl and alkyl are as defined above.

The term “cycloalkyloxy” refers to cycloalkyl-O—, wherein the cycloalkyl is as defined above.

The term “heterocyclyloxy” refers to heterocyclyl-O—, wherein the heterocyclyl is as defined above.

The term “alkylthio” refers to alkyl-S-, wherein the alkyl is as defined above.

The term “haloalkyl” refers to alkyl substituted with one or more halogens, wherein the alkyl is as defined above.

The term “haloalkoxy” refers to alkoxy substituted with one or more halogens, wherein the alkoxy is as defined above.

The term “alkoxyalkyl” refers to alkyl substituted with one or more alkoxy groups, wherein the alkyl and alkoxy are as defined above.

The term “hydroxyalkyl” refers to alkyl substituted with one or more hydroxyl groups, wherein the alkyl is as defined above.

The term “halogen” refers to fluorine, chlorine, bromine, or iodine.

The term “hydroxyl” refers to —OH.

The term “sulfhydryl” refers to —SH.

The term “amino” refers to —NH₂.

The term “cyano” refers to —CN.

The term “nitro” refers to —NO₂.

The term “oxo” refers to “═O”.

The term “carbonyl” refers to C═O.

The term “aldehyde group” refers to —C(O)H.

The term “carboxyl” refers to —C(O)OH.

The term “carboxylate ester group” refers to —C(O)O(alkyl), —C(O)O(cycloalkyl)(alkyl)C(O)O—, or (cycloalkyl)C(O)O—, wherein the alkyl and cycloalkyl are as defined above.

In another aspect, the compounds of the present disclosure may have particular geometric or stereoisomeric forms. The present disclosure contemplates all such compounds, including cis and trans isomers, (−)- and (+)-enantiomers, (R)- and (S)-enantiomers, diastereomers, (D)-isomers, (L)-isomers, and racemic mixtures and other mixtures thereof, such as enantiomerically or diastereomerically enriched mixtures, all of which fall within the scope of the present disclosure. Additional asymmetric carbon atoms may be present in the substituents such as alkyl. All such isomers and mixtures thereof are included within the scope of the present disclosure. Optically active (R)- and (S)-enantiomers and D- and L-isomers can be prepared by chiral synthesis, chiral reagents, or other conventional techniques. If one enantiomer of a certain compound of the present disclosure is desired, it may be prepared by asymmetric synthesis or derivatization with a chiral auxiliary, wherein the resulting mixture of diastereomers is separated and the auxiliary group is cleaved to provide the pure desired enantiomer. Alternatively, when the molecule contains a basic functional group (e.g., amino) or an acidic functional group (e.g., carboxyl), the compound reacts with an appropriate optically active acid or base to form a diastereomeric salt, which is then subjected to diastereomer resolution through conventional methods well known in the art to acquire the pure enantiomer. Furthermore, separation of enantiomers and diastereomers is generally accomplished by chromatography using a chiral stationary phase, optionally in combination with chemical derivatization (e.g., carbamate formation from amines).

In the chemical structure of the compound of the present disclosure, the bond [symbol] indicates an unspecified configuration; that is, if chiral isomers exist in the chemical structure, the bond [symbol] may be [symbol] or [symbol] or may include both the configurations. In the chemical structure of the compound of the present disclosure, the bond [symbol] does not specify a configuration; that is, the chemical structure may be in a Z configuration or an E configuration, or may include both the configurations.

In addition, the compounds and intermediates of the present disclosure may also exist in different tautomeric forms, and all such forms are included within the scope of the present disclosure. The term “tautomer” or “tautomeric form” refers to structural isomers of different energies that can interconvert via a low-energy barrier. For example, proton tautomers (also known as prototropic tautomers) include interconversion via proton migration, such as keto-enol isomerization and imine-enamine isomerization. An example of a lactam-lactim equilibrium is present between A and B as shown below.

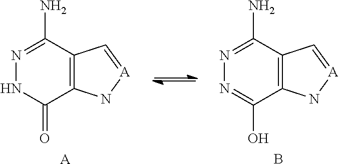

All compounds in the present disclosure can be drawn as form A or form B. All tautomeric forms are within the scope of the present disclosure. The nomenclature of the compounds does not exclude any tautomers.

The present disclosure also includes some isotopically labeled compounds of the present disclosure that are identical to those recited herein but have one or more atoms replaced by an atom having an atomic mass or mass number different from the atomic mass or mass number usually found in nature. Examples of isotopes that can be incorporated into the compounds of the present disclosure include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I, ³⁶Cl, etc.

The compound of the present disclosure may contain an unnatural proportion of atomic isotope at one or more of the atoms that constitute the compound. For example, the compound may be labeled with a radioisotope, such as tritium (³H). Hydrogen may be replaced by deuterium to form a deuterated drug. The bond formed between deuterium and carbon is firmer than that formed between ordinary hydrogen and carbon. Compared to an undeuterated drug, the deuterated drug has the advantages of reduced toxic and side effects, increased stability, enhanced efficacy, prolonged biological half-life, etc. All isotopic variations of the compounds of the present disclosure, whether radioactive or not, are encompassed within the scope of the present disclosure.

Furthermore, substitution with heavier isotopes such as deuterium (i.e., ²H) may provide certain therapeutic advantages (e.g., increased in vivo half-life or reduced dose requirement) resulting from greater metabolic stability and hence may be preferred in certain circumstances in which deuterium substitution may be partial or complete, wherein partial deuterium substitution refers to substitution of at least one hydrogen with at least one deuterium.

Unless otherwise specified, when a position is specifically designated as deuterium (D), that position shall be understood as deuterium with an abundance that is at least 1000 times greater than the natural abundance of deuterium (which is 0.015%) (i.e., at least 10% deuterium incorporation). The deuterium in the compounds of the examples with an abundance greater than the natural abundance of deuterium may be deuterium with an abundance that is at least 1000 times, at least 2000 times, at least 3000 times, at least 4000 times, at least 5000 times, at least 6000 times, or higher times the natural abundance. The present disclosure also includes various deuterated forms of the compound of formula (I). Each available hydrogen atom attached to a carbon atom may be independently replaced by a deuterium atom. Those skilled in the art are able to synthesize the deuterated forms of the compound of formula (I) with reference to the relevant literature. Commercially available deuterated starting materials can be used in preparing the deuterated forms of the compound of formula (I), or they can be synthesized using conventional techniques with deuterated reagents, including but not limited to deuterated borane, tri-deuterated borane in tetrahydrofuran, deuterated lithium aluminum hydride, deuterated iodoethane, deuterated iodomethane, etc.

“Optional” or “optionally” means that the event or circumstance subsequently described may, but does not necessarily, occur. This description includes instances where the event or circumstance occurs or does not occur. For example, “heterocyclyl group optionally substituted with alkyl” means that alkyl may, but does not necessarily, exist. This description includes the instance where the heterocyclyl group is substituted with alkyl and the instance where it is not. “Substituted” means that one or more, preferably 1-5, and more preferably 1-3, hydrogen atoms in the group are independently substituted with a corresponding number of substituents. Those skilled in the art are able to determine (experimentally or theoretically) possible or impossible substitutions without undue effort. For example, it may be unstable when an amino or hydroxyl group having free hydrogen is bound to a carbon atom having an unsaturated (e.g., olefinic) bond.

“Pharmaceutical composition” refers to a mixture containing one or more of the compounds or the physiologically/pharmaceutically acceptable salts or prodrugs thereof described herein, and other chemical components, as well as other components such as physiologically/pharmaceutically acceptable carriers and excipients. The pharmaceutical composition is intended to promote administration to an organism and facilitate the absorption of the active ingredient so that it can exert its biological activity.

“Pharmaceutically acceptable salt” refers to a salt of the compound of the present disclosure, which, when used in a mammal, possesses safety

and effectiveness and has the intended biological activity. The salt may be separately prepared during the final isolation and purification of the compound, or by reacting a suitable group with a suitable base or acid. Bases commonly used to form pharmaceutically acceptable salts include inorganic bases, such as sodium hydroxide and potassium hydroxide, and organic bases, such as ammonia. Acids commonly used to form pharmaceutically acceptable salts include inorganic acids and organic acids.

For drugs or pharmacologically active agents, the term “therapeutically effective amount”, “inhibitory effective amount”, or “prophylactically effective amount” refers to an amount of the drug or agent sufficient to achieve, or partially achieve, the desired effect. The determination of the effective amount varies from person to person. It depends on the age and general condition of a subject, as well as the specific active substance used. The appropriate effective amount in a case may be determined by those skilled in the art in the light of routine tests.

The term “pharmaceutically acceptable” as used herein refers to those compounds, materials, compositions, and/or dosage forms that are, within the scope of sound medical judgment, suitable for use in contact with the tissues of patients without excessive toxicity, irritation, allergic reaction, or other problems or complications, and are commensurate with a reasonable benefit/risk ratio and effective for the intended use.

As used herein, the singular forms “a”, “an”, and “the” include plural references and vice versa, unless the context clearly indicates otherwise.

When the term “about” or “approximately” is applied to parameters such as pH, concentration, temperature, etc., it means that the parameter may vary by +10%, and sometimes more preferably within ±5%. As will be understood by those skilled in the art, when the parameters are not critical, the numbers are generally given for illustrative purposes only and are not intended to be limiting.

It should be noted that in the case where the structure of a compound described in this specification is not consistent with the chemical formula, the structure shall prevail.

### Compounds of the Present Disclosure

In some aspects, the present disclosure provides a compound of formula (I) or a pharmaceutically acceptable salt, an ester, a stereoisomer, a tautomer, a polymorph, a hydrate, a solvate, an N-oxide, an isotopically labeled compound, a metabolite, or a prodrug thereof:

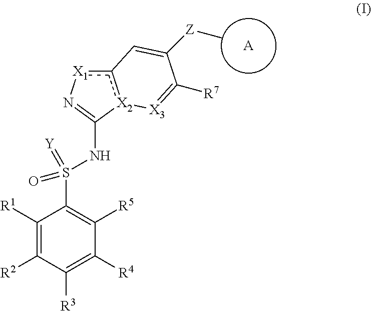

wherein:
the dashed line is an optional chemical bond;
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ halocycloalkyl, C₃₋₆ cycloalkoxy, C₃₋₆ halocycloalkoxy, 6- to 10-membered aryl, 5- to 10-membered heteroaryl, and C₁₋₆ hydroxyalkyl, or R⁴ and R⁵, together with the carbon atoms to which they are attached, form 4- to 10-membered heterocyclyl, 5- to 6-membered heteroaryl, 7- to 12-membered spiroheterocyclyl, or 8- to 15-membered fused heterocyclyl, wherein the 4- to 10-membered heterocyclyl, 5- to 6-membered heteroaryl, 7- to 12-membered spiroheterocyclyl, or 8- to 15-membered fused heterocyclyl is optionally substituted with one or more substituents selected from: hydroxyl, amino, halogen, C₁₋₆ alkyl, C₁₋₆ haloalkyl, C₁₋₆ hydroxyalkyl, C₁₋₆ alkoxy, C₁₋₆ haloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, oxo, ═CH₂, ═CHC₁₋₆ alkyl, ═C(C₁₋₆ alkyl)₂, ═CHC₁₋₆ haloalkyl, ═C(C₁₋₆ haloalkyl)₂, —NHC₁₋₆ alkyl, —N(C₁₋₆ alkyl)₂, cyano, benzyl, C₃₋₆ cycloalkyl, C₃₋₆ heterocyclyl, C₃₋₆ halocycloalkyl, C₃₋₆ haloheterocyclyl, C₃₋₆ cycloalkoxy, C₃₋₆ halocycloalkoxy, 6- to 10-membered aryl, 5- to 10-membered heteroaryl, and C₂₋₆ alkenyl; ring A is selected from 3- to 8-membered heterocyclyl, 6- to 10-membered aryl, and 5- to 10-membered heteroaryl and 6- to 12-membered fused heterocyclyl containing at least one nitrogen atom; ring A is optionally substituted with one or more substituents selected from: halogen, amino, hydroxyl, cyano, C₁₋₆ alkyl, C₁₋₆ haloalkyl, C₁₋₆ alkoxy, 3- to 8-membered cycloalkoxy, C₁₋₆ haloalkoxy, 3- to 8-membered halocycloalkoxy, oxo, and C₂₋₆ alkenyl;
X₁ is O, S, C, or N;
X₂ is N or C;
X₃ is N or CR₆;
R⁶ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy; R⁷ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy; and
Y is O or NH; and
Z is —CH₂—, —CF₂—, —C(O)—, O, —S(O)—, —S(O)₂—, or —NH—.

In some embodiments of the compound of formula (I), X₁ is O. In some embodiments of the compound of formula (I), X₁ is S. In some embodiments of the compound of formula (I), X₁ is C.

In some embodiments of the compound of formula (I), X₁ is N.

In some embodiments of the compound of formula (I), X₂ is N. In some embodiments of the compound of formula (I), X₂ is C.

In some embodiments of the compound of formula (I), X₃ is N.

In some embodiments of the compound of formula (I), X₃ is CR₆, wherein R⁶ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy.

In some embodiments of the compound of formula (I), Z is —CH₂—. Alternatively, in some embodiments of the compound of formula (I), Z is —CF₂—. Alternatively, in some embodiments of the compound of formula (I), Z is —C(O)-. Alternatively, in some embodiments of the compound of formula (I), Z is O. Alternatively, in some embodiments of the compound of formula (I), Z is —S(O)—. Alternatively, in some embodiments of the compound of formula (I), Z is —S(O)₂—.

Alternatively, in some embodiments of the compound of formula (I), Z is —NH—.

In some aspects, the present disclosure provides a compound of formula (II-1) or a pharmaceutically acceptable salt, an ester, a stereoisomer, a tautomer, a polymorph, a hydrate, a solvate, an N-oxide, an isotopically labeled compound, a metabolite, or a prodrug thereof,

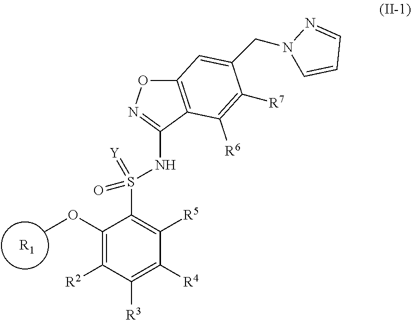

wherein:
R² and R³ are identical or different and are each independently selected from a hydrogen atom, halogen, C₁₋₄ alkyl, C₁₋₄ haloalkyl, C₁₋₄ alkoxy, and C₁, haloalkoxy;
R⁴ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ halocycloalkyl, C₃₋₆ cycloalkoxy, C₃₋₆ halocycloalkoxy, and C₁₋₆ hydroxyalkyl;
R⁵ is selected from a hydrogen atom, C₁₋₄ alkoxy, C₁₋₄ haloalkoxy, C₄₋₆ cycloalkoxy, and C₄₋₆ halocycloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
ring M is selected from 4- to 7-membered cycloalkyl and 4- to 7-membered halocycloalkyl; Y is O or NH.

In some embodiments of the compound of formula (II-1), R² and R³ are identical or different and are each independently selected from a hydrogen atom, halogen, C₁₋₄ alkyl, and C₁₋₄ alkoxy. Preferably, in some embodiments of the compound of formula (II-1), R² and R³ are identical or different and are each independently selected from a hydrogen atom and halogen. More preferably, in some embodiments of the compound of formula (II-1), both R² and R³ are hydrogen atoms.

In some embodiments of the compound of formula (II-1), R⁴ is selected from a hydrogen atom, C₁₋₆ alkyl, C₃₋₆ heterocyclyl, and C₁₋₆ hydroxyalkyl. Preferably, in some embodiments of the compound of formula (II-1), R⁴ is selected from a hydrogen atom, ethyl, isopropyl, and tert-butyl. More specifically, in some embodiments of the compound of formula (II-1), R⁴ is a hydrogen atom. In some other embodiments of the compound of formula (II-1), R⁴ is ethyl. In some other embodiments of the compound of formula (II-1), R⁴ is isopropyl. In some other embodiments of the compound of formula (II-1), R⁴ is tert-butyl.

In some embodiments of the compound of formula (II-1), Rⁿ is selected from a hydrogen atom, methoxy, difluoromethoxy, trifluoromethoxy, —OCH₂CHF₂, cyclobutyloxy, and

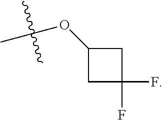 [SMILES: *OC1CC(F)(F)C1]

Preferably, in some embodiments of the compound of formula (II-1), R⁵ is a hydrogen atom or methoxy. More specifically, in some embodiments of the compound of formula (II-1), R⁵ is a hydrogen atom. In some other embodiments of the compound of formula (II-1), R⁵ is methoxy.

In some embodiments of the compound of formula (II-1), R⁶ and R⁷ are identical or different and are each independently selected from a hydrogen atom, a chlorine atom, a fluorine atom, methoxy, ethoxy, isopropoxy, difluoromethoxy, and cyclopropyl.

In some embodiments of the compound of formula (II-1), ring M is selected from cyclobutyl and

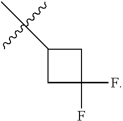 [SMILES: *C1CC(F)(F)C1]

More specifically, in some embodiments of the compound of formula (II-1), ring M is cyclobutyl. In some other embodiments of the compound of formula (II-1), ring M is

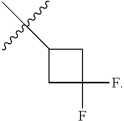 [SMILES: *C1CC(F)(F)C1]

In some embodiments of the compound of formula (II-1), Y is O. In some other embodiments of the compound of formula (II-1), Y is NH.

In some aspects, the present disclosure provides a compound of formula (II-2) or a pharmaceutically acceptable salt, an ester, a stereoisomer, a tautomer, a polymorph, a hydrate, a solvate, an N-oxide, an isotopically labeled compound, a metabolite, or a prodrug thereof,

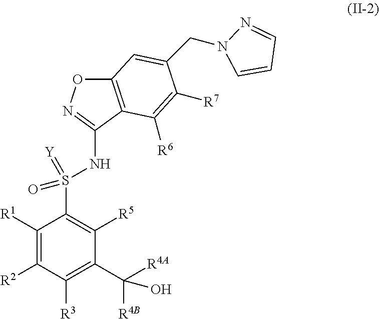

wherein:
R¹ is selected from C₁₋₄ alkoxy, C₁₋₄ haloalkoxy, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R^{4A} and R^{4B} are identical or different and are each independently selected from C₁₋₆ alkyl and C₁₋₆ haloalkyl;
R⁵ is selected from a hydrogen atom, C₁₋₄ alkoxy, C₁₋₄ haloalkoxy, C₄₋₆ cycloalkoxy, and C₄₋₆ halocycloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
ring M is selected from 4- to 7-membered cycloalkyl and 4- to 7-membered halocycloalkyl; Y is O or NH.

In some embodiments of the compound of formula (II-2), R¹ is selected from methoxy, difluoromethoxy, trifluoromethoxy, —OCH₂CHF₂, cyclobutyloxy, and

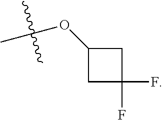 [SMILES: *OC1CC(F)(F)C1]

In some embodiments of the compound of formula (II-2), R² and R³ are identical or different and are each independently selected from a hydrogen atom, halogen, C₁₋₄ alkyl, and C₁₋₄ alkoxy.

Preferably, in some embodiments of the compound of formula (II-2), R² and R³ are identical or different and are each independently selected from a hydrogen atom and halogen. More preferably, in some embodiments of the compound of formula (II-2), both R² and R³ are hydrogen atoms.

In some embodiments of the compound of formula (II-2), both R^{4A} and R^{4B} are methyl.

In some embodiments of the compound of formula (II-2), R⁵ is selected from a hydrogen atom, methoxy, difluoromethoxy, trifluoromethoxy, —OCH₂CHF₂, cyclobutyloxy, and

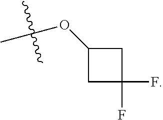 [SMILES: *OC1CC(F)(F)C1]

Preferably, in some embodiments of the compound of formula (II-2), R⁵ is selected from a hydrogen atom and methoxy. More specifically, in some embodiments of the compound of formula (II-2), R⁵ is a hydrogen atom. In some other embodiments of the compound of formula (II-2), R⁵ is methoxy.

In some embodiments of the compound of formula (II-2), R⁶ and R⁷ are identical or different and are each independently selected from a hydrogen atom, a chlorine atom, a fluorine atom, methoxy, ethoxy, isopropoxy, difluoromethoxy, and cyclopropyl.

In some embodiments of the compound of formula (II-2), ring M is cyclobutyl. In some other embodiments of the compound of formula (II-2), ring M is

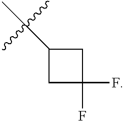 [SMILES: *C1CC(F)(F)C1]

In some embodiments of the compound of formula (II-2), Y is O. In some other embodiments of the compound of formula (II-2), Y is NH.

In some aspects, the present disclosure provides a compound of formula (II-3) or a pharmaceutically acceptable salt, an ester, a stereoisomer, a tautomer, a polymorph, a hydrate, a solvate, an N-oxide, an isotopically labeled compound, a metabolite, or a prodrug thereof,

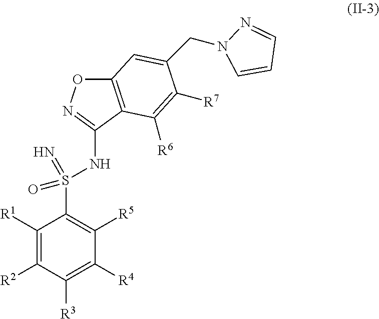

wherein:
R¹ is selected from C₁₋₄ alkoxy, C₁₋₄ haloalkoxy, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁴ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ halocycloalkyl, C₃₋₆ cycloalkoxy, C₃₋₆ halocycloalkoxy, and C₁₋₆ hydroxyalkyl;
R⁵ is selected from a hydrogen atom, C₁₋₄ alkoxy, C₁₋₄ haloalkoxy, C₄₋₆ cycloalkoxy, and C₄₋₆ halocycloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy.

In some embodiments of the compound of formula (II-3), R¹ is selected from methoxy, difluoromethoxy, trifluoromethoxy, —OCH₂CHF₂, cyclobutyloxy, and

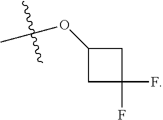 [SMILES: *OC1CC(F)(F)C1]

In some embodiments of the compound of formula (II-3), R² and R³ are identical or different and are each independently selected from a hydrogen atom, halogen, C₁₋₄ alkyl, and C₁₋₄ alkoxy. Preferably, in some embodiments of the compound of formula (II-3), R² and R³ are identical or different and are each independently selected from a hydrogen atom and halogen. More preferably, in some embodiments of the compound of formula (II-3), both R² and R³ are hydrogen atoms.

In some embodiments of the compound of formula (II-3), R⁴ is selected from a hydrogen atom, C₁₋₆ alkyl, C₃₋₆ heterocyclyl, and C₁₋₆ hydroxyalkyl. Preferably, in some embodiments of the compound of formula (II-3), R⁴ is selected from a hydrogen atom, ethyl, isopropyl, and tert-butyl.

In some embodiments of the compound of formula (II-3), R⁵ is selected from a hydrogen atom, methoxy, difluoromethoxy, trifluoromethoxy, —OCH₂CHF₂, cyclobutyloxy, and

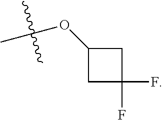 [SMILES: *OC1CC(F)(F)C1]

Preferably, in some embodiments of the compound of formula (II-3), R⁵ is a hydrogen atom or methoxy.

In some embodiments of the compound of formula (II-3), R⁶ and R⁷ are identical or different and are each independently selected from a hydrogen atom, a chlorine atom, a fluorine atom, methoxy, ethoxy, isopropoxy, difluoromethoxy, and cyclopropyl.

In some aspects, the present disclosure provides a compound of formula (III) or a pharmaceutically acceptable salt, an ester, a stereoisomer, a tautomer, a polymorph, a hydrate, a solvate, an N-oxide, an isotopically labeled compound, a metabolite, or a prodrug thereof,

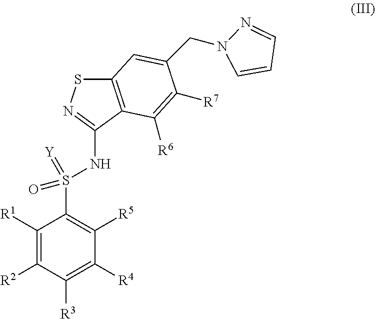

wherein:
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ cycloalkoxy, and C₁₋₆ hydroxyalkyl;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
Y is O or NH.

In some embodiments of the compound of formula (III), R¹ is selected from C₁₋₆ alkoxy, C₁₋₆ haloalkoxy, C₃₋₆ cycloalkoxy, and C₃₋₆ halocycloalkoxy.

In some embodiments of the compound of formula (III), R² and R³ are identical or different and are each independently selected from a hydrogen atom, halogen, and C₁₋₆ alkyl.

Preferably, in some embodiments of the compound of formula (III), R¹ is methoxy, and both R² and R³ are hydrogen atoms.

In some embodiments of the compound of formula (III), R⁴ is selected from a hydrogen atom, C₁₋₆ alkyl, C₃₋₆ heterocyclyl, and C₁₋₆ hydroxyalkyl.

Preferably, in some embodiments of the compound of formula (III), R⁴ is selected from a hydrogen atom, ethyl, isopropyl, tert-butyl, and

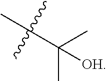 [SMILES: *C(C)(C)O]

More preferably, in some embodiments of the compound of formula (III), R⁴ is a hydrogen atom. In some embodiments of the compound of formula (III), R⁵ is selected from a hydrogen atom, C₁₋₄ alkoxy, C₁₋₄ haloalkoxy, C₃₋₆ cycloalkoxy, and C₃₋₆ halocycloalkoxy. Preferably, in some embodiments of the compound of formula (III), R⁵ is selected from a hydrogen atom, methoxy, difluoromethoxy, trifluoromethoxy, —OCH₂CHF₂, cyclobutyloxy, and

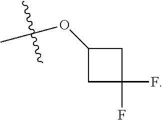 [SMILES: *OC1CC(F)(F)C1]

More specifically, in some embodiments of the compound of formula (III), R⁵ is a hydrogen atom. In some other embodiments of the compound of formula (III), R⁵ is methoxy.

In some embodiments of the compound of formula (III), R⁶ and R⁷ are identical or different and are each independently selected from a hydrogen atom, a chlorine atom, a fluorine atom, methoxy, ethoxy, isopropoxy, difluoromethoxy, and cyclopropyl.

In some embodiments of the compound of formula (III), Y is O. In some other embodiments of the compound of formula (III), Y is NH.

In some aspects, the present disclosure provides a compound of formula (IV) or a pharmaceutically acceptable salt, an ester, a stereoisomer, a tautomer, a polymorph, a hydrate, a solvate, an N-oxide, an isotopically labeled compound, a metabolite, or a prodrug thereof,

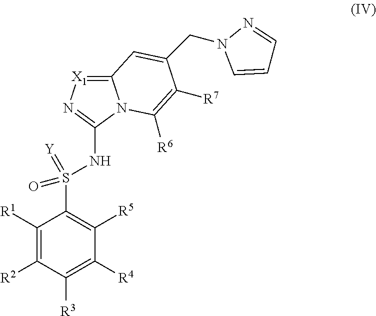

wherein:
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ cycloalkoxy, and C₁₋₆ hydroxyalkyl;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
X₁ is C or N;
Y is O or NH.

In some embodiments of the compound of formula (IV), R¹ is selected from C₁₋₆ alkoxy, C₁₋₆ haloalkoxy, C₃₋₆ cycloalkoxy, and C₃₋₆ halocycloalkoxy. Preferably, in some embodiments of the compound of formula (IV), R¹ is methoxy.

In some embodiments of the compound of formula (IV), R² and R³ are identical or different and are each independently selected from a hydrogen atom, halogen, and C₁₋₆ alkyl. Preferably, in some embodiments of the compound of formula (IV), both R² and R³ are hydrogen atoms.

In some embodiments of the compound of formula (IV), R⁴ is selected from a hydrogen atom, C₁₋₆ alkyl, C₃₋₆ heterocyclyl, and C₁₋₆ hydroxyalkyl. Preferably, in some embodiments of the compound of formula (IV), R⁴ is selected from a hydrogen atom, ethyl, isopropyl, tert-butyl, and

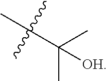 [SMILES: *C(C)(C)O]

More preferably, in some embodiments of the compound of formula (IV), R⁴ is a hydrogen atom.

In some embodiments of the compound of formula (IV), R⁵ is selected from a hydrogen atom, C₁₋₄ alkoxy, C₁₋₄ haloalkoxy, C₃₋₆ cycloalkoxy, and C₃₋₆ halocycloalkoxy. Preferably, in some embodiments of the compound of formula (IV), R⁵ is selected from a hydrogen atom, methoxy, difluoromethoxy, trifluoromethoxy, —OCH₂CHF₂, cyclobutyloxy, and

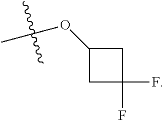 [SMILES: *OC1CC(F)(F)C1]

More specifically, in some embodiments of the compound of formula (IV), R⁵ is a hydrogen atom. In some other embodiments of the compound of formula (IV), R⁵ is methoxy.

In some embodiments of the compound of formula (IV), R⁶ and R⁷ are identical or different and are each independently selected from a hydrogen atom, a chlorine atom, a fluorine atom, methoxy, ethoxy, isopropoxy, difluoromethoxy, and cyclopropyl.

In some embodiments of the compound of formula (IV), X₁ is C. In some other embodiments of the compound of formula (IV), X₁ is N.

In some embodiments of the compound of formula (IV), Y is O. In some other embodiments of the compound of formula (IV), Y is NH.

In some aspects, the present disclosure provides a compound of formula (V) or a pharmaceutically acceptable salt, an ester, a stereoisomer, a tautomer, a polymorph, a hydrate, a solvate, an N-oxide, an isotopically labeled compound, a metabolite, or a prodrug thereof,

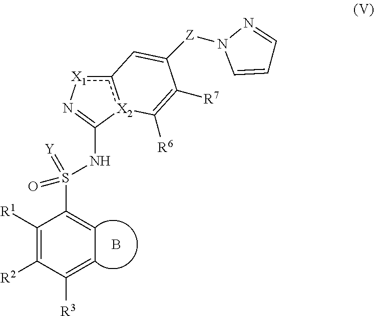

wherein:
the dashed line is an optional chemical bond;
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
ring B is selected from 4- to 10-membered heterocyclyl, 5- to 6-membered heteroaryl, and 7- to 12-membered spiroheterocyclyl; ring B is optionally substituted with one or more substituents selected from: hydroxyl, amino, halogen, C₁₋₆ alkyl, C₁₋₆ haloalkyl, C₁₋₆ alkoxy, C₁₋₆ haloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, oxo, ═CH₂, ═CHC₁₋₆ alkyl, ═C(C₁₋₆ alkyl)₂, ═CHC₁₋₆ haloalkyl, ═C(C₁₋₆ haloalkyl)₂, —NHC_{1.6} alkyl, —N(C₁₋₆ alkyl)₂, cyano, benzyl, C₃₋₆ cycloalkyl, C₃₋₆ heterocyclyl, C₃₋₆ halocycloalkyl, C₃₋₆ cycloalkoxy, C₃₋₆ halocycloalkoxy, 6- to 10-membered aryl, 5- to 10-membered heteroaryl, and C₂₋₆ alkenyl.
R⁶ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy;
R⁷ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy;
X₁ is O, S, C, or N;
X₂ is N or C;
Y is O or NH;
Z is —CH₂—, —CF₂—, —C(O)—, O, —S(O)—, —S(O)₂—, or —NH—.

In some embodiments of the compound of formula (V), X₁ is O. In some embodiments of the compound of formula (V), X₁ is S. In some embodiments of the compound of formula (V), X₁ is C. In some embodiments of the compound of formula (V), X₁ is N.

In some embodiments of the compound of formula (V), X₂ is N. In some other embodiments of the compound of formula (V), X₂ is C.

In some embodiments of the compound of formula (V), X₁ is O or S; X₂ is C. Preferably, in some embodiments of the compound of formula (V), X₁ is O; X₂ is C.

In some embodiments of the compound of formula (V), Y is 0. In some other embodiments of the compound of formula (V), Y is NH.

In some embodiments of the compound of formula (V), Z is —CH₂—. Alternatively, in some embodiments of the compound of formula (V), Z is —CF₂—. Alternatively, in some embodiments of the compound of formula (V), Z is —C(O)—. Alternatively, in some embodiments of the compound of formula (V), Z is O. Alternatively, in some embodiments of the compound of formula (V), Z is —S(O)—. Alternatively, in some embodiments of the compound of formula (V), Z is —S(O)₂—.

Alternatively, in some embodiments of the compound of formula (V), Z is —NH—.

In some embodiments of the compound of formula (V), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (V), R¹ is selected from a hydrogen atom, methoxy, difluoromethoxy, trifluoromethoxy, —OCH₂CHF₂, cyclobutyloxy, and

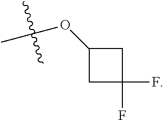 [SMILES: *OC1CC(F)(F)C1]

More specifically, in some embodiments of the compound of formula (V), R¹ is cyclobutyloxy. In some other embodiments of the compound of formula (V), R¹ is methoxy.

In some embodiments of the compound of formula (V), R² and R³ are each independently selected from a hydrogen atom, halogen, C₁₋₄ alkyl, and C₁₋₄ alkoxy. Preferably, in some embodiments of the compound of formula (V), both R² and R³ are hydrogen atoms.

In some embodiments of the compound of formula (V), R⁶ and R⁷ are identical or different and are each independently selected from a hydrogen atom, a chlorine atom, a fluorine atom, methoxy, ethoxy, isopropoxy, difluoromethoxy, and cyclopropyl. Preferably, in some embodiments of the compound of formula (V), R⁶ and R⁷ are identical or different and are each independently selected from a hydrogen atom, a chlorine atom, a fluorine atom, and methoxy.

More preferably, in some embodiments of the compound of formula (V), R⁶ is methoxy. In some embodiments of the compound of formula (V), R⁷ is a hydrogen atom. In some other embodiments of the compound of formula (V), R⁷ is a fluorine atom. In some embodiments of the compound of formula (V), ring B is

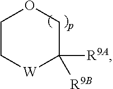

wherein the dashed line represents a chemical bond shared by ring B and the benzene ring; W is selected from —O—, —NR^{9C}—, —CR^{9D}R^{9E}—,

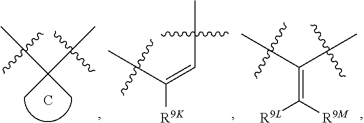

and —C(O)—, and W is connected to a carbon atom ortho to the carbon atom where R³ is located on the benzene ring; p is 0, 1, or 2; R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy; R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, benzyl, 6-to 10-membered aryl, 5- to 10-membered heteroaryl, and C₂₋₆ alkenyl; R^{9D} and R^{9E} are identical or different and are each independently selected from a hydrogen atom, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, —NHC₁₋₆ alkyl, —N(C₁₋₆ alkyl)₂, C₃₋₆ cycloalkyl, C₃₋₆ halocycloalkyl, C₃₋₆ cycloalkoxy, C₃₋₆ halocycloalkoxy, and hydroxyl; ring C is selected from 3- to 8-membered cycloalkyl, 3- to 8-membered heterocyclyl, 6- to 12-membered spirocyclyl, 6- to 12-membered spiroheterocyclyl, and 5- to 12-membered bridged heterocyclyl; ring C is optionally substituted with one or more substituents selected from halogen, hydroxyl, amino, C₁₋₆ alkyl, and C₁₋₆ haloalkyl; R^{9K} is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, 6- to 10-membered aryl, 5- to 10-membered heteroaryl, and hydroxyl, wherein the C₃₋₆ cycloalkyl and C₃₋₆ heterocyclyl are optionally substituted with one or more substituents selected from: halogen, hydroxyl, methyl, and methoxy; R^{9M} and R^{9L} are identical or different and are each independently selected from a hydrogen atom, C₁₋₆ alkyl, and C₁₋₆ haloalkyl. Preferably, W is selected from —O—, —NR^{9C}—,

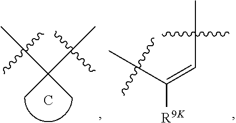

and —C(O)—. Relatively preferably, W is selected from —O—, —NR^{9C}—,

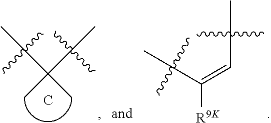

More preferably, W is selected from —O— and —NR^{9C}—. In some embodiments of the compound of formula (V), W is —O—. In some embodiments of the compound of formula (V), W is —NR^{9C},

Preferably, R^{9A} and R^{9B} are identical or different and are each independently selected from a hydrogen atom, a fluorine atom, and methyl. More preferably, both R^{9A} and R^{9B} are hydrogen atoms.

Preferably, R^{9C} is selected from a hydrogen atom, C₁₋₆ alkyl, C₁₋₆ haloalkoxy, —C(O)C₁₋₆ alkyl, —C(O)OC_{1.6} alkyl, —S(O)₂C₁₋₆ alkyl, heteroaryl, and

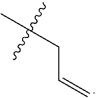 [SMILES: *CC=C]

More preferably, R^{9C} is selected from a hydrogen atom, methyl, deuterated methyl, ethyl, trifluoroethyl, benzyl, —C(O)OCH₃, —C(O)CH₃, and —S(O)₂CH₃.

Preferably, R^{9D} and R⁹E are identical or different and are each independently selected from a hydrogen atom, a fluorine atom, methyl, and hydroxyl. More preferably, R^{9D} and R^{9E} are identical or different and are each independently selected from a hydrogen atom, methyl, and hydroxyl.

Preferably, ring C is selected from

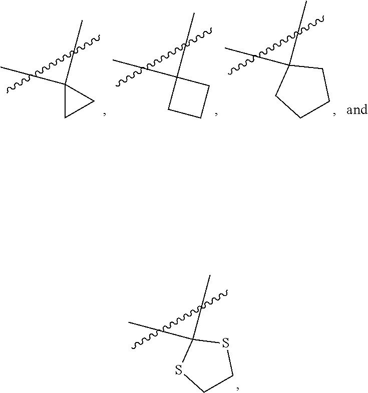

and ring C is optionally substituted with one or more substituents selected from halogen, hydroxyl, amino, C₁₋₆ alkyl, and C₁₋₆ haloalkyl. More preferably, ring C is selected from

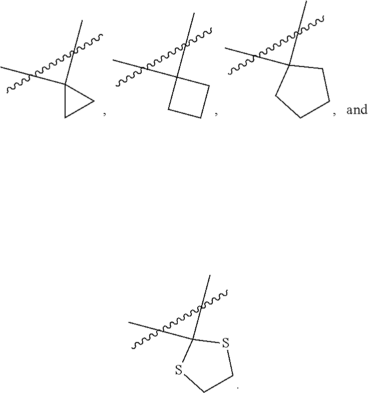

Preferably, in some embodiments of the compound of formula (V), p is 2. In some embodiments of the compound of formula (V), p is 0 or 1. In some embodiments of the compound of formula (V), p is 1. In some other embodiments of the compound of formula (V), p is 0.

In some aspects, the present disclosure provides a compound of formula (VI-1) or a pharmaceutically acceptable salt, an ester, a stereoisomer, a tautomer, a polymorph, a hydrate, a solvate, an N-oxide, an isotopically labeled compound, a metabolite, or a prodrug thereof,

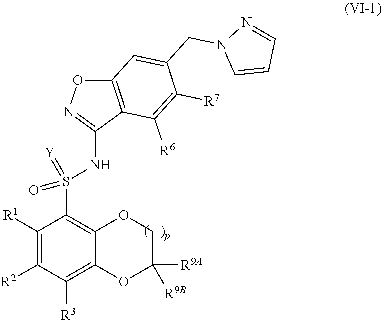

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
Y is O or NH;
p is 0, 1, or 2.

In some embodiments of the compound of formula (VI-1), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VI-1), R¹ is selected from a hydrogen atom, methoxy, difluoromethoxy, trifluoromethoxy, —OCH₂CHF₂, cyclobutyloxy, and

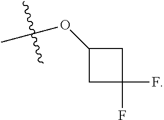 [SMILES: *OC1CC(F)(F)C1]

More specifically, in some embodiments of the compound of formula (VI-1), R¹ is cyclobutyloxy. In some other embodiments of the compound of formula (VI-1), R¹ is methoxy.

In some embodiments of the compound of formula (VI-1), R² and R³ are each independently selected from a hydrogen atom, halogen, C₁₋₄ alkyl, and C₁₋₄ alkoxy. Preferably, in some embodiments of the compound of formula (VI-1), both R² and R³ are hydrogen atoms.

In some embodiments of the compound of formula (VI-1), R⁶ and R⁷ are identical or different and are each independently selected from a hydrogen atom, a chlorine atom, a fluorine atom, methoxy, ethoxy, isopropoxy, difluoromethoxy, and cyclopropyl. Preferably, in some embodiments of the compound of formula (VI-1), R⁶ and R⁷ are identical or different and are each independently selected from a hydrogen atom, a chlorine atom, a fluorine atom, and methoxy. More preferably, in some embodiments of the compound of formula (VI-1), R⁶ is methoxy. In some embodiments of the compound of formula (VI-1), R⁷ is a hydrogen atom. In some other embodiments of the compound of formula (VI-1), R⁷ is a fluorine atom.

In some embodiments of the compound of formula (VI-1), R^{9A} and R^{9B} are identical or different and are each independently selected from a hydrogen atom, a fluorine atom, and methyl.

Preferably, in some embodiments of the compound of formula (VI-1), both R^{9A} and R^{9B} are hydrogen atoms.

In some embodiments of the compound of formula (VI-1), Y is O. In some other embodiments of the compound of formula (VI-1), Y is NH.

In some embodiments of the compound of formula (VI-1), p is 2. In some embodiments of the compound of formula (VI-1), p is 0 or 1. In some embodiments of the compound of formula (VI-1), p is 1. In some other embodiments of the compound of formula (VI-1), p is 0.

In some aspects, the present disclosure provides a compound of formula (VI-2) or a pharmaceutically acceptable salt, an ester, a stereoisomer, a tautomer, a polymorph, a hydrate, a solvate, an N-oxide, an isotopically labeled compound, a metabolite, or a prodrug thereof,

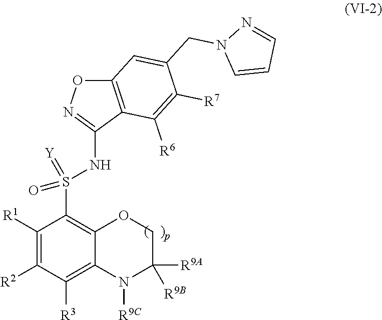

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC_{1.6} alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, benzyl, 6- to 10-membered aryl, 5- to 10-membered heteroaryl, and C₂₋₆ alkenyl;
Y is O or NH;
p is 0, 1, or 2.

In some embodiments of the compound of formula (VI-2), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VI-2), R¹ is selected from a hydrogen atom, methoxy, difluoromethoxy, trifluoromethoxy, —OCH₂CHF₂, cyclobutyloxy, and

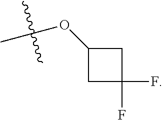 [SMILES: *OC1CC(F)(F)C1]

More specifically, in some embodiments of the compound of formula (VI-2), R¹ is cyclobutyloxy. In some other embodiments of the compound of formula (VI-2), R¹ is methoxy.

In some embodiments of the compound of formula (VI-2), R² and R³ are each independently selected from a hydrogen atom, halogen, C₁₋₄ alkyl, and C₁₋₄ alkoxy. Preferably, in some embodiments of the compound of formula (VI-2), both R² and R³ are hydrogen atoms.

In some embodiments of the compound of formula (VI-2), R⁶ and R⁷ are identical or different and are each independently selected from a hydrogen atom, a chlorine atom, a fluorine atom, methoxy, ethoxy, isopropoxy, difluoromethoxy, and cyclopropyl. Preferably, in some embodiments of the compound of formula (VI-2), R⁶ and R⁷ are identical or different and are each independently selected from a hydrogen atom, a chlorine atom, a fluorine atom, and methoxy. More preferably, in some embodiments of the compound of formula (VI-2), R⁶ is methoxy. In some embodiments of the compound of formula (VI-2), R⁷ is a hydrogen atom. In some other embodiments of the compound of formula (VI-2), R⁷ is a fluorine atom.

In some embodiments of the compound of formula (VI-2), R^{9A} and R^{9B} are identical or different and are each independently selected from a hydrogen atom, a fluorine atom, and methyl.

Preferably, in some embodiments of the compound of formula (VI-2), both R^{9A} and R^{9B} are hydrogen atoms.

In some embodiments of the compound of formula (VI-2), R^{9C} is selected from a hydrogen atom, C₁₋₆ alkyl, C₁₋₆ haloalkoxy, —C(O)C₁₋₆ alkyl, —C(O)OC_{1.6} alkyl, —S(O)₂C₁₋₆ alkyl, heteroaryl, and

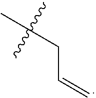 [SMILES: *CC=C]

Preferably, in some embodiments of the compound of formula (VI-2), R^{9C} is selected from a hydrogen atom, methyl, deuterated methyl, ethyl, trifluoroethyl, benzyl, —C(O)OCH₃, —C(O)CH₃, and —S(O)₂CH₃.

In some embodiments of the compound of formula (VI-2), R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, 6- to 10-membered aryl, 5- to 10-membered heteroaryl, and C₂₋₆ alkenyl; in some embodiments of the compound of formula (VI-2), R^{9C} is selected from C₁₋₆ alkyl, heteroaryl, and

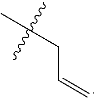 [SMILES: *CC=C]

Preferably, in some embodiments of the compound of formula (VI-2), R^{9C} is methyl.

In some embodiments of the compound of formula (VI-2), R^{9C} is selected from —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, and benzyl; in some embodiments of the compound of formula (VI-2), R^{9C} is selected from a hydrogen atom, C₁₋₆ haloalkoxy, —C(O)C₁₋₆ alkyl, —C(O)OC₁₋₆ alkyl, and —S(O)₂C₁₋₆ alkyl. Preferably, in some embodiments of the compound of formula (VI-2), R^{9C} is selected from a hydrogen atom, deuterated methyl, ethyl, trifluoroethyl, benzyl, —C(O)OCH₃, —C(O)CH₃, and —S(O)₂CH₃. In some embodiments of the compound of formula (VI-2), Y is O. In some other embodiments of the compound of formula (VI-2), Y is NH.

In some embodiments of the compound of formula (VI-2), p is 2. In some embodiments of the compound of formula (VI-2), p is 0 or 1. In some embodiments of the compound of formula (VI-2), p is 1. In some other embodiments of the compound of formula (VI-2), p is 0.

In some aspects, the present disclosure provides a compound of formula (VI-3) or a pharmaceutically acceptable salt, an ester, a stereoisomer, a tautomer, a polymorph, a hydrate, a solvate, an N-oxide, an isotopically labeled compound, a metabolite, or a prodrug thereof,

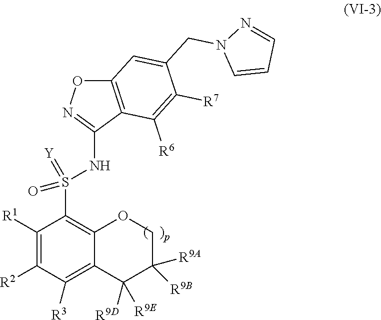

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9D} and R^{9E} are identical or different and are each independently selected from a hydrogen atom, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, —NHC₁₋₆ alkyl, —N(C₁₋₆ alkyl)₂, C₃₋₆ cycloalkyl, C₃₋₆ halocycloalkyl, C₃₋₆ cycloalkoxy, C₃₋₆ halocycloalkoxy, and hydroxyl;
Y is O or NH;
p is 0, 1, or 2.

In some embodiments of the compound of formula (VI-3), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VI-3), R¹ is selected from a hydrogen atom, methoxy, difluoromethoxy, trifluoromethoxy, —OCH₂CHF₂, cyclobutyloxy, and

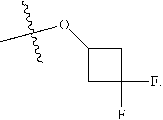 [SMILES: *OC1CC(F)(F)C1]

More specifically, in some embodiments of the compound of formula (VI-3), R¹ is cyclobutyloxy. In some other embodiments of the compound of formula (VI-3), R¹ is methoxy.

In some embodiments of the compound of formula (VI-3), R² and R³ are each independently selected from a hydrogen atom, halogen, C₁₋₄ alkyl, and C₁₋₄ alkoxy. Preferably, in some embodiments of the compound of formula (VI-3), both R² and R³ are hydrogen atoms.

In some embodiments of the compound of formula (VI-3), R⁶ and R⁷ are identical or different and are each independently selected from a hydrogen atom, a chlorine atom, a fluorine atom, methoxy, ethoxy, isopropoxy, difluoromethoxy, and cyclopropyl. Preferably, in some embodiments of the compound of formula (VI-3), R⁶ and R⁷ are identical or different and are each independently selected from a hydrogen atom, a chlorine atom, a fluorine atom, and methoxy. More preferably, in some embodiments of the compound of formula (VI-3), R⁶ is methoxy. In some embodiments of the compound of formula (VI-3), R⁷ is a hydrogen atom. In some other embodiments of the compound of formula (VI-3), R⁷ is a fluorine atom.

In some embodiments of the compound of formula (VI-3), R^{9A} and R^{9B} are identical or different and are each independently selected from a hydrogen atom, a fluorine atom, and methyl.

Preferably, in some embodiments of the compound of formula (VI-3), both R^{9A} and R^{9B} are hydrogen atoms.

In some embodiments of the compound of formula (VI-3), R^{9D} and R^{9E} are identical or different and are each independently selected from a hydrogen atom, a fluorine atom, methyl, and hydroxyl. In some embodiments of the compound of formula (VI-3), R^{9D} and R^{9E} are identical or different and are each independently selected from a fluorine atom and methyl. Specifically, in some embodiments of the compound of formula (VI-3), both R^{9D} and R^{9E} are fluorine atoms or methyl.

In some embodiments of the compound of formula (VI-3), Y is O. In some other embodiments of the compound of formula (VI-3), Y is NH.

In some embodiments of the compound of formula (VI-3), p is 2. In some embodiments of the compound of formula (VI-3), p is 0 or 1. In some embodiments of the compound of formula (VI-3), p is 1. In some other embodiments of the compound of formula (VI-3), p is 0.

In some aspects, the present disclosure provides a compound of formula (VI-4) or a pharmaceutically acceptable salt, an ester, a stereoisomer, a tautomer, a polymorph, a hydrate, a solvate, an N-oxide, an isotopically labeled compound, a metabolite, or a prodrug thereof,

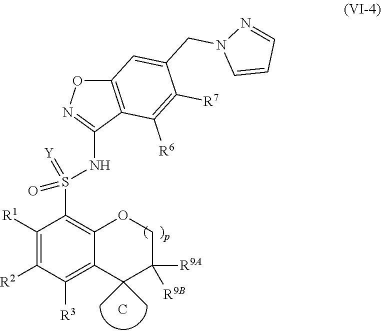

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
ring C is selected from 3- to 8-membered cycloalkyl, 3- to 8-membered heterocyclyl, 6- to 12-membered spirocyclyl, 6- to 12-membered spiroheterocyclyl, and 5- to 12-membered bridged heterocyclyl; ring C is optionally substituted with one or more substituents selected from halogen, hydroxyl, amino, C₁₋₆ alkyl, and C₁₋₆ haloalkyl;
Y is O or NH;
p is 0, 1, or 2.

In some embodiments of the compound of formula (VI-4), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VI-4), R¹ is selected from a hydrogen atom, methoxy, difluoromethoxy, trifluoromethoxy, —OCH₂CHF₂, cyclobutyloxy, and

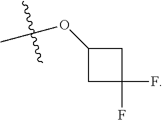 [SMILES: *OC1CC(F)(F)C1]

More specifically, in some embodiments of the compound of formula (VI-4), R¹ is cyclobutyloxy. In some other embodiments of the compound of formula (VI-4), R¹ is methoxy.

In some embodiments of the compound of formula (VI-4), R² and R³ are each independently selected from a hydrogen atom, halogen, C₁₋₄ alkyl, and C₁₋₄ alkoxy. Preferably, in some embodiments of the compound of formula (VI-4), both R² and R³ are hydrogen atoms.

In some embodiments of the compound of formula (VI-4), R⁶ and R⁷ are identical or different and are each independently selected from a hydrogen atom, a chlorine atom, a fluorine atom, methoxy, ethoxy, isopropoxy, difluoromethoxy, and cyclopropyl. Preferably, in some embodiments of the compound of formula (VI-4), R⁶ and R⁷ are identical or different and are each independently selected from a hydrogen atom, a chlorine atom, a fluorine atom, and methoxy. More preferably, in some embodiments of the compound of formula (VI-4), R⁶ is methoxy. In some embodiments of the compound of formula (VI-4), R⁷ is a hydrogen atom. In some other embodiments of the compound of formula (VI-4), R⁷ is a fluorine atom.

In some embodiments of the compound of formula (VI-4), R^{9A} and R^{9B} are identical or different and are each independently selected from a hydrogen atom, a fluorine atom, and methyl. Preferably, in some embodiments of the compound of formula (VI-4), both R^{9A} and R^{9B} are hydrogen atoms.

In some embodiments of the compound of formula (VI-4), ring C is selected from

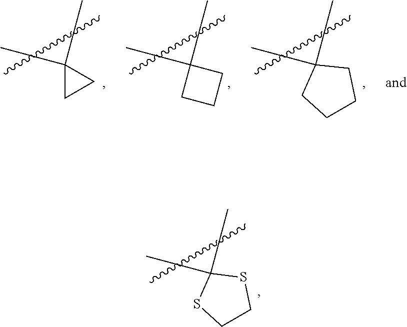

ring C is optionally substituted with one or more substituents selected from halogen, hydroxyl, amino, C₁₋₆ alkyl, and C₁₋₆ haloalkyl. Preferably, ring C is selected from

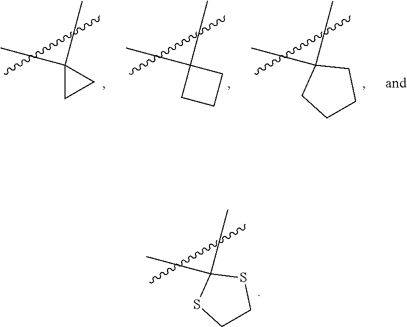

In some embodiments of the compound of formula (VI-4), Y is O. In some other embodiments of the compound of formula (VI-4), Y is NH.

In some embodiments of the compound of formula (VI-4), p is 2. In some embodiments of the compound of formula (VI-4), p is 0 or 1. In some embodiments of the compound of formula (VI-4), p is 1. In some other embodiments of the compound of formula (VI-4), p is 0.

In some aspects, the present disclosure provides a compound of formula (VII-1) or a pharmaceutically acceptable salt, an ester, a stereoisomer, a tautomer, a polymorph, a hydrate, a solvate, an N-oxide, an isotopically labeled compound, a metabolite, or a prodrug thereof,

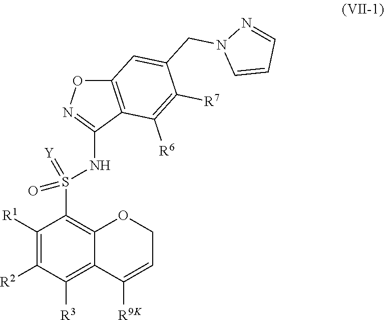

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9K} is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, 6- to 10-membered aryl, 5- to 10-membered heteroaryl, and hydroxyl, wherein the C₃₋₆ cycloalkyl and C₃₋₆ heterocyclyl are optionally substituted with one or more substituents selected from: halogen, hydroxyl, methyl, and methoxy;
Y is O or NH.

In some embodiments of the compound of formula (VII-1), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VII-1), R¹ is selected from a hydrogen atom, methoxy, difluoromethoxy, trifluoromethoxy, —OCH₂CHF₂, cyclobutyloxy, and

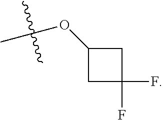 [SMILES: *OC1CC(F)(F)C1]

More preferably, in some embodiments of the compound of formula (VII-1), R¹ is methoxy. In some embodiments of the compound of formula (VII-1), R² and R³ are identical or different and are each independently selected from a hydrogen atom, halogen, C₁₋₄ alkyl, and C₁₋₄ alkoxy. Preferably, in some embodiments of the compound of formula (VII-1), R² and R³ are identical or different and are each independently selected from a hydrogen atom and halogen. More preferably, in some embodiments of the compound of formula (VII-1), both R² and R³ are hydrogen atoms.

In some embodiments of the compound of formula (VII-1), R⁶ and R⁷ are identical or different and are each independently selected from a hydrogen atom, a chlorine atom, a fluorine atom, methoxy, ethoxy, isopropoxy, difluoromethoxy, and cyclopropyl. In some embodiments of the compound of formula (VII-1), R⁶ is methoxy. In some embodiments of the compound of formula (VII-1), R⁷ is a hydrogen atom. In some other embodiments of the compound of formula (VII-1), R⁷ is a fluorine atom.

In some embodiments of the compound of formula (VII-1), R^{9K} is selected from a hydrogen atom, halogen, C₁₋₆ alkyl, C₁₋₆ haloalkyl, and hydroxyl. Preferably, in some embodiments of the compound of formula (VII-1), R^{9K} is selected from a hydrogen atom, methyl, and hydroxyl. In some embodiments of the compound of formula (VII-1), Y is O. In some other embodiments of the compound of formula (VII-1), Y is NH.

In some aspects, the present disclosure provides a compound of formula (VII-2) or a pharmaceutically acceptable salt, an ester, a stereoisomer, a tautomer, a polymorph, a hydrate, a solvate, an N-oxide, an isotopically labeled compound, a metabolite, or a prodrug thereof,

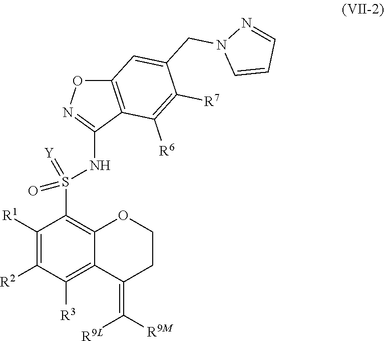

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9M} and R^{9L} are identical or different and are each independently selected from a hydrogen atom, C₁₋₆ alkyl, and C₁₋₆ haloalkyl;
Y is O or NH.

In some embodiments of the compound of formula (VII-2), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VII-2), R¹ is selected from a hydrogen atom, methoxy, difluoromethoxy, trifluoromethoxy, —OCH₂CHF₂, cyclobutyloxy, and

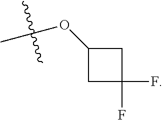 [SMILES: *OC1CC(F)(F)C1]

More preferably, in some embodiments of the compound of formula (VII-2), R¹ is methoxy.

In some embodiments of the compound of formula (VII-2), R² and R³ are identical or different and are each independently selected from a hydrogen atom, halogen, C₁₋₄ alkyl, and C₁₋₄ alkoxy.

Preferably, in some embodiments of the compound of formula (VII-2), R² and R³ are identical or different and are each independently selected from a hydrogen atom and halogen. More preferably, in some embodiments of the compound of formula (VII-2), both R² and R³ are hydrogen atoms.

In some embodiments of the compound of formula (VII-2), R⁶ and R⁷ are identical or different and are each independently selected from a hydrogen atom, a chlorine atom, a fluorine atom, methoxy, ethoxy, isopropoxy, difluoromethoxy, and cyclopropyl. In some embodiments of the compound of formula (VII-2), R⁶ is methoxy. In some embodiments of the compound of formula (VII-2), R⁷ is a hydrogen atom. In some other embodiments of the compound of formula (VII-2), R⁷ is a fluorine atom.

In some embodiments of the compound of formula (VII-2), R^{9M} and R^{9L} are identical or different and are each independently selected from a hydrogen atom and C₁₋₆ alkyl. Preferably, in some embodiments of the compound of formula (VII-2), R^{9M} and R^{9L} are identical or different and are each independently selected from a hydrogen atom, a fluorine atom, and methyl. In some embodiments of the compound of formula (VII-2), Y is O. In some other embodiments of the compound of formula (VII-2), Y is NH.

In some aspects, the present disclosure provides a compound of formula (VII-3) or a pharmaceutically acceptable salt, an ester, a stereoisomer, a tautomer, a polymorph, a hydrate, a solvate, an N-oxide, an isotopically labeled compound, a metabolite, or a prodrug thereof,

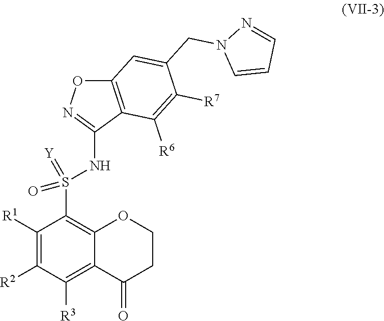

wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
Y is O or NH.

In some embodiments of the compound of formula (VII-3), R¹ is selected from a hydrogen atom, C₁₋₄ alkoxy, C₁₋₄ haloalkoxy, C₄₋₆ cycloalkoxy, and C₄₋₆ halocycloalkoxy. Preferably, in some embodiments of the compound of formula (VII-3), R¹ is selected from a hydrogen atom, methoxy, difluoromethoxy, trifluoromethoxy, —OCH₂CHF₂, cyclobutyloxy, and

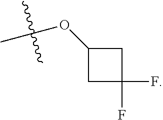 [SMILES: *OC1CC(F)(F)C1]

More preferably, in some embodiments of the compound of formula (VII-3), R¹ is methoxy. In some embodiments of the compound of formula (VII-3), R² and R³ are identical or different and are each independently selected from a hydrogen atom, halogen, C₁₋₄ alkyl, and C₁₋₄ alkoxy. Preferably, in some embodiments of the compound of formula (VII-3), R² and R³ are identical or different and are each independently selected from a hydrogen atom and halogen. More preferably, in some embodiments of the compound of formula (VII-3), both R² and R³ are hydrogen atoms.

In some embodiments of the compound of formula (VII-3), R⁶ and R⁷ are identical or different and are each independently selected from a hydrogen atom, a chlorine atom, a fluorine atom, methoxy, ethoxy, isopropoxy, difluoromethoxy, and cyclopropyl. In some embodiments of the compound of formula (VII-3), R⁶ is methoxy. In some embodiments of the compound of formula (VII-3), R⁷ is a hydrogen atom. In some other embodiments of the compound of formula (VII-3), R⁷ is a fluorine atom.

In some embodiments of the compound of formula (VII-3), Y is O. In some other embodiments of the compound of formula (VII-3), Y is NH.

In some embodiments, the present disclosure provides compounds listed in Table 1 below or pharmaceutically acceptable salts, esters, stereoisomers, tautomers, polymorphs, hydrates, solvates, N-oxides, isotopically labeled compounds, metabolites, or prodrugs thereof.

TABLE 1
Typical compounds of the present disclosure, including but not limited to:

| Compound | | |
|---|---|---|
| No. | Structural formula | Chemical name |
| 1 | 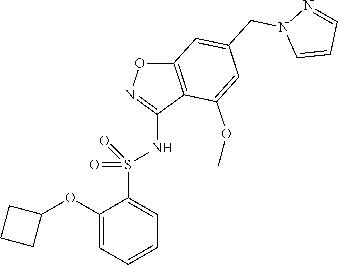 [SMILES: COc1cc(Cn2cccn2)cc2onc(NS(=O)(=O)c3ccccc3OC3CCC3)c12] | N-(6-((1H-Pyrazol-1-yl)methyl)-4-methoxybenzo[d]isoxazol- 3-yl)-2-cyclobutoxybenzenesulfonamide |
| 2 | 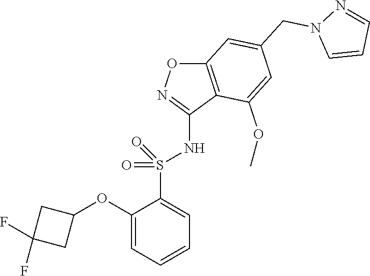 [SMILES: COc1cc(Cn2cccn2)cc2onc(NS(=O)(=O)c3ccccc3OC3CC(F)(F)C3)c12] | N-(6-((1H-Pyrazol-1-yl)methyl)-4-methoxybenzo[d]isoxazol- 3-yl)-2-(3,3-difluorocyclobutoxy)benzenesulfonamide |
| 3 | 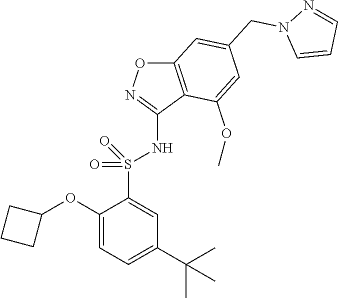 [SMILES: COc1cc(Cn2cccn2)cc2onc(NS(=O)(=O)c3cc(C(C)(C)C)ccc3OC3CCC3)c12] | N-(6-((1H-Pyrazol-1-yl)methyl)-4-methoxybenzo[d]isoxazol- 3-yl)-5-(tert-butyl)-2-cyclobutoxybenzenesulfonamide |
| 4 | 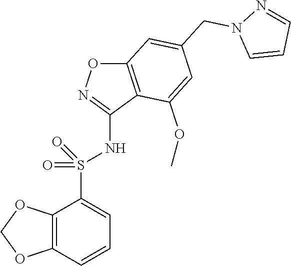 [SMILES: COc1cc(Cn2cccn2)cc2onc(NS(=O)(=O)c3cccc4c3OCO4)c12] | N-(6-((1H-Pyrazol-1-yl)methyl)-4-methoxybenzo[d]isoxazol- 3-yl)benzo[d][1,3]dioxole-4-sulfonamide |
| 5 | 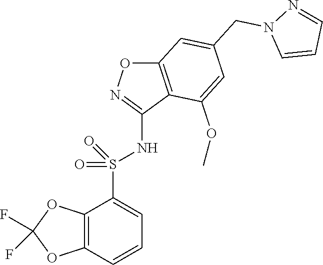 [SMILES: COc1cc(Cn2cccn2)cc2onc(NS(=O)(=O)c3cccc4c3OC(F)(F)O4)c12] | N-(6-((1H-Pyrazol-1-yl)methyl)-4-methoxybenzo[d]isoxazol- 3-yl)-2,2-difluorobenzo[d][1,3]dioxole-4-sulfonamide |
| 6 | 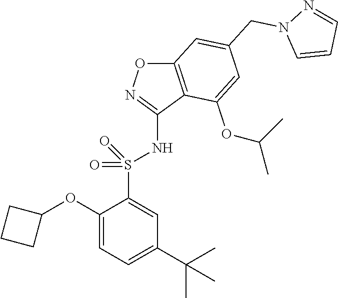 [SMILES: CC(C)Oc1cc(Cn2cccn2)cc2onc(NS(=O)(=O)c3cc(C(C)(C)C)ccc3OC3CCC3)c12] | N-(6-((1H-Pyrazol-1-yl)methyl)-4- isopropoxybenzo[d]isoxazol-3-yl)-5-(tert-butyl)-2- cyclobutoxybenzenesulfonamide |
| 7 | 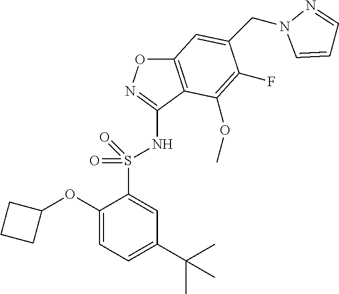 [SMILES: COc1c(F)c(Cn2cccn2)cc2onc(NS(=O)(=O)c3cc(C(C)(C)C)ccc3OC3CCC3)c12] | N-(6-((1H-Pyrazol-1-yl)methyl)-5-fluoro-4- methoxybenzo[d]isoxazol-3-yl)-5-(tert-butyl)-2- cyclobutoxybenzenesulfonamide |
| 8 | 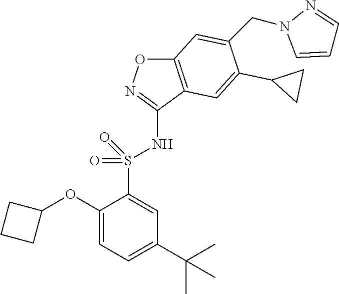 [SMILES: CC(C)(C)c1ccc(OC2CCC2)c(S(=O)(=O)Nc2noc3cc(Cn4cccn4)c(C4CC4)cc23)c1] | N-(6-((1H-Pyrazol-1-yl)methyl)-5- cyclopropylbenzo[d]isoxazol-3-yl)-5-(tert-butyl)-2- cyclobutoxybenzenesulfonamide |
| 9 | 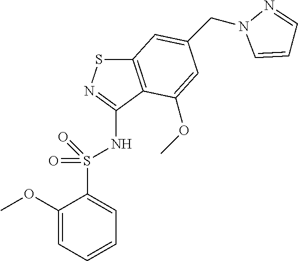 [SMILES: COc1ccccc1S(=O)(=O)Nc1nsc2cc(Cn3cccn3)cc(OC)c12] | N-(6-((1H-Pyrazol-1-yl)methyl)-4- methoxybenzo[d]isothiazol-3-yl)-2- methoxybenzenesulfonamide |
| 10 | 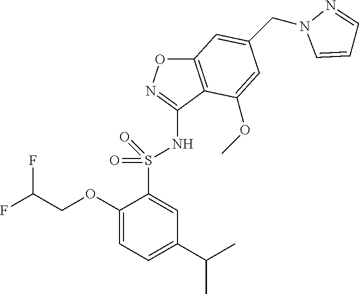 [SMILES: COc1cc(Cn2cccn2)cc2onc(NS(=O)(=O)c3cc(C(C)C)ccc3OCC(F)F)c12] | N-(6-((1H-Pyrazol-1-yl)methyl)-4-methoxybenzo[d]isoxazol- 3-yl)-2-(2,2-difluoroethoxy)-5-isopropylbenzenesulfonamide |
| 11 | 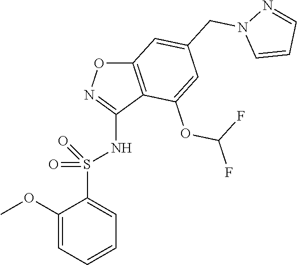 [SMILES: COc1ccccc1S(=O)(=O)Nc1noc2cc(Cn3cccn3)cc(OC(F)F)c12] | N-(6-((1H-Pyrazol-1-yl)methyl)-4- (difluoromethoxy)benzo[d]isoxazol-3-yl)-2- methoxybenzenesulfonamide |
| 12 | 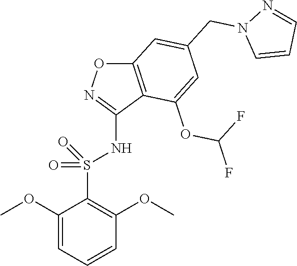 [SMILES: COc1cccc(OC)c1S(=O)(=O)Nc1noc2cc(Cn3cccn3)cc(OC(F)F)c12] | N-(6-((1H-Pyrazol-1-yl)methyl)-4- (difluoromethoxy)benzo[d]isoxazol-3-yl)-2,6- dimethoxybenzenesulfonamide |
| 13 | 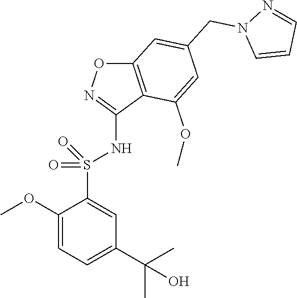 [SMILES: COc1ccc(C(C)(C)O)cc1S(=O)(=O)Nc1noc2cc(Cn3cccn3)cc(OC)c12] | N-(6-((1H-Pyrazol-1-yl)methyl)-4-methoxybenzo[d]isoxazol- 3-yl)-5-(2-hydroxypropan-2-yl)-2- methoxybenzenesulfonamide |
| 14 | 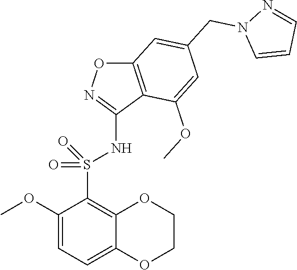 [SMILES: COc1ccc2c(c1S(=O)(=O)Nc1noc3cc(Cn4cccn4)cc(OC)c13)OCCO2] | N-(6-((1H-Pyrazol-1-yl)methyl)-4-methoxybenzo[d]isoxazol- 3-yl)-6-methoxy-2,3-dihydrobenzo[b][1,4]dioxine-5- sulfonamide |
| 15 | 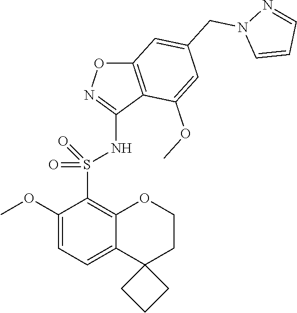 [SMILES: COc1ccc2c(c1S(=O)(=O)Nc1noc3cc(Cn4cccn4)cc(OC)c13)OCCC21CCC1] | N-(6-((1H-Pyrazol-1-yl)methyl)-4-methoxybenzo[d]isoxazol- 3-yl)-7-methoxyspiro[chromane-4,1′-cyclobutane]-8- sulfonamide |
| 16 | 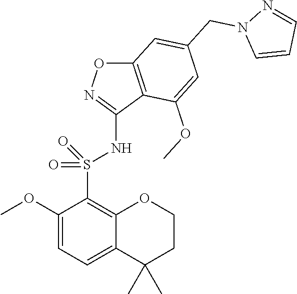 [SMILES: COc1ccc2c(c1S(=O)(=O)Nc1noc3cc(Cn4cccn4)cc(OC)c13)OCCC2(C)C] | N-(6-((1H-Pyrazol-1-yl)methyl)-4-methoxybenzo[d]isoxazol- 3-yl)-7-methoxy-4,4-dimethylchromane-8-sulfonamide |
| 17 | 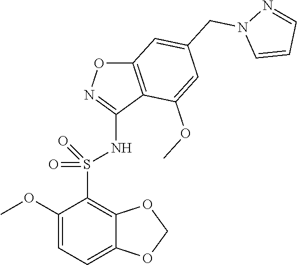 [SMILES: COc1ccc2c(c1S(=O)(=O)Nc1noc3cc(Cn4cccn4)cc(OC)c13)OCO2] | N-(6-((1H-Pyrazol-1-yl)methyl)-4-methoxybenzo[d]isoxazol- 3-yl)-5-methoxybenzo[d][1,3]dioxole-4-sulfonamide |
| 18 | 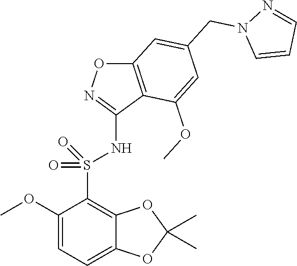 [SMILES: COc1ccc2c(c1S(=O)(=O)Nc1noc3cc(Cn4cccn4)cc(OC)c13)OC(C)(C)O2] | N-(6-((1H-Pyrazol-1-yl)methyl)-4-methoxybenzo[d]isoxazol- 3-yl)-5-methoxy-2,2-dimethylbenzo[d][1,3]dioxole-4- sulfonamide |
| 19 | 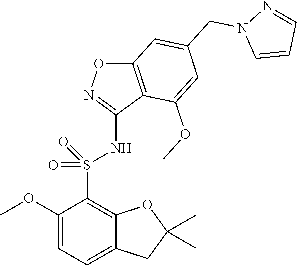 [SMILES: COc1ccc2c(c1S(=O)(=O)Nc1noc3cc(Cn4cccn4)cc(OC)c13)OC(C)(C)C2] | N-(6-((1H-Pyrazol-1-yl)methyl)-4-methoxybenzo[d]isoxazol- 3-yl)-6-methoxy-2,2-dimethyl-2,3-dihydrobenzofuran-7- sulfonamide |
| 20 | 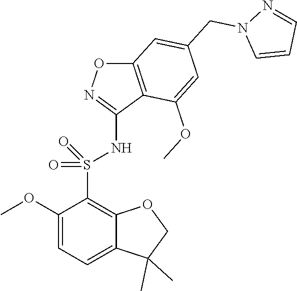 [SMILES: COc1ccc2c(c1S(=O)(=O)Nc1noc3cc(Cn4cccn4)cc(OC)c13)OCC2(C)C] | N-(6-((1H-Pyrazol-1-yl)methyl)-4-methoxybenzo[d]isoxazol- 3-yl)-6-methoxy-3,3-dimethyl-2,3-dihydrobenzofuran-7- sulfonamide |
| 21 | 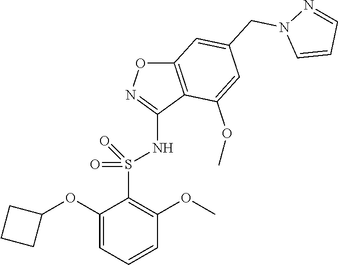 [SMILES: COc1cccc(OC2CCC2)c1S(=O)(=O)Nc1noc2cc(Cn3cccn3)cc(OC)c12] | N-(6-((1H-Pyrazol-1-yl)methyl)-4-methoxybenzo[d]isoxazol- 3-yl)-2-cyclobutoxy-6-methoxybenzenesulfonamide |
| 22 | 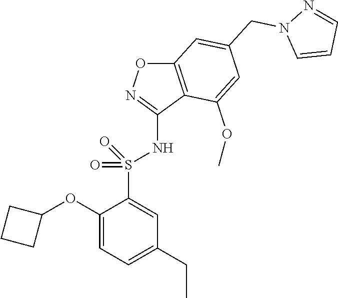 [SMILES: CCc1ccc(OC2CCC2)c(S(=O)(=O)Nc2noc3cc(Cn4cccn4)cc(OC)c23)c1] | N-(6-((1H-Pyrazol-1-yl)methyl)-4-methoxybenzo[d]isoxazol- 3-yl)-2-cyclobutoxy-5-ethylbenzenesulfonamide |
| 23 | 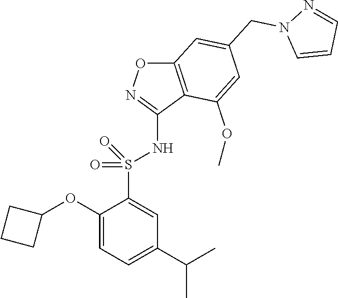 [SMILES: COc1cc(Cn2cccn2)cc2onc(NS(=O)(=O)c3cc(C(C)C)ccc3OC3CCC3)c12] | N-(6-((1H-Pyrazol-1-yl)methyl)-4-methoxybenzo[d]isoxazol- 3-yl)-2-cyclobutoxy-5-isopropylbenzenesulfonamide |
| 24 | 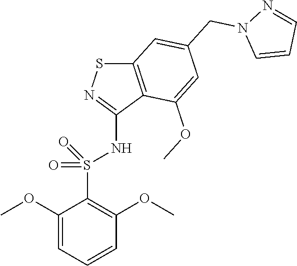 [SMILES: COc1cccc(OC)c1S(=O)(=O)Nc1nsc2cc(Cn3cccn3)cc(OC)c12] | N-(6-((1H-Pyrazol-1-yl)methyl)-4- methoxybenzo[d]isothiazol-3-yl)-2,6- dimethoxybenzenesulfonamide |
| 25 | 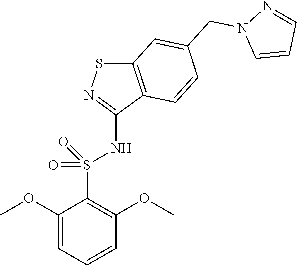 [SMILES: COc1cccc(OC)c1S(=O)(=O)Nc1nsc2cc(Cn3cccn3)ccc12] | N-(6-((1H-Pyrazol-1-yl)methyl)benzo[d]isothiazol-3-yl)-2,6- dimethoxybenzenesulfonamide |
| 26 | 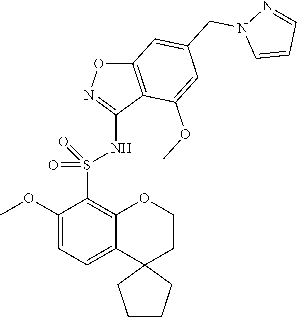 [SMILES: COc1ccc2c(c1S(=O)(=O)Nc1noc3cc(Cn4cccn4)cc(OC)c13)OCCC21CCCC1] | N-(6-((1H-Pyrazol-1-yl)methyl)-4-methoxybenzo[d]isoxazol- 3-yl)-7-methoxyspiro[chromane-4,l′-cyclopentane]-8- sulfonamide |
| 27 | 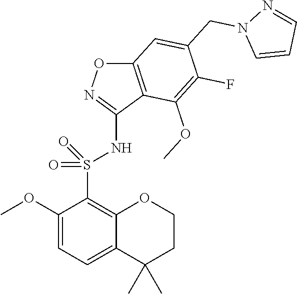 [SMILES: COc1ccc2c(c1S(=O)(=O)Nc1noc3cc(Cn4cccn4)c(F)c(OC)c13)OCCC2(C)C] | N-(6-((1H-Pyrazol-1-yl)methyl)-5-fluoro-4- methoxybenzo[d]isoxazol-3-yl)-7-methoxy-4,4- dimethylchromane-8-sulfonamide |
| 28 | 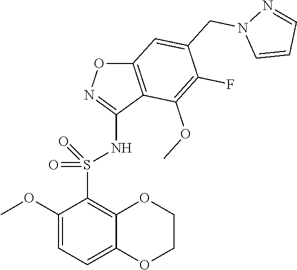 [SMILES: COc1ccc2c(c1S(=O)(=O)Nc1noc3cc(Cn4cccn4)c(F)c(OC)c13)OCCO2] | N-(6-((1H-Pyrazol-1-yl)methyl)-5-fluoro-4- methoxybenzo[d]isoxazol-3-yl)-6-methoxy-2,3- dihydrobenzo[b][1,4]dioxine-5-sulfonamide |
| 29 | 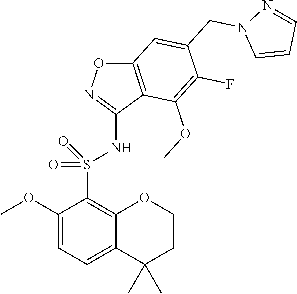 [SMILES: COc1ccc2c(c1S(=O)(=O)Nc1noc3cc(Cn4cccn4)c(F)c(OC)c13)OCCC2(C)C] | N-(6-((1H-Pyrazol-1-yl)methyl)-5-chloro-4- methoxybenzo[d]isoxazol-3-yl)-7-methoxy-4,4- dimethylchromane-8-sulfonamide |
| 30 | 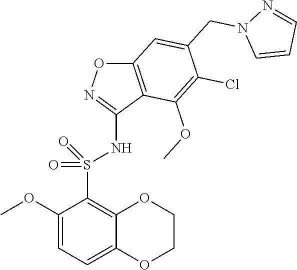 [SMILES: COc1ccc2c(c1S(=O)(=O)Nc1noc3cc(Cn4cccn4)c(Cl)c(OC)c13)OCCO2] | N-(6-((1H-Pyrazol-1-yl)methyl)-5-chloro-4- methoxybenzo[d]isoxazol-3-yl)-6-methoxy-2,3- dihydrobenzo[b][1,4]dioxine-5-sulfonamide |
| 31 | 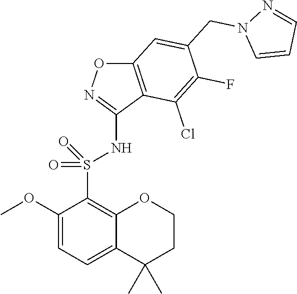 [SMILES: COc1ccc2c(c1S(=O)(=O)Nc1noc3cc(Cn4cccn4)c(F)c(Cl)c13)OCCC2(C)C] | N-(6-((1H-Pyrazol-1-yl)methyl)-4-chloro-5- fluorobenzo[d]isoxazol-3-yl)-7-methoxy-4,4- dimethylchromane-8-sulfonamide |
| 32 | 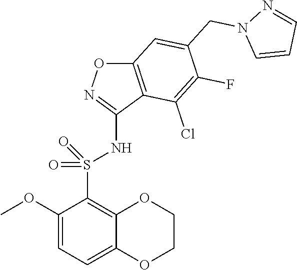 [SMILES: COc1ccc2c(c1S(=O)(=O)Nc1noc3cc(Cn4cccn4)c(F)c(Cl)c13)OCCO2] | N-(6-((1H-Pyrazol-1-yl)methyl)-4-chloro-5- fluorobenzo[d]isoxazol-3-yl)-6-methoxy-2,3- dihydrobenzo[b][1,4]dioxine-5-sulfonamide |
| 33 | 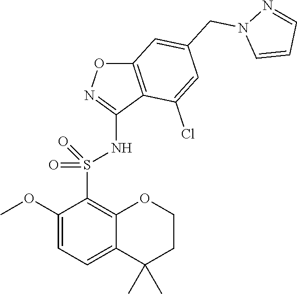 [SMILES: COc1ccc2c(c1S(=O)(=O)Nc1noc3cc(Cn4cccn4)cc(Cl)c13)OCCC2(C)C] | N-(6-((1H-Pyrazol-1-yl)methyl)-4-chlorobenzo[d]isoxazol-3- yl)-7-methoxy-4,4-dimethylchromane-8-sulfonamide |
| 34 | 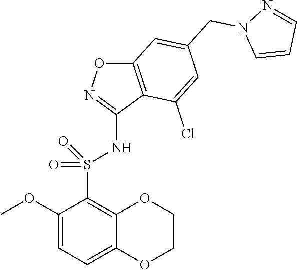 [SMILES: COc1ccc2c(c1S(=O)(=O)Nc1noc3cc(Cn4cccn4)cc(Cl)c13)OCCO2] | N-(6-((1H-Pyrazol-1-yl)methyl)-4-chlorobenzo[d]isoxazol-3- yl)-6-methoxy-2,3-dihydrobenzo[b][1,4]dioxine-5- sulfonamide |
| 35 | 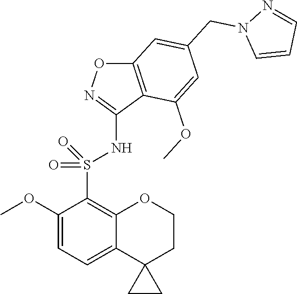 [SMILES: COc1ccc2c(c1S(=O)(=O)Nc1noc3cc(Cn4cccn4)cc(OC)c13)OCCC21CC1] | N-(6-((1H-Pyrazol-1-yl)methyl)-4-methoxybenzo[d]isoxazol- 3-yl)-7-methoxyspiro[chromane-4,l′-cyclopropane]-8- sulfonamide |
| 36 | 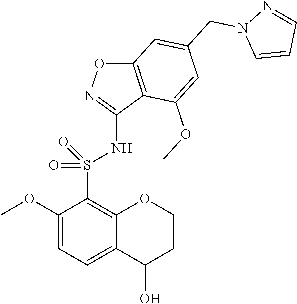 [SMILES: COc1ccc2c(c1S(=O)(=O)Nc1noc3cc(Cn4cccn4)cc(OC)c13)OCCC2O] | N-(6-((1H-Pyrazol-1-yl)methyl)-4-methoxybenzo[d]isoxazol- 3-yl)-4-hydroxy-7-methoxychromane-8-sulfonamide |
| 37 | 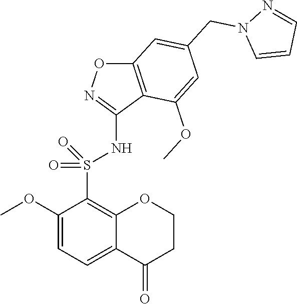 [SMILES: COc1ccc2c(c1S(=O)(=O)Nc1noc3cc(Cn4cccn4)cc(OC)c13)OCCC2=O] | N-(6-((1H-Pyrazol-1-yl)methyl)-4-methoxybenzo[d]isoxazol- 3-yl)-7-methoxy-4-oxochromane-8-sulfonamide |
| 38 | 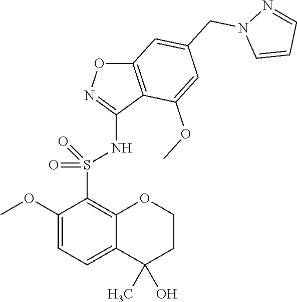 [SMILES: COc1ccc2c(c1S(=O)(=O)Nc1noc3cc(Cn4cccn4)cc(OC)c13)OCCC2(C)O] | N-(6-((1H-Pyrazol-1-yl)methyl)-4-methoxybenzo[d]isoxazol- 3-yl)-4-hydroxy-7-methoxy-4-methylchromane-8- sulfonamide |
| 39 | 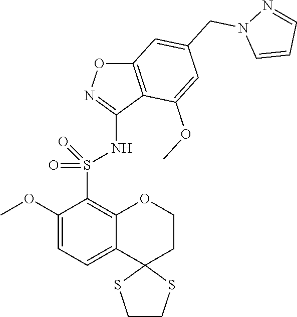 [SMILES: COc1ccc2c(c1S(=O)(=O)Nc1noc3cc(Cn4cccn4)cc(OC)c13)OCCC21SCCS1] | N-(6-((1H-Pyrazol-1-yl)methyl)-4-methoxybenzo[d]isoxazol- 3-yl)-7-methoxyspiro[chromane-4,2′-[1,3]dithiolane]-8- sulfonamide |
| 40 | 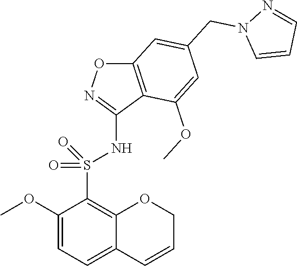 [SMILES: COc1ccc2c(c1S(=O)(=O)Nc1noc3cc(Cn4cccn4)cc(OC)c13)OCC=C2] | N-(6-((1H-Pyrazol-1-yl)methyl)-4-methoxybenzo[d]isoxazol- 3-yl)-7-methoxy-2H-chromene-8-sulfonamide |
| 41 | 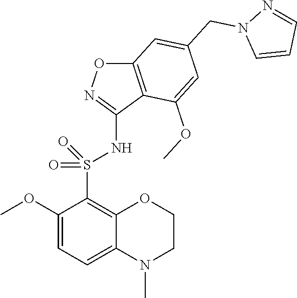 [SMILES: COc1ccc2c(c1S(=O)(=O)Nc1noc3cc(Cn4cccn4)cc(OC)c13)OCCN2C] | N-(6-((1H-Pyrazol-1-yl)methyl)-4-methoxybenzo[d]isoxazol- 3-yl)-7-methoxy-4-methyl-3,4-dihydro-2H- benzo[b][1,4]oxazine-8-sulfonamide |
| 42 | 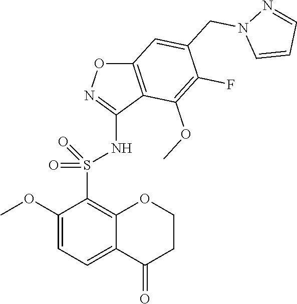 [SMILES: COc1ccc2c(c1S(=O)(=O)Nc1noc3cc(Cn4cccn4)c(F)c(OC)c13)OCCC2=O] | N-(6-((1H-Pyrazol-1-yl)methyl)-5-fluoro-4- methoxybenzo[d]isoxazol-3-yl)-7-methoxy-4-oxochromane- 8-sulfonamide |
| 43 | 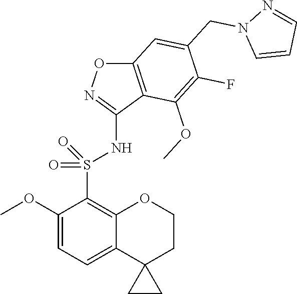 [SMILES: COc1ccc2c(c1S(=O)(=O)Nc1noc3cc(Cn4cccn4)c(F)c(OC)c13)OCCC21CC1] | N-(6-((1H-Pyrazol-1-yl)methyl)-5-fluoro-4- methoxybenzo[d]isoxazol-3-yl)-7-methoxyspiro[chromane- 4,1′-cyclopropane]-8-sulfonamide |
| 44 | 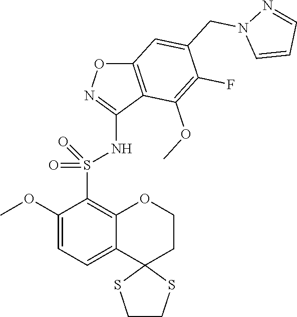 [SMILES: COc1ccc2c(c1S(=O)(=O)Nc1noc3cc(Cn4cccn4)c(F)c(OC)c13)OCCC21SCCS1] | N-(6-((1H-Pyrazol-1-yl)methyl)-5-fluoro-4- methoxybenzo[d]isoxazol-3-yl)-7-methoxyspiro[chromane- 4,2′-[1,3]dithiolane]-8-sulfonamide |
| 45 | 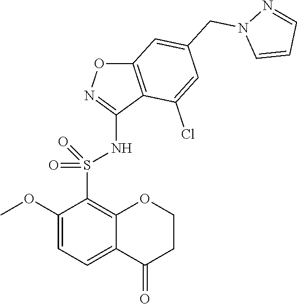 [SMILES: COc1ccc2c(c1S(=O)(=O)Nc1noc3cc(Cn4cccn4)cc(Cl)c13)OCCC2=O] | N-(6-((1H-Pyrazol-1-yl)methyl)-4-chlorobenzo[d]isoxazol-3- yl)-7-methoxy-4-oxochromane-8-sulfonamide |
| 46 | 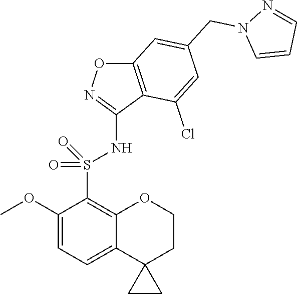 [SMILES: COc1ccc2c(c1S(=O)(=O)Nc1noc3cc(Cn4cccn4)cc(Cl)c13)OCCC21CC1] | N-(6-((1H-Pyrazol-1-yl)methyl)-4-chlorobenzo[d]isoxazol-3- yl)-7-methoxyspiro[chromane-4,1′-cyclopropane]-8- sulfonamide |
| 47 | 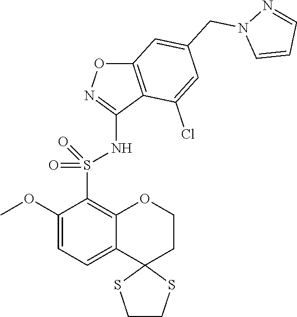 [SMILES: COc1ccc2c(c1S(=O)(=O)Nc1noc3cc(Cn4cccn4)cc(Cl)c13)OCCC21SCCS1] | N-(6-((1H-Pyrazol-1-yl)methyl)-4-chlorobenzo[d]isoxazol-3- yl)-7-methoxyspiro[chromane-4,2′-[1,3]dithiolane]-8- sulfonamide |
| 48 | 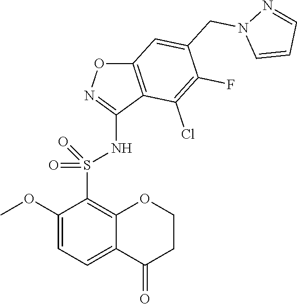 [SMILES: COc1ccc2c(c1S(=O)(=O)Nc1noc3cc(Cn4cccn4)c(F)c(Cl)c13)OCCC2=O] | N-(6-((1H-Pyrazol-1-yl)methyl)-4-chloro-5- fluorobenzo[d]isoxazol-3-yl)-7-methoxy-4-oxochromane-8- sulfonamide |
| 49 | 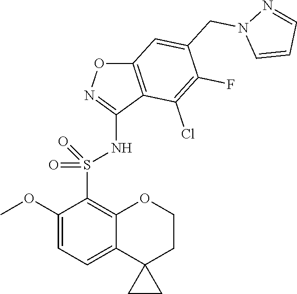 [SMILES: COc1ccc2c(c1S(=O)(=O)Nc1noc3cc(Cn4cccn4)c(F)c(Cl)c13)OCCC21CC1] | N-(6-((1H-Pyrazol-1-yl)methyl)-4-chloro-5- fluorobenzo[d]isoxazol-3-yl)-7-methoxyspiro[chromane-4,1′- cyclopropane]-8-sulfonamide |
| 50 | 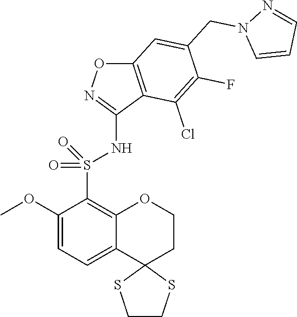 [SMILES: COc1ccc2c(c1S(=O)(=O)Nc1noc3cc(Cn4cccn4)c(F)c(Cl)c13)OCCC21SCCS1] | N-(6-((1H-Pyrazol-1-yl)methyl)-4-chloro-5- fluorobenzo[d]isoxazol-3-yl)-7-methoxyspiro[chromane-4,2′- [1,3]dithiolane]-8-sulfonamide |
| 51 | 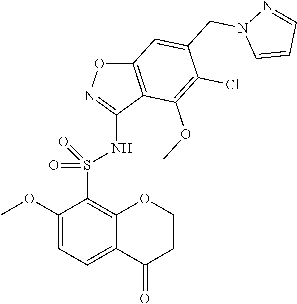 [SMILES: COc1ccc2c(c1S(=O)(=O)Nc1noc3cc(Cn4cccn4)c(Cl)c(OC)c13)OCCC2=O] | N-(6-((1H-Pyrazol-1-yl)methyl)-5-chloro-4- methoxybenzo[d]isoxazol-3-yl)-7-methoxy-4-oxochromane- 8-sulfonamide |
| 52 | 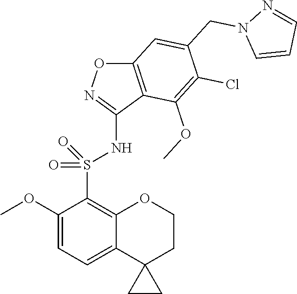 [SMILES: COc1ccc2c(c1S(=O)(=O)Nc1noc3cc(Cn4cccn4)c(Cl)c(OC)c13)OCCC21CC1] | N-(6-((1H-Pyrazol-1-yl)methyl)-5-chloro-4- methoxybenzo[d]isoxazol-3-yl)-7-methoxyspiro[chromane- 4,l′-cyclopropane]-8-sulfonamide |
| 53 | 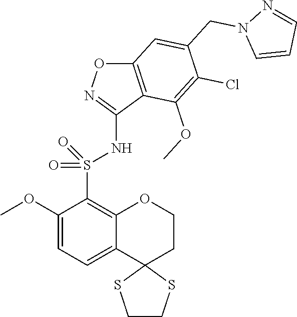 [SMILES: COc1ccc2c(c1S(=O)(=O)Nc1noc3cc(Cn4cccn4)c(Cl)c(OC)c13)OCCC21SCCS1] | N-(6-((1H-Pyrazol-1-yl)methyl)-5-chloro-4- methoxybenzo[d]isoxazol-3-yl)-7-methoxyspiro[chromane- 4,2′-[1,3]dithiolane]-8-sulfonamide |
| 54 | 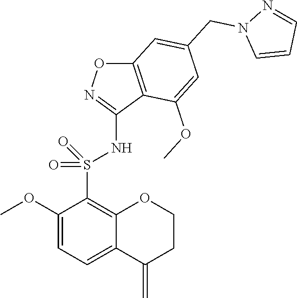 [SMILES: C=C1CCOc2c1ccc(OC)c2S(=O)(=O)Nc1noc2cc(Cn3cccn3)cc(OC)c12] | N-(6-((1H-Pyrazol-1-yl)methyl)-4-methoxybenzo[d]isoxazol- 3-yl)-7-methoxy-4-methylenechromane-8-sulfonamide |
| 55 | 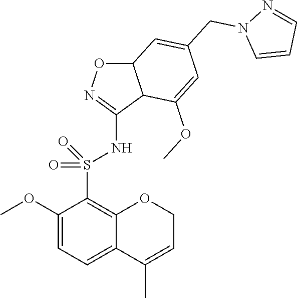 [SMILES: COC1=CC(Cn2cccn2)=CC2ON=C(NS(=O)(=O)c3c(OC)ccc4c3OCC=C4C)C12] | N-(6-((1H-Pyrazol-1-yl)methyl)-4-methoxy-3a,7a- dihydrobenzo[d]isoxazol-3-yl)-7-methoxy-4-methyl-2H- chromene-8-sulfonamide |
| 56 | 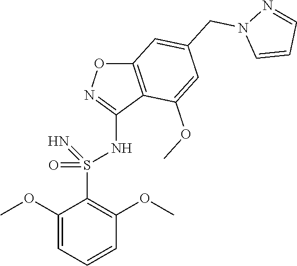 [SMILES: COc1cccc(OC)c1S(=N)(=O)Nc1noc2cc(Cn3cccn3)cc(OC)c12] | N-(6-((1H-Pyrazol-1-yl)methyl)-4-methoxybenzo[d]isoxazol- 3-yl)-2,6-dimethoxybenzenesulfonimidamide |
| 57 | 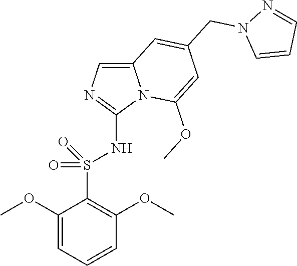 [SMILES: COc1cccc(OC)c1S(=O)(=O)Nc1ncc2cc(Cn3cccn3)cc(OC)n12] | N-(7-((1H-Pyrazol-1-yl)methyl)-5-methoxyimidazo[1,5- a]pyridin-3-yl)-2,6-dimethoxybenzenesulfonamide |
| 58 | 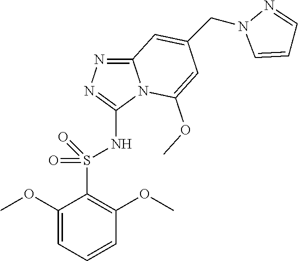 [SMILES: COc1cccc(OC)c1S(=O)(=O)Nc1nnc2cc(Cn3cccn3)cc(OC)n12] | N-(7-((1H-Pyrazol-1-yl)methyl)-5-methoxy- [1,2,4]triazolo[4,3-a]pyridin-3-yl)-2,6- dimethoxybenzenesulfonamide |
| 59 | 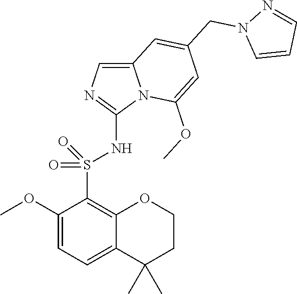 [SMILES: COc1ccc2c(c1S(=O)(=O)Nc1ncc3cc(Cn4cccn4)cc(OC)n13)OCCC2(C)C] | N-(7-((1H-Pyrazol-1-yl)methyl)-5-methoxyimidazo[1,5- a]pyridin-3-yl)-7-methoxy-4,4-dimethylchromane-8- sulfonamide |
| 60 | 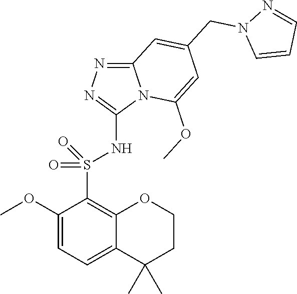 [SMILES: COc1ccc2c(c1S(=O)(=O)Nc1nnc3cc(Cn4cccn4)cc(OC)n13)OCCC2(C)C] | N-(7-((1H-Pyrazol-1-yl)methyl)-5-methoxy- [1,2,4]triazolo[4,3-a]pyridin-3-yl)-7-methoxy-4,4- dimethylchromane-8-sulfonamide |
| 61 | 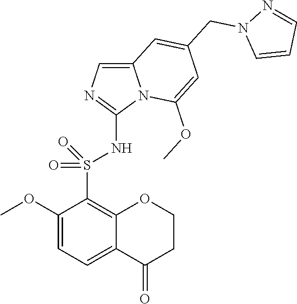 [SMILES: COc1ccc2c(c1S(=O)(=O)Nc1ncc3cc(Cn4cccn4)cc(OC)n13)OCCC2=O] | N-(7-((1H-Pyrazol-1-yl)methyl)-5-methoxyimidazo[1,5- a]pyridin-3-yl)-7-methoxy-4-oxochromane-8-sulfonamide |
| 62 | 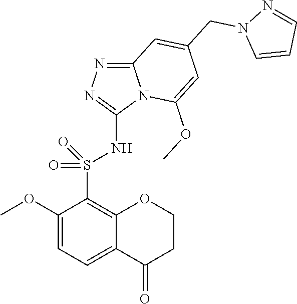 [SMILES: COc1ccc2c(c1S(=O)(=O)Nc1nnc3cc(Cn4cccn4)cc(OC)n13)OCCC2=O] | N-(7-((1H-Pyrazol-1-yl)methyl)-5-methoxy- [1,2,4]triazolo[4,3-a]pyridin-3-yl)-7-methoxy-4- oxochromane-8-sulfonamide |
| 63 | 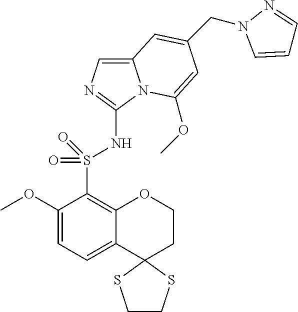 [SMILES: COc1ccc2c(c1S(=O)(=O)Nc1ncc3cc(Cn4cccn4)cc(OC)n13)OCCC21SCCS1] | N-(7-((1H-Pyrazol-1-yl)methyl)-5-methoxyimidazo[1,5- a]pyridin-3-yl)-7-methoxyspiro[chromane-4,2′- [1,3]dithiolane]-8-sulfonamide |
| 64 | 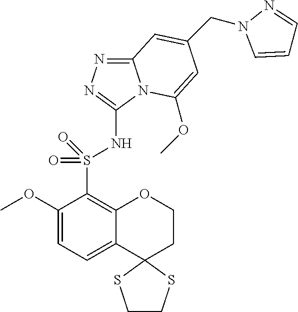 [SMILES: COc1ccc2c(c1S(=O)(=O)Nc1nnc3cc(Cn4cccn4)cc(OC)n13)OCCC21SCCS1] | N-(7-((1H-Pyrazol-1-yl)methyl)-5-methoxy- [1,2,4]triazolo[4,3-a]pyridin-3-yl)-7- methoxyspiro[chromane-4,2′-[1,3]dithiolane]-8-sulfonamide |
| 65 | 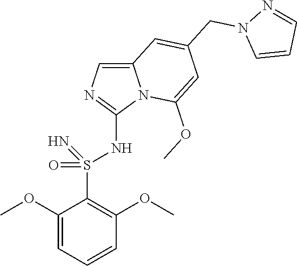 [SMILES: COc1cccc(OC)c1S(=N)(=O)Nc1ncc2cc(Cn3cccn3)cc(OC)n12] | N-(7-((1H-Pyrazol-1-yl)methyl)-5-methoxyimidazo[1,5- a]pyridin-3-yl)-2,6-dimethoxybenzenesulfonimidamide |
| 66 | 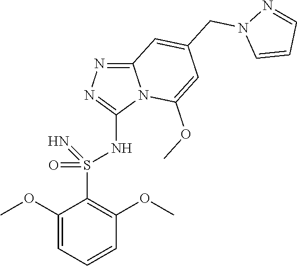 [SMILES: COc1cccc(OC)c1S(=N)(=O)Nc1nnc2cc(Cn3cccn3)cc(OC)n12] | N-(7-((1H-Pyrazol-1-yl)methyl)-5-methoxy- [1,2,4]triazolo[4,3-a]pyridin-3-yl)-2,6- dimethoxybenzenesulfonimidamide |
| 67 | 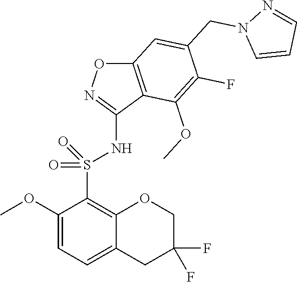 [SMILES: COc1ccc2c(c1S(=O)(=O)Nc1noc3cc(Cn4cccn4)c(F)c(OC)c13)OCC(F)(F)C2] | N-(6-((1H-Pyrazol-1-yl)methyl)-5-fluoro-4- methoxybenzo[d]isoxazol-3-yl)-3,3-difluoro-7- methoxychromane-8-sulfonamide |
| 68 | 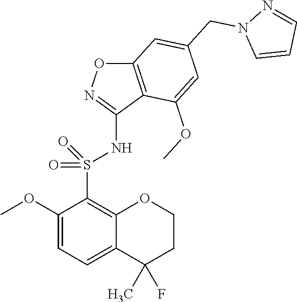 [SMILES: COc1ccc2c(c1S(=O)(=O)Nc1noc3cc(Cn4cccn4)cc(OC)c13)OCCC2(C)F] | N-(6-((1H-Pyrazol-1-yl)methyl)-4-methoxybenzo[d]isoxazol- 3-yl)-4-fluoro-7-methoxy-4-methylchromane-8-sulfonamide |
| 69 | 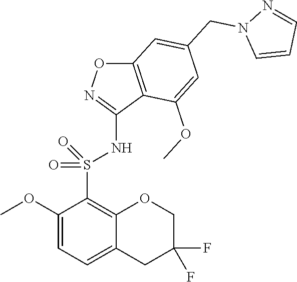 [SMILES: COc1ccc2c(c1S(=O)(=O)Nc1noc3cc(Cn4cccn4)cc(OC)c13)OCC(F)(F)C2] | N-(6-((1H-Pyrazol-1-yl)methyl)-4-methoxybenzo[d]isoxazol- 3-yl)-3,3-difluoro-7-methoxychromane-8-sulfonamide |
| 70 | 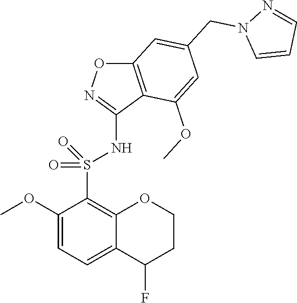 [SMILES: COc1ccc2c(c1S(=O)(=O)Nc1noc3cc(Cn4cccn4)cc(OC)c13)OCCC2F] | N-(6-((1H-Pyrazol-1-yl)methyl)-4-methoxybenzo[d]isoxazol- 3-yl)-4-fluoro-7-methoxychromane-8-sulfonamide |
| 71 | 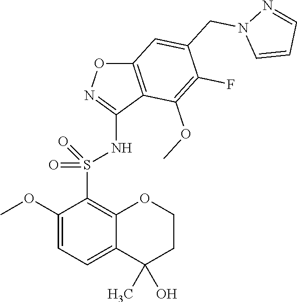 [SMILES: COc1ccc2c(c1S(=O)(=O)Nc1noc3cc(Cn4cccn4)c(F)c(OC)c13)OCCC2(C)O] | N-(6-((1H-Pyrazol-1-yl)methyl)-5-fluoro-4- methoxybenzo[d]isoxazol-3-yl)-4-hydroxy-7-methoxy-4- methylchromane-8-sulfonamide |
| 72 | 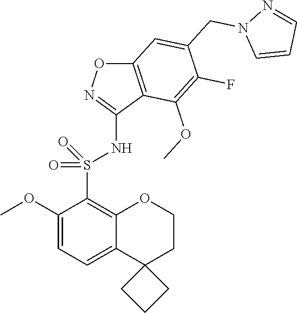 [SMILES: COc1ccc2c(c1S(=O)(=O)Nc1noc3cc(Cn4cccn4)c(F)c(OC)c13)OCCC21CCC1] | N-(6-((1H-Pyrazol-1-yl)methyl)-5-fluoro-4- methoxybenzo[d]isoxazol-3-yl)-7-methoxyspiro[chromane- 4,l′-cyclobutane]-8-sulfonamide |
| 73 | 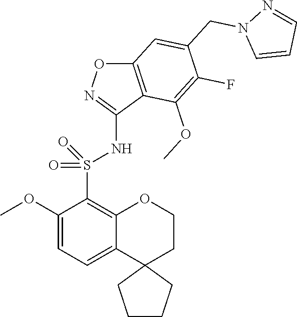 [SMILES: COc1ccc2c(c1S(=O)(=O)Nc1noc3cc(Cn4cccn4)c(F)c(OC)c13)OCCC21CCCC1] | N-(6-((1H-Pyrazol-1-yl)methyl)-5-fluoro-4- methoxybenzo[d]isoxazol-3-yl)-7-methoxyspiro[chromane- 4,l′-cyclopentane]-8-sulfonamide |
| 74 | 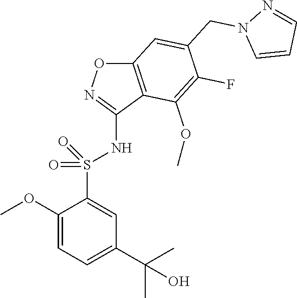 [SMILES: COc1ccc(C(C)(C)O)cc1S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-5-(2-hydroxypropan-2-yl)-2- methoxybenzenesulfonamide |
| 75 | 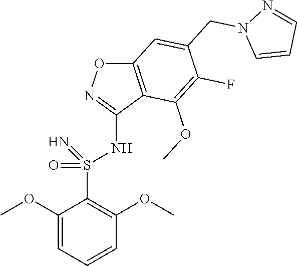 [SMILES: COc1cccc(OC)c1S(=N)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-2,6- dimethoxybenzenesulfonimidamide |
| 76 | 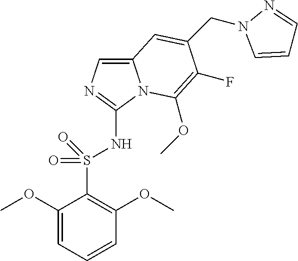 [SMILES: COc1cccc(OC)c1S(=O)(=O)Nc1ncc2cc(Cn3cccn3)c(F)c(OC)n12] | N-(7-((1H-Pyrazol-1-yl)methyl)-6-fluoro-5- methoxyimidazo[1,5-a]pyridin-3-yl)-2,6- dimethoxybenzenesulfonamide |
| 77 | 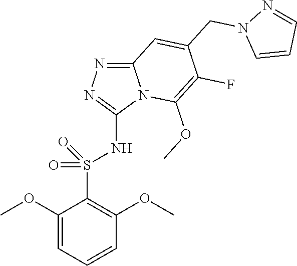 [SMILES: COc1cccc(OC)c1S(=O)(=O)Nc1nnc2cc(Cn3cccn3)c(F)c(OC)n12] | N-(7-((1H-Pyrazol-1-yl)methyl)-6-fluoro-5-methoxy- [1,2,4]triazolo[4,3-a]pyridin-3-yl)-2,6- dimethoxybenzenesulfonamide |
| 78 | 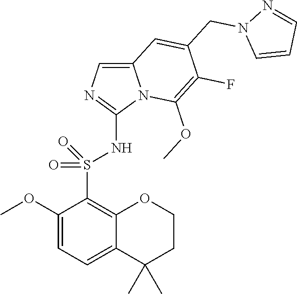 [SMILES: COc1ccc2c(c1S(=O)(=O)Nc1ncc3cc(Cn4cccn4)c(F)c(OC)n13)OCCC2(C)C] | N-(7-((1H-Pyrazol-1-yl)methyl)-6-fluoro-5- methoxyimidazo[1,5-a]pyridin-3-yl)-7-methoxy-4,4- dimethylchromane-8-sulfonamide |
| 79 | 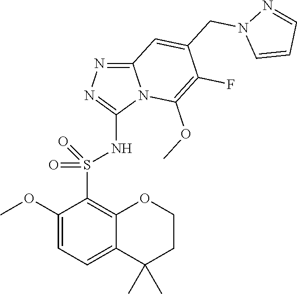 [SMILES: COc1ccc2c(c1S(=O)(=O)Nc1nnc3cc(Cn4cccn4)c(F)c(OC)n13)OCCC2(C)C] | N-(7-((1H-Pyrazol-1-yl)methyl)-6-fluoro-5-methoxy- [1,2,4]triazolo[4,3-a]pyridin-3-yl)-7-methoxy-4,4- dimethylchromane-8-sulfonamide |
| 80 | 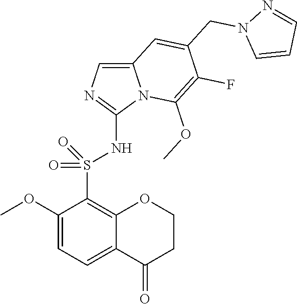 [SMILES: COc1ccc2c(c1S(=O)(=O)Nc1ncc3cc(Cn4cccn4)c(F)c(OC)n13)OCCC2=O] | N-(7-((1H-Pyrazol-1-yl)methyl)-6-fluoro-5- methoxyimidazo[1,5-a]pyridin-3-yl)-7-methoxy-4- oxochromane-8-sulfonamide |
| 81 | 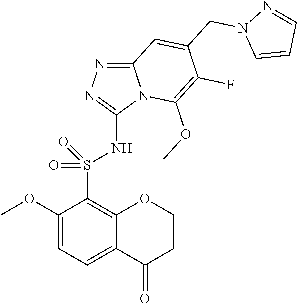 [SMILES: COc1ccc2c(c1S(=O)(=O)Nc1nnc3cc(Cn4cccn4)c(F)c(OC)n13)OCCC2=O] | N-(7-((1H-Pyrazol-1-yl)methyl)-6-fluoro-5-methoxy- [1,2,4]triazolo[4,3-a]pyridin-3-yl)-7-methoxy-4- oxochromane-8-sulfonamide |
| 82 | 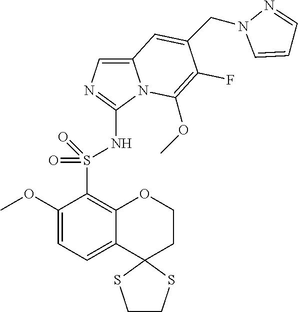 [SMILES: COc1ccc2c(c1S(=O)(=O)Nc1ncc3cc(Cn4cccn4)c(F)c(OC)n13)OCCC21SCCS1] | N-(7-((1H-Pyrazol-1-yl)methyl)-6-fluoro-5- methoxyimidazo[1,5-a]pyridin-3-yl)-7- methoxyspiro[chromane-4,2′-[1,3]dithiolane]-8-sulfonamide |
| 83 | 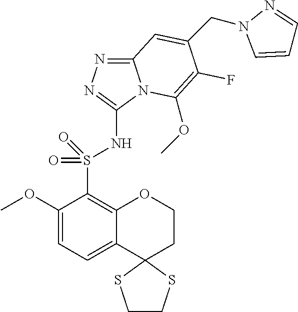 [SMILES: COc1ccc2c(c1S(=O)(=O)Nc1nnc3cc(Cn4cccn4)c(F)c(OC)n13)OCCC21SCCS1] | N-(7-((1H-Pyrazol-1-yl)methyl)-6-fluoro-5-methoxy- [1,2,4]triazolo[4,3-a]pyridin-3-yl)-7- methoxyspiro[chromane-4,2′-[1,3]dithiolane]-8-sulfonamide |
| 84 | 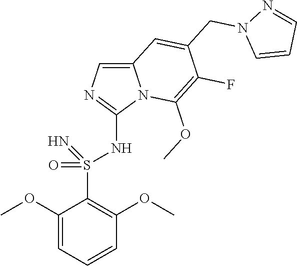 [SMILES: COc1cccc(OC)c1S(=N)(=O)Nc1ncc2cc(Cn3cccn3)c(F)c(OC)n12] | N-(7-((1H-Pyrazol-1-yl)methyl)-6-fluoro-5- methoxyimidazo[1,5-a]pyridin-3-yl)-2,6- dimethoxybenzenesulfonimidamide |
| 85 | 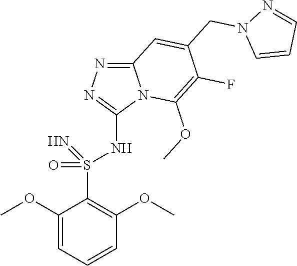 [SMILES: COc1cccc(OC)c1S(=N)(=O)Nc1nnc2cc(Cn3cccn3)c(F)c(OC)n12] | N-(7-((1H-Pyrazol-1-yl)methyl)-6-fluoro-5-methoxy- [1,2,4]triazolo[4,3-a]pyridin-3-yl)-2,6- dimethoxybenzenesulfonimidamide |
| 86 | 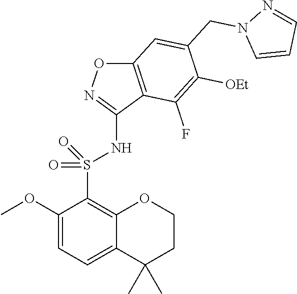 [SMILES: CCOc1c(Cn2cccn2)cc2onc(NS(=O)(=O)c3c(OC)ccc4c3OCCC4(C)C)c2c1F] | N-(6-((1H-Pyrazol-1-yl)methyl)-5-ethoxy-4- fluorobenzo[d]isoxazol-3-yl)-7-methoxy-4,4- dimethylchromane-8-sulfonamide |
| 87 | 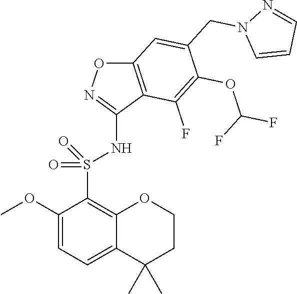 [SMILES: COc1ccc2c(c1S(=O)(=O)Nc1noc3cc(Cn4cccn4)c(OC(F)F)c(F)c13)OCCC2(C)C] | N-(6-((1H-Pyrazol-1-yl)methyl)-5-(difluoromethoxy)-4- fluorobenzo[d]isoxazol-3-yl)-7-methoxy-4,4- dimethylchromane-8-sulfonamide |
| 88 | 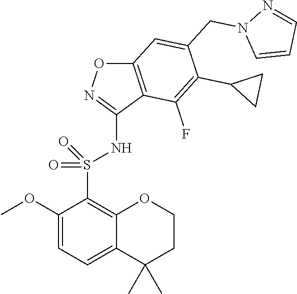 [SMILES: COc1ccc2c(c1S(=O)(=O)Nc1noc3cc(Cn4cccn4)c(C4CC4)c(F)c13)OCCC2(C)C] | N-(6-((1H-Pyrazol-1-yl)methyl)-5-cyclopropyl-4- fluorobenzo[d]isoxazol-3-yl)-7-methoxy-4,4- dimethylchromane-8-sulfonamide |
| 89 | 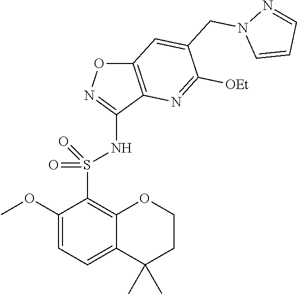 [SMILES: CCOc1nc2c(NS(=O)(=O)c3c(OC)ccc4c3OCCC4(C)C)noc2cc1Cn1cccn1] | N-(6-((1H-Pyrazol-1-yl)methyl)-5-ethoxyisoxazolo[4,5- b]pyridin-3-yl)-7-methoxy-4,4-dimethylchromane-8- sulfonamide |
| 90 | 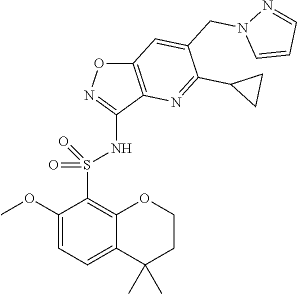 [SMILES: COc1ccc2c(c1S(=O)(=O)Nc1noc3cc(Cn4cccn4)c(C4CC4)nc13)OCCC2(C)C] | N-(6-((1H-Pyrazol-1-yl)methyl)-5-cyclopropylisoxazolo[4,5- b]pyridin-3-yl)-7-methoxy-4,4-dimethylchromane-8- sulfonamide |
| 91 | 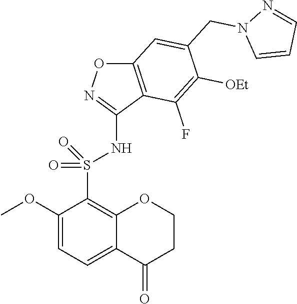 [SMILES: CCOc1c(Cn2cccn2)cc2onc(NS(=O)(=O)c3c(OC)ccc4c3OCCC4=O)c2c1F] | N-(6-((1H-Pyrazol-1-yl)methyl)-5-ethoxy-4- fluorobenzo[d]isoxazol-3-yl)-7-methoxy-4-oxochromane-8- sulfonamide |
| 92 | 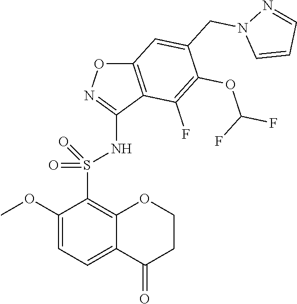 [SMILES: COc1ccc2c(c1S(=O)(=O)Nc1noc3cc(Cn4cccn4)c(OC(F)F)c(F)c13)OCCC2=O] | N-(6-((1H-Pyrazol-1-yl)methyl)-5-(difluoromethoxy)-4- fluorobenzo[d]isoxazol-3-yl)-7-methoxy-4-oxochromane-8- sulfonamide |
| 93 | 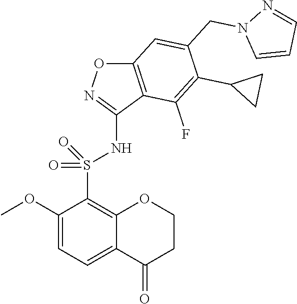 [SMILES: COc1ccc2c(c1S(=O)(=O)Nc1noc3cc(Cn4cccn4)c(C4CC4)c(F)c13)OCCC2=O] | N-(6-((1H-Pyrazol-1-yl)methyl)-5-cyclopropyl-4- fluorobenzo[d]isoxazol-3-yl)-7-methoxy-4-oxochromane-8- sulfonamide |
| 94 | 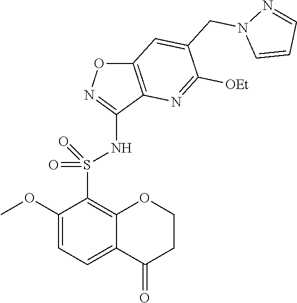 [SMILES: CCOc1nc2c(NS(=O)(=O)c3c(OC)ccc4c3OCCC4=O)noc2cc1Cn1cccn1] | N-(6-((1H-Pyrazol-1-yl)methyl)-5-ethoxyisoxazolo[4,5- b]pyridin-3-yl)-7-methoxy-4-oxochromane-8-sulfonamide |
| 95 | 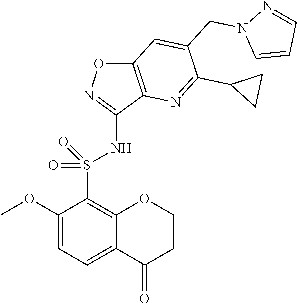 [SMILES: COc1ccc2c(c1S(=O)(=O)Nc1noc3cc(Cn4cccn4)c(C4CC4)nc13)OCCC2=O] | N-(6-((1H-Pyrazol-1-yl)methyl)-5-cyclopropylisoxazolo[4,5- b]pyridin-3-yl)-7-methoxy-4-oxochromane-8-sulfonamide |
| 96 | 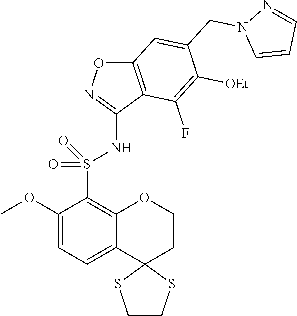 [SMILES: CCOc1c(Cn2cccn2)cc2onc(NS(=O)(=O)c3c(OC)ccc4c3OCCC43SCCS3)c2c1F] | N-(6-((1H-Pyrazol-1-yl)methyl)-5-ethoxy-4- fluorobenzo[d]isoxazol-3-yl)-7-methoxyspiro[chromane-4,2′- [1,3]dithiolane]-8-sulfonamide |
| 97 | 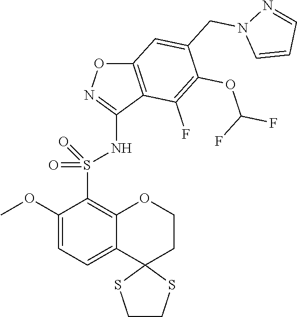 [SMILES: COc1ccc2c(c1S(=O)(=O)Nc1noc3cc(Cn4cccn4)c(OC(F)F)c(F)c13)OCCC21SCCS1] | N-(6-((1H-Pyrazol-1-yl)methyl)-5-(difluoromethoxy)-4- fluorobenzo[d]isoxazol-3-yl)-7-methoxyspiro[chromane-4,2′- [1,3]dithiolane]-8-sulfonamide |
| 98 | 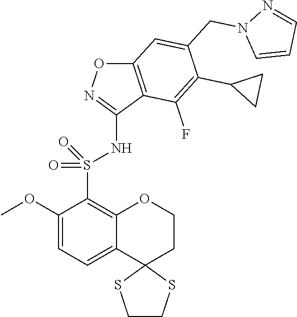 [SMILES: COc1ccc2c(c1S(=O)(=O)Nc1noc3cc(Cn4cccn4)c(C4CC4)c(F)c13)OCCC21SCCS1] | N-(6-((1H-Pyrazol-1-yl)methyl)-5-cyclopropyl-4- fluorobenzo[d]isoxazol-3-yl)-7-methoxyspiro[chromane-4,2′- [1,3]dithiolane]-8-sulfonamide |
| 99 | 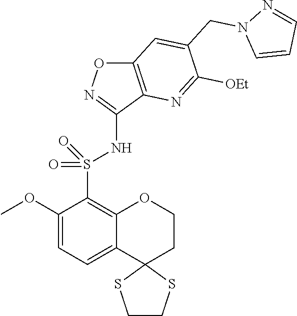 [SMILES: CCOc1nc2c(NS(=O)(=O)c3c(OC)ccc4c3OCCC43SCCS3)noc2cc1Cn1cccn1] | N-(6-((1H-Pyrazol-1-yl)methyl)-5-ethoxyisoxazolo[4,5- b]pyridin-3-yl)-7-methoxyspiro[chromane-4,2′- [1,3]dithiolane]-8-sulfonamide |
| 100 | 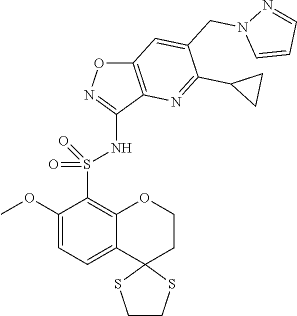 [SMILES: COc1ccc2c(c1S(=O)(=O)Nc1noc3cc(Cn4cccn4)c(C4CC4)nc13)OCCC21SCCS1] | N-(6-((1H-Pyrazol-1-yl)methyl)-5-cyclopropylisoxazolo[4,5- b]pyridin-3-yl)-7-methoxyspiro[chromane-4,2′- [1,3]dithiolane]-8-sulfonamide |
| 101 | 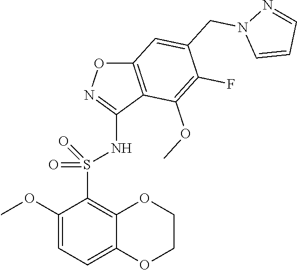 [SMILES: COc1ccc2c(c1S(=O)(=O)Nc1noc3cc(Cn4cccn4)c(F)c(OC)c13)OCCO2] | N-(6-((1H-Pyrazol-1-yl)methyl)-5-fluoro-4- methoxybenzo[d]isoxazol-3-yl)-7-methoxy-2H-chromene-8- sulfonamide |
| 102 | 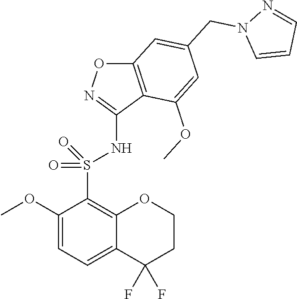 [SMILES: COc1ccc2c(c1S(=O)(=O)Nc1noc3cc(Cn4cccn4)cc(OC)c13)OCCC2(F)F] | N-(6-((1H-Pyrazol-1-yl)methyl)-4-methoxybenzo[d]isoxazol- 3-yl)-4,4-difluoro-7-methoxychromane-8-sulfonamide |
| 103 | 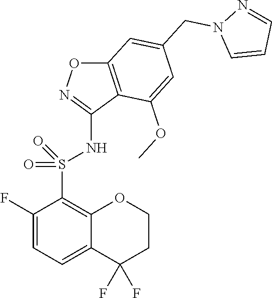 [SMILES: COc1cc(Cn2cccn2)cc2onc(NS(=O)(=O)c3c(F)ccc4c3OCCC4(F)F)c12] | N-(6-((1H-Pyrazol-1-yl)methyl)-4-methoxybenzo[d]isoxazol- 3-yl)-4,4,7-trifluorochromane-8-sulfonamide |
| 104 | 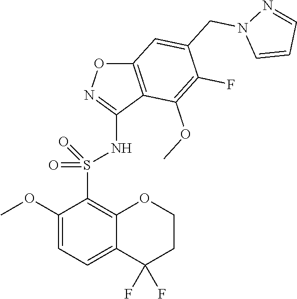 [SMILES: COc1ccc2c(c1S(=O)(=O)Nc1noc3cc(Cn4cccn4)c(F)c(OC)c13)OCCC2(F)F] | N-(6-((1H-Pyrazol-1-yl)methyl)-5-fluoro-4- methoxybenzo[d]isoxazol-3-yl)-4,4-difluoro-7- methoxychromane-8-sulfonamide |
| 105 | 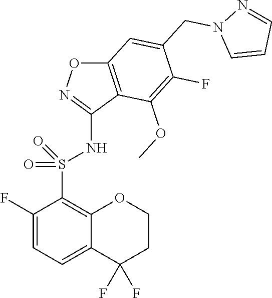 [SMILES: COc1c(F)c(Cn2cccn2)cc2onc(NS(=O)(=O)c3c(F)ccc4c3OCCC4(F)F)c12] | N-(6-((1H-Pyrazol-1-yl)methyl)-5-fluoro-4- methoxybenzo[d]isoxazol-3-yl)-4,4,7-trifluorochromane-8- sulfonamide |
| 106 | 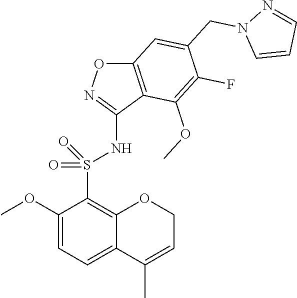 [SMILES: COc1ccc2c(c1S(=O)(=O)Nc1noc3cc(Cn4cccn4)c(F)c(OC)c13)OCC=C2C] | N-(6-((1H-Pyrazol-1-yl)methyl)-5-fluoro-4- methoxybenzo[d]isoxazol-3-yl)-7-methoxy-4-methyl-2H- chromene-8-sulfonamide |
| 107 | 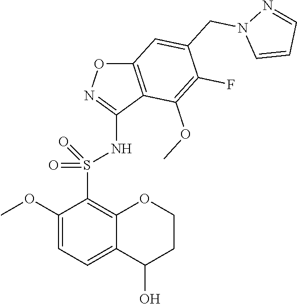 [SMILES: COc1ccc2c(c1S(=O)(=O)Nc1noc3cc(Cn4cccn4)c(F)c(OC)c13)OCCC2O] | N-(6-((1H-Pyrazol-1-yl)methyl)-5-fluoro-4- methoxybenzo[d]isoxazol-3-yl)-4-hydroxy-7- methoxychromane-8-sulfonamide |
| 108 | 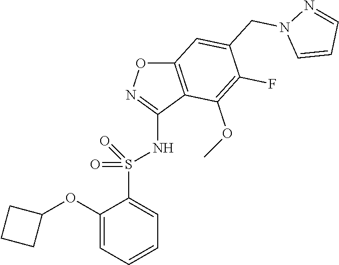 [SMILES: COc1c(F)c(Cn2cccn2)cc2onc(NS(=O)(=O)c3ccccc3OC3CCC3)c12] | N-(6-((1H-Pyrazol-1-yl)methyl)-5-fluoro-4- methoxybenzo[d]isoxazol-3-yl)-2- cyclobutoxybenzenesulfonamide |
| 109 | 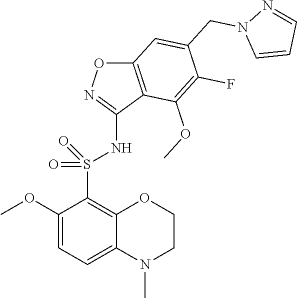 [SMILES: COc1ccc2c(c1S(=O)(=O)Nc1noc3cc(Cn4cccn4)c(F)c(OC)c13)OCCN2C] | N-(6-((1H-Pyrazol-1-yl)methyl)-5-fluoro-4- methoxybenzo[d]isoxazol-3-yl)-7-methoxy-4-methyl-3,4- dihydro-2H-benzo[b][1,4]oxazine-8-sulfonamide |
| 110 | 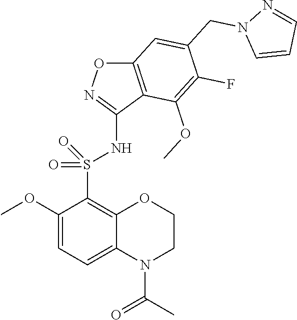 [SMILES: COc1ccc2c(c1S(=O)(=O)Nc1noc3cc(Cn4cccn4)c(F)c(OC)c13)OCCN2C(C)=O] | N-(6-((1H-Pyrazol-1-yl)methyl)-5-fluoro-4- methoxybenzo[d]isoxazol-3-yl)-4-acetyl-7-methoxy-3,4- dihydro-2H-benzo[b][1,4]oxazine-8-sulfonamide |
| 111 | 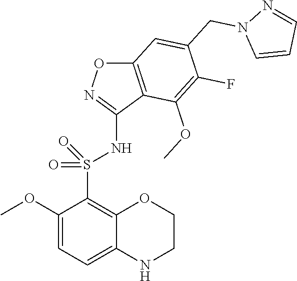 [SMILES: COc1ccc2c(c1S(=O)(=O)Nc1noc3cc(Cn4cccn4)c(F)c(OC)c13)OCCN2] | N-(6-((1/-Pyrazol-1-yl)methyl)-5-fluoro-4- methoxybenzo[d]isoxazol-3-yl)-7-methoxy-3,4-dihydro-2H- benzo[b][1,4]oxazine-8-sulfonamide |
| 112 | 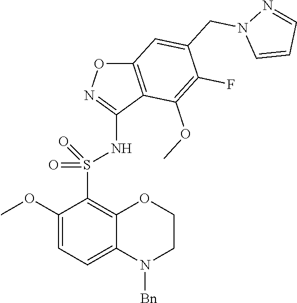 [SMILES: COc1ccc2c(c1S(=O)(=O)Nc1noc3cc(Cn4cccn4)c(F)c(OC)c13)OCCN2Cc1ccccc1] | N-(6-((1H-Pyrazol-1-yl)methyl)-5-fluoro-4- methoxybenzo[d]isoxazol-3-yl)-4-benzyl-7-methoxy-3,4- dihydro-2H-benzo[b][1,4]oxazine-8-sulfonamide |
| 113 | 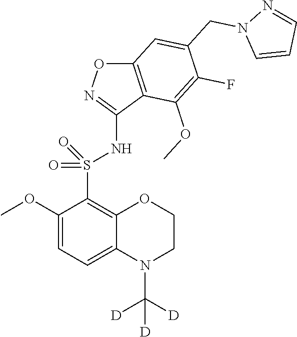 [SMILES: [2H]C([2H])([2H])N1CCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-7-methoxy-4-(methyl-d₃)- 3,4-dihydro-2H-benzo[b][1,4]oxazine-8-sulfonamide |
| 114 | 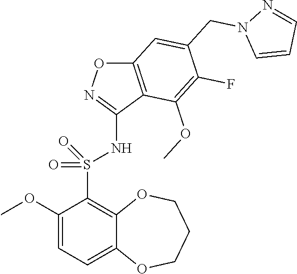 [SMILES: COc1ccc2c(c1S(=O)(=O)Nc1noc3cc(Cn4cccn4)c(F)c(OC)c13)OCCCO2] | N-(6-((1H-Pyrazol-1-yl)methyl)-5-fluoro-4- methoxybenzo[d]isoxazol-3-yl)-7-methoxy-3,4-dihydro-2H- benzo[b][1,4]dioxepine-6-sulfonamide |
| 115 | 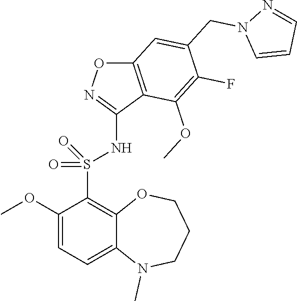 [SMILES: COc1ccc2c(c1S(=O)(=O)Nc1noc3cc(Cn4cccn4)c(F)c(OC)c13)OCCCN2C] | N-(6-((1H-Pyrazol-1-yl)methyl)-5-fluoro-4- methoxybenzo[d]isoxazol-3-yl)-8-methoxy-5-methyl-2,3,4,5- tetrahydrobenzo[b][1,4]oxazepine-9-sulfonamide |
| 116 | 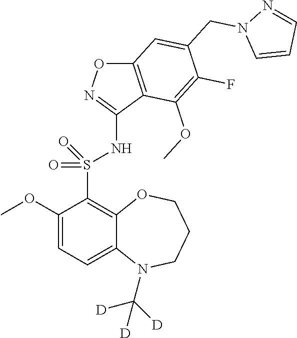 [SMILES: [2H]C([2H])([2H])N1CCCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-8-methoxy-5-(methyl-d₃)- 2,3,4,5-tetrahydrobenzo[b][1,4]oxazepine-9-sulfonamide |
| 117 | 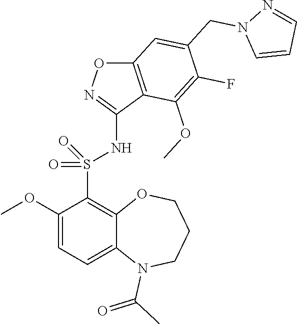 [SMILES: COc1ccc2c(c1S(=O)(=O)Nc1noc3cc(Cn4cccn4)c(F)c(OC)c13)OCCCN2C(C)=O] | N-(6-((1H-Pyrazol-1-yl)methyl)-5-fluoro-4- methoxybenzo[d]isoxazol-3-yl)-5-acetyl-8-methoxy-2,3,4,5- tetrahydrobenzo[b][1,4]oxazepine-9-sulfonamide |
| 118 | 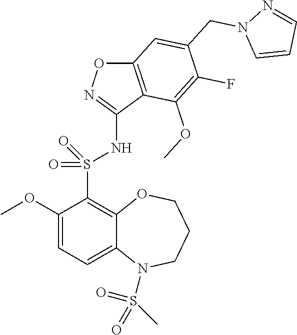 [SMILES: COc1ccc2c(c1S(=O)(=O)Nc1noc3cc(Cn4cccn4)c(F)c(OC)c13)OCCCN2S(C)(=O)=O] | N-(6-((1H-Pyrazol-1-yl)methyl)-5-fluoro-4- methoxybenzo[d]isoxazol-3-yl)-8-methoxy-5- (methylsulfonyl)-2,3,4,5-tetrahydrobenzo[b][1,4]oxazepine- 9-sulfonamide |
| 119 | 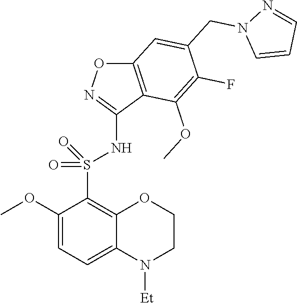 [SMILES: CCN1CCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-4-ethyl-7-methoxy-3,4- dihydro-2H-benzo[b][1,4]oxazine-8-sulfonamide |
| 120 | 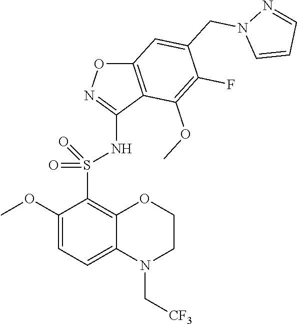 [SMILES: COc1ccc2c(c1S(=O)(=O)Nc1noc3cc(Cn4cccn4)c(F)c(OC)c13)OCCN2CC(F)(F)F] | N-(6-((1H-Pyrazol-1-yl)methyl)-5-fluoro-4- methoxybenzo[d]isoxazol-3-yl)-7-methoxy-4-(2,2,2- trifluoroethyl)-3,4-dihydro-2/-benzo[b][1,4]oxazine-8- sulfonamide |
| 121 | 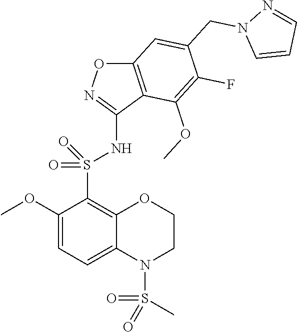 [SMILES: COc1ccc2c(c1S(=O)(=O)Nc1noc3cc(Cn4cccn4)c(F)c(OC)c13)OCCN2S(C)(=O)=O] | N-(6-((1H-Pyrazol-1-yl)methyl)-5-fluoro-4- methoxybenzo[d]isoxazol-3-yl)-7-methoxy-4- (methylsulfonyl)-3,4-dihydro-2H-benzo[b][1,4]oxazine-8- sulfonamide |
| 122 | 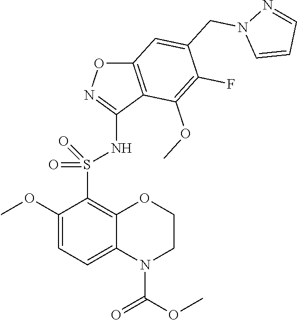 [SMILES: COC(=O)N1CCOc2c1ccc(OC)c2S(=O)(=O)Nc1noc2cc(Cn3cccn3)c(F)c(OC)c12] | Methyl 8-(N-(6-((1H-pyrazol-1-yl)methyl)-5-fluoro-4- methoxybenzo[d]isoxazol-3-yl)sulfamoyl)-7-methoxy-3,4- dihydro-4H-benzo[b][1,4]oxazine-4-carboxylate |
| 123 | 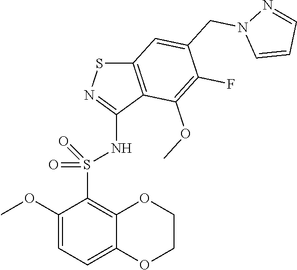 [SMILES: COc1ccc2c(c1S(=O)(=O)Nc1nsc3cc(Cn4cccn4)c(F)c(OC)c13)OCCO2] | N-(6-((1H-Pyrazol-1-yl)methyl)-5-fluoro-4- methoxybenzo[d]isothiazol-3-yl)-6-methoxy-2,3- dihydrobenzo[b][1,4]dioxine-5-sulfonamide |
| 124 | 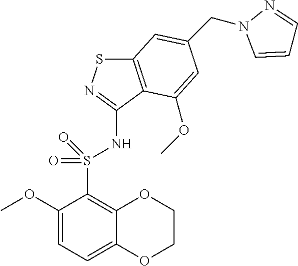 [SMILES: COc1ccc2c(c1S(=O)(=O)Nc1nsc3cc(Cn4cccn4)cc(OC)c13)OCCO2] | N-(6-((1H-Pyrazol-1-yl)methyl)-4- methoxybenzo[d]isothiazol-3-yl)-6-methoxy-2,3- dihydrobenzo[b][1,4]dioxine-5-sulfonamide |
| 125 | 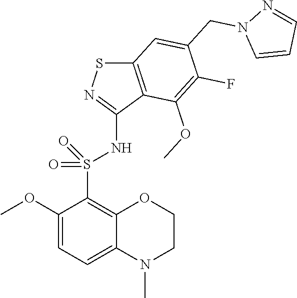 [SMILES: COc1ccc2c(c1S(=O)(=O)Nc1nsc3cc(Cn4cccn4)c(F)c(OC)c13)OCCN2C] | N-(6-((1H-Pyrazol-1-yl)methyl)-5-fluoro-4- methoxybenzo[d]isothiazol-3-yl)-7-methoxy-4-methyl-3,4- dihydro-2H-benzo[b][1,4]oxazine-8-sulfonamide |
| 126 | 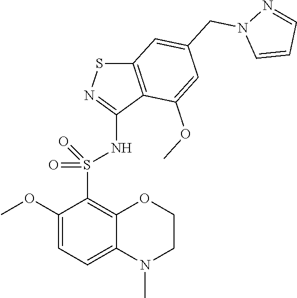 [SMILES: COc1ccc2c(c1S(=O)(=O)Nc1nsc3cc(Cn4cccn4)cc(OC)c13)OCCN2C] | N-(6-((1H-Pyrazol-1-yl)methyl)-4- methoxybenzo[d]isothiazol-3-yl)-7-methoxy-4-methyl-3,4- dihydro-2H-benzo[b][1,4]oxazine-8-sulfonamide |
| 127 | 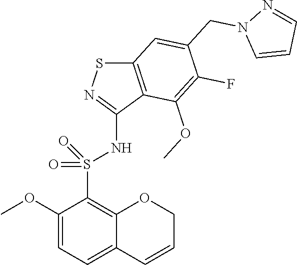 [SMILES: COc1ccc2c(c1S(=O)(=O)Nc1nsc3cc(Cn4cccn4)c(F)c(OC)c13)OCC=C2] | N-(6-((1H-Pyrazol-1-yl)methyl)-5-fluoro-4- methoxybenzo[d]isothiazol-3-yl)-7-methoxy-2H-chromene- 8-sulfonamide |
| 128 | 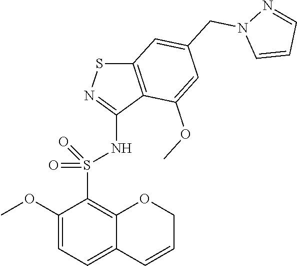 [SMILES: COc1ccc2c(c1S(=O)(=O)Nc1nsc3cc(Cn4cccn4)cc(OC)c13)OCC=C2] | N-(6-((1H-Pyrazol-1-yl)methyl)-4- methoxybenzo[d]isothiazol-3-yl)-7-methoxy-2H-chromene- 8-sulfonamide |
| 129 | 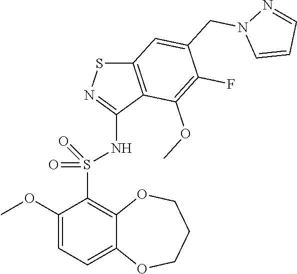 [SMILES: COc1ccc2c(c1S(=O)(=O)Nc1nsc3cc(Cn4cccn4)c(F)c(OC)c13)OCCCO2] | N-(6-((1H-Pyrazol-1-yl)methyl)-5-fluoro-4- methoxybenzo[d]isothiazol-3-yl)-7-methoxy-3,4-dihydro- 2H-benzo[b][1,4]dioxepine-6-sulfonamide |
| 130 | 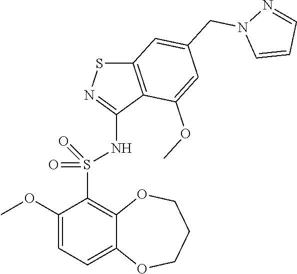 [SMILES: COc1ccc2c(c1S(=O)(=O)Nc1nsc3cc(Cn4cccn4)cc(OC)c13)OCCCO2] | N-(6-((1H-Pyrazol-1-yl)methyl)-4- methoxybenzo[d]isothiazol-3-yl)-7-methoxy-3,4-dihydro- 2H-benzo[b][1,4]dioxepine-6-sulfonamide |
| 131 | 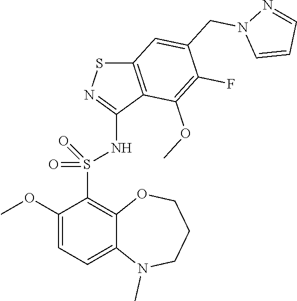 [SMILES: COc1ccc2c(c1S(=O)(=O)Nc1nsc3cc(Cn4cccn4)c(F)c(OC)c13)OCCCN2C] | N-(6-((1H-Pyrazol-1-yl)methyl)-5-fluoro-4- methoxybenzo[d]isothiazol-3-yl)-8-methoxy-5-methyl- 2,3,4,5-tetrahydrobenzo[b][1,4]oxazepine-9-sulfonamide |
| 132 | 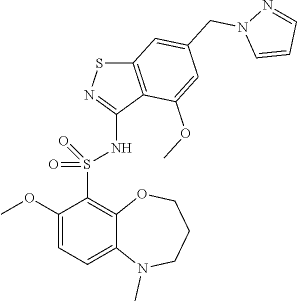 [SMILES: COc1ccc2c(c1S(=O)(=O)Nc1nsc3cc(Cn4cccn4)cc(OC)c13)OCCCN2C] | N-(6-((1H-Pyrazol-1-yl)methyl)-4- methoxybenzo[d]isothiazol-3-yl)-8-methoxy-5-methyl- 2,3,4,5-tetrahydrobenzo[b][1,4]oxazepine-9-sulfonamide |

The compounds of formula (I) (including compounds of any applicable sub-formulas as described herein or compounds listed in Table 1) may exist in the form of individual enantiomers, diastereomers, atropisomers, and/or geometric isomers (if applicable), or mixtures of stereoisomers (including racemic mixtures and mixtures enriched in one or more stereoisomers).

In some embodiments, where applicable, the compounds of formula (I) (including compounds of any applicable sub-formulas described herein or compounds listed in Table 1) may exist as mixtures of atropisomers in any ratio (including about 1:1). In some embodiments, where applicable, the compounds of formula (I) (including compounds of any applicable sub-formulas described herein or compounds listed in Table 1) may exist as isolated individual atropisomers, which are substantially free of other atropisomers (e.g., by weight, by HPLC area, or both, having less than 20%, less than 10%, less than 5%, less than 1%, or undetectable amounts of other atropisomers).

In some embodiments, the compounds described above are isotopically substituted. Preferably, in some embodiments, the isotopic substitution is a substitution with a deuterium atom.

Surprisingly, the compounds of the present disclosure exhibit significantly improved inhibitory activity against KAT6A/B, significantly improved pharmacodynamics and pharmacokinetics, and/or a significantly improved safety profile compared to KAT6A/B inhibitors known in the prior art.

### Synthetic Methods for Compounds of the Present Disclosure

In view of the present disclosure, those skilled in the art can readily synthesize the compounds of the present disclosure. Exemplary syntheses are shown in the “Examples” section.

The following synthetic methods for the compounds are exemplary. By using appropriate synthetic starting materials or intermediates, those skilled in the art can similarly apply the synthetic methods to synthesize other compounds.

To achieve the objectives of the present disclosure, the present disclosure adopts the following technical solutions:

A method for preparing the compound of formula (I) or the pharmaceutically acceptable salt thereof of the present disclosure, comprising the following steps:

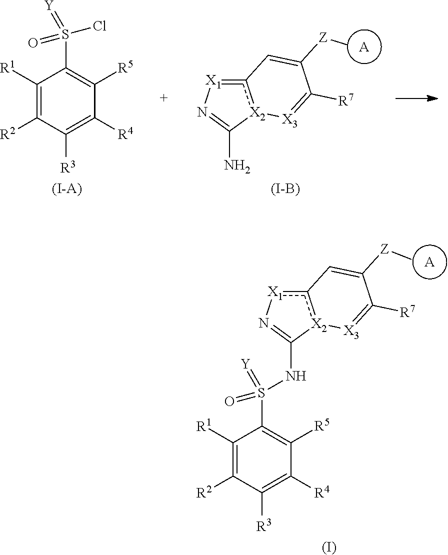

reacting a compound of formula (I-A) or a salt thereof with a compound of formula (I-B) or a salt thereof in the presence of a base to give the compound of formula (I) or the pharmaceutically acceptable salt thereof,
wherein:
the dashed line is an optional chemical bond, and
ring A, R¹, R², R³, R⁴, R⁵, R⁷, X₁, X₂, X₃, Y, and Z are as defined in the compound of formula (I) described above; or

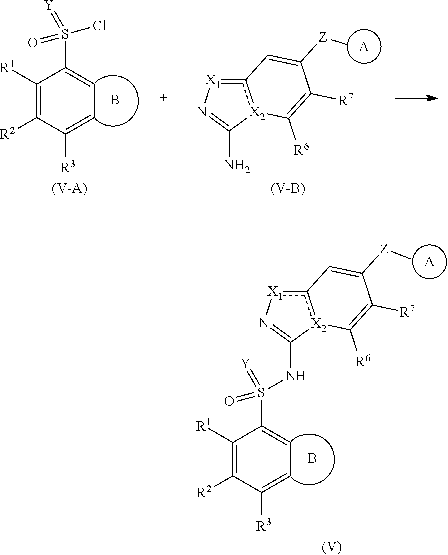

reacting a compound of formula (V-A) or a salt thereof with a compound of formula (V-B) or a salt thereof in the presence of a base to give a compound of formula (V) or a pharmaceutically acceptable salt thereof,
wherein:
the dashed line is an optional chemical bond, and
ring A, ring B, R¹, R², R³, R⁶, R⁷, X₁, X₂, Y, and Z are as defined in the compound of formula (V) described above; or

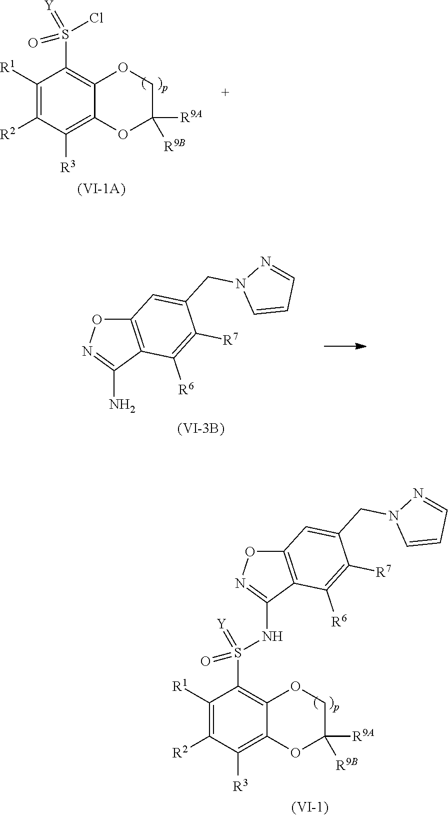

reacting a compound of formula (VI-1A) or a salt thereof with a compound of formula (VI-3B) or a salt thereof in the presence of a base to give a compound of formula (VI-1) or a pharmaceutically acceptable salt thereof,
wherein:
R¹, R², R³, R⁶, R⁷, R⁹A, R^{9B}, Y, and p are as defined in the compound of formula (VI-1) described above; or

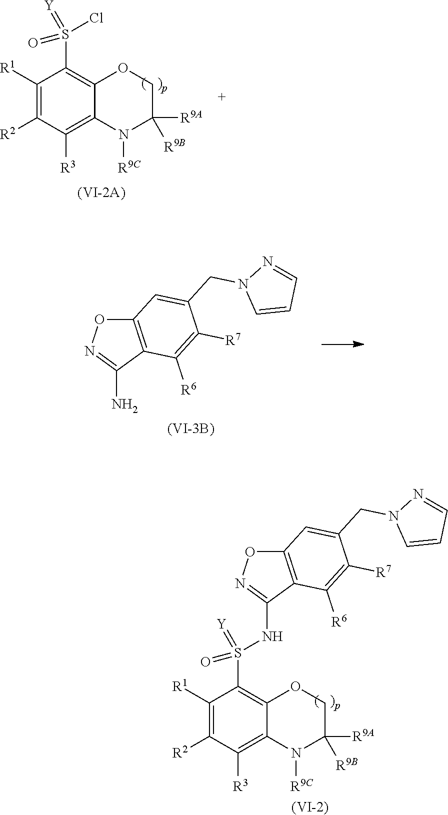

reacting a compound of formula (VI-2A) or a salt thereof with a compound of formula (VI-3B) or a salt thereof in the presence of a base to give a compound of formula (VI-2) or a pharmaceutically acceptable salt thereof,
wherein:
R¹, R², R³, R⁶, R⁷, R⁹A, R⁹B, R⁹c, Y, and p are as defined in the compound of formula (VI-2) described above; or

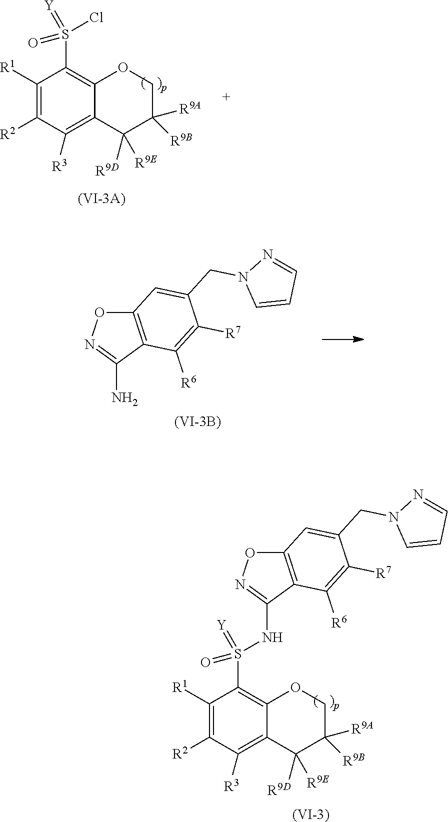

reacting a compound of formula (VI-3A) or a salt thereof with a compound of formula (VI-3B) or a salt thereof in the presence of a base to give a compound of formula (VI-3) or a pharmaceutically acceptable salt thereof,
wherein:
R¹, R², R³, R⁶, R⁷, R⁹A, R^{9B}, R^{9D}, R^{9E}, Y, and p are as defined in the compound of formula (VI-3) described above; or

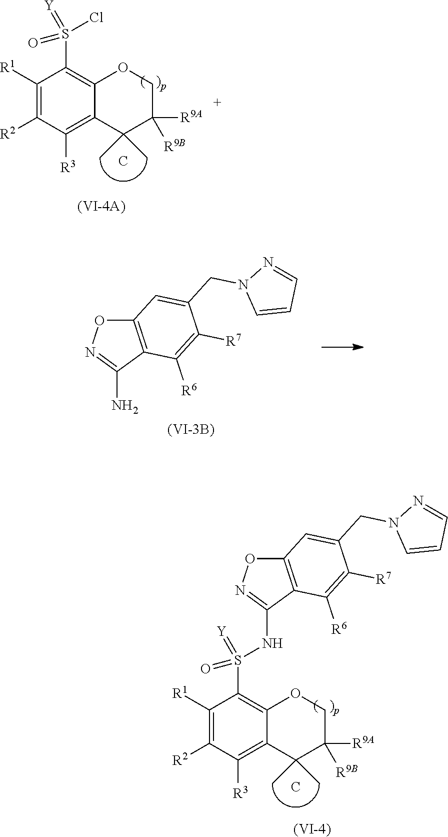

reacting a compound of formula (VI-4A) or a salt thereof with a compound of formula (VI-3B) or a salt thereof in the presence of a base to give a compound of formula (VI-4) or a pharmaceutically acceptable salt thereof,
wherein:
ring C, R¹, R², R³, R⁶, R⁷, R⁹A, R^{9B}, Y, and p are as defined in the compound of formula (VI-4) described above; or

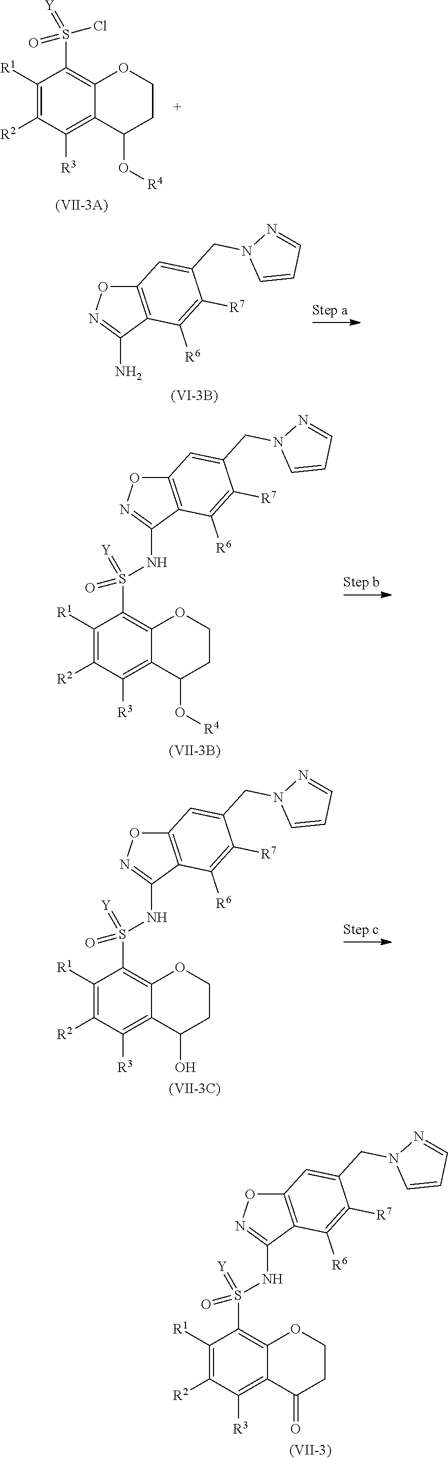

step a: reacting a compound of formula (VII-3A) or a salt thereof with a compound of formula (VI-3B) or a salt thereof in the presence of a base to give a compound of formula (VII-3B) or a pharmaceutically acceptable salt thereof;
step b: reacting the compound of formula (VII-3B) or the salt thereof with a deprotecting reagent to remove a hydroxyl protecting group to give a compound of formula (VII-3C) or a pharmaceutically acceptable salt thereof; and
step c: reacting the compound of formula (VII-3C) or the salt thereof with an oxidizing reagent for oxidation to give a compound of formula (VII-3) or a pharmaceutically acceptable salt thereof;
wherein:
R¹, R², R³, R⁶, R⁷, and Y are as defined in the compound of formula (VII-3) described above;
R^{a} is the hydroxyl protecting group, preferably (trimethylsilyl)ethoxymethyl; the deprotecting reagent in step b is an acid or a fluorine-containing reagent, preferably trifluoroacetic acid or tetrabutylammonium fluoride;
the oxidizing reagent in step c is a reagent for oxidizing a hydroxyl group to a ketone, preferably manganese dioxide or Dess-Martin reagent.

The base used in the reactions of the above schemes is selected from: triethylamine, diisopropylethylamine, pyridine, 2,4-dimethylpyridine, 2,6-dimethylpyridine, n-butyllithium, lithium bis(trimethylsilyl)amide, sodium hydride, sodium hydroxide, cesium carbonate, and potassium carbonate.

The reactions of the above schemes are preferably carried out in a solvent selected from: ethylene glycol dimethyl ether, methanol, ethanol, acetonitrile, n-butanol, toluene, tetrahydrofuran, dichloromethane, dimethyl sulfoxide, 1,4-dioxane, N,N-dimethylformamide, N,N-dimethylacetamide, 1,2-dibromoethane, toluene, pyridine, and a mixture thereof.

It will be apparent to those skilled in the art that conventional protecting groups may be required to prevent certain functional groups from undergoing undesired reactions. Suitable protecting groups for various functional groups and suitable conditions for protecting and deprotecting particular functional groups are well known in the art. For example, many protecting groups are described in “Protective Groups in Organic Synthesis”, 4th ed. P.G.M. Wuts; T.W. Greene, John Wiley, 2007, and the references cited therein. The reagents used in the reactions described herein are generally known compounds or can be prepared by known procedures or obvious modifications thereof. For example, some reagents suitable for use in the reactions described herein can be prepared by following the corresponding procedures described in WO2019/103952, the content of which is incorporated herein by reference in its entirety. Furthermore, many reagents are available from commercial suppliers, such as Aldrich Chemical Co. (Milwaukee, Wisconsin, USA) and Sigma (St. Louis, Missouri, USA). Other reagents can be prepared by procedures described in standard reference books or obvious modifications thereof, such as: Fieser and Fieser's Reagents for Organic Synthesis, Volumes 1-15 (John Wiley and Sons, 1991); Rodd's Chemistry of Carbon Compounds, Volumes 1-5 and Supplemental (Elsevier Science Publishers, 1989); Organic Reactions, Volumes 1-40 (John Wiley and Sons, 1991); March's Advanced Organic Chemistry, (Wiley, 7th ed.); and Larock's Comprehensive Organic Transformations (Wiley-VCH, 1999); as well as any updates available as of the present application.

### Pharmaceutical Composition

Certain embodiments relate to a pharmaceutical composition comprising one or more of the compounds of the present disclosure.

The pharmaceutical composition may optionally comprise a pharmaceutically acceptable excipient. In some embodiments, the pharmaceutical composition comprises the compound of the present disclosure (e.g., the compound of formula (I) to (VII-3), any one of Compound Nos. 1-132, or a pharmaceutically acceptable salt thereof) and a pharmaceutically acceptable excipient.

Pharmaceutically acceptable excipients are known in the art. Non-limiting suitable excipients include, for example, encapsulating materials or additives, such as absorption enhancers, antioxidants, binders, buffers, carriers, coating agents, colorants, diluents, disintegrants, emulsifiers, extenders, fillers, flavoring agents, humectants, lubricants, perfumes, preservatives, propellants, releasing agents, sterilizing agents, sweeteners, solubilizers, wetting agents, and mixtures thereof. See also Remington's The Science and Practice of Pharmacy, 21st ed., A.R. Gennaro (Lippincott, Williams & Wilkins, Baltimore, Md., 2005; incorporated herein by reference), which discloses various excipients for formulating pharmaceutical compositions and known techniques for preparing pharmaceutical compositions.

The pharmaceutical composition may comprise any one or more of the compounds of the present disclosure. For example, in some embodiments, the pharmaceutical composition comprises, for example, a therapeutically effective amount of the compound of formula (I) to (VII-3), any one of Compound Nos. 1-132, or a pharmaceutically acceptable salt thereof. In any of the embodiments described herein, the pharmaceutical composition may comprise a therapeutically effective amount of a compound selected from Compound Nos. 1-132, or a pharmaceutically acceptable salt thereof.

The pharmaceutical composition may also be formulated for delivery via any known route of delivery, including but not limited to oral delivery, parenteral delivery, inhalation, etc.

In some embodiments, the pharmaceutical composition may be formulated for oral administration. Oral formulations may be presented as: discrete units, such as capsules, pills, cachets, lozenges, or tablets, each containing a predetermined amount of the active compound; powders or granules; solutions or suspensions in aqueous or non-aqueous liquids; or oil-in-water or water-in-oil emulsions. Excipients for preparing orally administered compositions are known in the art. Non-limiting suitable excipients include, for example, agar, alginic acid, aluminum hydroxide, benzyl alcohol, benzyl benzoate, 1,3-butanediol, carbomer, castor oil, cellulose, cellulose acetate, cocoa butter, corn starch, corn oil, cottonseed oil, crospovidone, diglycerides, ethanol, ethyl cellulose, ethyl laurate, ethyl oleate, fatty acid esters, gelatin, germ oil, glucose, glycerol, peanut oil, hydroxypropyl methylcellulose, isopropanol, isotonic saline, lactose, magnesium hydroxide, magnesium stearate, malt, mannitol, monoglycerides, olive oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acid, stearyl fumarate, sucrose, surfactants, talc, tragacanth, tetrahydrofurfuryl alcohol, triglycerides, water, and mixtures thereof. In some embodiments, the pharmaceutical composition is formulated for parenteral administration (e.g., intravenous injection or infusion, or subcutaneous or intramuscular injection). Parenteral formulations may be, for example, aqueous solutions, suspensions, or emulsions. Excipients for preparing parenteral formulations are known in the art. Non-limiting suitable excipients include, for example, 1,3-butanediol, castor oil, corn oil, cottonseed oil, glucose, germ oil, peanut oil, liposomes, oleic acid, olive oil, Ringer's solution, safflower oil, sesame oil, soybean oil, U.S.P. or isotonic sodium chloride solution, water, and mixtures thereof. In some embodiments, the pharmaceutical composition is formulated for inhalation administration. For example, inhalable formulations may be formulated as nasal sprays, dry powders, or as aerosols that can be administered via metered dose inhalers. Excipients for preparing inhalation formulations are known in the art. Non-limiting suitable excipients include, for example, lactose, talc, silicic acid, aluminum hydroxide, calcium silicate, and polyamide powder, as well as mixtures of these substances. Sprays may also contain propellants, such as chlorofluorocarbons and volatile unsubstituted hydrocarbons, for example, butane and propane. The pharmaceutical composition may comprise various amounts of the compound of the present disclosure, depending on various factors, such as the intended use, potency, and selectivity of the compound. In some embodiments, the pharmaceutical composition comprises a therapeutically effective amount of the compound of the present disclosure (e.g., the compound of formula I to VII-3, any one of Compound Nos. 1-132, or a pharmaceutically acceptable salt thereof). In some embodiments, the pharmaceutical composition comprises a therapeutically effective amount of the compound of the present disclosure and a pharmaceutically acceptable excipient. As used herein, the therapeutically effective amount of the compound of the present disclosure is an amount effective for treating a cancer as described herein, such as lung cancer, breast cancer, rectal cancer, colon cancer, esophageal cancer, gastric cancer, liver cancer, gallbladder cancer, cholangiocarcinoma, kidney cancer, bladder cancer, urothelial cancer, head and neck cancer, nasopharyngeal cancer, prostate cancer, cervical cancer/endometrial cancer, ovarian cancer, pancreatic cancer, melanoma, bone cancer, mesothelioma, gastrointestinal stromal tumor, sarcoma, brain glioma, thyroid cancer, salivary gland tumor, glioblastoma, neuroblastoma, gastric myxoma, lymphoma, leukemia, plasmacytoma, sinoatrial node tumor, or tenosynovial giant cell tumor. This amount may depend on the recipient of treatment, the cancer being treated and the severity thereof, the composition containing the compound, the time of administration, the route of administration, the duration of treatment, the potency of the compound (e.g., the potency for inhibiting KAT6A/B), its clearance rate, and whether another drug is co-administered.

For veterinary use, the compounds of the present disclosure may be administered in appropriately acceptable formulations according to normal veterinary practice. A veterinarian can readily determine the most appropriate administration regimen and route of administration for a particular animal.

In some embodiments, all necessary ingredients for treating such diseases using the compound of the present disclosure, either alone or in combination with another agent or intervention conventionally used for treating a cancer associated with KAT6A/B, can be packaged into a kit. Specifically, in some embodiments, the present disclosure provides a kit for therapeutic intervention for a disease, comprising a packaged drug set, wherein the drug set comprises the compound disclosed herein, as well as a buffer and other components for preparing the drug in a deliverable form, and/or a device for delivering such drugs, and/or any agent for combination therapy with the compound of the present disclosure, and/or instructions for use packaged together with the drug for treating the disease. The instructions for use may be fixed in any tangible medium, such as printed paper or a computer-readable magnetic or optical medium, or may indicate reference to a remote computer data source, such as a World Wide Web webpage accessible via the Internet.

### Treatment Methods/Uses

The compounds of the present disclosure can be used as therapeutically active substances for treating and/or preventing cancers associated with KAT6A/B. In particular, the compounds of the present disclosure, by acting on KAT6A/B to mediate signal transduction, can be used to treat disorders associated with the modulation of KAT6A/B function, especially the inhibition of KAT6A/B function. Such disorders include diseases in which the pathogenic mechanisms are associated with these cytokines mediated by KAT6A/B, and they encompass any one of those diseases known in the art and those diseases described herein.

In some embodiments, the present disclosure provides a method for inhibiting KAT6A/B, comprising contacting a cell with an effective amount of one or more of the compounds of the present disclosure (e.g., the compound of formula I to VII-3, any one of Compound Nos. 1-132, or a pharmaceutically acceptable salt thereof).

In some embodiments, the present disclosure provides a method for inhibiting KAT6A/B function in a subject in need, comprising administering to the subject an effective amount of one or more of the compounds of the present disclosure (e.g., the compound of formula I to VII-3, any one of Compound Nos. 1-132, or a pharmaceutically acceptable salt thereof).

In some embodiments, the present disclosure provides a method for treating or preventing a KAT6A/B-mediated cancer in a subject in need, comprising administering to the subject an effective amount of one or more of the compounds of the present disclosure (e.g., the compound of formula I to VII-3, any one of Compound Nos. 1-132, or a pharmaceutically acceptable salt thereof). Suitable KAT6A/B-mediated cancers that can be treated using the method described herein include any of those cancers known in the art. Exemplary KAT6A/B-mediated cancers that can be treated using the method described herein also include, but are not limited to, lung cancer, breast cancer, rectal cancer, colon cancer, esophageal cancer, gastric cancer, liver cancer, gallbladder cancer, cholangiocarcinoma, kidney cancer, bladder cancer, urothelial cancer, head and neck cancer, nasopharyngeal cancer, prostate cancer, cervical cancer/endometrial cancer, ovarian cancer, pancreatic cancer, melanoma, bone cancer, mesothelioma, gastrointestinal stromal tumor, sarcoma, brain glioma, thyroid cancer, salivary gland tumor, glioblastoma, neuroblastoma, gastric myxoma, lymphoma, leukemia, plasmacytoma, sinoatrial node tumor, and tenosynovial giant cell tumor described herein.

In some embodiments, the present disclosure provides a method for treating or preventing, for example, the cancer described herein, in a subject in need, comprising administering to the subject an effective amount of one or more of the compounds of the present disclosure (e.g., the compound of formula I to VII-3, any one of Compound Nos. 1-132, or a pharmaceutically acceptable salt thereof).

In some embodiments, the present disclosure provides a method for treating or preventing a cancer in a subject in need, comprising administering to the subject an effective amount of one or more of the compounds of the present disclosure (e.g., the compound of formula I to VII-3, any one of Compound Nos. 1-132, or a pharmaceutically acceptable salt thereof), wherein the cancer may be one or more cancers selected from: lung cancer, breast cancer, rectal cancer, colon cancer, esophageal cancer, gastric cancer, liver cancer, gallbladder cancer, cholangiocarcinoma, kidney cancer, bladder cancer, urothelial cancer, head and neck cancer, nasopharyngeal cancer, prostate cancer, cervical cancer/endometrial cancer, ovarian cancer, pancreatic cancer, melanoma, bone cancer, mesothelioma, gastrointestinal stromal tumor, sarcoma, brain glioma, thyroid cancer, salivary gland tumor, glioblastoma, neuroblastoma, gastric myxoma, lymphoma, leukemia, plasmacytoma, sinoatrial node tumor, and tenosynovial giant cell tumor, wherein the breast cancer is preferably ER⁺ breast cancer or ER⁺/HER2⁻ breast cancer, the lung cancer is preferably non-small cell lung cancer, and the prostate cancer is preferably castration-resistant prostate cancer.

The administration described herein is not limited to any particular route of administration. For example, in some embodiments, the administration may be oral, nasal, transdermal, pulmonary, inhalation, buccal, sublingual, intraperitoneal, subcutaneous, intramuscular, intravenous, rectal, intrapleural, intrathecal, and parenteral. In some embodiments, the administration is oral administration.

The administration regimen, including the dose, can vary and can be adjusted, which may depend on the recipient of treatment, the cancer being treated and the severity thereof, the composition containing the compound, the time of administration, the route of administration, the duration of treatment, the potency of the compound, its clearance rate, and whether another drug is co-administered.

### EXAMPLES

The present disclosure is further described with reference to the following examples; however, these examples are not intended to limit the scope of the present disclosure.

The structures of the compounds were determined by nuclear magnetic resonance (NMR) spectroscopy or/and mass spectrometry (MS). The NMR shifts (δ) are given in 10⁻⁶ (ppm). The NMR analyses were performed using a Bruker AVANCE NEO 500M nuclear magnetic resonance system, with dimethyl sulfoxide-D6 (DMSO-d₆), chloroform-D (CDCl₃), and methanol-D4 (CD₃OD) as solvents and tetramethylsilane (TMS) as an internal standard.

The MS analyses were performed using an Agilent 1200/1290 DAD-6110/6120 Quadrupole MS liquid chromatography-mass spectrometry system (manufacturer: Agilent; MS model: 6110/6120 Quadrupole MS), Waters ACQuity UPLC-QD/SQD (manufacturer: Waters; MS model: Waters ACQuity Qda Detector/Waters SQ Detector), and THERMO Ultimate 3000-Q Exactive (manufacturer: THERMO; MS model: THERMO Q Exactive).

The high-performance liquid chromatography (HPLC) analyses were performed using Agilent HPLC 1200DAD, Agilent HPLC 1200VWD, and Waters HPLC e2695-2489 high-performance liquid chromatographs.

The chiral HPLC analyses were performed using an Agilent 1260 DAD high-performance liquid chromatograph.

The preparative high-performance liquid chromatography was performed using Waters 2767, Waters 2767-SQ Detecor2, Shimadzu LC-20AP, and Gilson-281 preparative chromatographs.

The preparative chiral chromatography was performed using a Shimadzu LC-20AP preparative chromatograph.

The CombiFlash preparative flash chromatograph used was CombiFlash Rf200 (TELEDYNE ISCO).

The thin-layer chromatography (TLC) silica gel plates used were Yantai Huanghai HSGF254 silica gel plates. The silica gel plates used in the TLC analyses had a layer thickness of 0.15 mm-0.2 mm, and those used in the TLC separation and purification for products had a layer thickness of 0.4 mm-0.5 mm.

Silica gel column chromatography generally used 200-300 mesh or 300-400 mesh silica gel as the carrier.

The mean kinase inhibition rates and IC₅₀ values were measured using a NovoStar microplate reader (BMG, Germany).

The known starting materials in the present disclosure may be synthesized by using or according to methods known in the art, or may be purchased from companies such as Shanghai Titan Scientific, ABCR GmbH & Co. KG, Acros Organics, Aldrich Chemical Company, Accela ChemBio Inc., Bide Pharmatech, etc.

In the examples, the reactions could all be performed under an argon atmosphere or a nitrogen atmosphere unless otherwise specified.

The argon atmosphere or nitrogen atmosphere means that the reaction flask was connected to a balloon containing about 1 μL of argon or nitrogen gas.

The hydrogen atmosphere means that the reaction flask was connected to a balloon containing about 1 μL of hydrogen gas.

The pressurized hydrogenation reactions were performed using a Parr 3916EKX hydrogenation apparatus and a Qinglan QL-500 hydrogen generator, or an HC2-SS hydrogenation apparatus.

The hydrogenation reactions generally involved 3 cycles of vacuumization and hydrogen filling.

The microwave reactions were performed using a CEM Discover-S 908860 microwave reactor.

In the examples, the solutions were aqueous solutions unless otherwise specified.

In the examples, the reaction temperature was room temperature, i.e., 20° C.-30° C., unless otherwise specified.

The monitoring of reaction progress in the examples was performed using thin-layer chromatography (TLC). The developing solvents used in the reactions, the eluent systems used in the column chromatography purification of compounds, and the developing solvent systems used in the thin-layer chromatography include: A: n-hexane/ethyl acetate system, and B: dichloromethane/methanol system. The volume ratio of the solvents was adjusted depending on the polarity of the compound, or a small amount of a basic or acidic reagent such as triethylamine and acetic acid could be added for adjustment.

### Example 1

### Synthesis of Intermediate 1

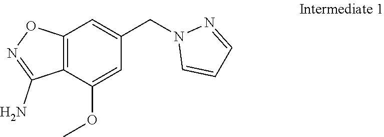 [SMILES: COc1cc(Cn2cccn2)cc2onc(N)c12]

### 6-((1H-Pyrazol-1-yl)methyl)-4-methoxybenzo[d]isoxazol-3-amine

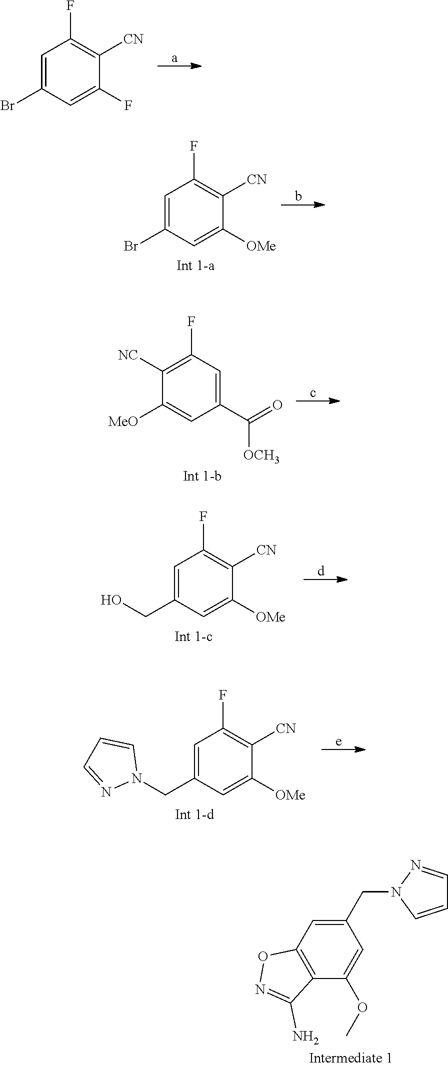 [SMILES: N#Cc1c(F)cc(Br)cc1F ; COc1cc(Br)cc(F)c1C#N ; COC(=O)c1cc(F)c(C#N)c(OC)c1 ; COc1cc(CO)cc(F)c1C#N ; COc1cc(Cn2cccn2)cc(F)c1C#N ; COc1cc(Cn2cccn2)cc2onc(N)c12]

### Step a:

### 4-Bromo-2-fluoro-6-methoxybenzonitrile

A sodium methoxide solution (9.92 g, 55.09 mmol, 30 wt % in methanol) was added to a solution of 4-bromo-2,6-difluorobenzonitrile (10.00 g, 45.87 mmol) in tetrahydrofuran (60 mL) at 0° C. The reaction mixture was stirred at 25° C. for 16 h, diluted with water, and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound Int 1-a (2.72 g, yield: 25.8%).

¹H NMR (300 MHz, CDCl₃): δ 7.02-6.99 (m, 1H), 6.94 (s, 1H), 3.96 (s, 3H).

### Step b:

### Methyl 4-cyano-3-fluoro-5-methoxybenzoate

Triethylamine (14 mL, 100.07 mmol) and [1,1′-bis(diphenylphosphino)ferrocene]palladium(II) dichloride (2.55 g, 3.49 mmol) were added to a solution of compound Int 1-a (8.00 g, 34.78 mmol) in methanol (80 mL). The reaction system was stirred at 80° C. for 16 h under a carbon monoxide atmosphere (3.0 MPa). After the reaction was completed, the reaction mixture was cooled to room temperature, diluted with water, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound Int 1-b (6.43 g, yield: 88.4%). ¹HNMR (300 MHz, CDCl₃): δ7.46-7.38 (m, 2H), 4.01 (s, 3H), 3.95 (s, 3H).

### Step c:

### 2-Fluoro-4-(hydroxymethyl)-6-methoxybenzonitrile

Lithium borohydride (0.74 g, 33.98 mmol) was added to a solution of compound Int 1-b (3.57 g, 17.07 mmol) in tetrahydrofuran (30 mL) at 0° C. The reaction system was heated to 80° C. and then stirred for 2 h. After the reaction was completed, the reaction mixture was cooled to room temperature, then quenched with a saturated aqueous sodium bicarbonate solution (20 mL), washed with water (20 mL), and extracted with ethyl acetate (20 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound Int 1-c (2.14 g, yield: 69.2%). ¹HNMR (300 MHz, DMSO-d₆): δ7.05 (s, 1H), 6.98 (d, 1H), 5.60 (t, 1H), 4.57 (d, 2H), 3.94 (s, 3H).

### Step d:

### 4-((1H-Pyrazol-1-yl)methyl)-2-fluoro-6-methoxybenzonitrile

Cesium carbonate (4.61 g, 14.15 mmol) was added to a solution of compound Int 1-c (2.14 g, 11.81 mmol) and 1-(methylsulfonyl)-1H-pyrazole (2.07 g, 14.16 mmol) in tetrahydrofuran (30 mL). The reaction system was heated to 70° C. and stirred for 1 h. After the reaction was completed, the reaction mixture was cooled to room temperature, then diluted with water, and extracted with ethyl acetate (25 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound Int 1-d (2.15 g, yield: 78.7%).

MS m/z (ESI): 232.0 [M+H]⁺.

### Step e:

### 6-((1H-Pyrazol-1-yl)methyl)-4-methoxybenzo[d]isoxazol-3-amine

Tetramethylguanidine (8.5 mL, 67.60 mmol) was added dropwise to compound Int 1-d (2.59 g, 11.20 mmol) and acetohydroxamic acid (2.52 g, 33.57 mmol) in a mixed solution (N,N-dimethylformamide and water in a ratio of 9:1, 30 mL). The reaction system was heated to 60° C. and stirred for 7 h. After the reaction was completed, the reaction mixture was cooled to room temperature, then diluted with water (50 mL), and extracted with ethyl acetate (30 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound intermediate 1 (2.17 g, yield: 79.3%). ¹HNMR (300 MHz, DMSO-d₆): δ 7.87 (d, 1H), 7.50 (d, 1H), 6.69 (s, 1H), 6.63 (s, 1H), 6.30 (t, 1H), 5.95 (s, 2H), 5.41 (s, 2H), 3.85 (s, 3H).

MS m/z (ESI): 245.1 [M+H]⁺.

### Example 2

### Synthesis of Intermediate 2

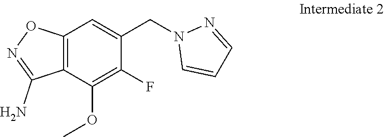 [SMILES: COc1c(F)c(Cn2cccn2)cc2onc(N)c12]

### 6-((1H-Pyrazol-1-yl)methyl)-5-fluoro-4-methoxybenzo[d]isoxazol-3-amine

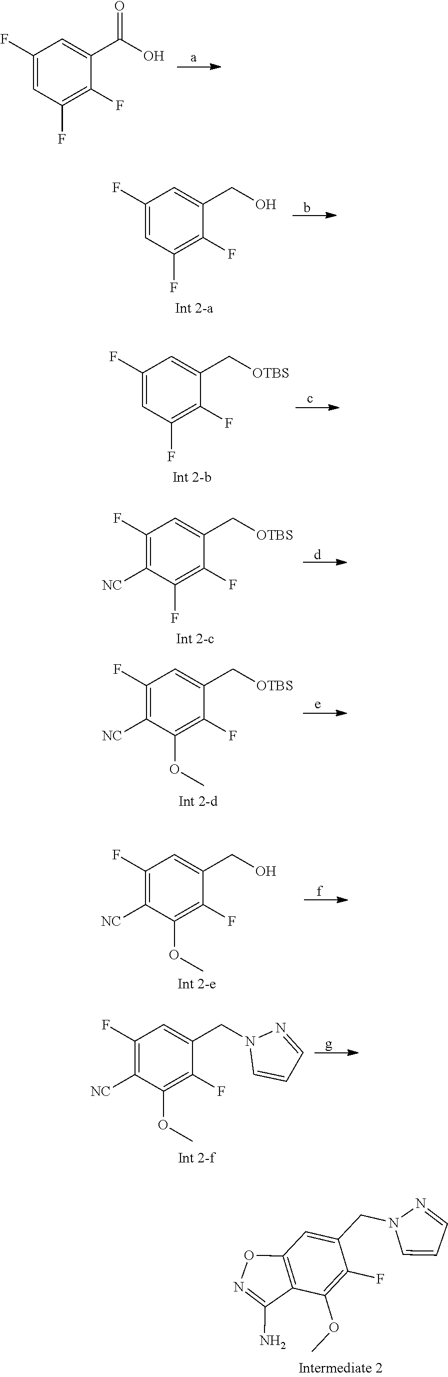

### Step a:

### (2,3,5-Trifluorophenyl)methanol

A solution of borane in tetrahydrofuran (1.0 M in tetrahydrofuran, 123 mL, 122.66 mmol) was added to a solution of 2,3,5-trifluorobenzoic acid (7.20 g, 40.89 mmol) in tetrahydrofuran (70 mL) at 25° C. The reaction system was heated to 50° C. and stirred for 4 h. After the reaction was completed, the mixture was cooled to room temperature, quenched with a 1 N aqueous hydrochloric acid solution (50 mL), and extracted with ethyl acetate (500 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound Int 2-a (6.03 g, yield: 91.0%).

¹H NMR (400 MHz, CDCl₃): δ7.01-6.97 (m, 1H), 6.89-6.82 (m, 1H), 4.79 (s, 2H).

### Step b:

### tert-Butyldimethyl((2,3, 5-trifluorobenzyl)oxy)silane

Compound Int 2-a (7.48 g, 46.14 mmol), tert-butyldimethylsilyl chloride (8.34 g, 55.33 mmol), triethylamine (7.00 g, 69.18 mmol), and 4-dimethylaminopyridine (0.56 g, 4.58 mmol) were dissolved in a solution of dichloromethane (80 mL). The reaction system was stirred at 25° C. for 16 h. After the reaction was completed, the reaction mixture was cooled to room temperature, diluted with water, and extracted with dichloromethane (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound Int 2-b (11.40 g, yield: 89.4%).

¹H NMR (400 MHz, CDCl₃): δ7.02-6.98 (m, 1H), 6.82-6.77 (m, 1H), 4.79 (s, 2H), 0.95 (s, 9H), 0.13 (s, 6H).

### Step c:

### 4-(((tert-Butyldimethylsilyl)oxy)methyl)-2,3,6-trifluorobenzonitrile

Lithium diisopropylamide (2.0 M in n-hexane, 18.50 mL, 37.00 mmol) was added to a solution of compound Int 2-b (6.80 g, 24.60 mmol) in tetrahydrofuran (80 mL) at −78° C. After the reaction system was stirred at −78° C. for 1 h, 4-toluenesulfonyl cyanide (4.90 g, 27.04 mmol) was added to the system, and stirring was continued for 2 h. After the reaction was completed, the reaction mixture was warmed to room temperature, quenched with a saturated ammonium chloride solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound Int 2-c (3.60 g, yield: 48.5%).

¹H NMR (400 MHz, CDCl₃): δ7.23-7.19 (m, 1H), 4.83 (s, 2H), 0.95 (s, 9H), 0.14 (s, 6H).

### Step d:

### 4-(((tert-Butyldimethylsilyl)oxy)methyl)-3,6-difluoro-2-methoxybenzonitrile

Compound Int 2-c (3.60 g, 11.94 mmol) and sodium methoxide (1.93 g, 35.72 mmol) were dissolved in a solution of tetrahydrofuran (40 mL). The reaction system was heated to 80° C. and stirred for 4 h. After the reaction was completed, the reaction mixture was warmed to room temperature, diluted with water, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound Int 2-d (1.60 g, yield: 42.7%).

¹H NMR (400 MHz, CDCl₃): δ6.91-6.87 (m, 1H), 4.78 (s, 2H), 4.14 (d, 3H), 0.95 (s, 9H), 0.13 (s, 6H).

### Step e:

### 3,6-Difluoro-4-(hydroxymethyl)-2-methoxybenzonitrile

Tetrabutylammonium fluoride (2.67 g, 10.21 mmol) was added to a solution of compound Int 2-d (1.60 g, 5.10 mmol) in tetrahydrofuran (20 mL). The reaction system was stirred at 25° C. for 1 h. After the reaction was completed, the reaction mixture was diluted with water (20 mL) and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound Int 2-e (0.79 g, yield: 77.7%).

¹H NMR (400 MHz, CDCl₃): δ7.06-7.03 (m, 1H), 4.81 (s, 2H), 4.15 (d, 3H).

### Step f:

### 4-((1H-Pyrazol-1-yl)methyl)-3, 6-difluoro-2-methoxybenzonitrile

Cesium carbonate (2.58 g, 7.92 mmol) was added to a solution of compound Int 2-e (0.79 g, 3.97 mmol) and 1-(methylsulfonyl)-1H-pyrazole (0.70 g, 4.76 mmol) in acetonitrile (20 mL). The reaction system was heated to 75° C. and stirred for 4 h. After the reaction was completed, the reaction mixture was cooled to room temperature, then diluted with water, and extracted with ethyl acetate (25 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound Int 2-f (0.81 g, yield: 81.9%).

MS m/z (ESI): 250.0 [M+H]⁺.

### Step g:

### 6-((1H-Pyrazol-1-yl)methyl)-5-fluoro-4-methoxybenzo[d]isoxazol-3-amine

Compound Int 2-f (810 mg, 3.25 mmol), acetohydroxamic acid (732 mg, 9.75 mmol), and 2-tert-butyl-1,1,3,3-tetramethylguanidine (557 mg, 3.25 mmol) were dissolved in acetonitrile (15 mL). The reaction system was heated to 80° C. and stirred for 16 h. After the reaction was completed, the reaction mixture was cooled to room temperature and concentrated under reduced pressure. The resulting crude product was purified by flash silica gel column chromatography (petroleum ether:ethyl acetate=2:1) to give compound intermediate 2 (440 mg, yield: 51.6%).

¹HNMR (400 MHz, DMSO-d₆): δ7.86 (d, 1H), 7.54 (d, 1H), 6.72 (d, 1H), 6.31 (t, 1H), 6.11 (s, 2H), 5.47 (s, 2H), 4.06 (d, 3H).

### Example 3

### Synthesis of Intermediate 3

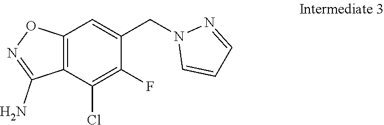 [SMILES: Nc1noc2cc(Cn3cccn3)c(F)c(Cl)c12]

### 6-((1H-Pyrazol-1-yl)methyl)-4-chloro-5-fluorobenzo[d]isoxazol-3-amine

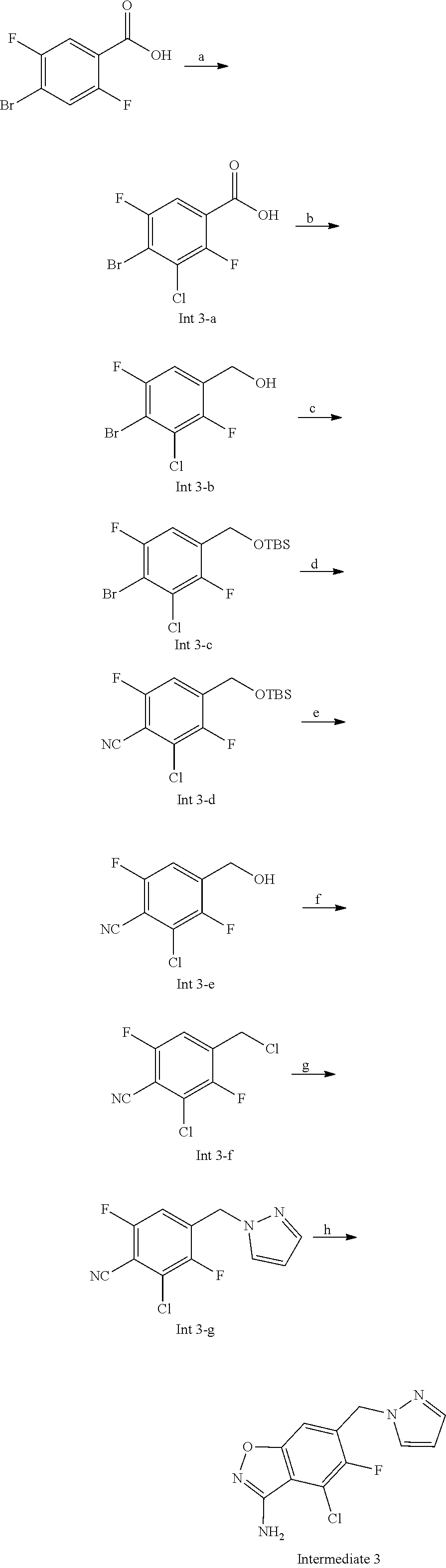

### Step a:

### 4-Bromo-3-chloro-2,5-difluorobenzoic acid

Under a nitrogen atmosphere, at 80° C., N-chlorosuccinimide (5.63 g, 42.16 mmol) was added to a solution of 4-bromo-2,5-difluorobenzoic acid (5.00 g, 21.10 mmol) in 98% concentrated sulfuric acid (30 mL). The reaction system was then stirred at 80° C. for 16 h. After the reaction was completed, the mixture was poured into ice water, and the resulting mixture was concentrated under reduced pressure. The resulting filter cake was purified by flash C18 reversed-phase column chromatography(methanol:water (0.1% trifluoroacetic acid)=3:2) to give compound Int 3-a (2.30 g, yield: 40.2%).

¹HNMR (400 MHz, DMSO-d₆): δ14.05 (brs, 1H), 7.80 (dd, 1H).

### Step b:

### (4-Bromo-3-chloro-2,5-difluorophenyl)methanol

Under a nitrogen atmosphere, at 0° C., a solution of borane in tetrahydrofuran (1.0 M in tetrahydrofuran, 28.74 mL, 28.74 mmol) was added dropwise to a solution of compound Int 3-a (3.90 g, 14.37 mmol) in tetrahydrofuran (60 mL). The reaction system was heated to 50° C. and stirred for 2.5 h. After the reaction was completed, the reaction system was quenched with methanol at 0° C. and extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=9:1) to give compound Int 3-b (3.26 g, yield: 88.1%). ¹HNMR (400 MHz, DMSO-d₆): δ7.44 (dd, 1H), 5.61 (t, 1H), 4.56 (d, 2H).

### Step c:

### ((4-Bromo-3-chloro-2,5-difluorobenzyl)oxy)(tert-butyl)dimethylsilane

tert-Butyldimethylsilyl chloride (2.10 g, 13.93 mmol) was added to a solution of compound Int 3-b (3.26 g, 12.66 mmol) and pyrazole (1.72 g, 25.32 mmol) in dichloromethane (60 mL) at 25° C. The reaction system was stirred at 25° C. for 2 h. After the reaction was completed, the reaction system was poured into ice water, and the resulting mixture was extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether) to give compound Int 3-c (4.56 g, yield: 96.9%). ¹HNMR (400 MHz, DMSO-d₆): δ7.38 (dd, 1H), 4.75 (s, 2H), 0.89 (s, 9H), 0.10 (s, 6H).

### Step d:

### 4-(((tert-Butyldimethylsilyl)oxy)methyl)-2-chloro-3,6-difluorobenzonitrile

Under a nitrogen atmosphere, compound Int 3-c (4.65 g, 12.51 mmol) and copper(I) cyanide (5.60 g, 62.55 mmol) were dissolved in a solution of N,N-dimethylformamide (25 mL). The reaction system was heated to 140° C. and stirred for 16 h. After the reaction was completed, the reaction system was quenched with water and extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=9:1) to give compound Int 3-d (1.89 g, yield: 47.5%). ¹HNMR (400 MHz, DMSO-d₆):7.48 (dd, 1H), 4.86 (s, 2H), 0.91 (s, 9H), 0.12 (s, 6H).

### Step e:

### 2-Chloro-3,6-difluoro-4-(hydroxymethyl)benzonitrile

Tetrabutylammonium fluoride (2.18 g, 8.33 mmol) was added to a solution of compound Int 3-d (1.89 g, 5.95 mmol) in tetrahydrofuran (38 mL) at 0° C. The reaction system was stirred at 0° C. for 1 h. After the reaction was completed, the reaction system was poured into ice water, and the resulting mixture was extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=4:1) to give compound Int 3-e (0.84 g, yield: 69.4%).

¹HNMR (400 MHz, DMSO-d₆): 7.55 (dd, 1H), 5.81 (t, 1H), 4.65 (d, 2H).

### Step f:

### 2-Chloro-4-(chloromethyl)-3,6-difluorobenzonitrile

Under a nitrogen atmosphere, N,N-dimethylformamide (0.15 g, 2.04 mmol) was added to a solution of compound Int 3-e (4.15 g, 20.39 mmol) and thionyl chloride (12.13 g, 101.96 mmol) in dichloromethane (40 mL). The reaction system was heated to 40° C. and stirred for 3 h. After the reaction was completed, the reaction system was diluted with water and extracted with ethyl acetate (300 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=9:1) to give compound Int 3-f (4.35 g, yield: 96.1%).

¹HNMR (400 MHz, DMSO-d₆): 7.84 (dd, 1H), 4.88 (s, 2H).

### Step g:

### 4-((1H-Pyrazol-1-yl)methyl)-2-chloro-3,6-difluorobenzonitrile

Under a nitrogen atmosphere, compound Int 3-f (2.25 g, 10.13 mmol), pyrazole (0.76 g, 11.16 mmol), 18-crown-6 (0.80 g, 3.03 mmol), and potassium carbonate (2.80 g, 20.26 mmol) were dissolved in acetonitrile (24 mL). The reaction system was heated to 80° C. and stirred for 1.5 h. After the reaction was completed, the reaction mixture was cooled to room temperature, then diluted with water (50 mL), and extracted with ethyl acetate (150 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=9:1) to give compound Int 3-g (1.00 g, yield: 38.9%).

¹HNMR (400 MHz, DMSO-d₆): δ 7.89 (d, 1H), 7.53 (d, 1H), 7.21 (dd, 1H), 6.33 (t, 1H), 5.54 (s, 2H).

### Step h:

### 6-((1H-Pyrazol-1-yl)methyl)-4-chloro-5-fluorobenzo[d]isoxazol-3-amine

Compound Int 3-g (21.34 g, 5.28 mmol), acetohydroxamic acid (1.19 g, 15.85 mmol), and potassium carbonate (2.19 g, 15.85 mmol) were dissolved in a mixed solution (N,N-dimethylformamide and water in a ratio of 5:1, 24 mL). The reaction system was heated to 80° C. and stirred for 1.5 h. After the reaction was completed, the reaction mixture was cooled to room temperature and poured into ice water, and the resulting mixture was extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=9:1) to give compound intermediate 3 (0.94 g, yield: 69.1%).

¹HNMR (400 MHz, DMSO-d₆): δ7.87 (d, 1H), 7.50 (d, 1H), 7.18 (d, 1H), 6.31 (t, 1H), 6.24 (s, 2H), 5.52 (s, 2H).

MS m/z (ESI): 267.2 [M+H]⁺.

### Example 4

### Synthesis of Intermediate 4

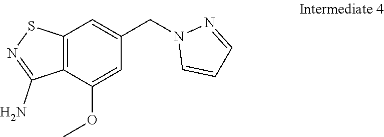 [SMILES: COc1cc(Cn2cccn2)cc2snc(N)c12]

### 6-((1H-Pyrazol-1-yl)methyl)-4-methoxybenzo[d]isothiazol-3-amine

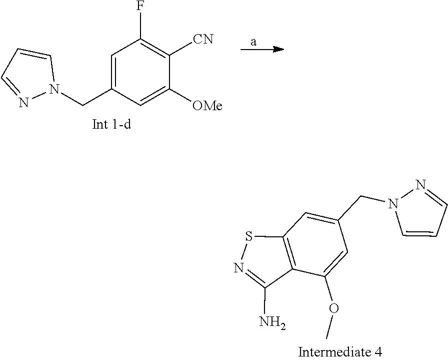 [SMILES: COc1cc(Cn2cccn2)cc(F)c1C#N ; COc1cc(Cn2cccn2)cc2snc(N)c12]

### Step a:

### 6-((1H-Pyrazol-1-yl)methyl)-4-methoxybenzo[d]isothiazol-3-amine

Under a nitrogen atmosphere, sodium sulfide (157 mg, 2.02 mmol) was added to a solution of compound Int 1-d (466 mg, 2.02 mmol) in dimethyl sulfoxide (15 mL). The reaction system was heated to 70° C. and stirred for 7 h. After the reaction was completed, the reaction system was cooled to 0° C. Subsequently, aqueous ammonia (6 mL) and an aqueous sodium hypochlorite solution (6 mL) were slowly added to the reaction system, and stirring was continued for 16 h.

The reaction mixture was diluted with water (100 mL) and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=9:1) to give compound intermediate 4 (210 mg, yield: 40.0%).

¹HNMR (300 MHz, DMSO-d₆): δ7.87 (d, 1H), 7.49 (d, 1H), 7.13 (s, 1H), 6.74 (s, 1H), 6.50 (s, 2H), 6.30 (t, 1H), 5.42 (s, 2H), 3.90 (s, 3H), 2.54 (s, 2H).

### Example 5

### Synthesis of Intermediate 5

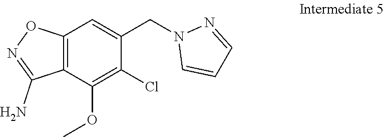 [SMILES: COc1c(Cl)c(Cn2cccn2)cc2onc(N)c12]

### 6-((1H-Pyrazol-1-yl)methyl)-5-chloro-4-methoxybenzo[d]isoxazol-3-amine

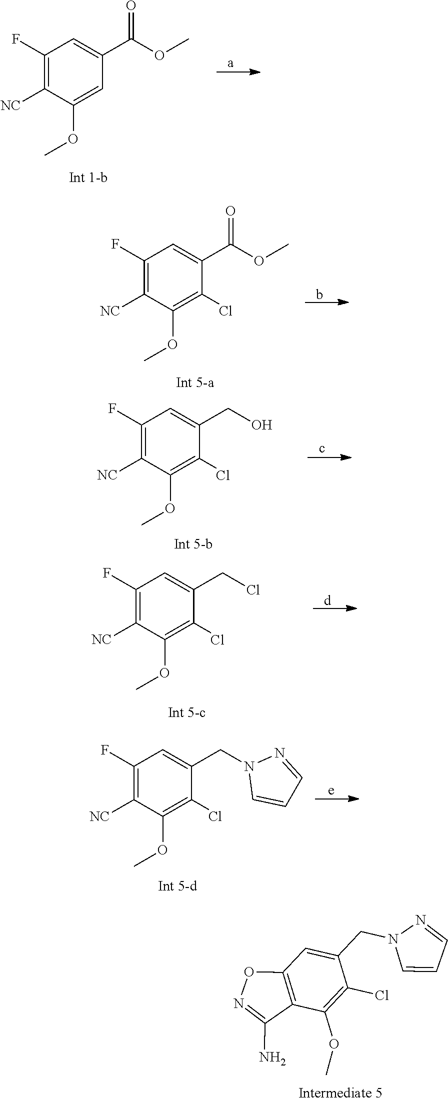

### Step a:

### Methyl 2-chloro-4-cyano-5-fluoro-3-methoxybenzoate

Compound Int 1-b (2.10 g, 10.04 mmol), trichloroisocyanuric acid (1.16 g, 4.99 mmol), and trifluoromethanesulfonic acid (0.09 mL, 1.00 mmol) were dissolved in a solution of hexafluoroisopropanol (20 mL). The reaction system was heated to 60° C. and stirred for 40 h. After the reaction was completed, the mixture was cooled to room temperature, quenched with water, and extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:dichloromethane=4:1) to give compound Int 5-a (1.45 g, yield: 59.3%).

¹H NMR (400 MHz, CDCl₃): δ7.34 (d, 1H), 4.14 (s, 3H), 3.97 (s, 3H).

### Step b:

### 3-Chloro-6-fluoro-4-(hydroxymethyl)-2-methoxybenzonitrile

Lithium borohydride (2.0 M in tetrahydrofuran, 6.6 mL, 13.20 mmol) was added dropwise to a solution of compound Int 5-a (1.60 g, 6.57 mmol) in tetrahydrofuran (50 mL) at 0° C. Subsequently, the reaction system was heated to 50° C. and then stirred for 1 h. After the reaction was completed, the reaction mixture was cooled to room temperature, then quenched with a saturated aqueous ammonium chloride solution, and extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound Int 5-b (1.37 g, yield: 96.7%).

¹HNMR (400 MHz, DMSO-d₆): δ7.28 (d, 1H), 4.82 (d, 2H), 4.10 (s, 3H).

### Step c:

### 3-Chloro-4-(chloromethyl)-6-fluoro-2-methoxybenzonitrile

Thionyl chloride (10 mL) and one drop of N,N-dimethylformamide were added to a solution of compound Int 5-b (610 mg, 2.83 mmol) in dichloromethane (10 mL). The reaction system was heated to 40° C. and stirred for 3 h. After the reaction was completed, the reaction mixture was cooled to room temperature, then diluted with a saturated aqueous sodium bicarbonate solution, and extracted with dichloromethane (50 mL×3). The organic layers were combined, washed with a saturated aqueous NaCl solution, and dried over anhydrous sodium sulfate. After filtration, the mother liquor was concentrated under reduced pressure to give compound Int 5-c (580 mg, yield: 87.7%).

¹HNMR (400 MHz, DMSO-d₆): δ7.19 (d, 1H), 7.67 (s, 2H), 7.13 (s, 3H).

### Step d:

### 4-((1H-Pyrazol-1-yl)methyl)-3-chloro-6-fluoro-2-methoxybenzonitrile

Compound Int 5-c (960 mg, 4.10 mmol), pyrazole (838 mg, 12.31 mmol), cesium fluoride (1.87 g, 12.31 mmol), and tetrabutylammonium iodide (152 mg, 0.41 mmol) were dissolved in a solution of acetonitrile (40 mL). The reaction system was heated to 40° C. and stirred for 16 h. After the reaction was completed, the mixture was poured into ice water, and the resulting mixture was extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=6:1) to give compound Int 5-d (770 mg, yield: 70.7%).

¹HNMR (400 MHz, DMSO-d₆): δ7.90 (d, 1H), 7.57 (d, 1H), 6.55 (d, 1H), 6.36 (t, 1H), 5.55 (s, 2H), 4.07 (s, 3H).

### Step e:

### 6-((1H-Pyrazol-1-yl)methyl)-5-chloro-4-methoxybenzo[d]isoxazol-3-amine

Compound Int 5-d (1.61 g, 6.06 mmol), acetohydroxamic acid (1.37 g, 18.25 mmol), and potassium carbonate (2.53 g, 18.31 mmol) were dissolved in a mixed solution (N,N-dimethylformamide and water in a ratio of 10:1, 55 mL). The reaction system was heated to 70° C. and stirred for 16 h. After the reaction was completed, the reaction mixture was cooled to room temperature, then diluted with water (50 mL), and extracted with ethyl acetate (150 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give intermediate 5 (1.10 g, yield: 65.1%).

¹HNMR (400 MHz, DMSO-d₆): δ7.88 (d, 1H), 7.55 (d, 1H), 6.60 (s, 1H), 6.34 (t, 1H), 6.20 (s, 2H), 5.53 (s, 2H), 3.94 (s, 3H).

### Example 6

### Synthesis of Intermediate 6

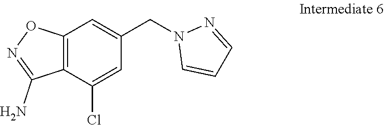 [SMILES: Nc1noc2cc(Cn3cccn3)cc(Cl)c12]

### 6-((1H-Pyrazol-1-yl)methyl)-4-chlorobenzo[d]isoxazol-3-amine

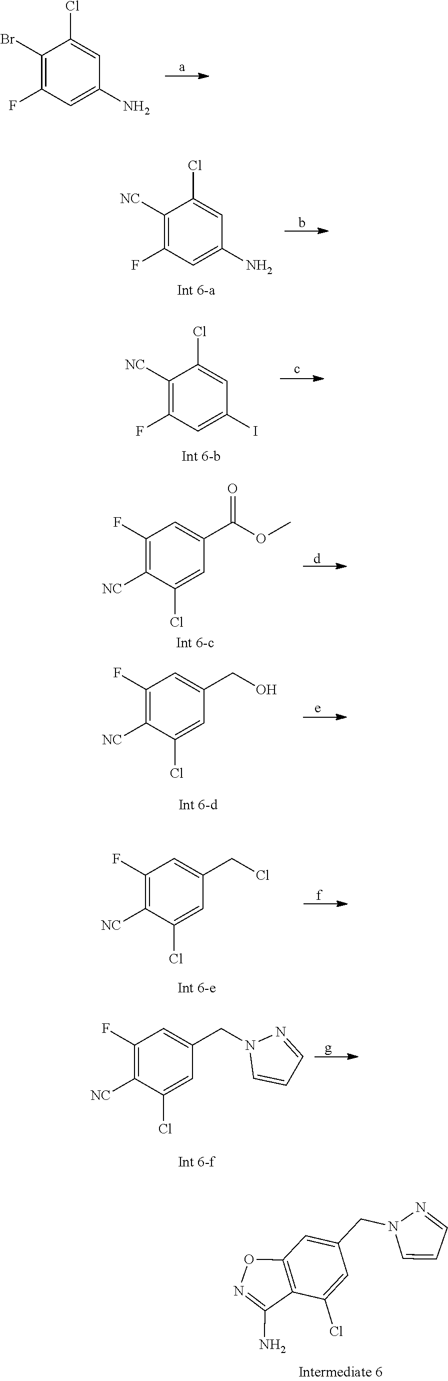

### Step a:

### 4-Amino-2-chloro-6-fluorobenzonitrile

Under a nitrogen atmosphere, 4-bromo-3-chloro-5-fluoroaniline (21.70 g, 96.68 mmol) and copper(I) cyanide (8.66 g, 96.69 mmol) were dissolved in a solution of N-methylpyrrolidinone (100 mL). The reaction system was heated to 190° C. and stirred for 2 h. After the reaction was completed, the reaction mixture was cooled to room temperature, quenched with 14% aqueous ammonia, and extracted with ethyl acetate (300 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=1:1) to give compound Int 6-a (16.40 g, yield: 99.5%).

¹H NMR (300 MHz, DMSO-d₆): δ6.87 (s, 2H), 6.60 (d, 1H), 6.44 (dd, 1H).

### Step b:

### 2-Chloro-6-fluoro-4-iodobenzonitrile

An aqueous solution (100 mL) of sodium nitrite (4.45 g, 64.46 mmol) was slowly added to a solution of compound Int 6-a (10.00 g, 58.63 mmol) and 98% concentrated sulfuric acid (9.4 mL, 175.80 mmol) in acetonitrile (300 mL) and water (90 mL) at 0° C. The reaction system was stirred at 10° C. for 15 min, then warmed to room temperature, and stirred for another 2 h. After the reaction was completed, the mixture was poured into an aqueous solution (200 mL), and the resulting mixture was extracted with ethyl acetate (300 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound Int 6-b (12.00 g, yield: 72.7%).

¹H NMR (300 MHz, CDCl₃): δ7.73 (s, 1H), 7.55 (dd, 1H).

### Step c:

### Methyl 3-chloro-4-cyano-5-fluorobenzoate

Triethylamine (15.21 g, 150.30 mmol) and [1,1′-bis(diphenylphosphino)ferrocene]palladium(II) dichloride (3.64 g, 4.97 mmol) were added to a solution of compound Int 6-b (14.10 g, 50.10 mmol) in methanol (140 mL). The reaction system was stirred at 40° C. for 18 h under a carbon monoxide atmosphere (2.0 MPa). After the reaction was completed, the reaction mixture was cooled to room temperature, diluted with water, and extracted with ethyl acetate (300 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=92:8) to give compound Int 6-c (8.40 g, yield: 78.5%).

¹HNMR (300 MHz, CDCl₃): δ7.99 (s, 1H), 7.77 (dd, 1H), 3.98 (s, 3H).

### Step d:

### 2-Chloro-6-fluoro-4-(hydroxymethyl)benzonitrile

Under a nitrogen atmosphere, a solution of lithium borohydride (39.3 mL, 78.60 mmol) in tetrahydrofuran (100 mL) was added to a solution of compound Int 6-c (8.40 g, 39.33 mmol) in tetrahydrofuran (100 mL). The reaction system was stirred at 25° C. for 1 h. After the reaction was completed, the reaction mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=7:3) to give compound Int 6-d (5.00 g, yield: 68.5%).

¹HNMR (300 MHz, CDCl₃): δ7.34 (s, 1H), 7.18 (dd, 1H), 4.78 (s, 2H), 1.86 (s, 1H).

### Step e:

### 2-Chloro-4-(chloromethyl)-6-fluorobenzonitrile

Under a nitrogen atmosphere, a solution of thionyl chloride (6.02 g, 50.61 mmol) in dichloromethane (10 mL) was added dropwise to a solution of compound Int 6-d (4.70 g, 25.33 mmol) and N,N-dimethylformamide (0.02 g, 0.25 mmol) in dichloromethane (70 mL). The reaction system was heated to 40° C. and stirred for 1 h. After the reaction was completed, the mixture was poured into ice water, and the resulting mixture was extracted with dichloromethane (200 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=92:8) to give compound Int 6-e (4.74 g, yield: 91.7%).

¹HNMR (400 MHz, CDCl₃): δ7.37 (s, 1H), 7.20 (dd, 1H), 4.55 (s, 2H).

### Step f:

### 4-((1H-Pyrazol-1-yl)methyl)-2-chloro-6-fluorobenzonitrile

Compound Int 6-e (4.70 g, 23.04 mmol), pyrazole (4.70 g, 69.04 mmol), cesium fluoride (10.48 g, 68.99 mmol), and tetrabutylammonium iodide (0.85 g, 2.30 mmol) were dissolved in a solution of acetonitrile (100 mL). The reaction system was heated to 50° C. and stirred for 18 h. After the reaction was completed, the reaction mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=82:18) to give compound Int 6-f (3.61 g, yield: 66.5%).

¹HNMR (400 MHz, DMSO-d₆): δ7.91 (d, 1H), 7.54 (d, 1H), 7.35 (s, 1H), 7.28 (d, 1H), 6.34 (t, 1H), 5.48 (s, 2H).

### Step g:

### 6-((1H-Pyrazol-1-yl)methyl)-4-chlorobenzo[d]isoxazol-3-amine

Compound Int 6-f (3.61 g, 15.32 mmol), acetohydroxamic acid (3.45 g, 45.96 mmol), and tetramethylguanidine (10.57 g, 91.77 mmol) were dissolved in a mixed solution (N,N-dimethylformamide and water in a ratio of 10:1, 55 mL). The reaction system was heated to 80° C. and stirred for 18 h. After the reaction was completed, the reaction mixture was cooled to room temperature, then diluted with water (100 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:2) to give intermediate 6 (2.90 g, yield: 71.0%).

¹HNMR (300 MHz, DMSO-d₆): δ7.90 (d, 1H), 7.51 (d, 1H), 7.24 (s, 1H), 7.10 (s, 1H), 6.31 (t, 1H), 6.19 (s, 2H), 5.46 (s, 2H).

### Example 7

### Synthesis of Compound 1

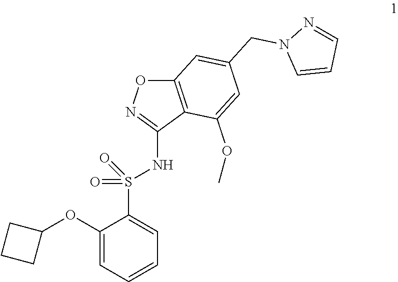 [SMILES: COc1cc(Cn2cccn2)cc2onc(NS(=O)(=O)c3ccccc3OC3CCC3)c12]

### N-(6-((1H-Pyrazol-1-yl)methyl)-4-methoxybenzo[d]isoxazol-3-yl)-2-cyclobutoxybenzenesulfonamide

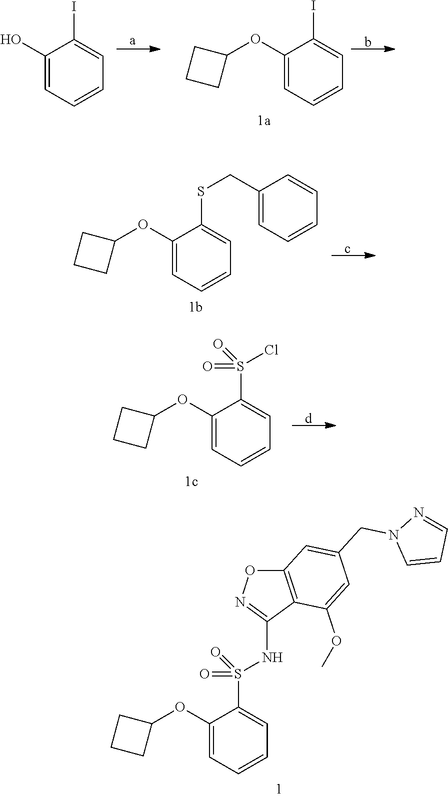 [SMILES: Oc1ccccc1I ; Ic1ccccc1OC1CCC1 ; c1ccc(CSc2ccccc2OC2CCC2)cc1 ; O=S(=O)(Cl)c1ccccc1OC1CCC1 ; COc1cc(Cn2cccn2)cc2onc(NS(=O)(=O)c3ccccc3OC3CCC3)c12]

### Step a:

### 1-Cyclobutoxy-2-iodobenzene

Under a nitrogen atmosphere, potassium carbonate (5.30 g, 38.35 mmol) was added to a solution of 2-iodophenol (2.80 g, 12.73 mmol) and cyclobutyl bromide (3.41 g, 25.26 mmol) in N,N-dimethylformamide (40 mL), and the reaction system was heated to 80° C. and stirred for 16 h. After the reaction was completed, the reaction mixture was cooled to room temperature, diluted with water (100 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 1a (2.60 g, yield: 74.5%).

¹H NMR (300 MHz, CDCl₃): δ7.81 (d, 1H), 7.32-7.27 (m, 1H), 6.76-6.71 (m, 2H), 4.76-4.68 (m, 1H), 2.57-2.48 (m, 2H), 2.38-2.25 (m, 2H), 1.99-1.89 (m, 1H), 1.96-1.62 (m, 1H).

### Step b:

### Benzyl(2-cyclobutoxyphenyl)sulfane

Under a nitrogen atmosphere, benzyl mercaptan (1.77 g, 14.25 mmol), cesium carbonate (6.18 g, 18.97 mmol), 1,4-diazabicyclo[2.2.2]octane (0.11 g, 0.98 mmol), and copper(I) iodide (0.91 g, 4.78 mmol) were added to a solution of compound 1a (2.60 g, 9.49 mmol) in N,N-dimethylformamide (50 mL). The reaction system was heated to 100° C. and stirred for 16 h. After the reaction was completed, the reaction mixture was cooled to room temperature, diluted with water (100 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 1b (2.10 g, yield: 81.9%).

MS m/z (ESI): 271.0 [M+H]⁺.

### Step c:

### 2-Cyclobutoxybenzenesulfonyl chloride

N-Chlorosuccinimide (3.10 g, 23.22 mmol) was added to compound 1b (2.10 g, 7.77 mmol) in a mixed solution (glacial acetic acid and water in a ratio of 10:1, 33 mL) at 0° C. The reaction system was stirred at 25° C. for 12 h. Subsequently, the reaction mixture was cooled to 0° C. with ice, stirred for 1 h, then filtered under reduced pressure, and dried under vacuum to give compound 1c (1.36 g, yield: 71.0%).

¹HNMR (300 MHz, CDCl₃): δ 7.59-7.49 (m, 1H), 7.43-7.34 (m, 2H), 7.03-6.91 (m, 1H), 4.85-4.73 (m, 1H), 2.53-2.35 (m, 2H), 2.36-2.16 (m, 2H), 1.95-1.80 (m, 1H), 1.77-1.57 (m, 1H).

### Step d:

### N-(6-((1H-Pyrazol-1-yl)methyl)-4-methoxybenzo[d]isoxazol-3-yl)-2-cyclobutoxybenzenesulfonamide

Compound 1c (870 mg, 3.53 mmol) and intermediate 1 (250 mg, 1.02 mmol) were dissolved in pyridine (2 mL). The reaction system was heated to 100° C. and stirred for 16 h. After the reaction was completed, the reaction mixture was cooled to room temperature and concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 1 (61 mg, yield: 13.1%).

¹HNMR (400 MHz, DMSO-d₆): δ9.82 (s, 1H), 7.87-7.84 (m, 2H), 7.58-7.50 (t, 1H), 7.49 (d, 1H), 7.10-7.06 (t, 1H), 6.97-6.95 (d, 1H), 6.83 (s, 1H), 6.75 (s, 1H), 6.30-6.29 (t, 1H), 5.44 (s, 2H), 4.73-4.69 (m, 1H), 3.81 (s, 3H), 2.26-2.22 (m, 2H), 1.82-1.77 (m, 2H), 1.57-1.49 (m, 2H).

MS m/z (ESI): 455.0 [M+H]⁺.

### Example 8

### Synthesis of Compound 2

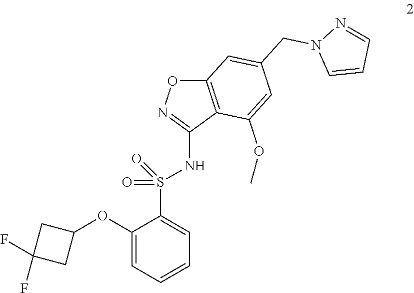 [SMILES: COc1cc(Cn2cccn2)cc2onc(NS(=O)(=O)c3ccccc3OC3CC(F)(F)C3)c12]

### N-(6-((1H-Pyrazol-1-yl)methyl)-4-methoxybenzo[d]isoxazol-3-yl)-2-(3, 3-difluorocyclobutoxy)benzenesulfonamide

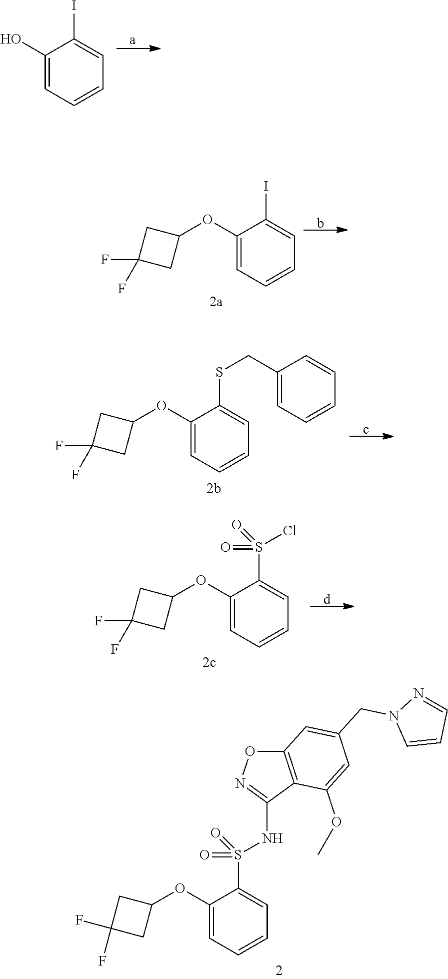 [SMILES: Oc1ccccc1I ; FC1(F)CC(Oc2ccccc2I)C1 ; FC1(F)CC(Oc2ccccc2SCc2ccccc2)C1 ; O=S(=O)(Cl)c1ccccc1OC1CC(F)(F)C1 ; COc1cc(Cn2cccn2)cc2onc(NS(=O)(=O)c3ccccc3OC3CC(F)(F)C3)c12]

### Step a:

### 1-(3,3-Difluorocyclobutoxy)-2-iodobenzene

Under a nitrogen atmosphere, triphenylphosphine (10.93 g, 41.67 mmol) and diisopropyl azodicarboxylate (5.62 g, 27.79 mmol) were added to a solution of 2-iodophenol (4.89 g, 22.23 mmol) and 3,3-difluorocyclobutanol (2.00 g, 18.50 mmol) in toluene (150 mL). The reaction system was heated to 110° C. and stirred for 16 h. After the reaction was completed, the reaction mixture was cooled to room temperature, diluted with water (150 mL), and extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 2a (2.70 g, yield: 47.1%).

¹H NMR (300 MHz, CDCl₃): δ7.81-7.78 (m, 1H), 7.37-7.33 (m, 1H), 6.92-6.90 (m, 1H), 6.81-6.77 (m, 1H), 4.85-4.81 (m, 1H), 3.32-3.19 (m, 2H), 2.76-2.64 (m, 2H).

### Step b:

### Benzyl(2-(3,3-difluorocyclobutoxy)phenyl)sulfane

Under a nitrogen atmosphere, benzyl mercaptan (1.63 g, 13.12 mmol), cesium carbonate (5.70 g, 17.49 mmol), 1,4-diazabicyclo[2.2.2]octane (0.10 g, 0.87 mmol), and copper(I) iodide (0.84 g, 4.37 mmol) were added to a solution of compound 2a (2.70 g, 8.71 mmol) in N,N-dimethylformamide (50 mL). The reaction system was heated to 100° C. and stirred for 16 h.

After the reaction was completed, the reaction mixture was cooled to room temperature, diluted with water (100 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 2b (2.00 g, yield: 75.0%).

¹H NMR (300 MHz, CDCl₃): δ7.40-7.28 (m, 6H), 7.27-7.16 (m, 1H), 6.99-6.88 (m, 2H), 4.84-4.79 (m, 1H), 4.19 (s, 2H), 3.29-3.19 (m, 2H), 2.76-2.66 (m, 2H).

### Step c:

### 2-(3,3-Difluorocyclobutoxy)benzenesulfonyl chloride

N-Chlorosuccinimide (2.62 g, 19.62 mmol) was added to compound 2b (2.00 g, 6.53 mmol) in a mixed solution (glacial acetic acid and water in a ratio of 10:1, 33 mL) at 0° C. The reaction system was stirred at 25° C. for 12 h. Subsequently, the reaction mixture was cooled to 0° C. with ice, stirred for 1 h, then filtered under reduced pressure, and dried under vacuum to give compound 2c (1.20 g, yield: 65.0%). ¹HNMR (300 MHz, CDCl₃): δ8.08-8.06 (d, 1H), 7.77-7.72 (m, 1H), 7.26-7.21 (m, 1H), 7.00-6.98 (d, 1H), 4.94 (m, 1H), 3.33-3.20 (m, 2H), 3.10-2.94 (m, 2H).

### Step d:

### N-(6-((1H-Pyrazol-1-yl)methyl)-4-methoxybenzo[d]isoxazol-3-yl)-2-(3, 3-difluorocyclobutoxy)benzenesulfonamide

Compound 2c (1.20 g, 4.24 mmol) and intermediate 1 (200 mg, 0.82 mmol) were dissolved in pyridine (2 mL). The reaction system was heated to 100° C. and stirred for 16 h. After the reaction was completed, the reaction mixture was cooled to room temperature and concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 2 (20 mg, yield: 5.0%).

¹HNMR (400 MHz, DMSO-d₆): δ10.35 (s, 1H), 7.99-7.77 (m, 2H), 7.69-7.41 (m, 2H), 7.24-6.98 (m, 2H), 6.78 (s, 1H), 6.70 (s, 1H), 6.37-6.21 (m, 1H), 5.43 (s, 2H), 4.90-4.74 (m, 1H), 3.78 (s, 3H), 3.17-2.92 (m, 2H), 2.74-2.56 (m, 2H).

MS m/z (ESI): 491.0 [M+H]⁺.

### Example 9

### Synthesis of Compound 3

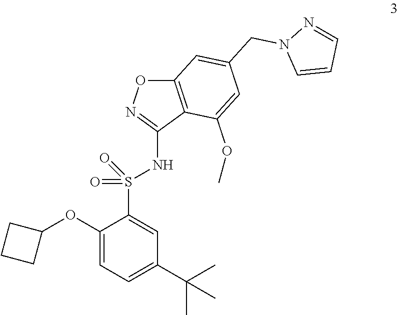 [SMILES: COc1cc(Cn2cccn2)cc2onc(NS(=O)(=O)c3cc(C(C)(C)C)ccc3OC3CCC3)c12]

### N-(6-((1H-Pyrazol-1-yl)methyl)-4-methoxybenzo[d]isoxazol-3-yl)-5-(tert-butyl)-2-cyclobutoxybenzenesulfonamide

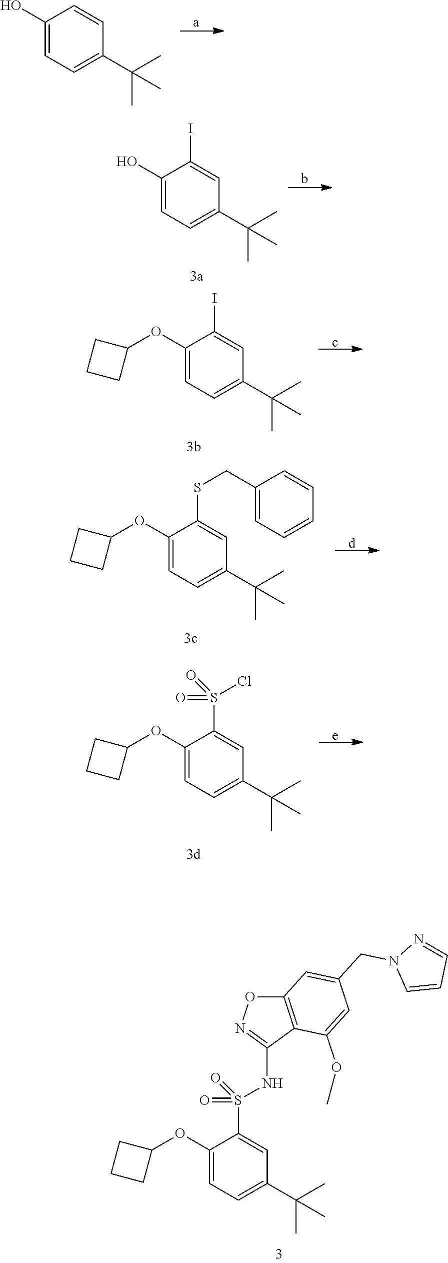

### Step a:

### 4-(tert-Butyl)-2-iodophenol

p-Toluenesulfonic acid monohydrate (6.33 g, 33.28 mmol) was added to a solution of 4-(tert-butyl)phenol (5.00 g, 33.28 mmol) in acetonitrile (75 mL) at 20° C. After the mixture was reacted for 30 min, N-iodosuccinimide (7.49 g, 33.29 mmol) was added, and the reaction system was stirred for 16 h. After the reaction was completed, the reaction mixture was quenched with an aqueous sodium sulfite solution (100 mL) and acidified with a hydrochloric acid solution (1.0 M), and then the phases were separated. The organic phase was collected, and the aqueous phase was extracted with ethyl acetate (100 mL×2). The organic layers were combined and dried over anhydrous sodium sulfate. After filtration, the mother liquor was concentrated under reduced pressure. The resulting crude product was purified by flash silica gel column chromatography (petroleum ether:ethyl acetate=20:1) to give compound 3a (8.20 g, yield: 89.2%).

¹H NMR (300 MHz, CDCl₃): δ 7.62 (d, 1H), 7.27-7.24 (m, 1H), 6.91 (d, 1H), 5.17 (s, 1H), 1.29 (s, 9H).

### Step b:

### 4-(tert-Butyl)-1-cyclobutoxy-2-iodobenzene

Bromocyclobutane (2.92 g, 21.63 mmol) was added to a solution of compound 3a (2.00 g, 7.24 mmol) and potassium carbonate (2.99 g, 21.63 mmol) in N,N-dimethylformamide (35 mL) at 20° C. The reaction system was heated to 70° C. and stirred for 16 h. After the reaction was completed, the reaction mixture was cooled to room temperature, diluted with water (50 mL), and extracted with ethyl acetate (30 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 3b (2.20 g, yield: 92.0%).

¹H NMR (300 MHz, CDCl₃): δ7.75 (d, 1H), 7.74-7.22 (m, 1H), 6.59 (d, 1H), 4.67-4.60 (m, 1H), 2.48-2.41 (m, 2H), 2.28-2.19 (m, 2H), 1.91-1.82 (m, 1H), 1.73-1.61 (m, 1H), 1.27 (s, 9H).

### Step c:

### Benzyl(5-(tert-butyl)-2-cyclobutoxyphenyl)sulfane

Under a nitrogen atmosphere, benzyl mercaptan (5.42 g, 43.64 mmol), cesium carbonate (14.21 g, 43.61 mmol), 1,4-diazabicyclo[2.2.2]octane (0.12 g, 1.06 mmol), and copper(I) iodide (0.21 g, 1.10 mmol) were added to a solution of compound 3b (7.20 g, 21.80 mmol) in N,N-dimethylformamide (35 mL). The reaction system was heated to 100° C. and stirred for 16 h. After the reaction was completed, the reaction mixture was cooled to room temperature, diluted with water (200 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=10:1) to give compound 3c (1.90 g, yield: 26.7%).

¹H NMR (400 MHz, CDCl₃): δ7.40-7.24 (m, 6H), 7.20-7.19 (m, 1H), 6.69 (d, 1H), 4.70-4.62 (m, 1H), 4.10 (s, 2H), 2.50-2.42 (m, 2H), 2.28-2.18 (m, 2H), 1.90-1.83 (m, 1H), 1.72-1.64 (m, 1H), 1.20 (s, 9H).

### Step d:

### 5-(tert-Butyl)-2-cyclobutoxybenzenesulfonyl chloride

N-Chlorosuccinimide (3.69 g, 27.63 mmol) was added to compound 3c (1.50 g, 4.59 mmol) in a mixed solution (glacial acetic acid and water in a ratio of 10:1, 18 mL) at 0° C. The reaction system was stirred at 10° C. for 1 h. After the reaction was completed, the mixture was quenched with water (20 mL) and filtered under reduced pressure. The filter cake was washed with water (3 mL×5) and dried under vacuum to give compound 3d (1.00 g, yield: 71.9%). ¹HNMR (300 MHz, CDCl₃): δ7.80 (d, 1H), 7.32-7.29 (m, 1H), 6.65 (d, 1H), 4.74-4.65 (m, 1H), 2.55-2.45 (m, 2H), 2.36-2.22 (m, 2H), 1.97-1.87 (m, 1H), 1.78-1.61 (m, 1H), 1.32 (s, 9H).

### Step e:

### N-(6-((1H-Pyrazol-1-yl)methyl)-4-methoxybenzo[d]isoxazol-3-yl)-5-(tert-butyl)-2-cyclobutoxybenzenesulfonamide

Compound 3d (743 mg, 2.45 mmol) was added to a solution of intermediate 1 (100 mg, 0.41 mmol) in pyridine (4 mL) at 10° C. The reaction system was heated to 100° C. and stirred for 16 h. After the reaction was completed, the reaction mixture was cooled to room temperature and concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 3 (64 mg, yield: 37.9%).

¹HNMR (400 MHz, CDCl₃): δ8.10 (d, 1H), 8.00 (s, 1H), 7.56 (d, 1H), 7.49-7.46 (m, 1H), 7.42 (d, 1H), 6.77 (s, 1H), 6.69 (d, 1H), 6.44 (s, 1H), 6.32 (t, 1H), 5.38 (s, 2H), 4.72-4.69 (m, 1H), 3.94 (s, 3H), 2.42-2.38 (m, 2H), 2.15-2.10 (m, 2H), 1.80-1.63 (m, 2H), 1.31 (s, 9H).

MS m/z (ESI): 511.1 [M+H]⁺.

### Example 10

### Synthesis of Compound 4

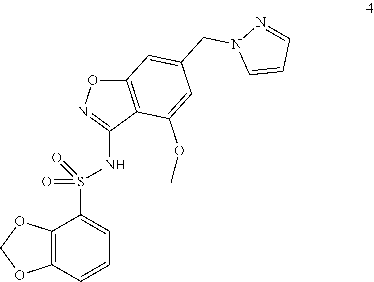 [SMILES: COc1cc(Cn2cccn2)cc2onc(NS(=O)(=O)c3cccc4c3OCO4)c12]

### N-(6-((1H-Pyrazol-1-yl)methyl)-4-methoxybenzo[d]isoxazol-3-yl)benzo[d][1,3]dioxole-4-sulfonamide

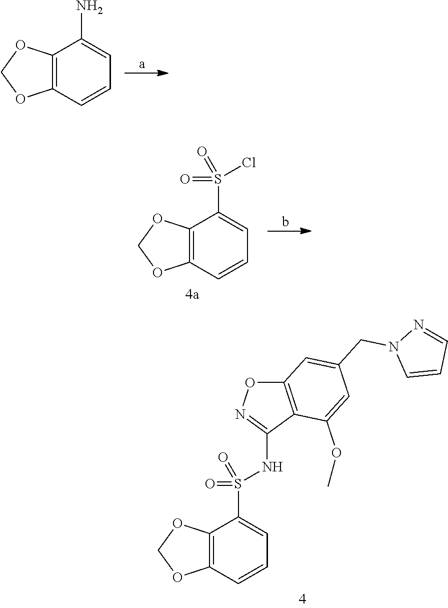 [SMILES: Nc1cccc2c1OCO2 ; O=S(=O)(Cl)c1cccc2c1OCO2 ; COc1cc(Cn2cccn2)cc2onc(NS(=O)(=O)c3cccc4c3OCO4)c12]

### Step a:

### Benzo[d][1,3]dioxole-4-sulfonyl chloride

Thionyl chloride (5.3 mL, 73.06 mmol) was added dropwise to water (30 mL) at 25° C., and the mixture was stirred for 6 h for later use. 4-Amino-1,3-benzodioxole (1.00 g, 7.29 mmol) was added dropwise to concentrated hydrochloric acid (7 mL) at 0° C. to give a hydrochloride, and then a solution of sodium nitrite (650 mg, 9.42 mmol) in water (2 mL) was added dropwise to the hydrochloride system. Subsequently, copper(I) chloride (70 mg, 0.71 mmol) was added to an aqueous solution containing thionyl chloride at a temperature below 5° C., and the prepared hydrochloride solution was slowly added. The mixture was stirred at 40° C. for 16 h and then filtered under reduced pressure. The filter cake was purified by flash silica gel column chromatography (petroleum ether:ethyl acetate=10:1) to give compound 4a (680 mg, yield: 42.3%).

### Step b:

### N-(6-((1H-Pyrazol-1-yl)methyl)-4-methoxybenzo[d]isoxazol-3-yl)benzo[d][1,3]dioxole-4-sulfonamide

Compound 4a (72 mg, 0.33 mmol) was added to a solution of intermediate 1 (80 mg, 0.33 mmol) in pyridine (2 mL). The reaction system was heated to 120° C. and stirred for 5 h. Compound 4a (72 mg, 0.33 mmol) was then added, and the mixture was stirred for 2 h. Subsequently, the last portion of compound 4a (72 mg, 0.33 mmol) was added, and stirring was continued for 2 h. After the reaction was completed, the reaction mixture was cooled to room temperature and concentrated under reduced pressure. The resulting mixture was purified by flash silica gel column chromatography (petroleum ether:ethyl acetate=10:1) to give a crude compound. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 4 (21 mg, yield: 15.0%).

¹HNMR (400 MHz, DMSO-d₆): δ 7.85 (d, 1H), 7.49 (d, 1H), 7.30-7.19 (m, 1H), 7.19-6.94 (m, 2H), 6.91-6.82 (m, 1H), 6.71 (s, 1H), 6.62 (s, 1H), 6.29 (t, 1H), 6.02 (s, 2H), 5.40 (s, 2H), 3.80 (s, 3H).

MS m/z (ESI): 428.9 [M+H]⁺.

### Example 11

### Synthesis of Compound 5

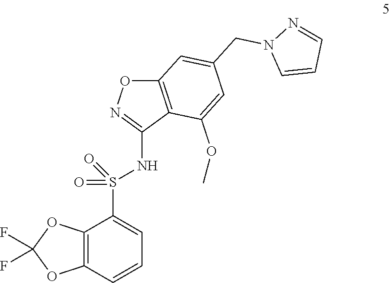 [SMILES: COc1cc(Cn2cccn2)cc2onc(NS(=O)(=O)c3cccc4c3OC(F)(F)O4)c12]

### N-(6-((1H-Pyrazol-1-yl)methyl)-4-methoxybenzo[d]isoxazol-3-yl)-2,2-difluorobenzo[d][1,3]dioxole-4-sulfonamide

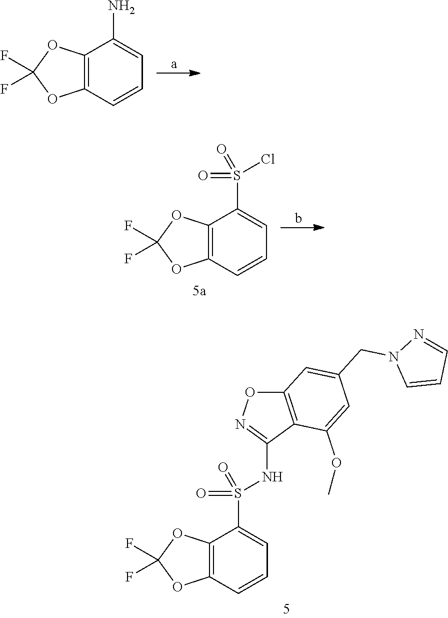 [SMILES: Nc1cccc2c1OC(F)(F)O2 ; O=S(=O)(Cl)c1cccc2c1OC(F)(F)O2 ; COc1cc(Cn2cccn2)cc2onc(NS(=O)(=O)c3cccc4c3OC(F)(F)O4)c12]

### Step a:

### 2,2-Difluorobenzo[d][1,3]dioxole-4-sulfonyl chloride

Thionyl chloride (5.96 mL, 82.16 mmol) was added dropwise to water (30 mL) at 25° C., and the mixture was stirred for 6 h for later use. 4-Amino-2,2-difluoro-1,3-benzodioxole (1.00 g, 5.78 mmol) was added dropwise to concentrated hydrochloric acid (7 mL) at 0° C. to give a hydrochloride, and then a solution of sodium nitrite (523 mg, 7.58 mmol) in water (2 mL) was added dropwise to the hydrochloride system. Subsequently, copper(I) chloride (10 mg, 0.10 mmol) was added to an aqueous solution containing thionyl chloride at a temperature below 5° C., and the prepared hydrochloride solution was slowly added. The mixture was stirred at 0° C. for 2 h and then extracted with dichloromethane (50 mL×2). The organic phases were combined and concentrated under reduced pressure to give compound 5a (1.00 g, yield: 67.5%).

### Step b:

### N-(6-((1H-Pyrazol-1-yl)methyl)-4-methoxybenzo[d]isoxazol-3-yl)-2,2-difluorobenzo[d][1,3]dioxole-4-sulfonamide

A solution of compound 5a (157 mg, 0.61 mmol) in pyridine (0.5 mL) was added to a solution of intermediate 1 (100 mg, 0.41 mmol) in pyridine (2 mL). The reaction system was heated to 80° C. and stirred for 2 h. Compound 5a (157 mg, 0.61 mmol) was then added, and the mixture was stirred for 2 h. After the reaction was completed, the reaction mixture was cooled to room temperature and concentrated under reduced pressure. The resulting mixture was purified by preparative thin-layer chromatography (dichloromethane:methanol=20:1) to give a crude compound. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 5 (30 mg, yield: 15.8%).

¹HNMR (400 MHz, DMSO-d₆): δ7.84 (d, 1H), 7.57-7.44 (m, 2H), 7.30-7.18 (m, 1H), 7.15-7.05 (m, 1H), 7.05-6.94 (m, 1H), 6.67 (s, 1H), 6.59 (s, 1H), 6.29 (t, 1H), 5.39 (s, 2H), 3.80 (s, 3H).

MS m/z (ESI): 464.9 [M+H]⁺.

### Example 12

### Synthesis of Compound 6

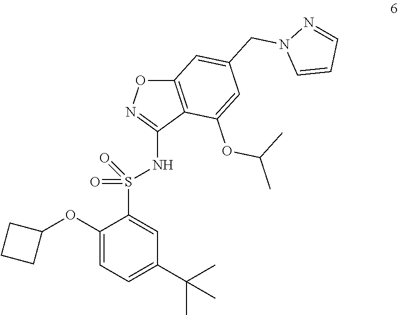 [SMILES: CC(C)Oc1cc(Cn2cccn2)cc2onc(NS(=O)(=O)c3cc(C(C)(C)C)ccc3OC3CCC3)c12]

### N-(6-((1H-Pyrazol-1-yl)methyl)-4-isopropoxybenzo[d]isoxazol-3-yl)-5-(tert-butyl)-2-cyclobutoxybenzenesulfonamide

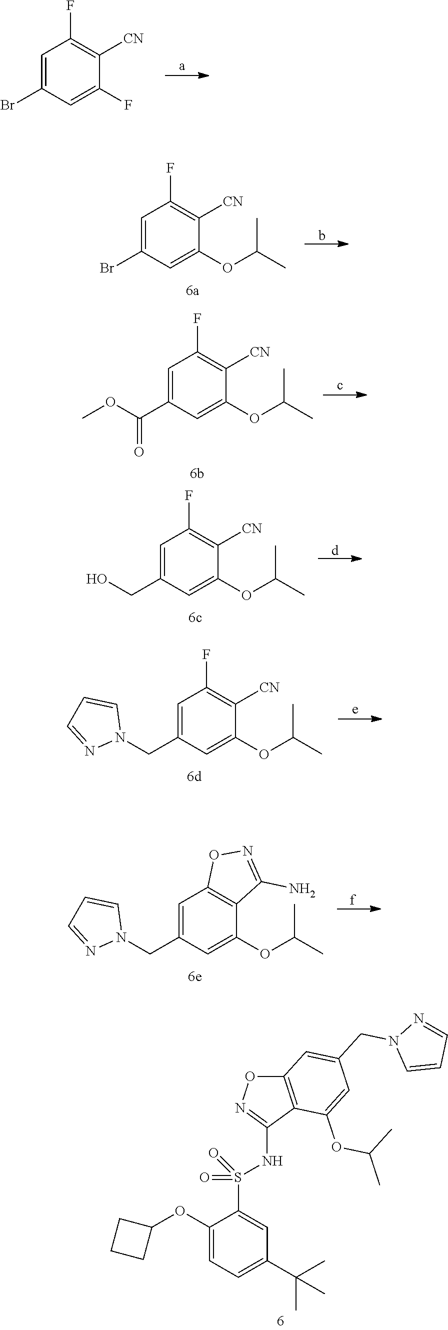

### Step a:

### 4-Bromo-2-fluoro-6-isopropoxybenzonitrile

Under a nitrogen atmosphere, 4-bromo-2,6-difluorobenzonitrile (2.40 g, 11.01 mmol) was added to a solution of sodium metal (280 mg, 11.67 mmol) in isopropanol (30 mL) at 0° C., and the reaction mixture was stirred at 20° C. for 16 h. Then, the reaction mixture was concentrated under reduced pressure. The resulting crude product was purified by flash silica gel column chromatography (petroleum ether:ethyl acetate=100:1) to give compound 6a (1.70 g, yield: 59.8%).

¹H NMR (400 MHz, CDCl₃): δ6.95 (d, 1H), 6.9 (s, 1H), 4.66-4.63 (m, 1H), 1.41 (d, 6H).

### Step b:

### Methyl 4-cyano-3-fluoro-5-isopropoxybenzoate

Triethylamine (4.6 mL, 34.09 mmol) and [1,1′-bis(diphenylphosphino)ferrocene]palladium(II) dichloride (0.24 g, 0.33 mmol) were added to a solution of compound 6a (1.70 g, 6.59 mmol) in methanol (20 mL). The reaction system was stirred at 80° C. for 16 h under a carbon monoxide atmosphere (1 atm). After the reaction was completed, the reaction mixture was cooled to room temperature and concentrated under reduced pressure. The resulting crude product was purified by flash silica gel column chromatography (petroleum ether:ethyl acetate=100:1) to give compound 6b (1.20 g, yield: 76.8%).

¹HNMR (400 MHz, CDCl₃): δ7.40 (s, 1H), 7.37 (d, 1H), 4.66-4.63 (m, 1H), 3.96 (s, 3H), 1.44 (d, 6H).

### Step c:

### 2-Fluoro-4-(hydroxymethyl)-6-isopropoxybenzonitrile

Lithium borohydride (0.40 g, 18.37 mmol) was added in portions to a solution of compound 6b (1.10 g, 4.64 mmol) in tetrahydrofuran (10 mL) at 10° C. The reaction system was heated to 70° C. and then stirred for 2 h. After the reaction was completed, the reaction mixture was cooled to room temperature, then quenched with diluted hydrochloric acid (1.0 M), and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 6c (907 mg, yield: 93.5%).

¹HNMR (300 MHz, CDCl₃): δ7.31 (s, 1H), 6.82 (s, 1H), 6.77 (d, 1H), 4.77 (s, 2H), 4.73-4.69 (m, 1H), 1.45 (d, 6H).

### Step d:

### 4-((1H-Pyrazol-1-yl)methyl)-2-fluoro-6-isopropoxybenzonitrile

Cesium carbonate (1.09 g, 3.35 mmol) was added to a solution of compound 6c (586 mg, 2.80 mmol) and 1-(methylsulfonyl)-1H-pyrazole (491 mg, 3.36 mmol) in acetonitrile (10 mL) at 20° C. The reaction system was heated to 70° C. and stirred for 1 h. After the reaction was completed, the reaction mixture was cooled to room temperature, then diluted with water, and extracted with ethyl acetate (25 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=5:1 to 2:1) to give compound 6d (700 mg, yield: 96.4%). ¹HNMR (400 MHz, CDCl₃): δ 7.60 (d, 1H), 7.45 (d, 1H), 6.48 (d, 1H), 6.45 (s, 1H), 6.36 (t, 1H), 5.32 (s, 2H), 4.61-4.49 (m, 1H), 1.35 (d, 6H).

### Step e:

### 6-((1H-Pyrazol-1-yl)methyl)-4-isopropoxybenzo[d]isoxazol-3-amine

Tetramethylguanidine (1.87 g, 16.20 mmol) was added dropwise to compound 6d (700 mg, 2.70 mmol) and acetohydroxamic acid (608 mg, 8.10 mmol) in a mixed solution (N,N-dimethylformamide and water in a ratio of 9:1, 8 mL). The reaction system was heated to 60° C. and stirred for 7 h. After the reaction was completed, the reaction mixture was cooled to room temperature, then diluted with water, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=1:1) to give compound 6e (500 mg, yield: 68.0%).

¹HNMR (400 MHz, DMSO-d₆): δ7.86 (d, 1H), 7.50 (d, 1H), 6.67 (s, 1H), 6.60 (s, 1H), 6.29 (t, 1H), 5.80 (s, 2H), 5.40 (s, 2H), 4.72-4.66 (m, 1H), 1.34 (d, 6H).

### Step f:

### N-(6-((1H-Pyrazol-1-yl)methyl)-4-isopropoxybenzo[d]isoxazol-3-yl)-5-(tert-butyl)-2-cyclobutoxybenzenesulfonamide

Compound 3d (336 mg, 1.11 mmol) was added to a solution of compound 6e (100 mg, 0.37 mmol) in pyridine (4 mL) at 10° C. The reaction system was heated to 100° C. and stirred for 16 h. After the reaction was completed, the reaction mixture was cooled to room temperature and concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 6 (20 mg, yield: 10.1%).

¹HNMR (400 MHz, DMSO-d₆): δ9.62 (s, 1H), 7.86 (d, 1H), 7.83 (d, 1H), 7.57 (d, 1H), 7.48 (d, 1H), 6.88 (s, 1H), 6.83 (d, 1H), 6.68 (s, 1H), 6.29 (t, 1H), 5.43 (s, 2H), 4.64-4.53 (m, 1H), 4.49-4.46 (m, 1H), 2.08-2.03 (m, 2H), 1.53-1.31 (m, 4H), 1.27 (s, 9H), 1.09 (d, 6H).

MS m/z (ESI): 539.2 [M+H]⁺.

### Example 13

### Synthesis of Compound 7

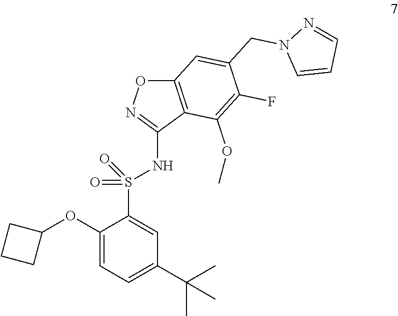 [SMILES: COc1c(F)c(Cn2cccn2)cc2onc(NS(=O)(=O)c3cc(C(C)(C)C)ccc3OC3CCC3)c12]

### N-(6-((1H-Pyrazol-1-yl)methyl)-5-fluoro-4-methoxybenzo[d]isoxazol-3-yl)-5-(tert-butyl)-2-cyclobutoxybenzenesulfonamide

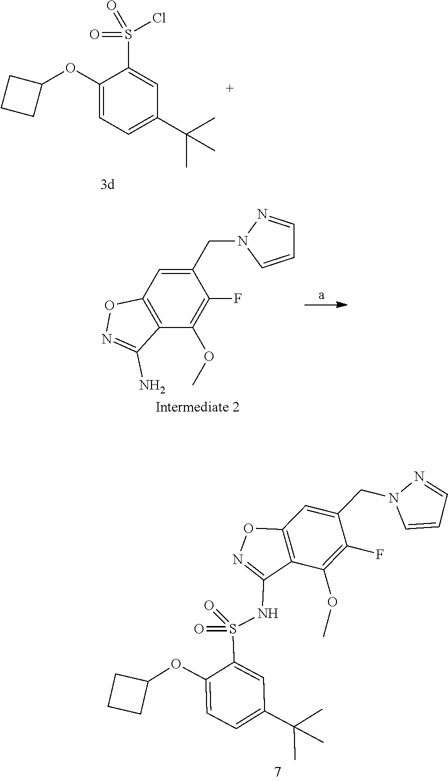 [SMILES: CC(C)(C)c1ccc(OC2CCC2)c(S(=O)(=O)Cl)c1 ; COc1c(F)c(Cn2cccn2)cc2onc(N)c12 ; COc1c(F)c(Cn2cccn2)cc2onc(NS(=O)(=O)c3cc(C(C)(C)C)ccc3OC3CCC3)c12]

### Step a:

### N-(6-((1H-Pyrazol-1-yl)methyl)-5-fluoro-4-methoxybenzo[d]isoxazol-3-yl)-5-(tert-butyl)-2-cyclobutoxybenzenesulfonamide

Sodium hydride (60%, 37 mg, 0.93 mmol) was added to a solution of intermediate 2 (80 mg, 0.31 mmol) in N,N-dimethylformamide (2 mL) at 0° C. The reaction system was stirred at 0° C. for 10 min. Then, compound 3d (139 mg, 0.46 mmol) was added to the system, and stirring was continued for 20 min. After the reaction was completed, the reaction mixture was warmed to room temperature, quenched with a saturated aqueous ammonium chloride solution (10 mL), and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 7 (22 mg, yield: 13.6%).

¹HNMR (400 MHz, DMSO-d₆): δ10.36 (s, 1H), 7.96-7.75 (m, 2H), 7.68-7.55 (m, 1H), 7.49 (d, 1H), 6.95 (d, 1H), 6.90 (d, 1H), 6.30 (t, 1H), 5.50 (s, 2H), 4.66-4.62 (m, 1H), 3.94 (d, 3H), 2.20-2.16 (m, 2H), 1.74-1.57 (m, 2H), 1.57-1.48 (m, 2H), 1.26 (s, 9H).

MS m/z (ESI): 529.0 [M+H]⁺.

### Example 14

### Synthesis of Compound 8

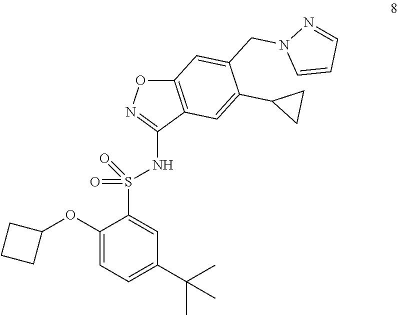 [SMILES: CC(C)(C)c1ccc(OC2CCC2)c(S(=O)(=O)Nc2noc3cc(Cn4cccn4)c(C4CC4)cc23)c1]

### N-(6-((1H-Pyrazol-1-yl)methyl)-5-cyclopropylbenzo[d]isoxazol-3-yl)-5-(tert-butyl)-2-cyclobutoxybenzenesulfonamide

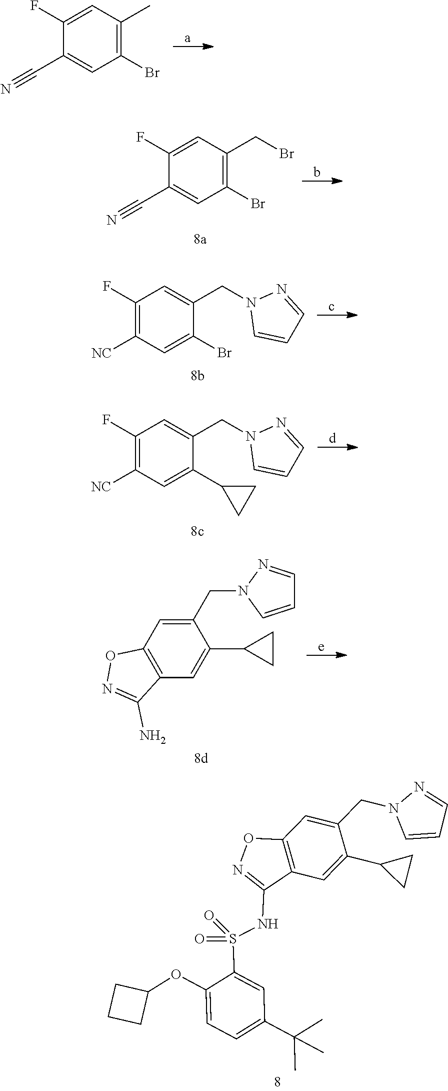 [SMILES: Cc1cc(F)c(C#N)cc1Br ; N#Cc1cc(Br)c(CBr)cc1F ; N#Cc1cc(Br)c(Cn2cccn2)cc1F ; N#Cc1cc(C2CC2)c(Cn2cccn2)cc1F ; Nc1noc2cc(Cn3cccn3)c(C3CC3)cc12 ; CC(C)(C)c1ccc(OC2CCC2)c(S(=O)(=O)Nc2noc3cc(Cn4cccn4)c(C4CC4)cc23)c1]

### Step a:

### 5-Bromo-4-(bromomethyl)-2-fluorobenzonitrile

1,3-Dibromo-5,5-dimethylimidazolidine-2,4-dione (0.67 g, 2.34 mmol) and azobisisobutyronitrile (0.10 g, 0.61 mmol) were added to a solution of 5-bromo-2-fluoro-4-methylbenzonitrile (1.00 g, 4.67 mmol) in acetonitrile (10 mL). The reaction system was heated to 80° C. and stirred for 16 h. After the reaction was completed, the reaction mixture was cooled to room temperature and concentrated under reduced pressure. The resulting crude product was purified by flash silica gel column chromatography (petroleum ether:ethyl acetate=9:1) to give compound 8a (0.85 g, yield: 62.1%).

¹H NMR (400 MHz, CDCl₃): δ7.83 (d, 1H), 7.37 (d, 1H), 4.53 (s, 2H).

### Step b:

### 4-((1H-Pyrazol-1-yl)methyl)-5-bromo-2-fluorobenzonitrile

Pyrazole (0.25 g, 3.67 mmol) and cesium carbonate (1.33 g, 4.08 mmol) were added to a solution of compound 8a (1.00 g, 3.41 mmol) in acetonitrile (16 mL). The reaction system was stirred at 25° C. for 5 h. After the reaction was completed, the mixture was diluted with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=5:1) to give compound 8b (0.73 g, yield: 76.3%).

¹H NMR (300 MHz, CDCl₃): δ7.82 (d, 1H), 7.64 (d, 1H), 7.52 (d, 1H), 6.50 (d, 1H), 6.39 (t, 1H), 5.42 (s, 2H).

MS m/z (ESI): 281.0 [M+H]⁺.

### Step c:

### 4-((1H-Pyrazol-1-yl)methyl)-5-cyclopropyl-2-fluorobenzonitrile

Under a nitrogen atmosphere, cesium carbonate (1.58 g, 4.85 mmol), cyclopropylboronic acid (0.42 g, 4.89 mmol), tris(dibenzylideneacetone)dipalladium (0.16 g, 0.17 mmol), and 2-(di-tert-butylphosphino)biphenyl (0.14 g, 0.47 mmol) were added to a solution of compound 8b (0.68 g, 2.43 mmol) in 1,4-dioxane (15 mL). The reaction system was heated to 90° C. and stirred for 16 h. After the reaction was completed, the mixture was diluted with water and extracted with ethyl acetate (80 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=5:1) to give compound 8c (0.54 g, yield: 92.2%).

¹HNMR (300 MHz, CDCl₃): δ7.62 (d, 1H), 7.46 (d, 1H), 7.33 (d, 1H), 6.49 (d, 1H), 6.38 (t, 1H), 5.56 (s, 2H), 1.87-1.80 (m, 1H), 1.07-1.03 (m, 2H), 0.71-0.67 (m, 2H).

MS m/z (ESI): 240.1 [M−H]⁺.

### Step d:

### 6-((1H-Pyrazol-1-yl)methyl)-5-cyclopropylbenzo[d]isoxazol-3-amine

Acetohydroxamic acid (0.46 g, 6.13 mmol) and tetramethylguanidine (2.08 g, 18.06 mmol) were added to compound 8c (0.49 g, 2.03 mmol) in a mixed solution (acetonitrile and water in a ratio of 9:1, 10 mL). The reaction system was heated to 60° C. and stirred for 16 h under a nitrogen atmosphere. After the reaction was completed, the reaction mixture was cooled to room temperature, then diluted with water, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=1:1) to give compound 8d (0.35 g, yield: 67.8%).

¹HNMR (400 MHz, DMSO-d₆): δ7.84 (d, 1H), 7.58 (s, 1H), 7.53 (d, 1H), 6.71 (s, 1H), 6.36-6.28 (m, 3H), 5.64 (s, 2H), 2.10-1.99 (m, 1H), 1.02-0.90 (m, 2H), 0.69-0.56 (m, 2H).

MS m/z (ESI): 255.2 [M+H]⁺.

### Step e:

### N-(6-((1H-Pyrazol-1-yl)methyl)-5-cyclopropylbenzo[d]isoxazol-3-yl)-5-(tert-butyl)-2-cyclobutoxybenzenesulfonamide

Compound 8d (100 mg, 0.39 mmol) and compound 3d (357 mg, 1.18 mmol) were dissolved in pyridine (6 mL). The reaction system was heated to 120° C. and stirred for 56 h under a nitrogen atmosphere. After the reaction was completed, the reaction mixture was cooled to room temperature and concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 8 (35 mg, yield: 17.1%).

¹HNMR (400 MHz, DMSO-d₆): δ11.48 (s, 1H), 7.88-7.76 (m, 3H), 7.56 (d, 1H), 7.52 (d, 1H), 6.86 (d, 1H), 6.82 (s, 1H), 6.32 (t, 1H), 5.65 (s, 2H), 4.74-4.60 (m, 1H), 2.25-2.12 (m, 2H), 2.12-2.10 (m, 1H), 1.78-1.64 (m, 2H), 1.53-1.37 (m, 2H), 1.25 (s, 9H), 1.02-0.93 (m, 2H), 0.64-0.54 (m, 2H).

MS m/z (ESI): 521.1 [M+H]⁺.

### Example 15

### Synthesis of Compound 9

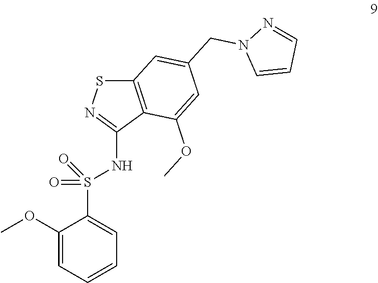 [SMILES: COc1ccccc1S(=O)(=O)Nc1nsc2cc(Cn3cccn3)cc(OC)c12]

### N-(6-((1H-Pyrazol-1-yl)methyl)-4-methoxybenzo[d]isothiazol-3-yl)-2-methoxybenzenesulfonamide

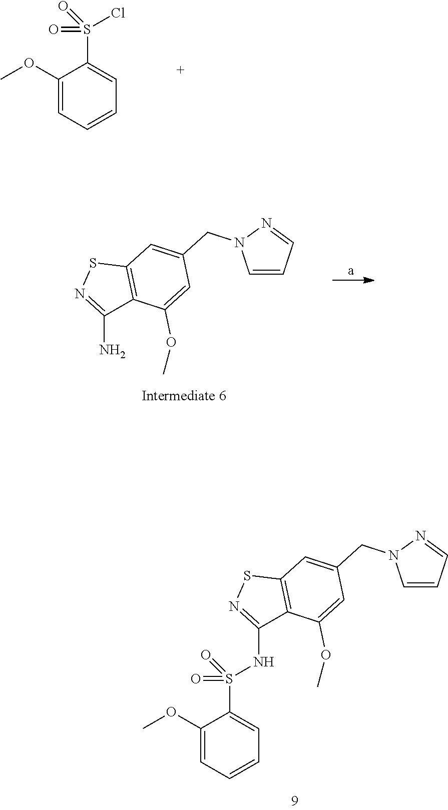 [SMILES: COc1ccccc1S(=O)(=O)Cl ; COc1cc(Cn2cccn2)cc2snc(N)c12 ; COc1ccccc1S(=O)(=O)Nc1nsc2cc(Cn3cccn3)cc(OC)c12]

### Step a:

### N-(6-((1H-Pyrazol-1-yl)methyl)-4-methoxybenzo[d]isothiazol-3-yl)-2-methoxybenzenesulfonamide

Under a nitrogen atmosphere, 2-methoxybenzenesulfonyl chloride (64 mg, 0.31 mmol) was added to a solution of intermediate 6 (80 mg, 0.31 mmol) in pyridine (5 mL), and the reaction system was heated to 70° C. and stirred for 16 h. After the reaction was completed, the reaction mixture was cooled to room temperature and concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 9 (23 mg, yield: 17.4%). ¹HNMR (400 MHz, DMSO-d₆): δ9.41 (brs, 1H), 7.91-7.88 (m, 2H), 7.62-7.58 (m, 1H), 7.49 (d, 1H), 7.28 (s, 1H), 7.15-7.09 (m, 2H), 6.94 (s, 1H), 6.30 (t, 1H), 5.45 (s, 2H), 4.05 (s, 3H), 3.78 (s, 3H).

MS m/z (ESI): 430.9 [M+H]⁺.

### Example 16

### Synthesis of Compound 10

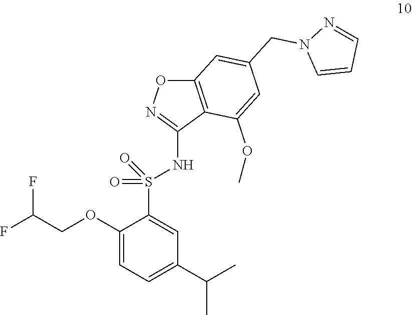 [SMILES: COc1cc(Cn2cccn2)cc2onc(NS(=O)(=O)c3cc(C(C)C)ccc3OCC(F)F)c12]

### N-(6-((1H-Pyrazol-1-yl)methyl)-4-methoxybenzo[d]isoxazol-3-yl)-2-(2,2-difluoroethoxy)-5-isopropylbenzenesulfonamide

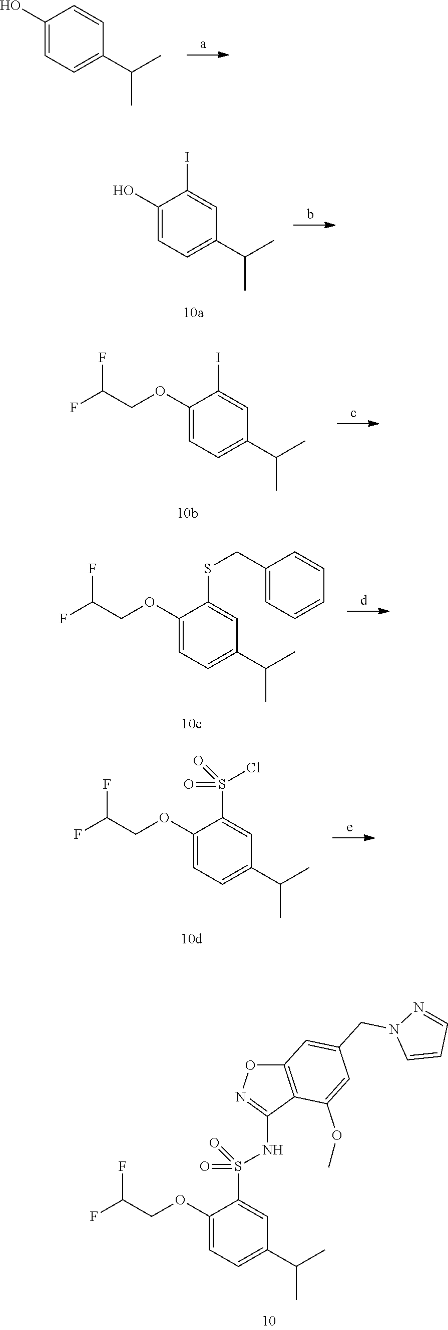

### Step a:

### 2-Iodo-4-isopropylphenol

p-Toluenesulfonic acid (0.17 g, 0.99 mmol) and N-iodosuccinimide (2.25 g, 9.99 mmol) were added to a solution of 4-isopropylphenol (1.36 g, 9.99 mmol) in dichloromethane (40 mL) at 10° C. The reaction system was stirred for 16 h. After the reaction was completed, the mixture was diluted with water and extracted with dichloromethane (150 mL×3). The organic layers were combined and dried over anhydrous sodium sulfate. After filtration, the mother liquor was concentrated under reduced pressure. The resulting crude product was purified by flash silica gel column chromatography (petroleum ether:ethyl acetate=30:1) to give compound 10a (1.72 g, yield: 65.7%).

¹H NMR (400 MHz, CDCl₃): δ7.49 (d, 1H), 7.11-7.08 (m, 1H), 6.91 (d, 1H), 5.13 (s, 1H), 2.83-2.80 (m, 1H), 1.21 (s, 3H), 1.20 (s, 3H).

### Step b:

### 1-(2,2-Difluoroethoxy)-2-iodo-4-isopropylbenzene

Compound 10a (1.60 g, 6.10 mmol) and potassium carbonate (1.69 g, 12.20 mmol) were dissolved in a solution of N,N-dimethylformamide (15 mL). The reaction system was stirred at 25° C. for 5 min, and then 1,1-difluoro-2-iodoethane (1.29 g, 6.72 mmol) was added. The reaction system was heated to 100° C. and stirred for 16 h. After the reaction was completed, the reaction mixture was cooled to room temperature, diluted with water (50 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 10b (1.80 g, yield: 90.4%).

¹H NMR (400 MHz, CDCl₃): δ7.64 (d, 1H), 7.17-7.14 (m, 1H), 6.76 (d, 1H), 6.29-5.99 (m, 1H), 4.23-4.16 (m, 2H), 2.86-2.79 (m, 1H), 1.22 (s, 3H), 1.20 (s, 3H).

### Step c:

### Benzyl(2-(2,2-difluoroethoxy)-5-isopropylphenyl)sulfane

Under a nitrogen atmosphere, benzyl mercaptan (0.78 g, 6.20 mmol), potassium carbonate (1.33 g, 9.60 mmol), 1,4-diazabicyclo[2.2.2]octane (0.05 g, 0.48 mmol), and copper(I) iodide (0.05 g, 0.24 mmol) were added to a solution of compound 10b (1.56 g, 4.78 mmol) in N,N-dimethylformamide (15 mL). The reaction system was heated to 100° C. and stirred for 16 h.

After the reaction was completed, the reaction mixture was cooled to room temperature, diluted with water (50 mL), and extracted with dichloromethane (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:dichloromethane=30:1) to give compound 10c (1.49 g, yield: 96.6%).

¹H NMR (400 MHz, CDCl₃) δ 7.32-7.27 (m, 3H), 7.26-7.19 (m, 2H), 7.10 (d, 1H), 7.04-7.01 (m, 1H), 6.77 (d, 1H), 6.26-5.97 (m, 1H), 4.23-4.15 (m, 2H), 4.08 (s, 2H), 2.82-2.75 (m, 1H), 1.16 (s, 3H), 1.15 (s, 3H).

MS m/z (ESI): 321.1 [M−H]⁺.

### Step d:

### 2-(2,2-Difluoroethoxy)-5-isopropylbenzenesulfonyl chloride

N-Chlorosuccinimide (3.69 g, 27.60 mmol) was added to compound 10c (1.49 g, 4.62 mmol) in a mixed solution (glacial acetic acid and water in a ratio of 10:1, 22 mL) at 25° C. The reaction system was stirred at 25° C. for 2 h. After the reaction was completed, water (100 mL) was added for dilution, and the mixture was extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=10:1) to give compound 10d (1.12 g, yield: 81.1%).

¹HNMR (400 MHz, CDCl₃): δ7.82 (d, 1H), 7.56-7.54 (m, 1H), 7.04 (d, 1H), 6.37-6.07 (m, 1H), 4.42-4.34 (m, 2H), 3.00-2.93 (m, 1H), 1.28 (s, 3H), 1.26 (s, 3H).

### Step e:

### N-(6-((1H-Pyrazol-1-yl)methyl)-4-methoxybenzo[d]isoxazol-3-yl)-2-(2,2-difluoroethoxy)-5-isopropylbenzenesulfonamide

Intermediate 1 (50 mg, 0.20 mmol) and compound 10d (306 mg, 1.02 mmol) were dissolved in a solution of pyridine (5 mL). The reaction system was heated to 120° C. and stirred for 24 h. After the reaction was completed, the reaction mixture was concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 10 (57 mg, yield: 55.0%). ¹HNMR (400 MHz, DMSO-d₆): δ9.93 (s, 1H), 7.87 (d, 1H), 7.69 (d, 1H), 7.52-7.48 (m, 2H), 7.22 (s, 1H), 6.82-6.73 (m, 2H), 6.52-6.23 (m, 2H), 5.43 (s, 2H), 4.41-4.35 (m, 2H), 3.80 (s, 3H), 2.95-2.91 (m, 1H), 1.18 (s, 3H), 1.16 (s, 3H).

MS m/z (ESI): 507.0 [M+H]⁺.

### Example 17

### Synthesis of Compound 11

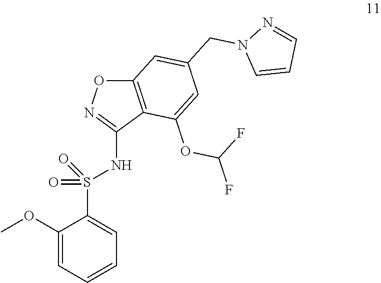 [SMILES: COc1ccccc1S(=O)(=O)Nc1noc2cc(Cn3cccn3)cc(OC(F)F)c12]

### N-(6-((1H-Pyrazol-1-yl)methyl)-4-(difluoromethoxy)benzo[d]isoxazol-3-yl)-2-methoxybenzenesulfonamide

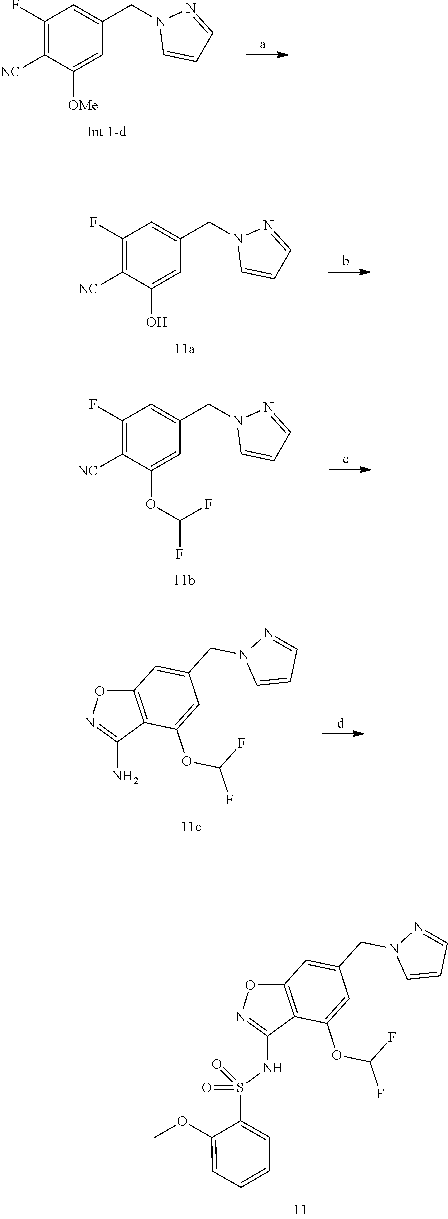 [SMILES: COc1cc(Cn2cccn2)cc(F)c1C#N ; N#Cc1c(O)cc(Cn2cccn2)cc1F ; N#Cc1c(F)cc(Cn2cccn2)cc1OC(F)F ; Nc1noc2cc(Cn3cccn3)cc(OC(F)F)c12 ; COc1ccccc1S(=O)(=O)Nc1noc2cc(Cn3cccn3)cc(OC(F)F)c12]

### Step a:

### 4-((1H-Pyrazol-1-yl)methyl)-2-fluoro-6-hydroxybenzonitrile

Compound Int 1-d (1.10 g, 4.76 mmol) was dissolved in a solution of pyridine in hydrochloric acid (7 mL). The reaction system was heated to 180° C. and stirred for 2.5 h. After the reaction was completed, the reaction mixture was cooled to room temperature and poured into ice water, and the resulting mixture was extracted with ethyl acetate (20 mL×3). The organic layers were combined, washed with a saturated aqueous NaCl solution, and dried over anhydrous sodium sulfate. After filtration, the mother liquor was concentrated under reduced pressure to give compound 11a (0.78 g, yield: 75.5%).

MS m/z (ESI): 218.0 [M+H]⁺.

### Step b:

### 4-((1H-Pyrazol-1-yl)methyl)-2-(difluoromethoxy)-6-fluorobenzonitrile

Sodium chlorodifluoroacetate (821 mg, 5.31 mmol) and potassium carbonate (745 mg, 5.39 mmol) were added to compound 11a (780 mg, 3.59 mmol) in a mixed solution (N,N-dimethylformamide and water in a ratio of 30:1, 20 mL). The reaction system was heated to 120° C. and stirred for 4 h under a nitrogen atmosphere. After the reaction was completed, the reaction mixture was cooled to room temperature, diluted with water, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:tetrahydrofuran=4:1) to give compound 11b (650 mg, yield: 67.7%).

¹H NMR (300 MHz, CDCl₃): δ8.11 (d, 1H), 7.98 (d, 1H), 7.78-6.91 (m, 3H), 6.87 (t, 1H), 5.88 (s, 2H).

### Step c:

### 6-((1H-Pyrazol-1-yl)methyl)-4-(difluoromethoxy)benzo[d]isoxazol-3-amine

Compound 11b (0.65 g, 2.43 mmol), acetohydroxamic acid (0.55 g, 7.30 mmol), and tetramethylguanidine (1.68 g, 14.60 mmol) were dissolved in a mixed solution (acetonitrile and water in a ratio of 15:1, 5 mL). The reaction system was heated to 60° C. and stirred for 7 h under a nitrogen atmosphere. After the reaction was completed, the reaction mixture was cooled to room temperature and then concentrated under reduced pressure. The resulting crude product was purified by flash silica gel column chromatography (dichloromethane:methanol=20:1) to give compound 11c (0.42 g, yield: 61.6%).

MS m/z (ESI): 281.0 [M+H]⁺.

### Step d:

### N-(6-((1H-Pyrazol-1-yl)methyl)-4-(difluoromethoxy)benzo[d]isoxazol-3-yl)-2-methoxybenzenesulfonamide

Under a nitrogen atmosphere, at 0° C., sodium hydride (60%, 71 mg, 1.78 mmol) was added to a solution of 11c (100 mg, 0.36 mmol) in N,N-dimethylformamide (4 mL), and the reaction system was stirred for 10 min. Then, 2-methoxybenzenesulfonyl chloride (111 mg, 0.54 mmol) was added to the system. The mixture was stirred at 0° C. for 1 h, then warmed to 25° C., and stirred for another 1 h. After the reaction was completed, the mixture was quenched with water and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 11 (28 mg, yield: 17.4%).

¹HNMR (400 MHz, DMSO-d₆): δ10.67 (s, 1H), 7.97-7.87 (m, 1H), 7.77 (d, 1H), 7.75-7.58 (m, 1H), 7.51 (s, 1H), 6.37-6.92 (m, 5H), 6.37-6.28 (m, 1H), 5.50 (s, 2H), 3.75 (s, 3H).

MS m/z (ESI): 450.9 [M+H]⁺.

### Example 18

### Synthesis of Compound 12

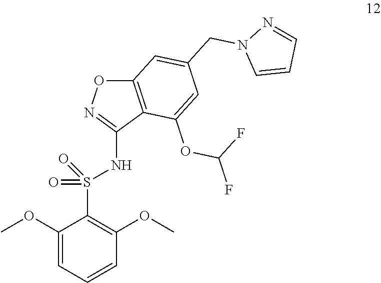 [SMILES: COc1cccc(OC)c1S(=O)(=O)Nc1noc2cc(Cn3cccn3)cc(OC(F)F)c12]

### N-(6-((1H-Pyrazol-1-yl)methyl)-4-(difluoromethoxy)benzo[d]isoxazol-3-yl)-2,6-dimethoxybenzenesulfonamide

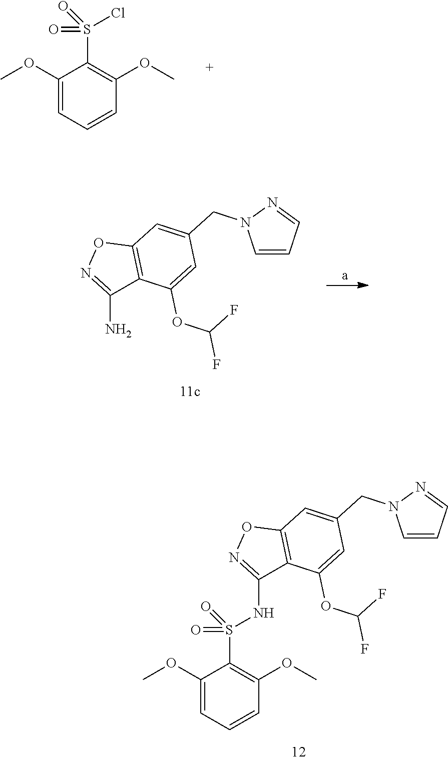

### Step a:

### N-(6-((1H-Pyrazol-1-yl)methyl)-4-(difluoromethoxy)benzo[d]isoxazol-3-yl)-2,6-dimethoxybenzenesulfonamide

Under a nitrogen atmosphere, at 0° C., sodium hydride (60%, 71 mg, 1.78 mmol) was added to a solution of 11c (100 mg, 0.36 mmol) in N,N-dimethylformamide (4 mL), and the reaction system was stirred for 10 min. Then, 2,6-dimethoxybenzenesulfonyl chloride (127 mg, 0.53 mmol) was added to the system. The mixture was stirred at 0° C. for 1 h, then warmed to 25° C., and stirred for another 1 h. After the reaction was completed, the mixture was quenched with water and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 m; mobile phase A: 5 mM aqueous ammonium bicarbonate solution, mobile phase B: acetonitrile; gradient ratio:phase B 5%-95%) to give compound 12 (54 mg, yield: 31.5%).

¹HNMR (400 MHz, DMSO-d₆): δ10.22 (s, 1H), 7.89 (d, 1H), 7.51-7.11 (m, 4H), 7.00 (s, 1H), 6.77 (d, 2H), 6.36-6.26 (m, 1H), 5.50 (s, 2H), 3.74 (s, 6H).

MS m/z (ESI): 481.0 [M+H]⁺.

### Example 19

### Synthesis of Compound 13

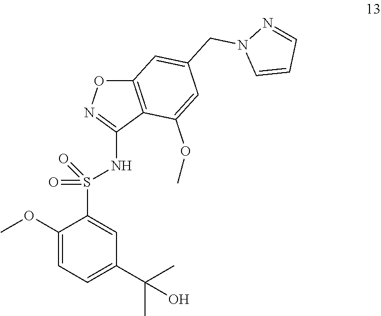 [SMILES: COc1ccc(C(C)(C)O)cc1S(=O)(=O)Nc1noc2cc(Cn3cccn3)cc(OC)c12]

### N-(6-((1H-Pyrazol-1-yl)methyl)-4-methoxybenzo[d]isoxazol-3-yl)-5-(2-hydroxypropan-2-yl)-2-methoxybenzenesulfonamide

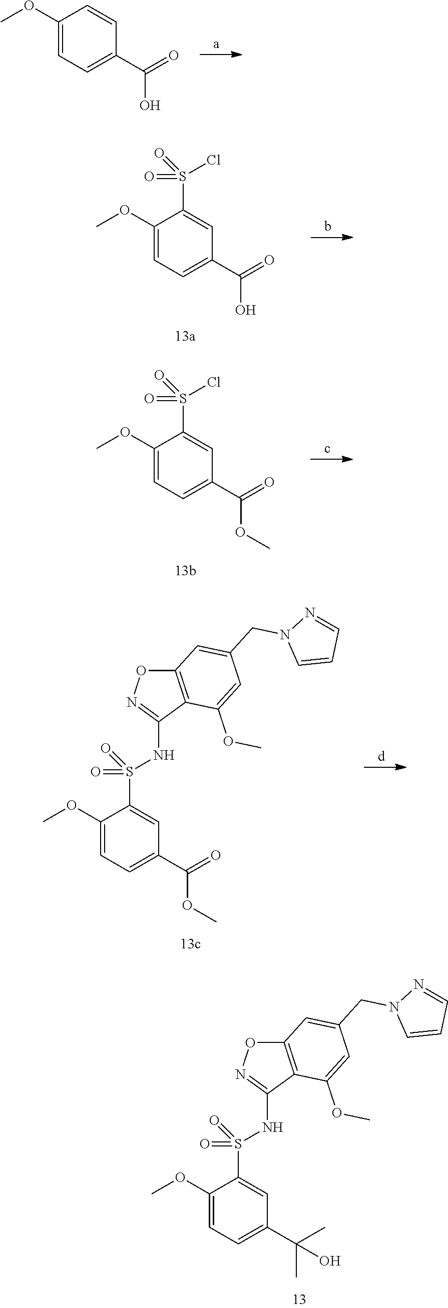

### Step a:

### 3-(Chlorosulfonyl)-4-methoxybenzoic acid

p-Methoxybenzoic acid (500 mg, 3.29 mmol) was dissolved in chlorosulfonic acid (2 mL). The reaction system was heated to 65° C. and stirred for 5 h under a nitrogen atmosphere. After the reaction was completed, the reaction mixture was slowly poured into ice water, and the resulting mixture was filtered under reduced pressure to give compound 13a (610 mg, yield: 74.1%).

¹H NMR (400 MHz, DMSO-d₆): δ13.64 (s, 1H), 8.30 (d, 1H), 7.92-7.88 (m, 1H), 7.06 (d, 1H), 3.83 (s, 3H).

MS m/z (ESI): 248.9 [M−H]⁺.

### Step b:

### Methyl 3-(chlorosulfonyl)-4-methoxybenzoate

Under a nitrogen atmosphere, compound 13a (4.00 g, 15.96 mmol) was dissolved in thionyl chloride (80 mL) at 20° C., and the reaction system was stirred at 20° C. for 18 h. Subsequently, methanol (10 mL) was added to the reaction system, and stirring was continued for 2 h. After the reaction was completed, the mixture was diluted with water and extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 13b (2.90 g, yield: 68.7%).

¹H NMR (400 MHz, CDCl₃): δ8.64 (d, 1H), 8.39-8.35 (m, 1H), 7.18 (d, 1H), 4.14 (s, 3H), 3.95 (s, 3H).

### Step c:

### Methyl 3-(N-(6-((1H-pyrazol-1-yl)methyl)-4-methoxybenzo[d]isoxazol-3-yl)sulfamoyl)-4-methoxybenzoate

Under a nitrogen atmosphere, at 0° C., sodium hydride (60%, 82 mg, 2.05 mmol) was added to a solution of intermediate 1 (100 mg, 0.41 mmol) in N,N-dimethylformamide (2 mL), and the reaction system was stirred for 1 h. Then, compound 13b (217 mg, 0.82 mmol) was added to the system, and stirring was continued at 0° C. for 1 h. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:tetrahydrofuran=10:1) to give compound 13c (68 mg, yield: 35.2%).

¹HNMR (400 MHz, DMSO-d₆): δ 10.67 (s, 1H), 8.36 (d, 1H), 8.22-8.18 (m, 1H), 7.87 (d, 1H), 7.50 (d, 1H), 7.35 (d, 1H), 6.85 (s, 1H), 6.75 (s, 1H), 6.30 (t, 1H), 5.44 (s, 2H), 3.86 (s, 3H), 3.85 (s, 3H), 3.79 (s, 3H).

MS m/z (ESI): 473.1 [M+H]⁺.

### Step d:

### N-(6-((1H-Pyrazol-1-yl)methyl)-4-methoxybenzo[d]isoxazol-3-yl)-5-(2-hydroxypropan-2-yl)-2-methoxybenzenesulfonamide

Under a nitrogen atmosphere, methylmagnesium iodide (237 mg, 3.18 mmol) was added to a solution of compound 13c (150 mg, 0.32 mmol) in tetrahydrofuran (7 mL) at 20° C., and the reaction system was stirred at 20° C. for 2 h. After the reaction was completed, the mixture was quenched with a saturated aqueous ammonium chloride solution and extracted with dichloromethane (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 13 (21 mg, yield: 14.0%).

¹HNMR (400 MHz, DMSO-d₆): δ10.02 (s, 1H), 7.92 (d, 1H), 7.87 (d, 1H), 7.64 (d, 1H), 7.49 (d, 1H), 7.10 (d, 1H), 6.83 (s, 1H), 6.73 (s, 1H), 6.30 (t, 1H), 5.43 (s, 2H), 5.17 (s, 1H), 3.80 (s, 3H), 3.74 (s, 3H), 1.39 (s, 6H).

MS m/z (ESI): 473.1 [M+H]⁺.

### Example 20

### Synthesis of Compound 14

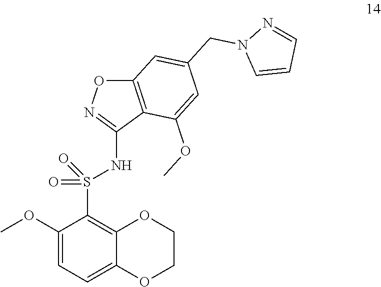 [SMILES: COc1ccc2c(c1S(=O)(=O)Nc1noc3cc(Cn4cccn4)cc(OC)c13)OCCO2]

### N-(6-((1H-Pyrazol-1-yl)methyl)-4-methoxybenzo[d]isoxazol-3-yl)-6-methoxy-2,3-dihydrobenzo[b][1,4]dioxine-5-sulfonamide

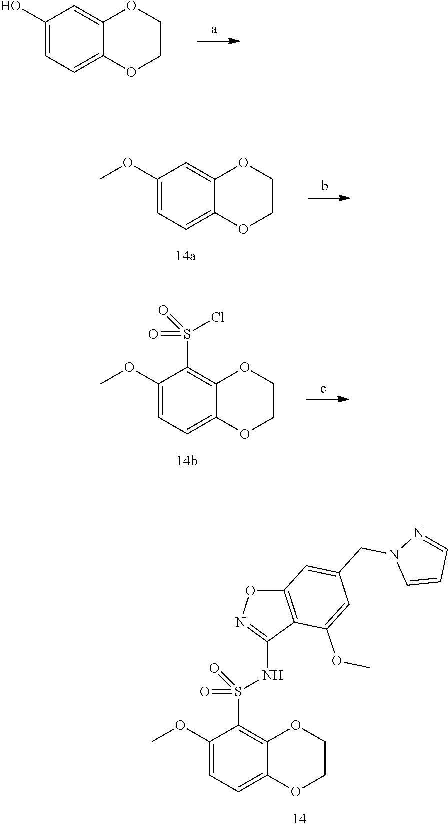 [SMILES: Oc1ccc2c(c1)OCCO2 ; COc1ccc2c(c1)OCCO2 ; COc1ccc2c(c1S(=O)(=O)Cl)OCCO2 ; COc1ccc2c(c1S(=O)(=O)Nc1noc3cc(Cn4cccn4)cc(OC)c13)OCCO2]

### Step a:

### 6-Methoxy-2,3-dihydrobenzo[b][1,4]dioxine

6-Hydroxy-1,4-benzodioxane (500 mg, 3.29 mmol), iodomethane (700 mg, 4.93 mmol), and potassium carbonate (908 mg, 6.57 mmol) were dissolved in a solution of N,N-dimethylformamide (20 mL). The reaction system was stirred at 20° C. for 16 h. After the reaction was completed, the reaction mixture was filtered, and the filtrate was concentrated under reduced pressure. The resulting crude product was purified by flash silica gel column chromatography (petroleum ether:ethyl acetate=3:1) to give compound 14a (410 mg, yield: 75.1%).

¹H NMR (400 MHz, CDCl₃): δ6.79 (d, 1H), 6.46-6.41 (m, 2H), 4.26-4.20 (m, 4H), 3.75 (s, 3H).

### Step b:

### 6-Methoxy-2,3-dihydrobenzo[b][1,4]dioxine-5-sulfonyl chloride

Under a nitrogen atmosphere, n-butyllithium (2.5 M in n-hexane, 3.5 mL, 8.75 mmol) was added dropwise to a solution of compound 14a (1.20 g, 7.22 mmol) in tetrahydrofuran (25 mL) at 0° C., and the reaction system was stirred at 0° C. for 30 min and then cooled to −78° C. Subsequently, sulfuryl chloride (1.46 g, 10.82 mmol) was added, and stirring was continued at −78° C. for 30 min. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 14b (0.25 g, yield: 13.1%).

¹H NMR (400 MHz, CDCl₃): δ7.15 (d, 1H), 6.57 (d, 1H), 4.47-4.45 (m, 2H), 4.31-4.29 (m, 2H), 3.86 (s, 3H).

### Step c:

### N-(6-((1H-Pyrazol-1-yl)methyl)-4-methoxybenzo[d]isoxazol-3-yl)-6-methoxy-2,3-dihydrobenzo[b][1,4]dioxine-5-sulfonamide

Under a nitrogen atmosphere, at 25° C., sodium hydride (60%, 246 mg, 6.14 mmol) was added to a solution of intermediate 1 (150 mg, 0.61 mmol) in N,N-dimethylformamide (2 mL), and the reaction system was stirred for 1 h. Then, compound 14b (195 mg, 0.73 mmol) was added to the system, and stirring was continued at 25° C. for 1 h. After the reaction was completed, the mixture was diluted with water and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 14 (21 mg, yield: 7.2%).

¹HNMR (400 MHz, DMSO-d₆): δ 9.73 (s, 1H), 7.87 (d, 1H), 7.50 (d, 1H), 7.04 (d, 1H), 6.84 (s, 1H), 6.75 (s, 1H), 6.63 (d, 1H), 6.31-6.30 (m, 1H), 5.45 (s, 2H), 4.20-4.15 (m, 4H), 3.86 (s, 3H), 3.69 (s, 3H).

MS m/z (ESI): 473.0 [M+H]⁺.

### Example 21

### Synthesis of Compound 15

### N-(6-((1H-Pyrazol-1-yl)methyl)-4-methoxybenzo[d]isoxazol-3-yl)-7-methoxyspiro[chromane-4,1′-cyclobutane]-8-sulfonamide

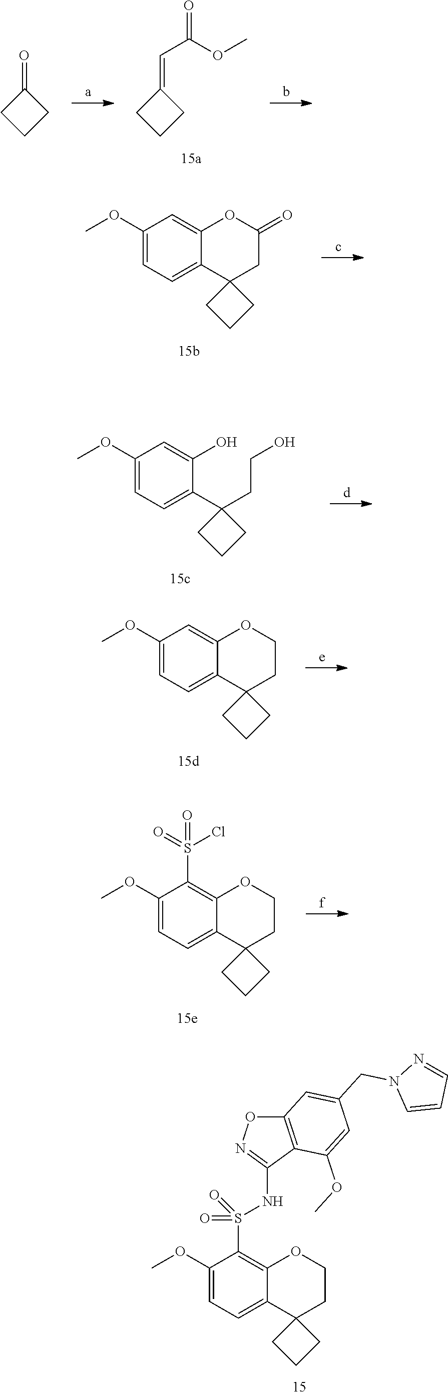

### Step a:

### Methyl 2-cyclobutylideneacetate

Sodium hydride (60%, 2.08 g, 52.00 mmol) was added to a solution of trimethyl phosphonoacetate (9.46 g, 51.95 mmol) in tetrahydrofuran (80 mL) at 0° C. The reaction system was stirred at 0° C. for 1 h. Then, cyclobutanone (2.60 g, 37.10 mmol) was added, and the reaction system was stirred at 25° C. for another 2 h. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 15a (3.00 g, yield: 64.1%).

¹H NMR (400 MHz, CDCl₃) δ5.59-5.58 (m, 1H), 3.67 (s, 3H), 3.12 (t, 2H), 2.83 (t, 2H), 2.12-2.04 (m, 2H)

### Step b:

### 7-Methoxyspiro[chromane-4,1′-cyclobutan]-2-one

3-Methoxyphenol (6.88 g, 55.42 mmol), compound 15a (3.50 g, 27.74 mmol), and 98% concentrated sulfuric acid (0.54 g, 5.40 mmol) were mixed. The reaction system was then heated to 130° C. and stirred for 2 h. After the reaction was completed, the mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 15b (0.74 g, yield: 12.2%).

¹H NMR (400 MHz, CDCl₃): δ7.38 (d, 1H), 6.78-6.75 (m, 1H), 6.64 (d, 1H), 3.82 (s, 3H), 2.89 (s, 2H), 2.40-2.37 (m, 2H), 2.13-2.03 (m, 4H).

### Step c:

### 2-(1-(2-Hydroxyethyl)cyclobutyl)-5-methoxyphenol

Lithium aluminum hydride (136 mg, 3.39 mmol) was added to a solution of compound 15b (740 mg, 3.39 mmol) in tetrahydrofuran (20 mL) at 0° C. The reaction system was stirred at 0° C. for 1 h. After the reaction was completed, the mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 15c (570 mg, yield: 75.6%).

¹H NMR (400 MHz, CDCl₃): δ6.85 (d, 1H), 6.50-6.47 (m, 1H), 6.42 (d, 1H), 3.79 (s, 3H), 3.67 (t, 2H), 2.55-2.47 (m, 2H), 2.34-2.29 (m, 2H), 2.22-2.07 (m, 3H). 1.88-1.85 (m, 1H).

### Step d:

### 7-Methoxyspiro[chromane-4,1′-cyclobutane]

Compound 15c (570 mg, 2.56 mmol) and p-toluenesulfonic acid (88 mg, 0.51 mmol) were dissolved in a solution of toluene (10 mL). The reaction system was heated to 110° C. and stirred for 3 h. After the reaction was completed, the reaction mixture was concentrated under reduced pressure. The resulting crude product was purified by flash silica gel column chromatography (petroleum ether:ethyl acetate=9:1) to give compound 15d (360 mg, yield: 68.7%).

¹H NMR (400 MHz, CDCl₃): δ 7.39 (d, 1H), 6.55-6.53 (m, 1H), 6.32 (d, 1H), 4.12 (t, 2H), 3.75 (s, 3H), 2.43-2.38 (m, 2H), 2.09-1.93 (m, 6H).

### Step e:

### 7-Methoxyspiro[chromane-4,1′-cyclobutane]-8-sulfonyl chloride

Under a nitrogen atmosphere, n-butyllithium (2.5 M in n-hexane, 0.9 mL, 2.25 mmol) was added dropwise to a solution of compound 15d (300 mg, 1.47 mmol) in tetrahydrofuran (10 mL) at 0° C., and the reaction system was stirred at 0° C. for 1 h and then cooled to −78° C. Subsequently, sulfuryl chloride (297 mg, 2.20 mmol) was added, and stirring was continued at −78° C. for 30 min. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 15e (190 mg, yield: 42.7%).

¹H NMR (400 MHz, DMSO-d₆): δ 7.47 (d, 1H), 6.58 (d, 1H), 4.04-4.02 (m, 2H), 3.67 (s, 3H), 2.32-2.30 (m, 2H), 2.01-1.88 (m, 6H).

### Step f:

### N-(6-((1H-Pyrazol-1-yl)methyl)-4-methoxybenzo[d]isoxazol-3-yl)-7-methoxyspiro[chromane-4,1′-cyclobutane]-8-sulfonamide

Sodium hydride (60%, 82 mg, 2.05 mmol) was added to a solution of intermediate 1 (100 mg, 0.41 mmol) in N,N-dimethylformamide (1 mL) at 0° C. The reaction system was stirred for 15 min. Then, compound 15e (186 mg, 0.61 mmol) was added to the system, and stirring was continued at 0° C. for 30 min. After the reaction was completed, the reaction system was diluted with water, adjusted to pH=7 with a 1 N aqueous hydrochloric acid solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 15 (100 mg, yield: 47.8%).

¹HNMR (400 MHz, DMSO-d₆): δ9.49 (s, 1H), 7.87 (d, 1H), 7.73 (d, 1H), 7.49 (d, 1H), 6.83 (s, 1H), 6.76-6.72 (m, 2H), 6.30 (s, 1H), 5.45 (s, 2H), 4.05 (t, 2H), 3.87 (s, 3H), 3.73 (s, 3H), 2.33-2.28 (m, 2H), 2.02-1.87 (m, 6H).

MS m/z (ESI): 511.0 [M+H]⁺.

### Example 22

### Synthesis of Compound 16

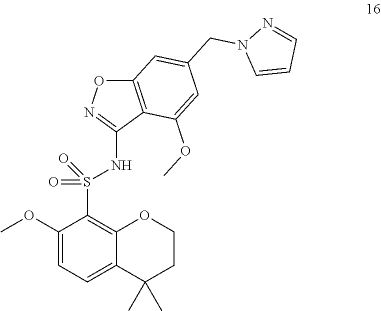 [SMILES: COc1ccc2c(c1S(=O)(=O)Nc1noc3cc(Cn4cccn4)cc(OC)c13)OCCC2(C)C]

### N-(6-((1H-Pyrazol-1-yl)methyl)-4-methoxybenzo[d]isoxazol-3-yl)-7-methoxy-4,4-dimethylchromane-8-sulfonamide

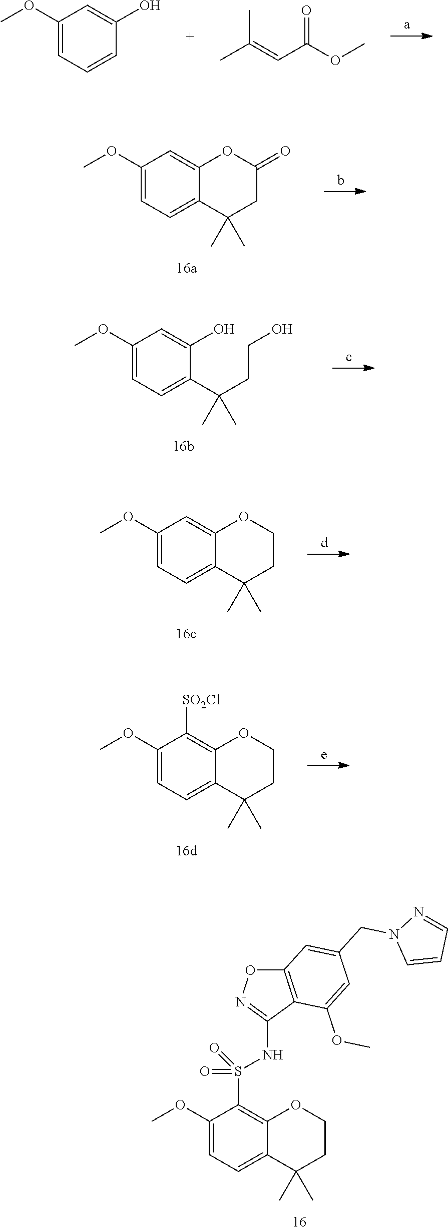

### Step a:

### 7-Methoxy-4,4-dimethylchroman-2-one

3-Methoxyphenol (8.69 g, 70.00 mmol), methyl 3,3-dimethylacrylate (4.00 g, 35.04 mmol), and 98% concentrated sulfuric acid (0.69 g, 6.90 mmol) were mixed. The reaction system was then heated to 130° C. and stirred for 3 h. After the reaction was completed, the mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 16a (2.30 g, yield: 31.8%).

¹H NMR (400 MHz, CDCl₃): δ7.22 (d, 1H), 6.74-6.71 (m, 1H), 6.64 (d, 1H), 3.82 (s, 3H), 2.62 (s, 2H), 1.35 (s, 6H).

### Step b:

### 2-(4-Hydroxy-2-methylbutan-2-yl)-5-methoxyphenol

Lithium aluminum hydride (0.33 g, 8.73 mmol) was added to a solution of compound 16a (1.80 g, 8.73 mmol) in tetrahydrofuran (20 mL) at 0° C. The reaction system was stirred at 0° C. for 1 h. After the reaction was completed, the mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 16b (1.39 g, yield: 75.7%).

¹H NMR (400 MHz, CDCl₃): δ7.09 (d, 1H), 6.41-6.39 (m, 1H), 6.24 (d, 1H), 3.74 (s, 3H), 3.54 (t, 2H), 2.18 (t, 2H), 1.38 (s, 6H).

### Step c:

### 7-Methoxy-4,4-dimethylchromane

Compound 16b (320 mg, 1.52 mmol) and p-toluenesulfonic acid (53 mg, 0.31 mmol) were dissolved in a solution of toluene (10 mL). The reaction system was heated to 120° C. and stirred for 2 h. After the reaction was completed, the reaction mixture was concentrated under reduced pressure. The resulting crude product was purified by flash silica gel column chromatography (petroleum ether:ethyl acetate=9:1) to give compound 16c (150 mg, yield: 51.3%).

¹H NMR (400 MHz, CDCl₃): δ 7.15 (d, 1H), 6.49-6.46 (m, 1H), 6.34 (d, 1H), 4.18 (t, 2H), 3.74 (s, 3H), 1.81 (t, 2H), 1.30 (s, 6H).

### Step d:

### 7-Methoxy-4,4-dimethylchromane-8-sulfonyl chloride

Under a nitrogen atmosphere, n-butyllithium (2.5 M in n-hexane, 1.1 mL, 2.75 mmol) was added dropwise to a solution of compound 16c (450 mg, 2.34 mmol) in tetrahydrofuran (3 mL) at 0° C., and the reaction system was stirred at 0° C. for 1 h and then cooled to −78° C. Subsequently, sulfuryl chloride (474 mg, 3.51 mmol) was added, and stirring was continued at −78° C. for 1 h.

After the reaction was completed, the mixture was quenched with a saturated aqueous ammonium chloride solution and extracted with ethyl acetate (70 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 16d (125 mg, yield: 18.4%).

¹H NMR (400 MHz, CDCl₃): δ 7.42 (d, 1H), 6.50 (d, 1H), 4.32 (t, 2H), 3.87 (s, 3H), 1.81 (t, 2H), 1.26 (s, 6H).

### Step e:

### N-(6-((1H-Pyrazol-1-yl)methyl)-4-methoxybenzo[d]isoxazol-3-yl)-7-methoxy-4,4-dimethylchromane-8-sulfonamide

Under a nitrogen atmosphere, at 25° C., sodium hydride (60%, 62 mg, 1.55 mmol) was added to a solution of intermediate 1 (75 mg, 0.31 mmol) in N,N-dimethylformamide (3 mL), and the reaction system was stirred for 1 h. Then, compound 16d (112 mg, 0.38 mmol) was added to the system, and stirring was continued at 25° C. for 1 h. After the reaction was completed, the mixture was diluted with water and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 16 (30 mg, yield: 19.6%).

¹HNMR (400 MHz, DMSO-d₆): δ 9.51 (s, 1H), 7.87 (d, 1H), 7.50-7.48 (m, 2H), 6.83 (s, 1H), 6.75 (s, 1H), 6.67 (d, 1H), 6.31 (t, 1H), 5.45 (s, 2H), 4.08 (t, 2H), 3.87 (s, 3H), 3.71 (s, 3H), 1.67 (t, 2H), 1.20 (s, 6H).

MS m/z (ESI): 499.0 [M+H]⁺.

### Example 23

### Synthesis of Compound 17

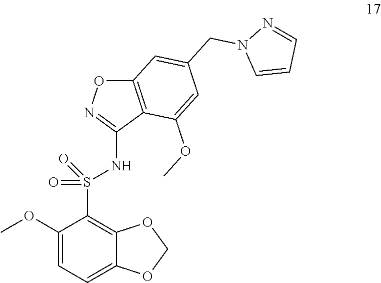 [SMILES: COc1ccc2c(c1S(=O)(=O)Nc1noc3cc(Cn4cccn4)cc(OC)c13)OCO2]

### N-(6-((1H-Pyrazol-1-yl)methyl)-4-methoxybenzo[d]isoxazol-3-yl)-5-methoxybenzo[d][1,3]dioxole-4-sulfonamide

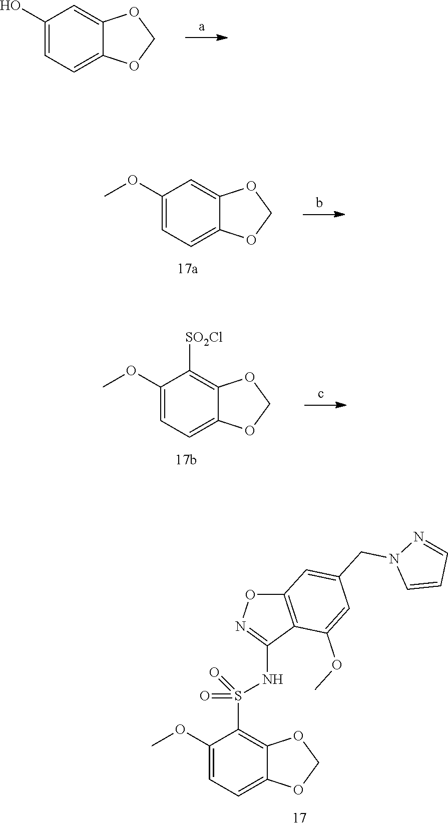 [SMILES: Oc1ccc2c(c1)OCO2 ; COc1ccc2c(c1)OCO2 ; COc1ccc2c(c1S(=O)(=O)Cl)OCO2 ; COc1ccc2c(c1S(=O)(=O)Nc1noc3cc(Cn4cccn4)cc(OC)c13)OCO2]

### Step a:

### 5-Methoxybenzo[d][1,3]dioxole

Sesamol (2.00 g, 14.48 mmol), iodomethane (3.09 g, 21.77 mmol), and potassium carbonate (4.10 g, 29.67 mmol) were dissolved in acetone (30 mL). The reaction system was stirred at 25° C. for 16 h. After the reaction was completed, the reaction system was diluted with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 17a (1.60 g, yield: 72.6%).

¹H NMR (400 MHz, CDCl₃): δ6.71 (d, 1H), 6.49 (d, 1H), 6.33-6.31 (m, 1H), 5.91 (s, 2H), 3.75 (s, 3H).

### Step b:

### 5-Methoxybenzo[d][1,3]dioxole-4-sulfonyl chloride

Under a nitrogen atmosphere, n-butyllithium (1.6 M in n-hexane, 0.98 mL, 1.58 mmol) was added dropwise to a solution of compound 17a (200 mg, 1.31 mmol) in tetrahydrofuran (10 mL) at 0° C., and the reaction system was stirred at 0° C. for 1 h and then cooled to −78° C. Subsequently, sulfuryl chloride (355 mg, 2.63 mmol) was added, and stirring was continued at −78° C. for 1 h. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 17b (110 mg, yield: 33.4%).

¹H NMR (400 MHz, DMSO-d₆): δ6.76 (d, 1H), 6.34 (d, 1H), 5.91 (s, 2H), 3.66 (s, 3H).

### Step c:

### N-(6-((1H-Pyrazol-1-yl)methyl)-4-methoxybenzo[d]isoxazol-3-yl)-5-methoxybenzo[d][1,3]dioxole-4-sulfonamide

Sodium hydride (60%, 164 mg, 4.09 mmol) was added to a solution of intermediate 1 (200 mg, 0.82 mmol) in N,N-dimethylformamide (2 mL) at 0° C. The reaction system was stirred at 0° C. for 10 min. Then, compound 17b (411 mg, 1.64 mmol) was added, and stirring was continued for 50 min. After the reaction was completed, the reaction mixture was warmed to room temperature, quenched with a saturated aqueous ammonium chloride solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 17 (92 mg, yield: 24.5%).

¹HNMR (400 MHz, DMSO-d₆): δ10.28 (s, 1H), 7.88 (d, 1H), 7.50 (d, 1H), 7.08 (d, 1H), 6.86 (s, 1H), 6.75 (s, 1H), 6.49 (d, 1H), 6.31-6.30 (m, 1H), 6.07 (s, 2H), 5.45 (s, 2H), 3.82 (s, 3H), 3.66 (s, 3H).

MS m/z (ESI): 459.0 [M+H]⁺.

### Example 24

### Synthesis of Compound 18

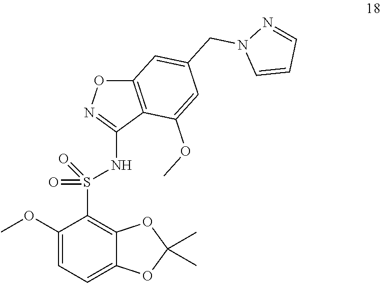 [SMILES: COc1ccc2c(c1S(=O)(=O)Nc1noc3cc(Cn4cccn4)cc(OC)c13)OC(C)(C)O2]

### N-(6-((1H-Pyrazol-1-yl)methyl)-4-methoxybenzo[d]isoxazol-3-yl)-5-methoxy-2,2-dimethylbenzo[d][1,3]dioxole-4-sulfonamide

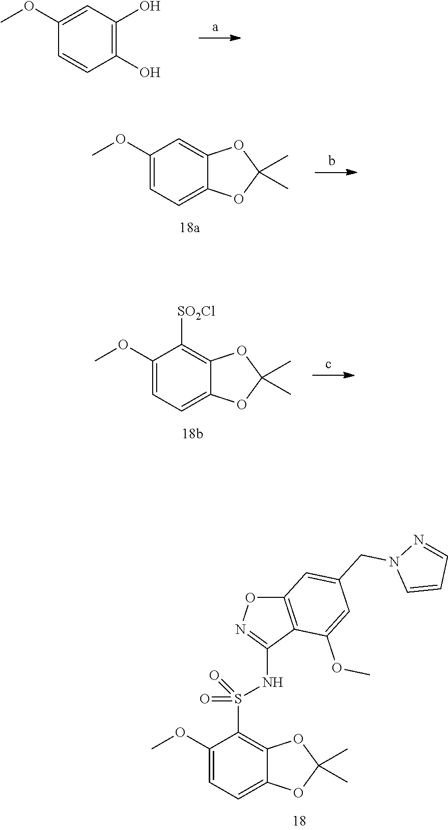

### Step a:

### 5-Methoxy-2,2-dimethylbenzo[d][1,3]dioxole

4-Methoxybenzene-1,2-diol (1.00 g, 7.14 mmol), 2,2-dimethoxypropane (1.49 g, 14.27 mmol), and p-toluenesulfonic acid (0.12 g, 0.71 mmol) were dissolved in a solution of toluene (30 mL). The reaction system was heated to 120° C. and stirred for 16 h. After the reaction was completed, the reaction mixture was concentrated under reduced pressure. The resulting crude product was purified by flash silica gel column chromatography (petroleum ether:ethyl acetate=20:1) to give compound 18a (0.69 g, yield: 53.7%).

¹H NMR (400 MHz, DMSO-d₆): δ6.69 (d, 1H), 6.50 (d, 1H), 6.31-6.28 (m, 1H), 3.66 (s, 3H), 1.60 (s, 6H).

### Step b:

### 5-Methoxy-2,2-dimethylbenzo[d][1,3]dioxole-4-sulfonyl chloride

Under a nitrogen atmosphere, n-butyllithium (2.5 M in n-hexane, 4.3 mL, 10.75 mmol) was added dropwise to a solution of compound 18a (1.60 g, 8.84 mmol) in tetrahydrofuran (20 mL) at 0° C., and the reaction system was stirred at 0° C. for 1 h and then cooled to −78° C. Subsequently, sulfuryl chloride (2.40 g, 17.78 mmol) was added, and stirring was continued at −78° C. for 1 h. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 18b (0.47 g, yield: 19.0%).

¹H NMR (400 MHz, CDCl₃): δ6.92 (d, 1H), 6.39 (d, 1H), 3.96 (s, 3H), 1.78 (s, 6H).

### Step c:

### N-(6-((1H-Pyrazol-1-yl)methyl)-4-methoxybenzo[d]isoxazol-3-yl)-5-methoxy-2,2-dimethylbenzo[d][1,3]dioxole-4-sulfonamide

Sodium hydride (60%, 115 mg, 2.87 mmol) was added to a solution of intermediate 1 (140 mg, 0.57 mmol) in N,N-dimethylformamide (2 mL) at 0° C. The reaction system was stirred at 0° C. for 10 min. Then, compound 18b (320 mg, 1.15 mmol) was added, and stirring was continued for 50 min. After the reaction was completed, the reaction mixture was warmed to room temperature, quenched with a saturated aqueous ammonium chloride solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=9:1) to give compound 18 (30 mg, yield: 10.8%).

¹HNMR (400 MHz, DMSO-d₆): δ10.18 (s, 1H), 7.81 (d, 1H), 7.50 (d, 1H), 6.97 (d, 1H), 6.85 (s, 1H), 6.74 (s, 1H), 6.45 (d, 1H), 6.31-6.30 (m, 1H), 5.45 (s, 2H), 3.83 (s, 3H), 3.66 (s, 3H), 1.53 (s, 6H).

MS m/z (ESI): 487.0 [M+H]⁺.

### Example 25

### Synthesis of Compound 19

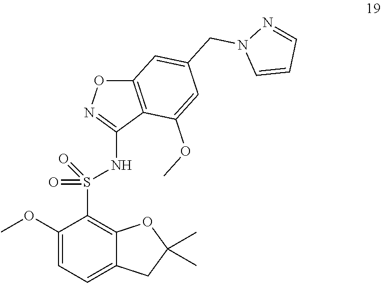 [SMILES: COc1ccc2c(c1S(=O)(=O)Nc1noc3cc(Cn4cccn4)cc(OC)c13)OC(C)(C)C2]

### N-(6-((1H-Pyrazol-1-yl)methyl)-4-methoxybenzo[d]isoxazol-3-yl)-6-methoxy-2,2-dimethyl-2,3-dihydrobenzofuran-7-sulfonamide

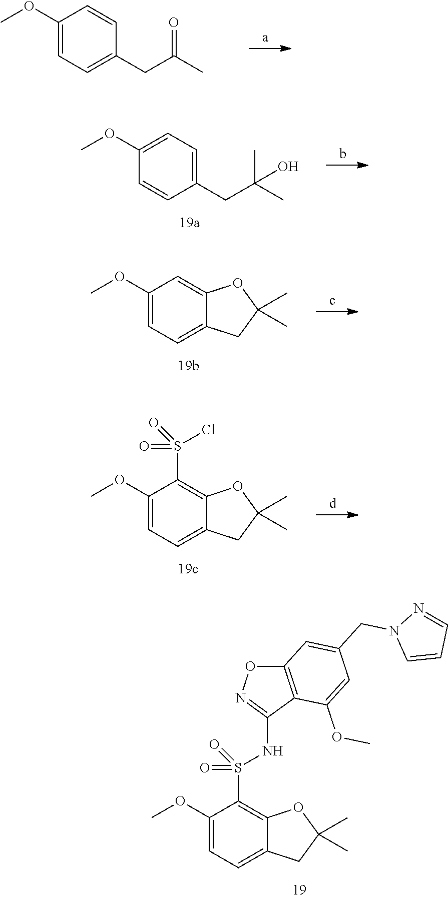

### Step a:

### 1-(4-Methoxyphenyl)-2-methylpropan-2-ol

Under a nitrogen atmosphere, methylmagnesium chloride (3.0 M in tetrahydrofuran, 9.10 mL, 27.30 mmol) was added dropwise to a solution of p-methoxyphenylacetone (1.50 g, 9.14 mmol) in tetrahydrofuran (30 mL), and the reaction system was stirred at 0° C. for 16 h. After the reaction was completed, the mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 19a (1.05 g, yield: 63.8%).

¹H NMR (400 MHz, CDCl₃) δ 7.15 (d, 2H), 6.87 (d, 2H), 3.82 (s, 3H), 2.72 (s, 2H), 1.20 (s, 6H).

### Step b:

### 6-Methoxy-2,2-dimethyl-2,3-dihydrobenzofuran

Under a hydrogen atmosphere, lithium carbonate (1.23 g, 16.65 mmol), (diacetoxyiodo)benzene (5.36 g, 16.64 mmol), and palladium acetate (0.12 g, 0.53 mmol) were added to a solution of compound 19a (2.00 g, 11.10 mmol) in perfluorobenzene (25 mL), and the reaction system was heated to 120° C. and stirred for 36 h. After the reaction was completed, the reaction mixture was filtered under reduced pressure, and the filtrate was concentrated under reduced pressure. The resulting crude product was purified by flash silica gel column chromatography (petroleum ether:ethyl acetate=9:1) to give compound 19b (0.55 g, yield: 27.8%).

¹H NMR (400 MHz, CDCl₃): δ6.92 (d, 1H), 6.32-6.26 (m, 2H), 3.68 (s, 3H), 2.87 (s, 2H), 1.39 (s, 6H).

### Step c:

### 6-Methoxy-2,2-dimethyl-2,3-dihydrobenzofuran-7-sulfonyl chloride

Under a nitrogen atmosphere, n-butyllithium (2.5 M in n-hexane, 0.8 mL, 2.00 mmol) was added dropwise to a solution of compound 19b (250 mg, 1.40 mmol) in tetrahydrofuran (5 mL) at 0° C., and the reaction system was stirred at 0° C. for 1 h and then cooled to −78° C. Subsequently, sulfuryl chloride (227 mg, 1.68 mmol) was added, and stirring was continued at −78° C. for 1 h. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 19c (50 mg, yield: 12.9%).

¹H NMR (400 MHz, CDCl₃): δ 7.30 (d, 1H), 6.43 (d, 1H), 3.96 (s, 3H), 2.98 (s, 2H), 1.55 (s, 6H).

### Step d:

### N-(6-((1H-Pyrazol-1-yl)methyl)-4-methoxybenzo[d]isoxazol-3-yl)-6-methoxy-2,2-dimethyl-2,3-dihydrobenzofuran-7-sulfonamide

Under a nitrogen atmosphere, at 0° C., sodium hydride (60%, 50 mg, 1.24 mmol) was added to a solution of intermediate 1 (100 mg, 0.41 mmol) in N,N-dimethylformamide (2 mL), and the reaction system was stirred for 1 h. Then, compound 19c (170 mg, 0.61 mmol) was added to the system, and the reaction mixture was warmed to room temperature and stirred for 1 h. After the reaction was completed, the mixture was diluted with water and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 19 (5 mg, yield: 2.5%).

¹HNMR (400 MHz, DMSO-d₆): δ 9.55 (s, 1H), 7.87 (d, 1H), 7.50 (d, 1H), 7.29 (d, 1H), 6.83 (s, 1H), 6.76 (s, 1H), 6.53 (d, 1H), 6.30 (t, 1H), 5.45 (s, 2H), 3.88 (s, 3H), 3.73 (s, 3H), 2.88 (s, 2H), 1.20 (s, 6H).

MS m/z (ESI): 485.0 [M+H]⁺.

### Example 26

### Synthesis of Compound 20

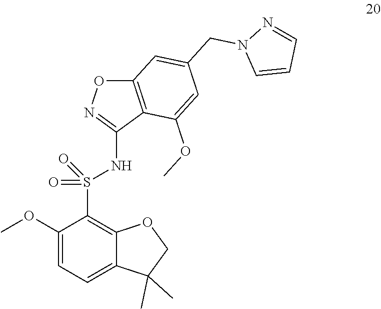 [SMILES: COc1ccc2c(c1S(=O)(=O)Nc1noc3cc(Cn4cccn4)cc(OC)c13)OCC2(C)C]

### N-(6-((1H-Pyrazol-1-yl)methyl)-4-methoxybenzo[d]isoxazol-3-yl)-6-methoxy-3,3-dimethyl-2,3-dihydrobenzofuran-7-sulfonamide

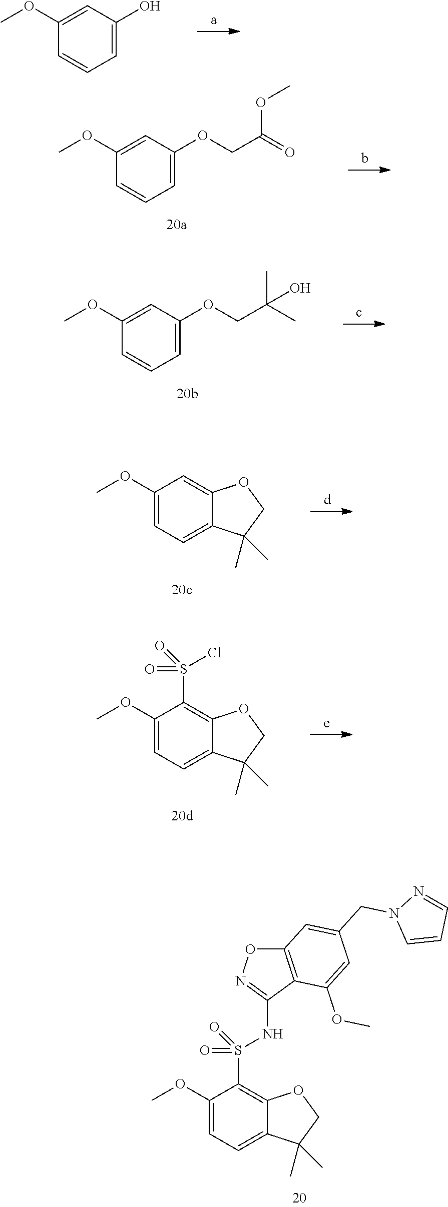

### Step a:

### Methyl 2-(3-methoxyphenoxy)acetate

3-Methoxyphenol (8.00 g, 64.44 mmol), ethyl bromoacetate (16.13 g, 96.59 mmol), and potassium carbonate (17.80 g, 128.79 mmol) were dissolved in acetone (100 mL). The reaction system was heated to 60° C. and stirred for 16 h. After the reaction was completed, the reaction mixture was filtered under reduced pressure, and the filtrate was concentrated under reduced pressure. The resulting crude product was then purified by flash silica gel column chromatography (petroleum ether:ethyl acetate=3:1) to give compound 20a (12.30 g, yield: 97.3%).

¹H NMR (400 MHz, CDCl₃): δ7.20 (t, 1H), 6.59-6.49 (m, 3H), 4.62 (s, 2H), 4.29 (q, 2H), 3.80 (s, 3H) 1.32 (t, 3H).

### Step b:

### 1-(3-Methoxyphenoxy)-2-methylpropan-2-ol

Methylmagnesium chloride (3.0 M in n-hexane, 4.76 mL, 14.27 mmol) was added dropwise to a solution of compound 20a (1.00 g, 4.76 mmol) in tetrahydrofuran (10 mL) at 0° C., and the reaction system was stirred at 0° C. for 1 h. After the reaction was completed, the reaction mixture was quenched with water and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 20b (0.82 g, yield: 82.0%).

¹H NMR (400 MHz, CDCl₃): δ7.18 (t, 1H), 6.54-6.48 (m, 3H), 3.80 (s, 3H), 3.78 (s, 2H), 1.34 (s, 6H).

### Step c:

### 6-Methoxy-3,3-dimethyl-2,3-dihydrobenzofuran

Under a nitrogen atmosphere, phosphorus pentoxide (19.23 g, 135.48 mmol) was added to a solution of compound 20b (7.60 g, 38.73 mmol) in methanesulfonic acid (150 mL), and the reaction system was stirred at 25° C. for 2 h. After the reaction was completed, the reaction mixture was quenched with ice water and extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 20c (1.90 g, yield: 27.5%).

MS m/z (ESI): 179.1 [M+H]⁺.

### Step d:

### 6-Methoxy-3,3-dimethyl-2,3-dihydrobenzofuran-7-sulfonyl chloride

Under a nitrogen atmosphere, n-butyllithium (2.5 M in n-hexane, 0.54 mL, 1.35 mmol) was added dropwise to a solution of compound 20c (200 mg, 1.12 mmol) in tetrahydrofuran (10 mL) at 0° C., and the reaction system was stirred at 0° C. for 1 h and then cooled to −78° C. Subsequently, sulfuryl chloride (227 mg, 1.68 mmol) was added, and stirring was continued at −78° C. for 1 h. After the reaction was completed, the mixture was quenched with a saturated aqueous ammonium chloride solution and extracted with ethyl acetate (10 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 20d (30 mg, yield: 9.7%).

¹H NMR (400 MHz, CDCl₃): δ7.23 (d, 1H), 6.36 (d, 1H), 3.89 (s, 3H), 2.90 (s, 2H), 1.48 (s, 6H).

### Step e:

### N-(6-((1H-Pyrazol-1-yl)methyl)-4-methoxybenzo[d]isoxazol-3-yl)-6-methoxy-3,3-dimethyl-2,3-dihydrobenzofuran-7-sulfonamide

Sodium hydride (60%, 106 mg, 2.66 mmol) was added to a solution of intermediate 1 (130 mg, 0.53 mmol) in N,N-dimethylformamide (4 mL) at 0° C. The reaction system was stirred at 25° C. for 1 h. Then, compound 20d (198 mg, 0.72 mmol) was added, and stirring was continued for 1 h. After the reaction was completed, the reaction mixture was quenched with water and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 20 (75 mg, yield: 29.1%).

¹HNMR (400 MHz, DMSO-d₆): δ9.56 (s, 1H), 7.69 (d, 1H), 7.50 (d, 1H), 7.29 (d, 1H), 6.83 (s, 1H), 6.75 (s, 1H), 6.52 (d, 1H), 6.30 (t, 1H), 5.45 (s, 2H), 3.88 (s, 3H), 3.73 (s, 3H), 2.88 (s, 2H), 1.20 (s, 6H).

MS m/z (ESI): 485.0 [M+H]⁺.

### Example 27

### Synthesis of Compound 21

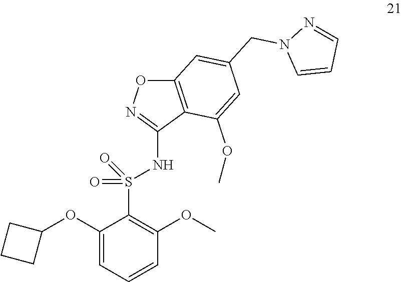 [SMILES: COc1cccc(OC2CCC2)c1S(=O)(=O)Nc1noc2cc(Cn3cccn3)cc(OC)c12]

### 15 N-(6-((1H-Pyrazol-1-yl)methyl)-4-methoxybenzo[d]isoxazol-3-yl)-2-cyclobutoxy-6-methoxybenzenesulfonamide

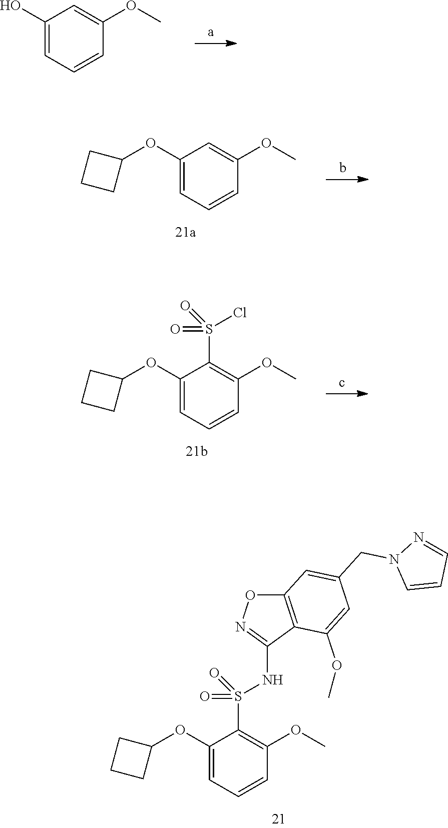

### Step a:

### 1-Cyclobutoxy-3-methoxybenzene

Potassium carbonate (2.24 g, 16.21 mmol) and bromocyclobutane (1.31 g, 9.78 mmol) were added to a solution of 3-methoxyphenol (1.00 g, 8.06 mmol) in N,N-dimethylformamide (10 mL). The reaction system was heated to 100° C. and stirred for 40 h. After the reaction was completed, the reaction mixture was cooled to room temperature, diluted with water (50 mL), and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=20:1) to give compound 21a (1.00 g, yield: 69.7%).

¹H NMR (400 MHz, DMSO-d₆): δ7.18-7.12 (m, 1H), 6.51-6.48 (m, 1H), 6.43-6.36 (m, 2H), 4.71-4.61 (m, 1H), 3.72 (s, 3H), 2.46-2.36 (m, 2H), 2.08-1.95 (m, 2H), 1.82-1.58 (m, 2H).

### Step b:

### 2-Cyclobutoxy-6-methoxybenzenesulfonyl chloride

Under a nitrogen atmosphere, n-butyllithium (2.5 M in n-hexane, 2.4 mL, 6.00 mmol) was added dropwise to a solution of compound 21a (900 mg, 5.05 mmol) in tetrahydrofuran (20 mL) at 0° C., and the reaction system was stirred at 0° C. for 1 h and then cooled to −78° C. Subsequently, sulfuryl chloride (1.36 g, 10.08 mmol) was added, and stirring was continued at −78° C. for 2 h. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=5:1) to give compound 21b (480 mg, yield: 34.3%).

¹H NMR (400 MHz, DMSO-d₆): δ7.25-7.19 (m, 1H), 6.63 (d, 1H), 6.47 (d, 1H), 4.64 (t, 1H), 3.72 (s, 3H), 2.39-2.29 (m, 2H), 2.13-2.00 (m, 2H), 1.80-1.54 (m, 2H).

### Step c:

### N-(6-((1H-Pyrazol-1-yl)methyl)-4-methoxybenzo[d]isoxazol-3-yl)-2-cyclobutoxy-6-methoxybenzenesulfonamide

Sodium hydride (60%, 82 mg, 2.05 mmol) was added to a solution of intermediate 1 (100 mg, 0.41 mmol) in N,N-dimethylformamide (3 mL) at 0° C. The reaction system was stirred at 0° C. for 1 h. Then, compound 21b (227 mg, 0.82 mmol) was added, and stirring was continued for 2 h. After the reaction was completed, the reaction mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 21 (45 mg, yield: 22.7%).

¹HNMR (400 MHz, DMSO-d₆): δ9.53 (s, 1H), 7.88 (d, 1H), 7.50 (d, 1H), 7.45 (t, 1H), 6.82 (s, 1H), 6.77-6.74 (m, 2H), 6.54 (d, 1H), 6.31 (t, 1H), 5.45 (s, 2H), 4.73-4.64 (m, 1H), 3.88 (s, 3H), 3.78 (s, 3H), 2.27-2.21 (m, 2H), 1.94-1.81 (m, 2H), 1.58-1.46 (m, 2H).

MS m/z (ESI): 485.0 [M+H]⁺.

### Example 28

### Synthesis of Compound 22

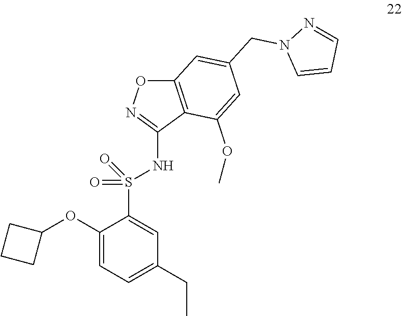 [SMILES: CCc1ccc(OC2CCC2)c(S(=O)(=O)Nc2noc3cc(Cn4cccn4)cc(OC)c23)c1]

### N-(6-((1H-Pyrazol-1-yl)methyl)-4-methoxybenzo[d]isoxazol-3-yl)-2-cyclobutoxy-5-ethylbenzenesulfonamide

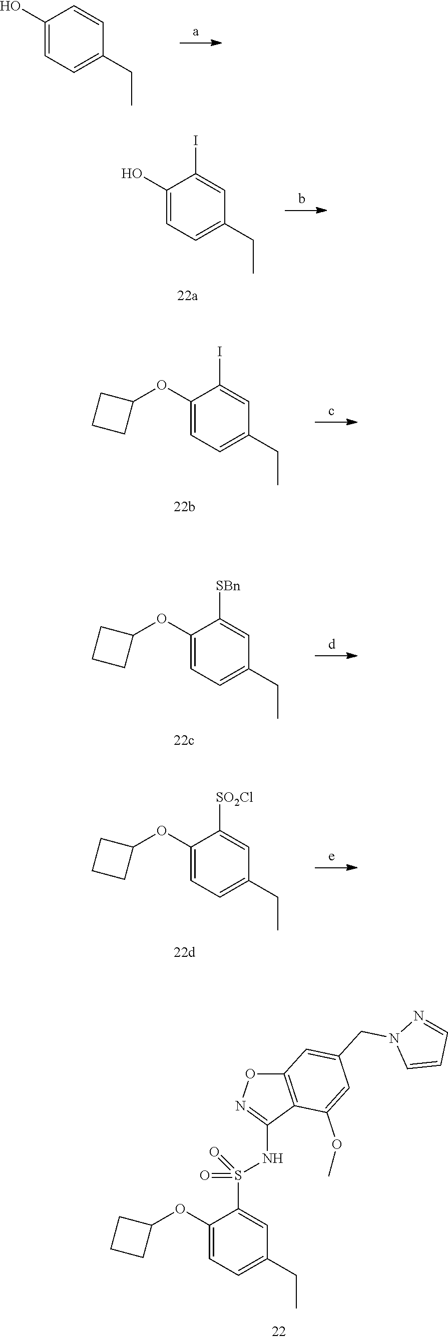

### Step a:

### 4-Ethyl-2-iodophenol

p-Toluenesulfonic acid (2.82 g, 16.38 mmol) was added to a solution of 4-ethylphenol (2.00 g, 16.37 mmol) in acetonitrile (50 mL) at 25° C. After the mixture was reacted for 10 min, N-iodosuccinimide (3.69 g, 16.40 mmol) was added, and the reaction system was stirred for 16 h. After the reaction was completed, the reaction mixture was quenched with an aqueous sodium sulfite solution (100 mL) and acidified with a hydrochloric acid solution (1 M), and then the phases were separated. The organic phase was collected, and the aqueous phase was extracted with ethyl acetate (100 mL×2). The organic layers were combined and dried over anhydrous sodium sulfate. After filtration, the mother liquor was concentrated under reduced pressure. The resulting crude product was purified by flash silica gel column chromatography (petroleum ether:ethyl acetate=9:1) to give compound 22a (2.80 g, yield: 68.9%).

¹H NMR (400 MHz, DMSO-d₆): δ10.00 (s, 1H), 7.49 (d, 1H), 7.03-7.01 (m, 1H), 6.79 (d, 1H), 2.49 (q, 2H), 1.11 (t, 3H).

### Step b:

### 1-Cyclobutoxy-4-ethyl-2-iodobenzene

Compound 22a (1.00 g, 4.03 mmol), bromocyclobutane (0.66 g, 4.84 mmol), and cesium carbonate (2.63 g, 8.06 mmol) were dissolved in a solution of N,N-dimethylformamide (15 mL). The reaction system was heated to 100° C. and stirred for 16 h. After the reaction was completed, the reaction mixture was cooled to room temperature, diluted with water (30 mL), and extracted with ethyl acetate (50 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 22b (1.10 g, yield: 90.3%).

¹H NMR (400 MHz, CDCl₃): δ7.60 (d, 1H), 7.07-7.05 (m, 1H), 6.59 (d, 1H), 4.66-4.63 (m, 1H), 2.54 (q, 2H), 2.47-2.43 (m, 2H), 2.27-2.22 (m, 2H), 1.89-1.85 (m, 1H), 1.71-1.66 (m, 1H), 1.19 (t, 3H).

### Step c:

### Benzyl(2-cyclobutoxy-5-ethylphenyl)sulfane

Under a nitrogen atmosphere, benzyl mercaptan (90 mg, 0.73 mmol), potassium carbonate (182 mg, 1.32 mmol), 1,4-diazabicyclo[2.2.2]octane (7.4 mg, 0.066 mmol), and copper(I) iodide (6.1 mg, 0.033 mmol) were added to a solution of compound 22b (200 mg, 0.66 mmol) in N,N-dimethylformamide (6 mL). The reaction system was heated to 100° C. and stirred for 16 h. After the reaction was completed, the reaction mixture was cooled to room temperature, diluted with water (20 mL), and extracted with ethyl acetate (20 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 22c (140 mg, yield: 70.9%).

¹H NMR (400 MHz, CDCl₃): δ7.24-7.14 (m, 5H), 6.98 (s, 1H), 6.87 (d, 1H), 6.54 (d, 1H), 4.60-4.56 (m, 1H), 4.03 (s, 2H), 2.47-2.35 (m, 4H), 2.17-2.12 (m, 2H), 1.79-1.77 (m, 1H), 1.61-1.59 (m, 1H), 1.07 (t, 3H).

### Step d:

### 2-Cyclobutoxy-5-ethylbenzenesulfonyl chloride

N-Chlorosuccinimide (1.05 g, 7.84 mmol) was added to compound 22c (780 mg, 2.61 mmol) in a mixed solution (glacial acetic acid and water in a ratio of 10:1, 10 mL) at 25° C. The reaction system was stirred at 25° C. for 3 h. After the reaction was completed, the mixture was quenched with water (10 mL) and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 22d (460 mg, yield: 64.1%).

¹HNMR (400 MHz, CDCl₃): δ7.75 (d, 1H), 7.44-7.41 (m, 1H), 6.85 (d, 1H), 4.87-4.82 (m, 1H), 2.65 (q, 2H), 2.53-2.47 (m, 2H), 2.38-2.30 (m, 2H), 1.95-1.93 (m, 1H), 1.76-1.72 (m, 1H), 1.24 (t, 3H).

### Step e:

### N-(6-((1H-Pyrazol-1-yl)methyl)-4-methoxybenzo[d]isoxazol-3-yl)-2-cyclobutoxy-5-ethylbenzenesulfonamide

Sodium hydride (60%, 82 mg, 2.05 mmol) was added to a solution of intermediate 1 (100 mg, 0.41 mmol) in N,N-dimethylformamide (2 mL) at 0° C. The reaction system was stirred at 0° C. for 10 min. Then, compound 22d (169 mg, 0.61 mmol) was added, and stirring was continued for 50 min. After the reaction was completed, the reaction mixture was quenched with a saturated aqueous ammonium chloride solution, and the system was adjusted to pH=5 with a 1 N aqueous hydrochloric acid solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=20:1) to give compound 22 (39 mg, yield: 19.7%).

¹HNMR (400 MHz, DMSO-d₆): δ9.73 (s, 1H), 7.87-7.81 (m, 1H), 7.67 (d, 1H), 7.49-7.48 (m, 1H), 7.42-7.39 (m, 1H), 6.88 (d, 1H), 6.84 (s, 1H), 6.75 (s, 1H), 6.30-6.29 (m, 1H), 5.44 (s, 2H), 4.69-4.66 (m, 1H), 3.81 (s, 3H), 2.60 (q, 2H), 2.23-2.20 (m, 2H), 1.80-1.75 (m, 2H), 1.55-1.51 (m, 2H), 1.15 (t, 3H).

MS m/z (ESI): 483.0 [M+H]⁺.

### Example 29

### Synthesis of Compound 23

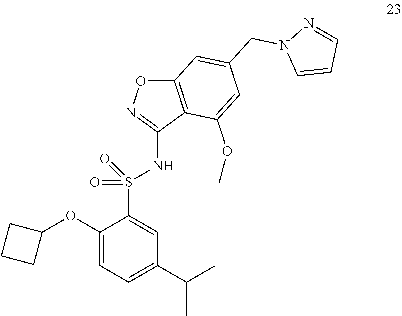 [SMILES: COc1cc(Cn2cccn2)cc2onc(NS(=O)(=O)c3cc(C(C)C)ccc3OC3CCC3)c12]

### N-(6-((1H-Pyrazol-1-yl)methyl)-4-methoxybenzo[d]isoxazol-3-yl)-2-cyclobutoxy-5-isopropylbenzenesulfonamide

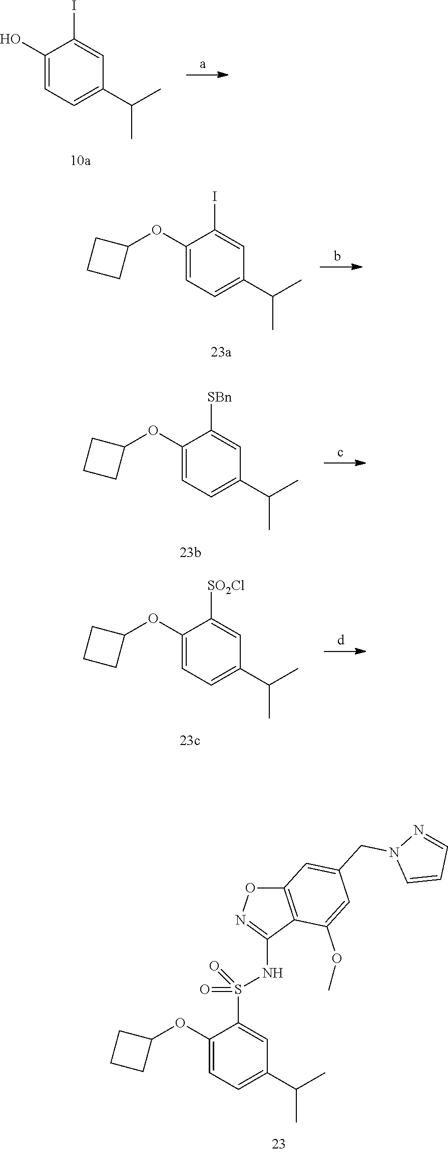

### Step a:

### 1-Cyclobutoxy-2-iodo-4-isopropylbenzene

Bromocyclobutane (525 mg, 3.89 mmol) and cesium carbonate (2.11 g, 6.49 mmol) were added to a solution of compound 10a (850 mg, 3.24 mmol) in N,N-dimethylformamide (10 mL). The reaction system was heated to 100° C. and stirred for 16 h. After the reaction was completed, the reaction mixture was cooled to room temperature, diluted with water (50 mL), and extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=30:1) to give compound 23a (884 mg, yield: 86.2%).

¹H NMR (400 MHz, DMSO-d₆): δ7.60 (d, 1H), 7.19-7.16 (m, 1H), 6.75 (d, 1H), 4.74-4.65 (m, 1H), 2.85-2.73 (m, 1H), 2.48-2.38 (m, 2H), 2.11-1.98 (m, 2H), 1.84-1.58 (m, 2H), 1.16 (s, 3H), 1.14 (s, 3H).

### Step b:

### Benzyl(2-cyclobutoxy-5-isopropylphenyl)sulfane

Under a nitrogen atmosphere, benzyl mercaptan (0.52 g, 4.16 mmol), potassium carbonate (0.88 g, 6.40 mmol), 1,4-diazabicyclo[2.2.2]octane (0.04 g, 0.32 mmol), and copper(I) iodide (0.03 g, 0.16 mmol) were added to a solution of compound 23a (1.00 g, 3.16 mmol) in N,N-dimethylformamide (10 mL). The reaction system was heated to 100° C. and stirred for 16 h. After the reaction was completed, the reaction mixture was cooled to room temperature, diluted with water (50 mL), and extracted with dichloromethane (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:dichloromethane=10:1) to give compound 23b (0.63 g, yield: 63.7%).

¹H NMR (400 MHz, DMSO-d₆): δ7.37-7.20 (m, 5H), 7.06 (d, 1H), 6.97-6.94 (m, 1H), 6.71 (d, 1H), 4.71-4.62 (m, 1H), 4.15 (s, 2H), 2.81-2.72 (m, 1H), 2.45-2.36 (m, 2H), 2.09-1.96 (m, 2H), 1.83-1.58 (m, 2H), 1.12 (s, 3H), 1.10 (s, 3H).

### Step c:

### 2-Cyclobutoxy-5-isopropylbenzenesulfonyl chloride

N-Chlorosuccinimide (95 mg, 0.71 mmol) was added to compound 23b (37 mg, 0.12 mmol) in a mixed solution (glacial acetic acid and water in a ratio of 10:1, 3 mL) at 25° C. The reaction system was stirred at 25° C. for 3 h. After the reaction was completed, water (50 mL) was added, and stirring was continued for 20 min. The reaction mixture was then filtered under reduced pressure to give compound 23c (20 mg, yield: 58.5%).

¹HNMR (400 MHz, DMSO-d₆): δ7.57 (d, 1H), 7.14-7.12 (m, 1H), 6.73 (d, 1H), 4.70-4.61 (m, 1H), 2.86-2.78 (m, 1H), 2.37-2.35 (m, 2H), 2.12-2.02 (m, 2H), 1.81-1.56 (m, 2H), 1.17 (s, 3H), 1.15 (s, 3H).

### Step d:

### N-(6-((1H-Pyrazol-1-yl)methyl)-4-methoxybenzo[d]isoxazol-3-yl)-2-cyclobutoxy-5-isopropylbenzenesulfonamide

Sodium hydride (60%, 82 mg, 2.05 mmol) was added to a solution of intermediate 1 (100 mg, 0.41 mmol) in N,N-dimethylformamide (3 mL) at 0° C. The reaction system was stirred at 0° C. for 1 h. Then, compound 23c (236 mg, 0.82 mmol) was added, and stirring was continued for 2 h. After the reaction was completed, the reaction mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 23 (40 mg, yield: 19.7%).

¹HNMR (400 MHz, DMSO-d₆): δ9.79 (s, 1H), 7.87 (d, 1H), 7.69 (d, 1H), 7.50-7.44 (m, 2H), 6.91-6.85 (m, 2H), 6.76 (s, 1H), 6.31-6.30 (m, 1H), 5.45 (s, 2H), 4.69-4.64 (m, 1H), 3.79 (s, 3H), 2.94-2.90 (m, 1H), 2.23-2.17 (m, 2H), 1.80-1.73 (m, 2H), 1.57-1.48 (m, 2H), 1.19 (s, 3H), 1.17 (s, 3H).

MS m/z (ESI): 497.0 [M+H]⁺.

### Example 30

### Synthesis of Compound 24

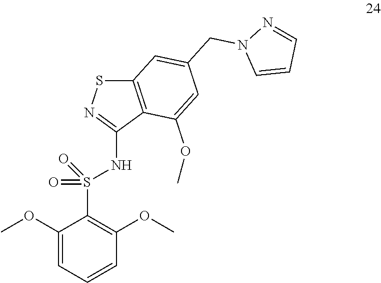 [SMILES: COc1cccc(OC)c1S(=O)(=O)Nc1nsc2cc(Cn3cccn3)cc(OC)c12]

### N-(6-((1H-Pyrazol-1-yl)methyl)-4-methoxybenzo[d]isothiazol-3-yl)-2,6-dimethoxybenzenesulfonamide

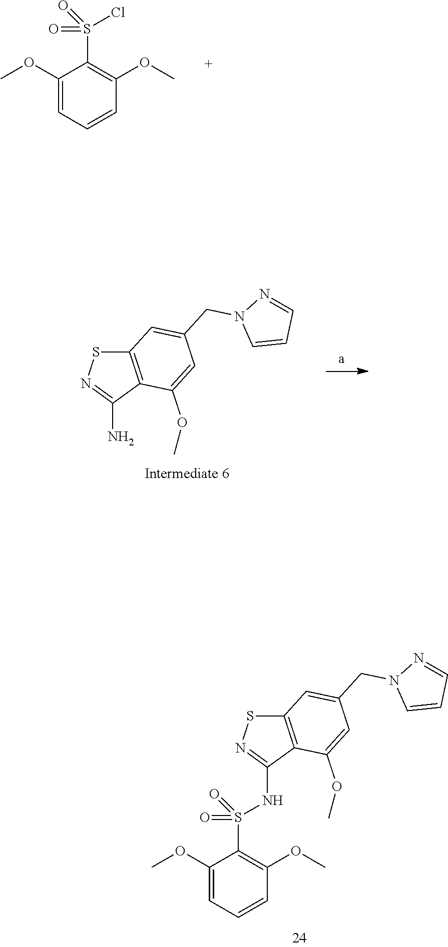

### Step a:

### N-(6-((1H-Pyrazol-1-yl)methyl)-4-methoxybenzo[d]isothiazol-3-yl)-2,6-dimethoxybenzenesulfonamide

Sodium hydride (60%, 111 mg, 2.77 mmol) was added to a solution of intermediate 6 (120 mg, 0.46 mmol) in N,N-dimethylformamide (2 mL) at 0° C. The reaction system was stirred at 0° C. for 1 h. Then, 2,6-dimethoxybenzenesulfonyl chloride (148 mg, 0.62 mmol) was added, and the mixture was warmed to room temperature and stirred for another 1 h. After the reaction was completed, the reaction mixture was quenched with water and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 24 (20 mg, yield: 9.4%).

¹HNMR (400 MHz, DMSO-d₆): δ9.38 (s, 1H), 7.89 (d, 1H), 7.50 (d, 1H), 7.45 (t, 1H), 7.27 (s, 1H), 6.95 (s, 1H), 6.72 (d, 2H), 6.31 (t, 1H), 5.46 (s, 2H), 4.06 (s, 3H), 3.73 (s, 6H).

MS m/z (ESI): 460.9 [M+H]⁺.

### Example 31

### Synthesis of Compound 25

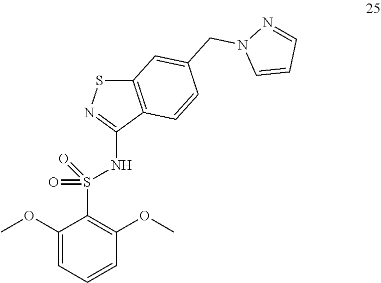 [SMILES: COc1cccc(OC)c1S(=O)(=O)Nc1nsc2cc(Cn3cccn3)ccc12]

### N-(6-((1H-Pyrazol-1-yl)methyl)benzo[d]isothiazol-3-yl)-2,6-dimethoxybenzenesulfonamide

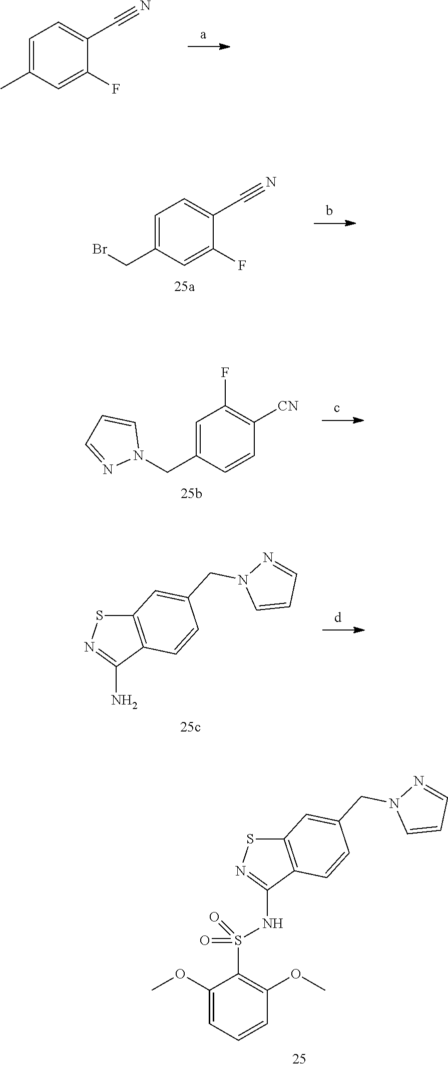

### Step a:

### 4-(Bromomethyl)-2-fluorobenzonitrile

Dibenzoyl peroxide (0.09 g, 0.37 mmol) and N-bromosuccinimide (1.98 g, 11.12 mmol) were added to a solution of 2-fluoro-4-methylbenzonitrile (1.00 g, 7.40 mmol) in chloroform (20 mL). The reaction system was heated to 75° C. and stirred for 16 h. After the reaction was completed, the reaction mixture was diluted with water (100 mL) and extracted with dichloromethane (300 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=15:1) to give compound 25a (0.78 g, yield: 49.2%).

¹H NMR (400 MHz, DMSO-d₆): δ7.97-7.92 (m, 1H), 7.65 (d, 1H), 7.53-7.50 (m, 1H), 4.77 (s, 2H).

### Step b:

### 4-((1H-Pyrazol-1-yl)methyl)-2-fluorobenzonitrile

Pyrazole (201 mg, 2.95 mmol) and cesium carbonate (963 mg, 2.96 mmol) were added to a solution of compound 25a (575 mg, 2.69 mmol) in acetonitrile (15 mL) at 25° C. The reaction system was stirred at 25° C. for 16 h. After the reaction was completed, the reaction mixture was diluted with water (50 mL) and extracted with dichloromethane (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 25b (522 mg, yield: 96.6%).

¹H NMR (400 MHz, DMSO-d₆): δ7.92-7.89 (m, 2H), 7.53-7.52 (m, 1H), 7.27 (d, 1H), 7.15-7.12 (m, 1H), 6.33 (d, 1H), 5.48 (s, 2H).

### Step c:

### 6-((1H-Pyrazol-1-yl)methyl)benzo[d]isothiazol-3-amine

Under a nitrogen atmosphere, sodium sulfide (27 mg, 0.35 mmol) was added to a solution of compound 25b (70 mg, 0.35 mmol) in dimethyl sulfoxide (2 mL). The reaction system was heated to 70° C. and stirred for 7 h. After the reaction was completed, the reaction system was cooled to 0° C. Subsequently, aqueous ammonia (1 mL) and an aqueous sodium hypochlorite solution (1 mL) were slowly added to the reaction system, and stirring was continued for 16 h. The reaction mixture was diluted with water (50 mL) and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:tetrahydrofuran=8:1) to give compound 25c (49 mg, yield: 61.1%).

¹HNMR (400 MHz, CDCl₃): δ7.66 (d, 1H), 7.61-7.55 (m, 2H), 7.46 (d, 1H), 7.22 (d, 1H), 6.33 (t, 1H), 5.48 (s, 2H).

MS m/z (ESI): 231.2 [M+H]⁺.

### Step d:

### N-(6-((1H-Pyrazol-1-yl)methyl)benzo[d]isothiazol-3-yl)-2,6-dimethoxybenzenesulfonamide

Sodium hydride (60%, 87 mg, 2.17 mmol) was added to a solution of compound 25c (100 mg, 0.43 mmol) in N,N-dimethylformamide (3 mL) at 0° C. The reaction system was stirred at 0° C. for 1 h. Then, 2,6-dimethoxybenzenesulfonyl chloride (206 mg, 0.87 mmol) was added, and stirring was continued for 1 h. After the reaction was completed, the reaction mixture was quenched with a saturated aqueous ammonium chloride solution and extracted with dichloromethane (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 25 (84 mg, yield: 44.9%).

¹HNMR (400 MHz, DMSO-d₆): δ11.30 (s, 1H), 8.36 (d, 1H), 7.88 (d, 1H), 7.82 (s, 1H), 7.49 (d, 1H), 7.45 (t, 1H), 7.29 (d, 1H), 6.72 (s, 1H), 6.70 (s, 1H), 6.30 (t, 1H), 5.50 (s, 2H), 3.67 (s, 6H).

MS m/z (ESI): 430.9 [M+H]⁺.

### Example 32

### Synthesis of Compound 26

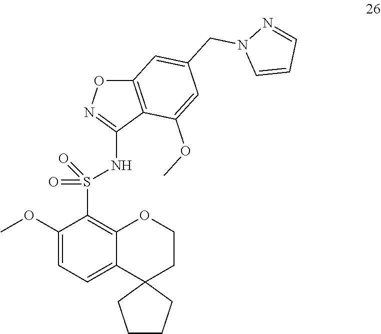 [SMILES: COc1ccc2c(c1S(=O)(=O)Nc1noc3cc(Cn4cccn4)cc(OC)c13)OCCC21CCCC1]

### N-(6-((1H-Pyrazol-1-yl)methyl)-4-methoxybenzo[d]isoxazol-3-yl)-7-methoxyspiro[chromane-4, 1′-cyclopentane]-8-sulfonamide

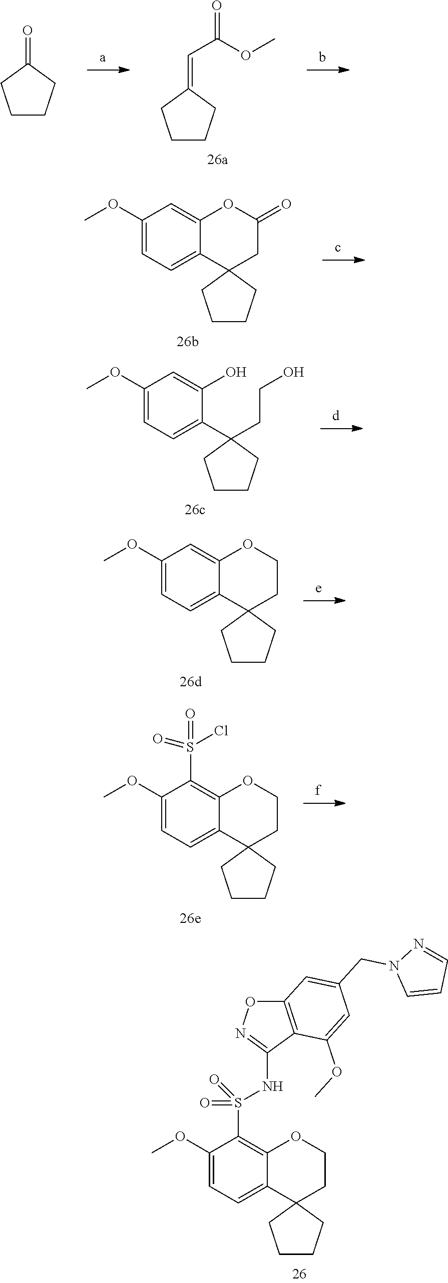

### Step a:

### Methyl 2-cyclopentylideneacetate

Under a nitrogen atmosphere, sodium hydride (60%, 3.21 g, 80.25 mmol) was added to a solution of trimethyl phosphonoacetate (15.14 g, 83.14 mmol) in tetrahydrofuran (150 mL) at 0° C., and the reaction system was stirred at 0° C. for 1 h. Then, a solution of cyclopentanone (5.00 g, 59.44 mmol) in tetrahydrofuran (50 mL) was added dropwise, and the reaction system was warmed to 25° C. and stirred for another 2 h. After the reaction was completed, the mixture was quenched with a saturated aqueous ammonium chloride solution and extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=20:1) to give compound 26a (3.66 g, yield: 43.9%).

¹H NMR (300 MHz, CDCl₃) δ5.81-5.78 (m, 1H), 3.68 (s, 3H), 2.76 (t, 2H), 2.43 (t, 2H), 1.79-1.61 (m, 4H)

### Step b:

### 7-Methoxyspiro[chromane-4,1′-cyclopentan]-2-one

3-Methoxyphenol (6.48 g, 52.20 mmol), compound 26a (3.66 g, 26.11 mmol), and 98% concentrated sulfuric acid (0.51 g, 5.10 mmol) were mixed. The reaction system was then heated to 130° C. and stirred for 3 h. After the reaction was completed, the mixture was quenched with water and extracted with ethyl acetate (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=20:1) to give compound 26b (1.90 g, yield: 31.3%).

¹H NMR (300 MHz, CDCl₃): δ7.14 (d, 1H), 6.70-6.62 (m, 2H), 3.79 (s, 3H), 2.66 (s, 2H), 1.93-1.59 (m, 8H).

### Step c:

### 2-(1-(2-Hydroxyethyl)cyclopentyl)-5-methoxyphenol

Under a nitrogen atmosphere, lithium aluminum hydride (0.27 g, 7.24 mmol) was added to a solution of compound 26b (1.60 g, 6.89 mmol) in tetrahydrofuran (30 mL) at 0° C., and the reaction system was stirred at 0° C. for 1 h. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=1:1) to give compound 26c (1.60 g, yield: 98.3%).

¹H NMR (300 MHz, CDCl₃):7.06 (d, 1H), 6.43-6.37 (m, 1H), 6.28 (d, 1H), 3.74 (s, 3H), 3.52 (t, 2H), 2.04-1.93 (m, 6H), 1.72-1.63 (m, 4H).

### Step d:

### 7-Methoxyspiro[chromane-4,1′-cyclopentane]

Compound 26c (1.60 g, 6.77 mmol) and p-toluenesulfonic acid (0.23 g, 1.34 mmol) were dissolved in a solution of toluene (32 mL). The reaction system was heated to 110° C. and stirred for 2 h. After the reaction was completed, the reaction mixture was concentrated under reduced pressure. The resulting crude product was purified by flash silica gel column chromatography (petroleum ether:tetrahydrofuran=20:1) to give compound 26d (1.26 g, yield: 85.2%).

¹H NMR (300 MHz, CDCl₃): δ7.13 (d, 1H), 6.49 (dd, 1H), 6.33 (d, 1H), 4.17 (t, 2H), 3.75 (s, 3H), 1.93-1.69 (m, 1OH).

### Step e:

### 7-Methoxyspiro[chromane-4,1′-cyclopentane]-8-sulfonyl chloride

Under a nitrogen atmosphere, n-butyllithium (2.5 M in n-hexane, 2.5 mL, 6.25 mmol) was added dropwise to a solution of compound 26d (1.26 g, 5.77 mmol) in tetrahydrofuran (25 mL) at 0° C., and the reaction system was stirred at 0° C. for 1 h and then cooled to −78° C. Subsequently, sulfuryl chloride (1.01 g, 7.48 mmol) was added, and stirring was continued at −78° C. for 15 min. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=2:1) to give compound 26e (0.77 g, yield: 42.1%).

¹H NMR (400 MHz, CDCl₃): δ7.45 (d, 1H), 6.57 (d, 1H), 4.37 (t, 2H), 3.94 (s, 3H), 1.88-1.77 (m, 1OH).

### Step f:

### N-(6-((1H-Pyrazol-1-yl)methyl)-4-methoxybenzo[d]isoxazol-3-yl)-7-methoxyspiro[chromane-4,1′-cyclopentane]-8-sulfonamide

Sodium hydride (60%, 82 mg, 2.05 mmol) was added to a solution of intermediate 1 (100 mg, 0.41 mmol) in N,N-dimethylformamide (2 mL) at 0° C. The reaction system was stirred for 30 min. Then, compound 26e (195 mg, 0.62 mmol) was added to the system, and stirring was continued at 0° C. for 1 h. After the reaction was completed, the mixture was diluted with a 1 N aqueous hydrochloric acid solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 26 (105 mg, yield: 48.9%). ¹HNMR (400 MHz, DMSO-d₆): δ9.47 (s, 1H), 7.87 (d, 1H), 7.50 (d, 1H), 7.44 (d, 1H), 6.85 (s, 1H), 6.76 (s, 1H), 6.69 (d, 1H), 6.30 (t, 1H), 5.45 (s, 2H), 4.07 (t, 2H), 3.87 (s, 3H), 3.72 (s, 3H), 1.77-1.60 (m, 1OH).

MS m/z (ESI): 525.0 [M+H]⁺.

### Example 33

### Synthesis of Compound 27

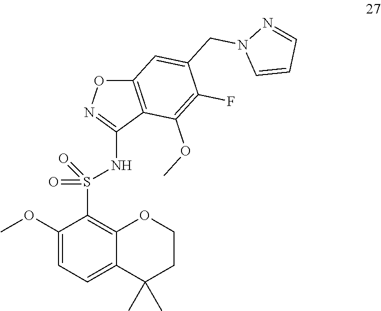 [SMILES: COc1ccc2c(c1S(=O)(=O)Nc1noc3cc(Cn4cccn4)c(F)c(OC)c13)OCCC2(C)C]

### N-(6-((1H-Pyrazol-1-yl)methyl)-5-fluoro-4-methoxybenzo[d]isoxazol-3-yl)-7-methoxy-4,4-dimethylchromane-8-sulfonamide

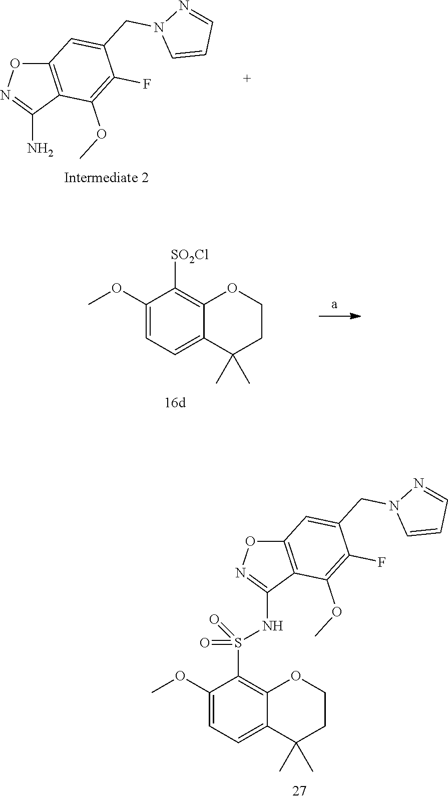 [SMILES: COc1c(F)c(Cn2cccn2)cc2onc(N)c12 ; COc1ccc2c(c1S(=O)(=O)Cl)OCCC2(C)C ; COc1ccc2c(c1S(=O)(=O)Nc1noc3cc(Cn4cccn4)c(F)c(OC)c13)OCCC2(C)C]

### Step a:

### N-(6-((1H-Pyrazol-1-yl)methyl)-5-fluoro-4-methoxybenzo[d]isoxazol-3-yl)-7-methoxy-4,4-dimethylchromane-8-sulfonamide

Sodium hydride (60%, 61 mg, 1.52 mmol) was added to a solution of intermediate 2 (80 mg, 0.31 mmol) in N,N-dimethylformamide (1 mL) at 0° C. The reaction system was stirred at 0° C. for 15 min. Then, compound 16d (133 mg, 0.46 mmol) was added to the system, and stirring was continued for 15 min. After the reaction was completed, the reaction mixture was warmed to room temperature, quenched with a 1 N aqueous hydrochloric acid solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 27 (88 mg, yield: 55.8%). ¹HNMR (400 MHz, DMSO-d₆): δ10.00 (s, 1H), 7.85 (d, 1H), 7.52-7.50 (m, 2H), 6.91 (d, 1H), 6.69 (d, 1H), 6.32-6.31 (m, 1H), 5.50 (s, 2H), 4.10 (t, 2H), 4.03 (d, 3H), 3.72 (s, 3H), 1.70 (t, 2H), 1.23 (s, 6H).

MS m/z (ESI): 517.1 [M+H]⁺.

### Example 34

### Synthesis of Compound 28

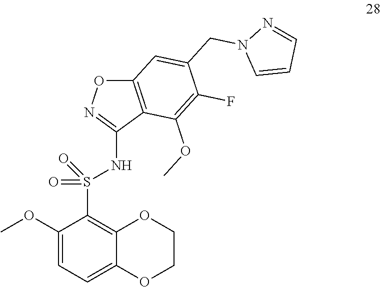 [SMILES: COc1ccc2c(c1S(=O)(=O)Nc1noc3cc(Cn4cccn4)c(F)c(OC)c13)OCCO2]

### N-(6-((1H-Pyrazol-1-yl)methyl)-5-fluoro-4-methoxybenzo[d]isoxazol-3-yl)-6-methoxy-2,3-dihydrobenzo[b][1,4]dioxine-5-sulfonamide

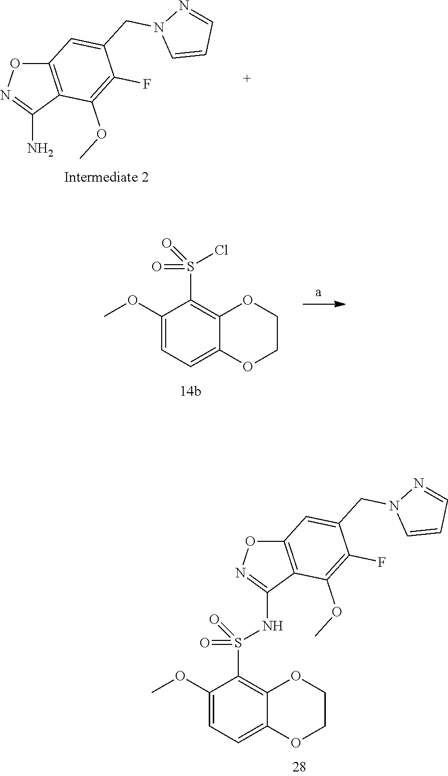 [SMILES: COc1c(F)c(Cn2cccn2)cc2onc(N)c12 ; COc1ccc2c(c1S(=O)(=O)Cl)OCCO2 ; COc1ccc2c(c1S(=O)(=O)Nc1noc3cc(Cn4cccn4)c(F)c(OC)c13)OCCO2]

### Step a:

### N-(6-((1H-Pyrazol-1-yl)methyl)-5-fluoro-4-methoxybenzo[d]isoxazol-3-yl)-6-methoxy-2,3-dihydrobenzo[b][1,4]dioxine-5-sulfonamide

Sodium hydride (60%, 61 mg, 1.52 mmol) was added to a solution of intermediate 2 (80 mg, 0.31 mmol) in N,N-dimethylformamide (1 mL) at 0° C. The reaction system was stirred at 0° C. for 15 min. Then, compound 14b (121 mg, 0.46 mmol) was added to the system, and stirring was continued for 15 min. After the reaction was completed, the reaction mixture was warmed to room temperature, quenched with a 1 N aqueous hydrochloric acid solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 28 (90 mg, yield: 60.2%). ¹HNMR (400 MHz, DMSO-d₆): δ10.23 (s, 1H), 7.86 (d, 1H), 7.50 (d, 1H), 7.07 (d, 1H), 6.92 (d, 1H), 6.65 (d, 1H), 6.31-6.30 (m, 1H), 5.51 (s, 2H), 4.22-4.16 (m, 4H), 4.04 (d, 3H), 3.70 (s, 3H).

MS m/z (ESI): 491.1 [M+H]⁺.

### Example 35

### Synthesis of Compound 29

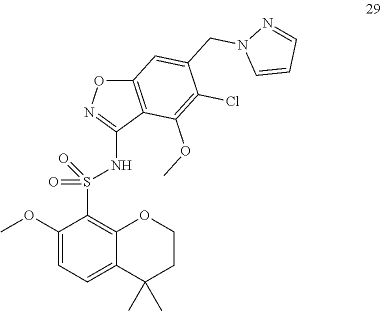 [SMILES: COc1ccc2c(c1S(=O)(=O)Nc1noc3cc(Cn4cccn4)c(Cl)c(OC)c13)OCCC2(C)C]

### N-(6-((1H-Pyrazol-1-yl)methyl)-5-chloro-4-methoxybenzo[d]isoxazol-3-yl)-7-methoxy-4,4-dimethylchromane-8-sulfonamide

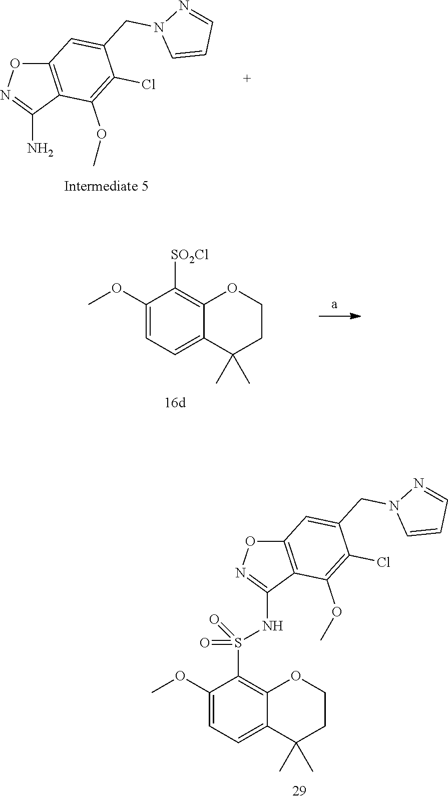 [SMILES: COc1c(Cl)c(Cn2cccn2)cc2onc(N)c12 ; COc1ccc2c(c1S(=O)(=O)Cl)OCCC2(C)C ; COc1ccc2c(c1S(=O)(=O)Nc1noc3cc(Cn4cccn4)c(Cl)c(OC)c13)OCCC2(C)C]

### Step a:

### N-(6-((1H-Pyrazol-1-yl)methyl)-5-chloro-4-methoxybenzo[d]isoxazol-3-yl)-7-methoxy-4,4-dimethylchromane-8-sulfonamide

Sodium hydride (60%, 50 mg, 1.25 mmol) was added to a solution of intermediate 5 (70 mg, 0.25 mmol) in N,N-dimethylformamide (1 mL) at 0° C. The reaction system was stirred at 0° C. for 5 min. Then, compound 16d (110 mg, 0.38 mmol) was added to the system, and stirring was continued for 10 min. After the reaction was completed, the reaction mixture was warmed to room temperature, quenched with a saturated aqueous ammonium chloride solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 29 (89 mg, yield: 66.6%).

¹HNMR (400 MHz, DMSO-d₆): δ10.30 (s, 1H), 7.88 (d, 1H), 7.54 (d, 1H), 7.52 (d, 1H), 6.79 (s, 1H), 6.70 (d, 1H), 6.34 (t, 1H), 5.57 (s, 2H), 4.12 (t, 2H), 3.91 (s, 3H), 3.72 (s, 3H), 1.71 (t, 2H), 1.25 (s, 6H).

MS m/z (ESI): 533.1 [M+H]⁺.

### Example 36

### Synthesis of Compound 30

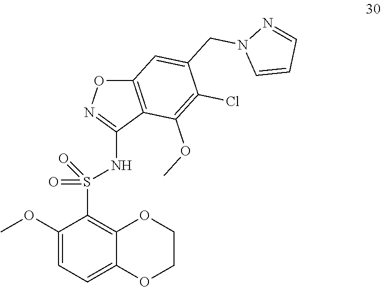 [SMILES: COc1ccc2c(c1S(=O)(=O)Nc1noc3cc(Cn4cccn4)c(Cl)c(OC)c13)OCCO2]

### N-(6-((1H-Pyrazol-1-yl)methyl)-5-chloro-4-methoxybenzo[d]isoxazol-3-yl)-6-methoxy-2,3-dihydrobenzo[b][1,4]dioxine-5-sulfonamide

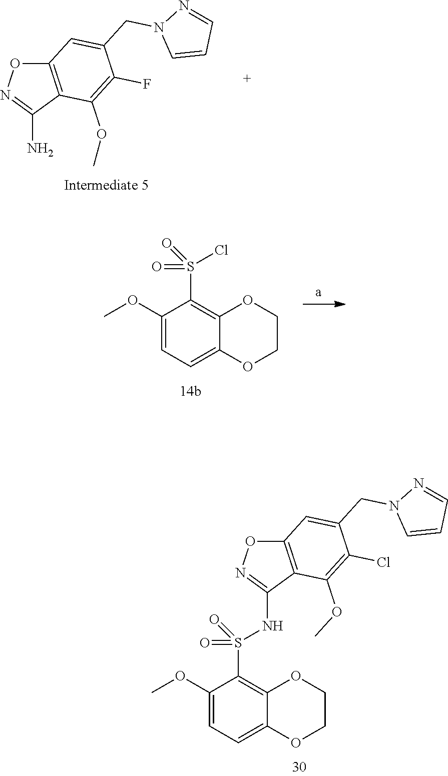 [SMILES: COc1c(F)c(Cn2cccn2)cc2onc(N)c12 ; COc1ccc2c(c1S(=O)(=O)Cl)OCCO2 ; COc1ccc2c(c1S(=O)(=O)Nc1noc3cc(Cn4cccn4)c(Cl)c(OC)c13)OCCO2]

### Step a:

### N-(6-((1H-Pyrazol-1-yl)methyl)-5-chloro-4-methoxybenzo[d]isoxazol-3-yl)-6-methoxy-2,3-dihydrobenzo[b][1,4]dioxine-5-sulfonamide

Sodium hydride (60%, 37 mg, 0.93 mmol) was added to a solution of intermediate 5 (51 mg, 0.18 mmol) in N,N-dimethylformamide (1 mL) at 0° C. The reaction system was stirred at 0° C. for 5 min. Then, compound 14b (73 mg, 0.28 mmol) was added to the system, and stirring was continued for 10 min. After the reaction was completed, the reaction mixture was warmed to room temperature, quenched with a saturated aqueous ammonium chloride solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 30 (63 mg, yield: 67.9%).

¹HNMR (400 MHz, DMSO-d₆): δ10.53 (s, 1H), 7.88 (d, 1H), 7.55 (d, 1H), 7.08 (d, 1H), 6.79 (s, 1H), 6.67 (d, 1H), 6.34 (t, 1H), 5.57 (s, 2H), 5.20-5.16 (m, 4H), 3.94 (s, 3H), 3.71 (s, 3H).

MS m/z (ESI): 507.0 [M+H]⁺.

### Example 37

### Synthesis of Compound 31

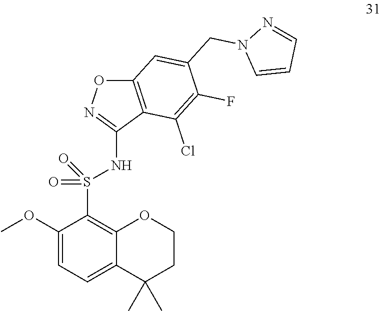 [SMILES: COc1ccc2c(c1S(=O)(=O)Nc1noc3cc(Cn4cccn4)c(F)c(Cl)c13)OCCC2(C)C]

### N-(6-((1H-Pyrazol-1-yl)methyl)-4-chloro-5-fluorobenzo[d]isoxazol-3-yl)-7-methoxy-4,4-dimethylchromane-8-sulfonamide

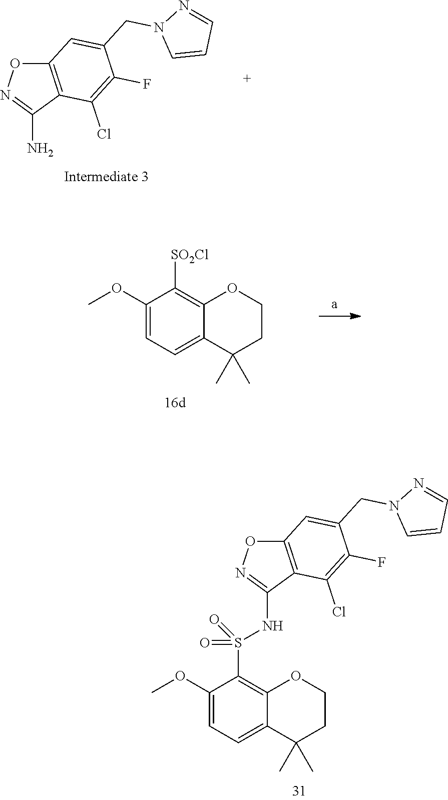 [SMILES: Nc1noc2cc(Cn3cccn3)c(F)c(Cl)c12 ; COc1ccc2c(c1S(=O)(=O)Cl)OCCC2(C)C ; COc1ccc2c(c1S(=O)(=O)Nc1noc3cc(Cn4cccn4)c(F)c(Cl)c13)OCCC2(C)C]

### Step a:

### N-(6-((1H-Pyrazol-1-yl)methyl)-4-chloro-5-fluorobenzo[d]isoxazol-3-yl)-7-methoxy-4,4-dimethylchromane-8-sulfonamide

Sodium hydride (60%, 45 mg, 1.13 mmol) was added to a solution of intermediate 3 (60 mg, 0.23 mmol) in N,N-dimethylformamide (1 mL) at 0° C. The reaction system was stirred at 0° C. for 15 min. Then, compound 16d (73 mg, 0.25 mmol) was added to the system, and stirring was continued for 15 min. After the reaction was completed, the reaction mixture was warmed to room temperature, quenched with a 1 N aqueous hydrochloric acid solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 31 (19 mg, yield: 16.2%).

¹HNMR (400 MHz, DMSO-d₆): δ10.28 (s, 1H), 7.88 (d, 1H), 7.54-7.44 (m, 3H), 6.69 (d, 1H), 6.31 (t, 1H), 5.57 (s, 2H), 4.07 (t, 2H), 3.71 (s, 3H), 1.69 (t, 2H), 1.25 (s, 6H).

MS m/z (ESI): 521.1 [M+H]⁺.

### Example 38

### Synthesis of Compound 32

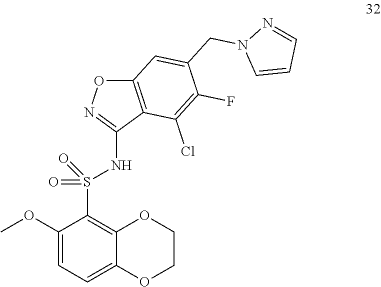 [SMILES: COc1ccc2c(c1S(=O)(=O)Nc1noc3cc(Cn4cccn4)c(F)c(Cl)c13)OCCO2]

### N-(6-((1H-Pyrazol-1-yl)methyl)-4-chloro-5-fluorobenzo[d]isoxazol-3-yl)-6-methoxy-2,3-dihydrobenzo[b][1,4]dioxine-5-sulfonamide

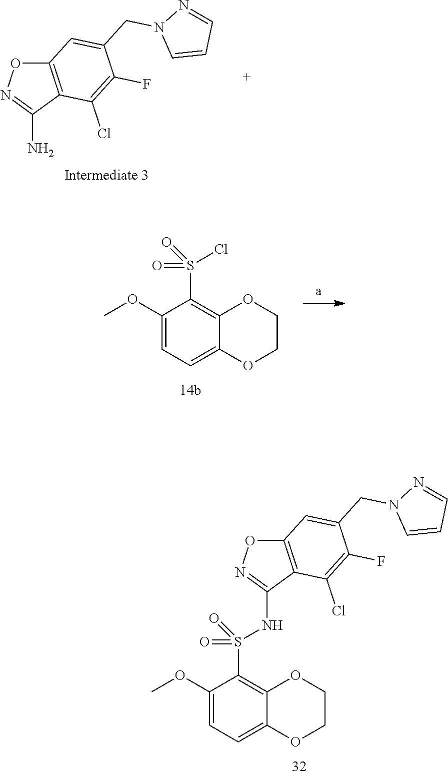 [SMILES: Nc1noc2cc(Cn3cccn3)c(F)c(Cl)c12 ; COc1ccc2c(c1S(=O)(=O)Cl)OCCO2 ; COc1ccc2c(c1S(=O)(=O)Nc1noc3cc(Cn4cccn4)c(F)c(Cl)c13)OCCO2]

### Step a:

### N-(6-((1H-Pyrazol-1-yl)methyl)-4-chloro-5-fluorobenzo[d]isoxazol-3-yl)-6-methoxy-2,3-dihydrobenzo[b][1,4]dioxine-5-sulfonamide

Sodium hydride (60%, 45 mg, 1.13 mmol) was added to a solution of intermediate 3 (60 mg, 0.23 mmol) in N,N-dimethylformamide (1 mL) at 0° C. The reaction system was stirred at 0° C. for 15 min. Then, compound 14b (89 mg, 0.34 mmol) was added to the system, and stirring was continued for 15 min. After the reaction was completed, the reaction mixture was warmed to room temperature, quenched with a 1 N aqueous hydrochloric acid solution, and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 32 (33 mg, yield: 29.6%).

¹HNMR (400 MHz, DMSO-d₆): δ10.48 (s, 1H), 7.88 (d, 1H), 7.50 (d, 1H), 7.44 (d, 1H), 7.08 (d, 1H), 6.66 (d, 1H), 6.31 (t, 1H), 5.57 (s, 2H), 4.20-4.16 (m, 4H), 3.68 (s, 3H).

MS m/z (ESI): 495.0 [M+H]⁺.

### Example 39

### Synthesis of Compound 33

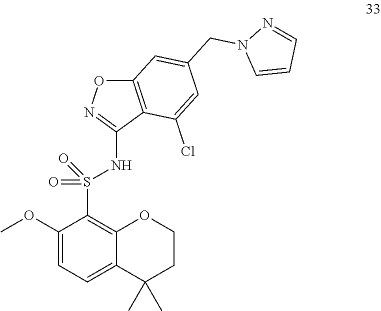 [SMILES: COc1ccc2c(c1S(=O)(=O)Nc1noc3cc(Cn4cccn4)cc(Cl)c13)OCCC2(C)C]

### N-(6-((1H-Pyrazol-1-yl)methyl)-4-chlorobenzo[d]isoxazol-3-yl)-7-methoxy-4,4-dimethylchromane-8-sulfonamide

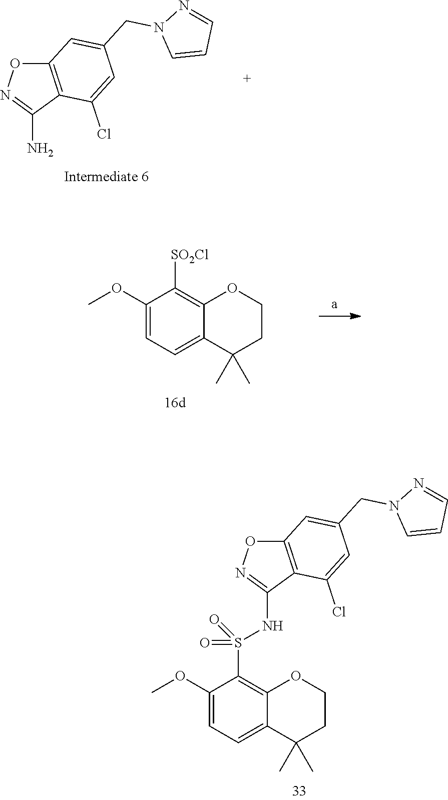 [SMILES: Nc1noc2cc(Cn3cccn3)cc(Cl)c12 ; COc1ccc2c(c1S(=O)(=O)Cl)OCCC2(C)C ; COc1ccc2c(c1S(=O)(=O)Nc1noc3cc(Cn4cccn4)cc(Cl)c13)OCCC2(C)C]

### Step a:

### N-(6-((1H-Pyrazol-1-yl)methyl)-4-chlorobenzo[d]isoxazol-3-yl)-7-methoxy-4,4-dimethylchromane-8-sulfonamide

Sodium hydride (60%, 48 mg, 1.20 mmol) was added to a solution of intermediate 6 (56 mg, 0.23 mmol) in N,N-dimethylformamide (1 mL) at 0° C. The reaction system was stirred at 0° C. for 15 min. Then, compound 16d (105 mg, 0.36 mmol) was added to the system, and stirring was continued for 15 min. After the reaction was completed, the reaction mixture was warmed to room temperature, quenched with water, and extracted with ethyl acetate (30 mL×2). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 33 (79 mg, yield: 69.7%).

¹HNMR (400 MHz, DMSO-d₆): δ10.09 (s, 1H), 7.90 (d, 1H), 7.52-7.45 (m, 3H), 7.25 (s, 1H), 6.68 (d, 1H), 6.31 (t, 1H), 5.50 (s, 2H), 4.06 (t, 2H), 3.70 (s, 3H), 1.68 (t, 2H), 1.24 (s, 6H).

MS m/z (ESI): 503.1 [M+H]⁺.

### Example 40

### Synthesis of Compound 34

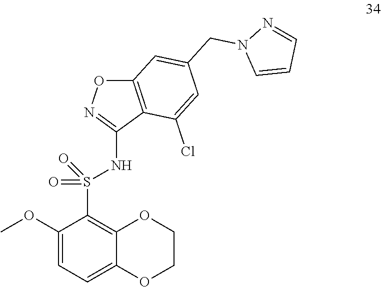 [SMILES: COc1ccc2c(c1S(=O)(=O)Nc1noc3cc(Cn4cccn4)cc(Cl)c13)OCCO2]

### N-(6-((1H-Pyrazol-1-yl)methyl)-4-chlorobenzo[d]isoxazol-3-yl)-6-methoxy-2,3-dihydrobenzo[b][1,4]dioxine-5-sulfonamide

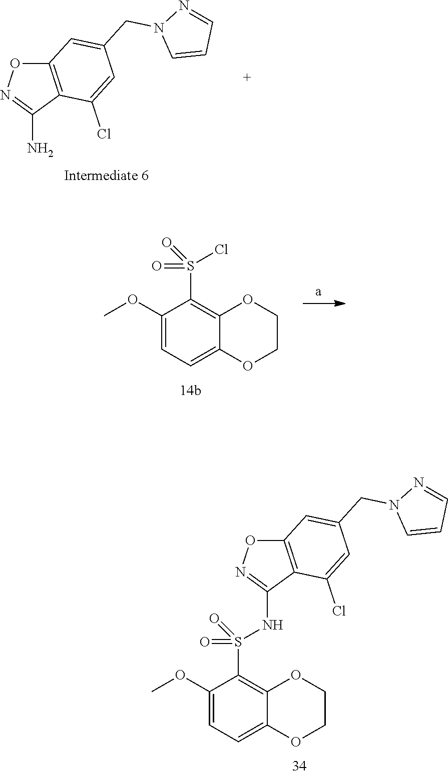 [SMILES: Nc1noc2cc(Cn3cccn3)cc(Cl)c12 ; COc1ccc2c(c1S(=O)(=O)Cl)OCCO2 ; COc1ccc2c(c1S(=O)(=O)Nc1noc3cc(Cn4cccn4)cc(Cl)c13)OCCO2]

### Step a:

### N-(6-((1H-Pyrazol-1-yl)methyl)-4-chlorobenzo[d]isoxazol-3-yl)-6-methoxy-2,3-dihydrobenzo[b][1,4]dioxine-5-sulfonamide

Sodium hydride (60%, 48 mg, 1.20 mmol) was added to a solution of intermediate 6 (56 mg, 0.23 mmol) in N,N-dimethylformamide (1 mL) at 0° C. The reaction system was stirred at 0° C. for 15 min. Then, compound 14b (96 mg, 0.36 mmol) was added to the system, and stirring was continued for 15 min. After the reaction was completed, the reaction mixture was warmed to room temperature, quenched with water, and extracted with ethyl acetate (30 mL×2). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 34 (52 mg, yield: 48.4%).

¹HNMR (400 MHz, DMSO-d₆): δ10.31 (s, 1H), 7.91 (d, 1H), 7.52 (d, 1H), 7.47 (s, 1H), 7.27 (s, 1H), 7.07 (d, 1H), 6.66 (d, 1H), 6.32 (t, 1H), 5.51 (s, 2H), 4.20-4.15 (m, 4H), 3.68 (s, 3H).

MS m/z (ESI): 477.0 [M+H]⁺.

### Example 41

### Synthesis of Compound 35

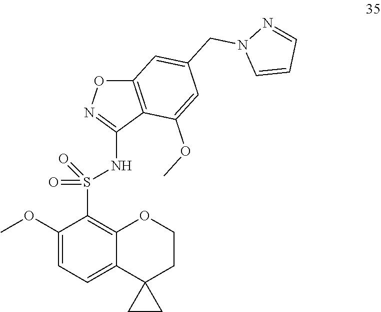 [SMILES: COc1ccc2c(c1S(=O)(=O)Nc1noc3cc(Cn4cccn4)cc(OC)c13)OCCC21CC1]

### N-(6-((1H-Pyrazol-1-yl)methyl)-4-methoxybenzo[d]isoxazol-3-yl)-7-methoxyspiro[chromane-4,1′-cyclopropane]-8-sulfonamide

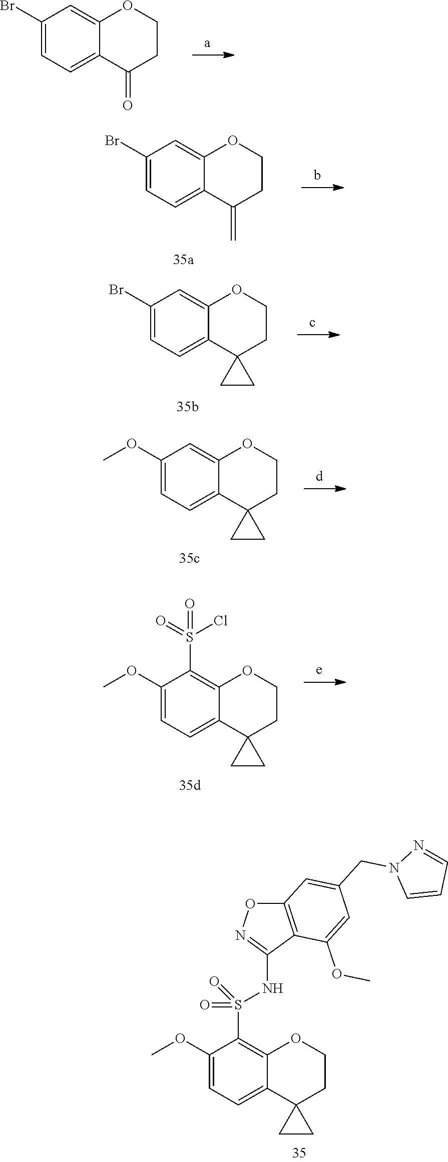

### Step a:

### 7-Bromo-4-methylenechromane

Under a nitrogen atmosphere, at 0° C., n-butyllithium (2.5 M in n-hexane, 13.74 mL, 34.35 mmol) was slowly added dropwise to a solution of methyltriphenylphosphonium bromide (12.59 g, 35.24 mmol) in tetrahydrofuran (80 mL), and the reaction system was stirred for 1 h. Then, a solution of 7-bromo-2,3-dihydrochromen-4-one (4.00 g, 17.62 mmol) in tetrahydrofuran (20 mL) was added dropwise to the system, and stirring was continued for 1 h. After the reaction was completed, the reaction mixture was quenched with a saturated aqueous ammonium chloride solution and extracted with ethyl acetate (200 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether) to give compound 35a (2.43 g, yield: 61.3%).

¹HNMR (400 MHz, DMSO-d₆): δ7.60 (d, 1H), 7.08-7.05 (m, 2H), 5.63 (s, 1H), 4.99 (d, 1H), 4.19 (t, 2H), 2.64 (t, 2H).

### Step b:

### 7-Bromospiro[chromane-4,1′-cyclopropane]

Under a nitrogen atmosphere, diiodomethane (11.57 g, 43.18 mmol) was slowly added dropwise to a solution of diethylzinc (1.0 M in n-hexane, 21.59 mL, 21.59 mmol) in dichloromethane (40 mL) at −78° C., and the reaction system was warmed to 0° C. and stirred for 20 min. At 0° C., trifluoroacetic acid (3.69 g, 32.39 mmol) was then added to the reaction system, and stirring was continued for 15 min. Then, a solution of compound 35a (2.43 g, 10.80 mmol) in dichloromethane (12 mL) was added dropwise. After the reaction was completed, the reaction mixture was quenched with a saturated aqueous sodium bicarbonate solution and extracted with dichloromethane (150 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=99:1) to give compound 35b (1.60 g, yield: 62.0%).

¹HNMR (400 MHz, CDCl₃): δ6.97-6.91 (m, 2H), 6.50 (d, 1H), 4.27 (t, 2H), 1.84 (t, 2H), 1.04-1.01 (m, 2H), 0.86-0.83 (m, 2H).

### Step c:

### 7-Methoxyspiro[chromane-4,1′-cyclopropane]

Sodium methoxide (30% in methanol, 16.26 g, 90.34 mmol) was added to a solution of compound 35b (1.80 g, 7.53 mmol) and copper(I) bromide (0.43 g, 3.01 mmol) in N,N-dimethylformamide (10 mL). Subsequently, the reaction system was heated to 100° C. and stirred for 4 h. After the reaction was completed, the reaction mixture was quenched with an aqueous solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=96:4) to give compound 35c (1.40 g, yield: 97.8%).

¹HNMR (400 MHz, DMSO-d₆): δ6.61 (d, 1H), 6.38 (dd, 1H), 6.31 (d, 1H), 4.19 (t, 2H), 3.66 (s, 3H), 1.77 (t, 2H), 0.93-0.91 (m, 2H), 0.80-0.77 (m, 2H).

### Step d:

### 7-Methoxyspiro[chromane-4,1′-cyclopropane]-8-sulfonyl chloride

Under a nitrogen atmosphere, at 0° C., n-butyllithium (2.5 M in n-hexane, 0.64 mL, 1.16 mmol) was slowly added dropwise to a solution of compound 35c (0.20 g, 1.05 mmol) in tetrahydrofuran (4 mL), and the reaction system was stirred for 1 h. Then, sulfuryl chloride (213 mg, 1.58 mmol) was added dropwise to the system, and stirring was continued for 30 min. After the reaction was completed, the reaction mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=3:1) to give compound 35d (60 mg, yield: 19.8%).

¹HNMR (400 MHz, CDCl₃): δ6.86 (d, 1H), 6.50 (d, 1H), 4.49 (t, 2H), 3.92 (s, 3H), 1.89 (t, 2H), 1.03-1.00 (m, 2H), 0.90-0.87 (m, 2H).

### Step e:

### N-(6-((1H-Pyrazol-1-yl)methyl)-4-methoxybenzo[d]isoxazol-3-yl)-7-methoxyspiro[chromane-4,1′-cyclopropane]-8-sulfonamide

Sodium hydride (60%, 41 mg, 1.03 mmol) was added to a solution of intermediate 1 (50 mg, 0.20 mmol) in N,N-dimethylformamide (1 mL) at 0° C. The reaction system was stirred at 0° C. for 15 min. Then, compound 35d (59 mg, 0.20 mmol) was added to the system, and stirring was continued for 30 min. After the reaction was completed, the reaction mixture was warmed to room temperature, quenched with a 1 N aqueous hydrochloric acid solution, and extracted with ethyl acetate (30 mL×2). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 35 (22 mg, yield: 21.6%).

¹HNMR (400 MHz, DMSO-d₆): δ7.87 (d, 1H), 7.50 (d, 1H), 6.88 (d, 1H), 6.82 (s, 1H), 6.75 (s, 1H), 6.60 (d, 1H), 6.30 (t, 1H), 5.45 (s, 2H), 4.17 (t, 2H), 3.88 (s, 3H), 3.70 (s, 3H), 1.70 (t, 2H), 0.96-0.94 (m, 2H), 0.82-0.79 (m, 2H).

MS m/z (ESI): 497.1 [M+H]⁺.

### Example 42

### Synthesis of Compound 36

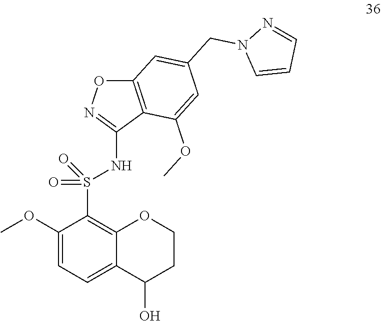 [SMILES: COc1ccc2c(c1S(=O)(=O)Nc1noc3cc(Cn4cccn4)cc(OC)c13)OCCC2O]

### N-(6-((1H-Pyrazol-1-yl)methyl)-4-methoxybenzo[d]isoxazol-3-yl)-4-hydroxy-7-methoxychromane-8-sulfonamide

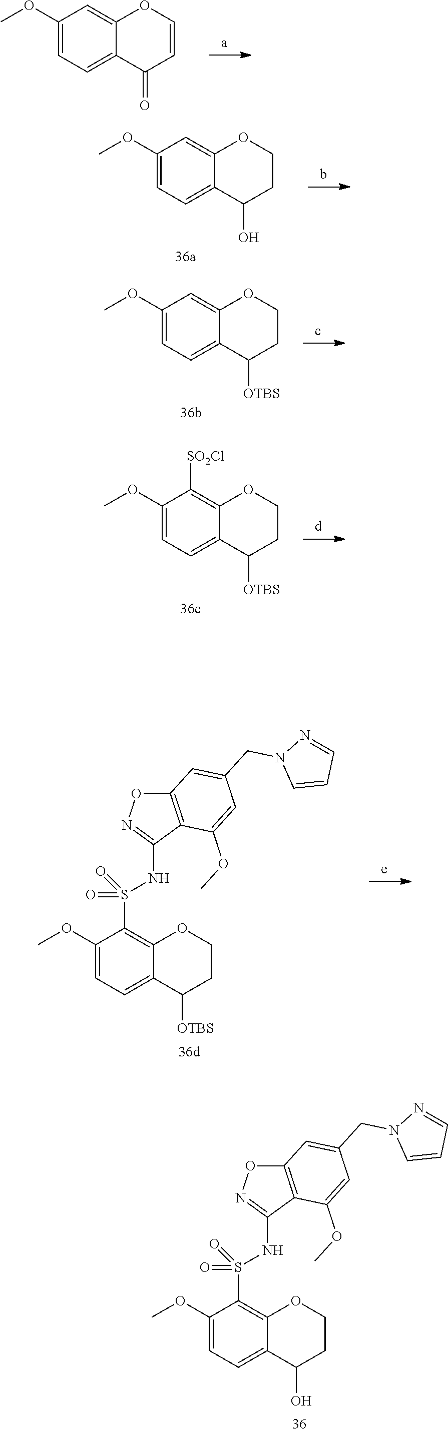

### Step a:

### 7-Methoxychroman-4-ol

Under a hydrogen atmosphere, palladium on carbon (900 mg, 10% w/w) was added to a solution of 7-methoxy-4H-chromen-4-one (9.10 g, 51.70 mmol) in methanol (100 mL), and the reaction system was stirred at 25° C. for 16 h. After the reaction was completed, the reaction mixture was filtered under reduced pressure, and the filtrate was concentrated under reduced pressure. The resulting crude product was purified by flash silica gel column chromatography (petroleum ether:ethyl acetate=84:16) to give compound 36a (7.90 g, yield: 84.9%).

### Step b:

### tert-Butyl((7-methoxychroman-4-yl)oxy)dimethylsilane

tert-Butyldimethylsilyl chloride (1.25 g, 8.32 mmol) was added to a solution of compound 36a (1.00 g, 5.55 mmol) and 1H-imidazole (604 mg, 8.88 mmol) in N,N-dimethylformamide (20 mL). The reaction system was stirred for 16 h. After the reaction was completed, the reaction mixture was quenched with an aqueous solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=91:9) to give compound 36b (1.40 g, yield: 85.7%).

¹HNMR (400 MHz, DMSO-d₆): δ 7.08 (d, 1H), 6.48 (dd, 1H), 6.31 (d, 1H), 4.78 (t, 1H), 4.21-4.16 (m, 2H), 3.70 (s, 3H), 2.04-1.96 (m, 1H), 1.85-1.79 (m, 1H), 0.87 (s, 9H), 0.15 (s, 3H), 0.12 (s, 3H).

### Step c:

### 4-((tert-Butyldimethylsilyl)oxy)-7-methoxychromane-8-sulfonyl chloride

Under a nitrogen atmosphere, at 0° C., n-butyllithium (2.5 M in n-hexane, 4.89 mL, 12.23 mmol) was slowly added dropwise to a solution of compound 36b (3.00 g, 10.19 mmol) in tetrahydrofuran (60 mL), and the reaction system was stirred for 1 h. Then, the reaction system was cooled to −78° C. Subsequently, sulfuryl chloride (1.65 g, 12.22 mmol) was added dropwise to the system, and stirring was continued for 15 min. After the reaction was completed, the reaction mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=85:15) to give compound 36c (1.40 g, yield: 35.0%).

¹HNMR (400 MHz, CDCl₃): δ7.43 (d, 1H), 6.58 (d, 1H), 4.75 (t, 1H), 4.48-4.45 (m, 2H), 3.96 (s, 3H), 2.12-2.08 (m, 1H), 2.07-1.97 (m, 1H), 0.90 (s, 9H), 0.16 (s, 3H), 0.14 (s, 3H).

### Step d:

### N-(6-((1H-Pyrazol-1-yl)methyl)-4-methoxybenzo[d]isoxazol-3-yl)-4-((tert-butyldimethylsilyl)oxy)-7-methoxychromane-8-sulfonamide

Sodium hydride (60%, 491 mg, 12.28 mmol) was added to a solution of intermediate 1 (600 mg, 2.46 mmol) in N,N-dimethylformamide (3 mL) at 0° C. The reaction system was stirred at 0° C. for 15 min. Then, compound 36c (1.35 g, 3.44 mmol) was added to the system, and stirring was continued for 30 min. After the reaction was completed, the reaction mixture was warmed to room temperature, adjusted to pH=6 with a 1 N aqueous hydrochloric acid solution, and extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:tetrahydrofuran=4:1) to give compound 36d (680 mg, yield: 46.1%).

MS m/z (ESI): δ01.1 [M+H]⁺.

### Step e:

### N-(6-((1H-Pyrazol-1-yl)methyl)-4-methoxybenzo[d]isoxazol-3-yl)-4-hydroxy-7-methoxychromane-8-sulfonamide

Tetrabutylammonium fluoride trihydrate (70 mg, 0.25 mmol) was added to a solution of compound 36d (50 mg, 0.08 mmol) in tetrahydrofuran (5 mL). The reaction system was stirred for 16 h. After the reaction was completed, the reaction mixture was quenched with a saturated aqueous ammonium chloride solution and extracted with ethyl acetate (20 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 36 (30 mg, yield: 74.1%).

¹HNMR (400 MHz, DMSO-d₆): δ7.87 (d, 1H), 7.50 (d, 1H), 7.42 (d, 1H), 6.81 (s, 1H), 6.75 (s, 1H), 6.70 (d, 1H), 6.30 (t, 1H), 5.42 (s, 2H), 5.37 (d, 1H), 4.57-4.52 (m, 1H), 4.21-4.15 (m, 1H) 4.12-4.07 (m, 1H), 3.89 (s, 3H), 3.73 (s, 3H), 1.87-1.74 (m, 2H).

MS m/z (ESI): 487.0 [M+H]⁺.

### Example 43

### Synthesis of Compound 37

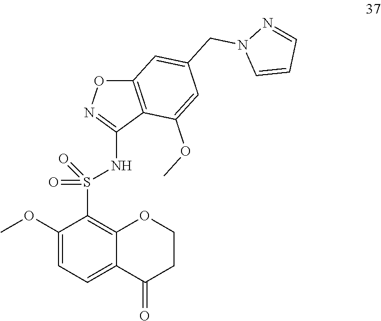 [SMILES: COc1ccc2c(c1S(=O)(=O)Nc1noc3cc(Cn4cccn4)cc(OC)c13)OCCC2=O]

### N-(6-((1H-Pyrazol-1-yl)methyl)-4-methoxybenzo[d]isoxazol-3-yl)-7-methoxy-4-oxochromane-8-sulfonamide

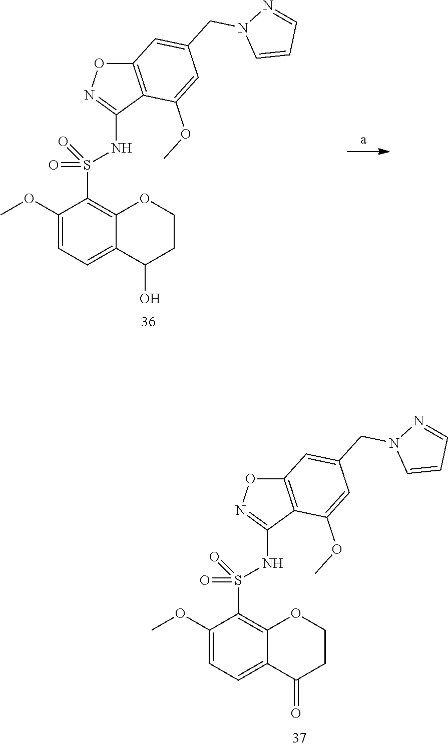 [SMILES: COc1ccc2c(c1S(=O)(=O)Nc1noc3cc(Cn4cccn4)cc(OC)c13)OCCC2O ; COc1ccc2c(c1S(=O)(=O)Nc1noc3cc(Cn4cccn4)cc(OC)c13)OCCC2=O]

### Step a:

### N-(6-((1H-Pyrazol-1-yl)methyl)-4-methoxybenzo[d]isoxazol-3-yl)-7-methoxy-4-oxochromane-8-sulfonamide

Manganese dioxide (26.8 mg, 0.31 mmol) was added to a solution of compound 36 (50 mg, 0.10 mmol) in chloroform (5 mL). The reaction system was heated to 60° C. and stirred for 16 h. After the reaction was completed, the reaction mixture was cooled to room temperature and filtered under reduced pressure, and the filtrate was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 37 (2.9 mg, yield: 5.8%).

¹HNMR (400 MHz, DMSO-d₆): δ10.24 (s, 1H), 7.95 (d, 1H), 7.87 (d, 1H), 7.50-7.49 (m, 1H), 6.92 (d, 1H), 6.82 (s, 1H), 6.73 (s, 1H), 6.30 (t, 1H), 5.44 (s, 2H), 4.47 (t, 2H), 3.85 (s, 3H), 3.83 (s, 3H), 2.72 (t, 2H).

MS m/z (ESI): 485.1 [M+H]⁺.

### Example 44

### Synthesis of Compound 38

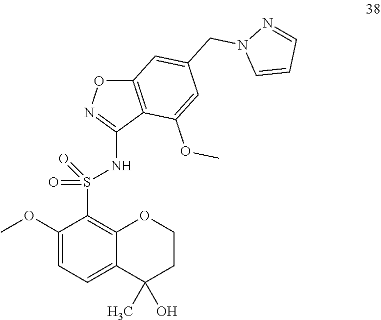 [SMILES: COc1ccc2c(c1S(=O)(=O)Nc1noc3cc(Cn4cccn4)cc(OC)c13)OCCC2(C)O]

### N-(6-((1H-Pyrazol-1-yl)methyl)-4-methoxybenzo[d]isoxazol-3-yl)-4-hydroxy-7-methoxy-4-methylchromane-8-sulfonamide

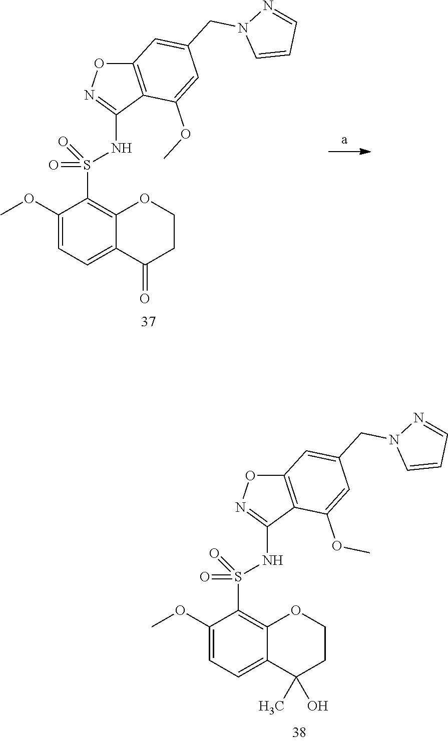 [SMILES: COc1ccc2c(c1S(=O)(=O)Nc1noc3cc(Cn4cccn4)cc(OC)c13)OCCC2=O ; COc1ccc2c(c1S(=O)(=O)Nc1noc3cc(Cn4cccn4)cc(OC)c13)OCCC2(C)O]

### Step a:

### N-(6-((1H-Pyrazol-1-yl)methyl)-4-methoxybenzo[d]isoxazol-3-yl)-4-hydroxy-7-methoxy-4-methylchromane-8-sulfonamide

Under a nitrogen atmosphere, methylmagnesium bromide (1.0 M in tetrahydrofuran, 3.1 mL, 3.10 mmol) was added to a solution of compound 37 (150 mg, 0.31 mmol) in tetrahydrofuran (10 mL) at 0° C., and the reaction system was stirred at 0° C. for 1 h. After the reaction was completed, the reaction mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 38 (60 mg, yield: 38.7%).

¹HNMR (400 MHz, DMSO-d₆): δ9.39 (s, 1H), 7.87 (d, 1H), 7.61 (d, 1H), 7.50 (d, 1H), 6.82 (s, 1H), 6.76 (s, 1H), 6.71 (d, 1H), 6.30 (t, 1H), 5.45 (s, 2H), 5.18 (s, 1H), 4.18-4.13 (m, 2H), 3.88 (s, 3H), 3.73 (s, 3H), 1.87-1.81 (m, 2H), 1.39 (s, 3H).

MS m/z (ESI): 501.1 [M+H]⁺.

### Example 45

### Synthesis of Compound 39

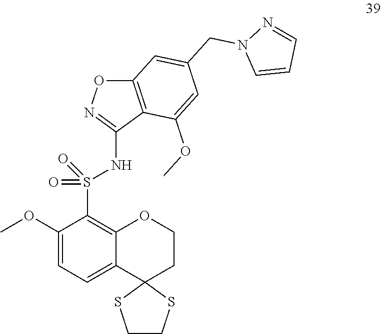 [SMILES: COc1ccc2c(c1S(=O)(=O)Nc1noc3cc(Cn4cccn4)cc(OC)c13)OCCC21SCCS1]

### N-(6-((1H-Pyrazol-1-yl)methyl)-4-methoxybenzo[d]isoxazol-3-yl)-7-methoxyspiro[chromane-4,2′-[1,3]dithiolane]-8-sulfonamide

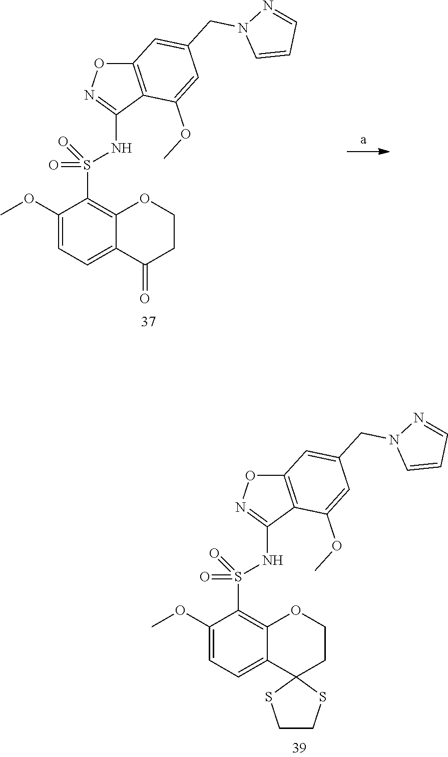 [SMILES: COc1ccc2c(c1S(=O)(=O)Nc1noc3cc(Cn4cccn4)cc(OC)c13)OCCC2=O ; COc1ccc2c(c1S(=O)(=O)Nc1noc3cc(Cn4cccn4)cc(OC)c13)OCCC21SCCS1]

### Step a:

### N-(6-((1H-Pyrazol-1-yl)methyl)-4-methoxybenzo[d]isoxazol-3-yl)-7-methoxyspiro[chromane-4,2′-[1,3]dithiolane]-8-sulfonamide

1,2-Ethanedithiol (12 mg, 0.12 mmol) and boron trifluoride diethyl etherate (18 mg, 0.12 mmol) were added to a solution of compound 37 (30 mg, 0.06 mmol) in dichloromethane (4 mL) at 30° C. The reaction system was stirred at 30° C. for 16 h. After the reaction was completed, the reaction mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 39 (8.9 mg, yield: 25.6%).

¹HNMR (400 MHz, DMSO-d₆): δ9.77 (s, 1H), 7.94 (d, 1H), 7.87 (d, 1H), 7.50 (d, 1H), 6.85 (s, 1H), 6.79-6.75 (m, 2H), 6.30 (t, 1H), 5.45 (s, 2H), 4.20-4.19 (m, 2H), 3.84 (s, 3H), 3.74 (s, 3H), 3.64-3.57 (m, 2H), 3.51-3.45 (m, 2H), 2.41-2.38 (m, 2H).

MS m/z (ESI): 561.1 [M+H]⁺.

### Example 46

### Synthesis of Compound 40

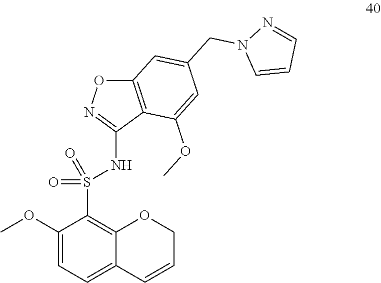 [SMILES: COc1ccc2c(c1S(=O)(=O)Nc1noc3cc(Cn4cccn4)cc(OC)c13)OCC=C2]

### N-(6-((1H-Pyrazol-1-yl)methyl)-4-methoxybenzo[d]isoxazol-3-yl)-7-methoxy-2H-chromene-8-sulfonamide

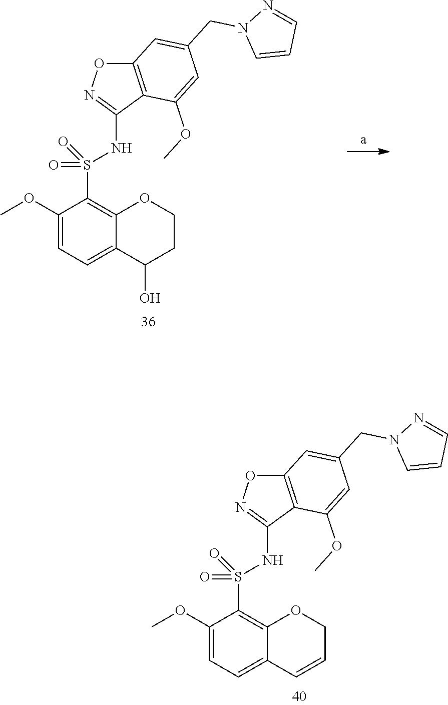 [SMILES: COc1ccc2c(c1S(=O)(=O)Nc1noc3cc(Cn4cccn4)cc(OC)c13)OCCC2O ; COc1ccc2c(c1S(=O)(=O)Nc1noc3cc(Cn4cccn4)cc(OC)c13)OCC=C2]

### Step a:

### N-(6-((1H-Pyrazol-1-yl)methyl)-4-methoxybenzo[d]isoxazol-3-yl)-7-methoxy-2H-chromene-8-sulfonamide

Burgess reagent (88 mg, 0.37 mmol) was added to a solution of compound 36 (60 mg, 0.12 mmol) in toluene (2 mL). The reaction system was heated to 80° C. and stirred for 3 h. After the reaction was completed, the reaction mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 40 (4.3 mg, yield: 7.4%).

¹HNMR (400 MHz, DMSO-d₆): δ7.87 (d, 1H), 7.50 (d, 1H), 7.17 (d, 1H), 6.79 (s, 1H), 6.71 (s, 1H), 6.63 (d, 1H), 6.45-6.43 (m, 1H), 6.30 (t, 1H), 5.78-5.74 (m, 1H), 5.44 (s, 2H), 4.63-4.61 (m, 2H), 3.86 (s, 3H), 3.72 (s, 3H).

MS m/z (ESI): 469.1 [M+H]⁺.

### Example 47

### Synthesis of Compound 41

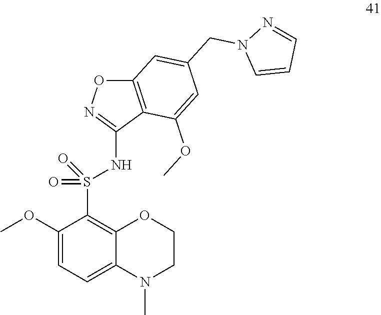 [SMILES: COc1ccc2c(c1S(=O)(=O)Nc1noc3cc(Cn4cccn4)cc(OC)c13)OCCN2C]

### N-(6-((1H-Pyrazol-1-yl)methyl)-4-methoxybenzo[d]isoxazol-3-yl)-7-methoxy-4-methyl-3,4-dihydro-2H-benzo[b][1,4]oxazine-8-sulfonamide

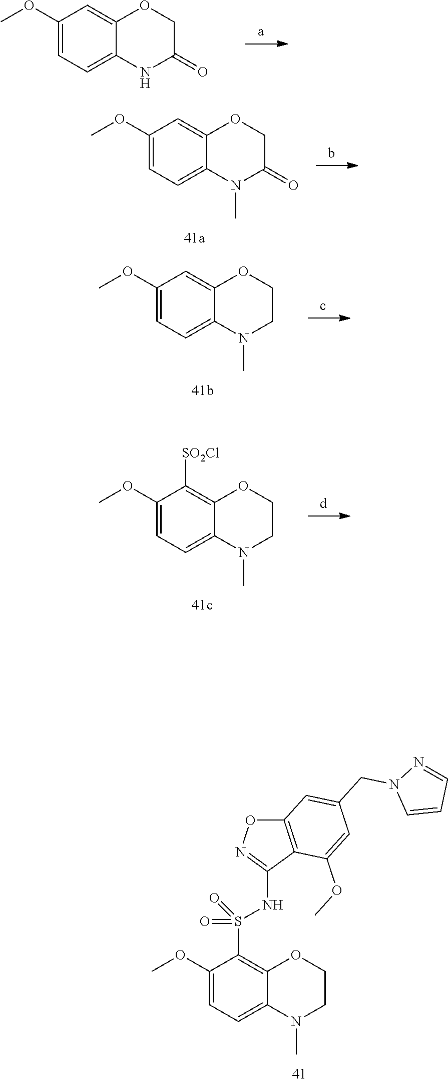 [SMILES: COc1ccc2c(c1)OCC(=O)N2 ; COc1ccc2c(c1)OCC(=O)N2C ; COc1ccc2c(c1)OCCN2C ; COc1ccc2c(c1S(=O)(=O)Cl)OCCN2C ; COc1ccc2c(c1S(=O)(=O)Nc1noc3cc(Cn4cccn4)cc(OC)c13)OCCN2C]

### Step a:

### 7-Methoxy-4-methyl-2H-benzo[b][1,4]oxazin-3(4H)-one

Under a nitrogen atmosphere, sodium hydride (60%, 0.58 g, 14.50 mmol) was added to a solution of 7-methoxy-2H-benzo[b][1,4]oxazin-3(4H)-one (1.84 g, 10.27 mmol) in N,N-dimethylformamide (30 mL) at 0° C., and the reaction system was stirred at 0° C. for 30 min. Then, iodomethane (1.61 g, 11.34 mmol) was added to the system, and stirring was continued for 3 h. After the reaction was completed, the reaction mixture was warmed to room temperature, quenched with ice water (50 mL), and extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 41a (1.86 g, yield: 93.7%).

¹HNMR (400 MHz, DMSO-d₆): δ7.06 (d, 1H), 6.66-6.61 (m, 2H), 4.61 (s, 2H), 3.72 (s, 3H), 3.24 (s, 3H).

### Step b:

### 7-Methoxy-4-methyl-3,4-dihydro-2H-benzo[b][1,4]oxazine

Under a nitrogen atmosphere, a borane-dimethyl sulfide complex solution (19 mL) was added to a solution of compound 41a (1.86 g, 9.63 mmol) in tetrahydrofuran (40 mL) at 30° C., and the reaction system was heated to 75° C., stirred for 3 h, and then cooled to 0° C. The reaction mixture was quenched with ice water (40 mL) and extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=6:1) to give compound 41b (1.54 g, yield: 89.3%).

¹HNMR (400 MHz, CDCl₃): δ6.62 (d, 1H), 6.45-6.41 (m, 2H), 4.32-4.30 (m, 2H), 3.73 (s, 3H), 3.18-3.15 (m, 2H), 2.82 (s, 3H).

### Step c:

### 7-Methoxy-4-methyl-3,4-dihydro-2H-benzo[b][1,4]oxazine-8-sulfonyl chloride

Under a nitrogen atmosphere, n-butyllithium (2.5 M in n-hexane, 0.67 mL, 1.68 mmol) was added dropwise to a solution of compound 41b (200 mg, 1.16 mmol) in tetrahydrofuran (6 mL) at 0° C., and the reaction system was stirred at 0° C. for 1 h and then cooled to −78° C. Subsequently, sulfuryl chloride (181 mg, 1.34 mmol) was added, and stirring was continued at −78° C. for 15 min. After the reaction was completed, the mixture was quenched with a saturated aqueous ammonium chloride solution and extracted with ethyl acetate (20 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound 41c (80 mg, yield: 25.8%).

¹HNMR (400 MHz, CDCl₃): δ6.87 (d, 1H), 6.53 (d, 1H), 4.49-4.47 (m, 2H), 3.89 (s, 3H), 3.29-3.27 (m, 2H), 2.88 (s, 3H).

### Step d:

### N-(6-((1H-Pyrazol-1-yl)methyl)-4-methoxybenzo[d]isoxazol-3-yl)-7-methoxy-4-methyl-3,4-dihydro-2H-benzo[b][1,4]oxazine-8-sulfonamide

Sodium hydride (60%, 49 mg, 1.23 mmol) was added to a solution of intermediate 1 (60 mg, 0.25 mmol) in N,N-dimethylformamide (1 mL) at 0° C. The reaction system was stirred at 0° C. for 15 min. Then, compound 41c (82 mg, 0.30 mmol) was added to the system, and stirring was continued for 15 min. After the reaction was completed, the mixture was warmed to room temperature. A 1 N aqueous hydrochloric acid solution (20 mL) was added to the reaction mixture, and the resulting mixture was extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 41 (9.2 mg, yield: 7.7%).

¹HNMR (400 MHz, DMSO-d₆): δ7.87 (d, 1H), 7.50 (d, 1H), 6.84-6.81 (m, 2H), 6.74 (s, 1H), 6.57 (d, 1H), 6.30 (t, 1H), 5.44 (s, 2H), 4.16 (t, 2H), 3.89 (s, 3H), 3.65 (s, 3H), 3.10 (t, 2H), 2.76 (s, 3H).

MS m/z (ESI): 486.1 [M+H]⁺.

### Example 48

### Synthesis of Compound 42

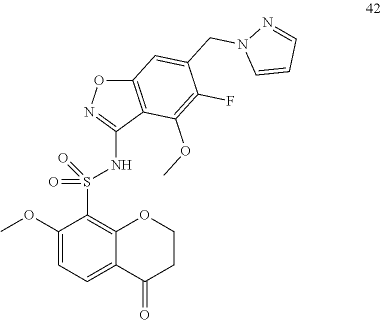 [SMILES: COc1ccc2c(c1S(=O)(=O)Nc1noc3cc(Cn4cccn4)c(F)c(OC)c13)OCCC2=O]

### N-(6-((1H-Pyrazol-1-yl)methyl)-5-fluoro-4-methoxybenzo[d]isoxazol-3-yl)-7-methoxy-4-oxochromane-8-sulfonamide

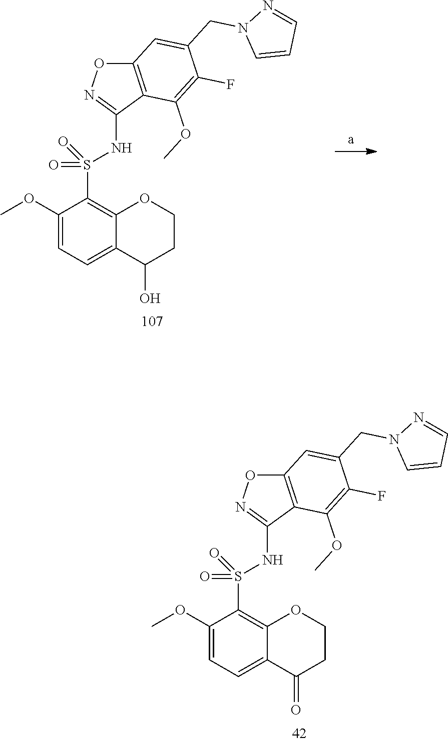 [SMILES: COc1ccc2c(c1S(=O)(=O)Nc1noc3cc(Cn4cccn4)c(F)c(OC)c13)OCCC2O ; COc1ccc2c(c1S(=O)(=O)Nc1noc3cc(Cn4cccn4)c(F)c(OC)c13)OCCC2=O]

### Step a:

### N-(6-((1H-Pyrazol-1-yl)methyl)-5-fluoro-4-methoxybenzo[d]isoxazol-3-yl)-7-methoxy-4-oxochromane-8-sulfonamide

Manganese dioxide (258.5 mg, 2.97 mmol) was added to a solution of compound 107 (100 mg, 0.20 mmol) in chloroform (10 mL). The reaction system was heated to 60° C. and stirred for 16 h. After the reaction was completed, the reaction mixture was cooled to room temperature and filtered under reduced pressure, and the filtrate was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 42 (80 mg, yield: 80.3%).

¹HNMR (400 MHz, DMSO-d₆): δ10.64 (s, 1H), 7.96 (s, 1H), 7.85 (d, 1H), 7.49 (d, 1H), 6.93-6.91 (m, 2H), 6.30 (t, 1H), 5.50 (s, 2H), 4.49-4.47 (m, 2H), 4.04 (s, 3H), 3.85 (s, 3H), 2.67 (t, 2H).

MS m/z (ESI): 503.1 [M+H]⁺.

### Example 49

### Synthesis of Compound 43

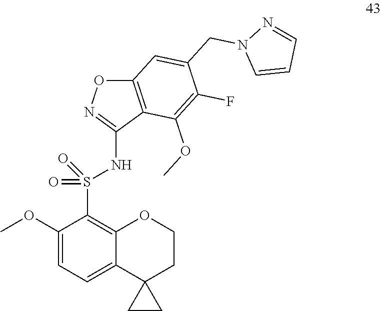 [SMILES: COc1ccc2c(c1S(=O)(=O)Nc1noc3cc(Cn4cccn4)c(F)c(OC)c13)OCCC21CC1]

### N-(6-((1H-Pyrazol-1-yl)methyl)-5-fluoro-4-methoxybenzo[d]isoxazol-3-yl)-7-methoxyspiro[chromane-4,1′-cyclopropane]-8-sulfonamide

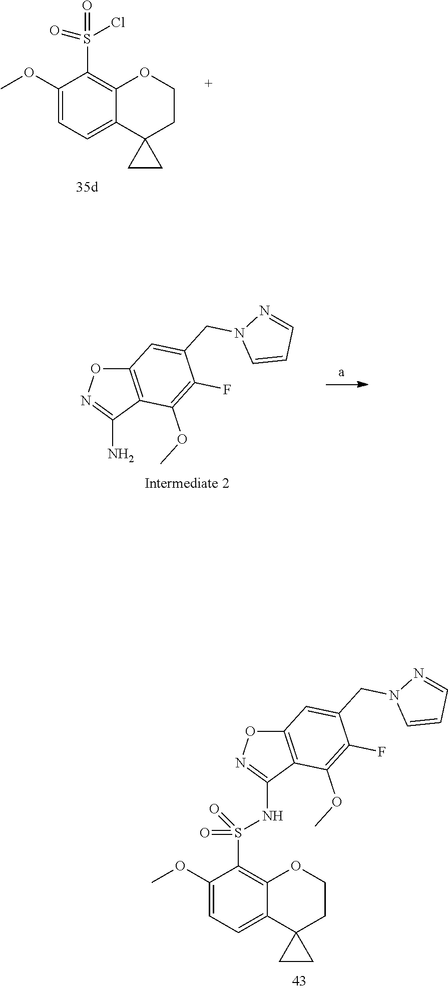 [SMILES: COc1ccc2c(c1S(=O)(=O)Cl)OCCC21CC1 ; COc1c(F)c(Cn2cccn2)cc2onc(N)c12 ; COc1ccc2c(c1S(=O)(=O)Nc1noc3cc(Cn4cccn4)c(F)c(OC)c13)OCCC21CC1]

### Step a:

### N-(6-((1H-Pyrazol-1-yl)methyl)-5-fluoro-4-methoxybenzo[d]isoxazol-3-yl)-7-methoxyspiro[chromane-4,1′-cyclopropane]-8-sulfonamide

Sodium hydride (60%, 38 mg, 0.95 mmol) was added to a solution of intermediate 2 (50 mg, 0.19 mmol) in N,N-dimethylformamide (1 mL) at 0° C. The reaction system was stirred at 0° C. for 15 min. Then, compound 35d (66 mg, 0.23 mmol) was added to the system, and stirring was continued for 15 min. After the reaction was completed, the mixture was quenched with a saturated aqueous ammonium chloride solution, adjusted to pH=5 with a 1 N aqueous hydrochloric acid solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 43 (29.3 mg, yield: 29.9%).

¹HNMR (400 MHz, DMSO-d₆): δ10.01 (s, 1H), 7.86 (d, 1H), 7.50-7.49 (m, 1H), 6.91-6-89 (m, 2H), 6.61 (d, 1H), 6.31 (t, 1H), 5.50 (s, 2H), 4.19-4.17 (m, 2H), 4.06 (d, 3H), 3.70 (s, 3H), 6.92 (d, 1H), 1.73 (t, 2H), 0.98-0.96 (m, 2H), 0.83-0.81 (m, 2H).

MS m/z (ESI): 515.1 [M+H]⁺.

### Example 50

### Synthesis of Compound 44

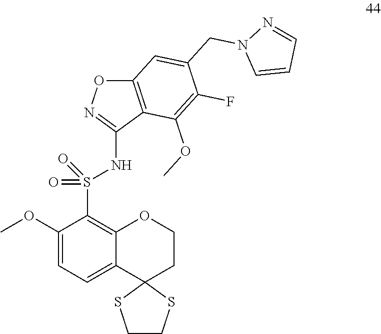 [SMILES: COc1ccc2c(c1S(=O)(=O)Nc1noc3cc(Cn4cccn4)c(F)c(OC)c13)OCCC21SCCS1]

### N-(6-((1H-Pyrazol-1-yl)methyl)-5-fluoro-4-methoxybenzo[d]isoxazol-3-yl)-7-methoxyspiro[chromane-4,2′-[1,3]dithiolane]-8-sulfonamide

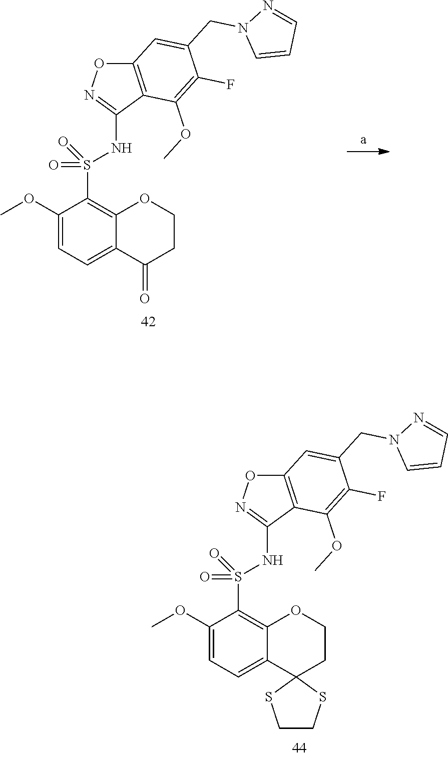 [SMILES: COc1ccc2c(c1S(=O)(=O)Nc1noc3cc(Cn4cccn4)c(F)c(OC)c13)OCCC2=O ; COc1ccc2c(c1S(=O)(=O)Nc1noc3cc(Cn4cccn4)c(F)c(OC)c13)OCCC21SCCS1]

### Step a:

### N-(6-((1H-Pyrazol-1-yl)methyl)-5-fluoro-4-methoxybenzo[d]isoxazol-3-yl)-7-methoxyspiro[chromane-4,2′-[1,3]dithiolane]-8-sulfonamide

1,2-Ethanedithiol (24 mg, 0.24 mmol) and boron trifluoride diethyl etherate (34 mg, 0.24 mmol) were added to a solution of compound 42 (60 mg, 0.12 mmol) in dichloromethane (5 mL) at 25° C. The reaction system was stirred at 25° C. for 16 h. After the reaction was completed, the reaction mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 44 (25 mg, yield: 36.2%).

¹HNMR (400 MHz, DMSO-d₆): δ10.25 (s, 1H), 7.96 (d, 1H), 7.86 (d, 1H), 7.50 (d, 1H), 6.93-6.91 (m, 1H), 6.79 (d, 1H), 6.31 (t, 1H), 5.51 (s, 2H), 4.22 (t, 2H), 4.01 (d, 3H), 3.75 (s, 3H), 3.65-3.58 (m, 2H), 3.52-3.46 (m, 2H), 2.42 (t, 2H).

MS m/z (ESI): 579.1 [M+H]⁺.

### Example 51

### Synthesis of Compound 45

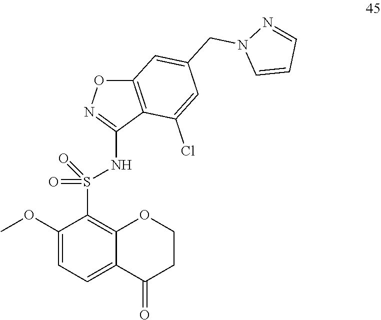 [SMILES: COc1ccc2c(c1S(=O)(=O)Nc1noc3cc(Cn4cccn4)cc(Cl)c13)OCCC2=O]

### N-(6-((1H-Pyrazol-1-yl)methyl)-4-chlorobenzo[d]isoxazol-3-yl)-7-methoxy-4-oxochromane-8-sulfonamide

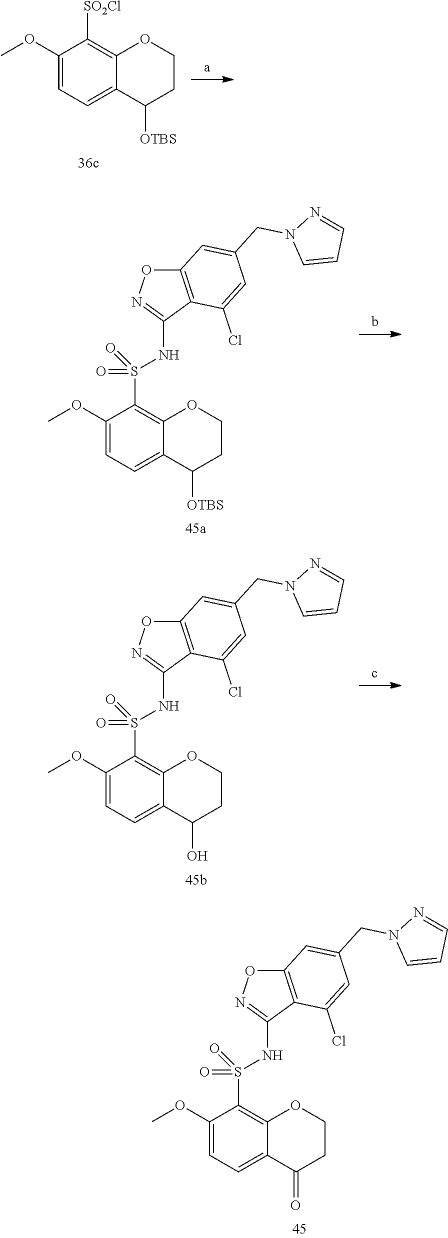 [SMILES: COc1ccc2c(c1S(=O)(=O)Cl)OCCC2O[Si](C)(C)C(C)(C)C ; COc1ccc2c(c1S(=O)(=O)Nc1noc3cc(Cn4cccn4)cc(Cl)c13)OCCC2O[Si](C)(C)C(C)(C)C ; COc1ccc2c(c1S(=O)(=O)Nc1noc3cc(Cn4cccn4)cc(Cl)c13)OCCC2O ; COc1ccc2c(c1S(=O)(=O)Nc1noc3cc(Cn4cccn4)cc(Cl)c13)OCCC2=O]

### Step a:

### N-(6-((1H-Pyrazol-1-yl)methyl)-4-chlorobenzo[d]isoxazol-3-yl)-4-((tert-butyldimethylsilyl)oxy)-7-methoxychromane-8-sulfonamide

Sodium hydride (60%, 113 mg, 2.83 mmol) was added to a solution of intermediate 6 (140 mg, 0.56 mmol) in N,N-dimethylformamide (1.4 mL) at 0° C. The reaction system was stirred at 0° C. for 15 min. Then, compound 36c (288 mg, 0.73 mmol) was added to the system, and stirring was continued for 15 min. After the reaction was completed, the reaction mixture was warmed to room temperature, quenched with a saturated aqueous ammonium chloride solution, and extracted with ethyl acetate (100 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=1:1) to give compound 45a (225 mg, yield: 66.0%).

MS m/z (ESI): δ05.6 [M+H]⁺.

### Step b:

### N-(6-((1H-Pyrazol-1-yl)methyl)-4-chlorobenzo[d]isoxazol-3-yl)-4-hydroxy-7-methoxychromane-8-sulfonamide

Tetrabutylammonium fluoride trihydrate (312 mg, 1.12 mmol) was added to a solution of compound 45a (225 mg, 0.37 mmol) in tetrahydrofuran (10 mL). The reaction system was stirred for 16 h. After the reaction was completed, the reaction mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound 45b (160 mg, yield: 87.7%).

MS m/z (ESI): 491.0 [M+H]⁺.

### Step c:

### N-(6-((1H-Pyrazol-1-yl)methyl)-4-chlorobenzo[d]isoxazol-3-yl)-7-methoxy-4-oxochromane-8-sulfonamide

Manganese dioxide (425 mg, 4.89 mmol) was added to a solution of compound 45b (160 mg, 0.33 mmol) in chloroform (10 mL). The reaction system was heated to 60° C. and stirred for 4 h. After the reaction was completed, the reaction mixture was cooled to room temperature and filtered under reduced pressure, and the filtrate was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 45 (90 mg, yield: 56.5%). ¹HNMR (400 MHz, DMSO-d₆): δ10.66 (s, 1H), 7.97 (d, 1H), 7.90 (d, 1H), 7.51 (d, 1H), 7.45-7.43 (m, 1H), 7.25 (s, 1H), 6.93 (d, 1H), 6.31 (t, 1H), 5.50 (s, 2H), 4.47 (t, 2H), 3.83 (s, 3H), 2.72 (t, 2H).

MS m/z (ESI): 489.0 [M+H]⁺.

### Example 52

### Synthesis of Compound 46

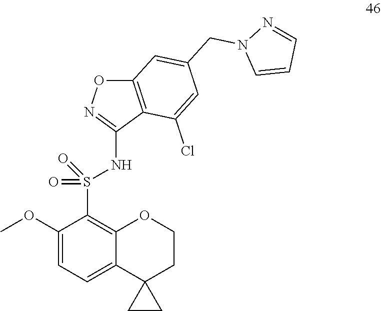 [SMILES: COc1ccc2c(c1S(=O)(=O)Nc1noc3cc(Cn4cccn4)cc(Cl)c13)OCCC21CC1]

### N-(6-((1H-Pyrazol-1-yl)methyl)-4-chlorobenzo[d]isoxazol-3-yl)-7-methoxyspiro[chromane-4,1′-cyclopropane]-8-sulfonamide

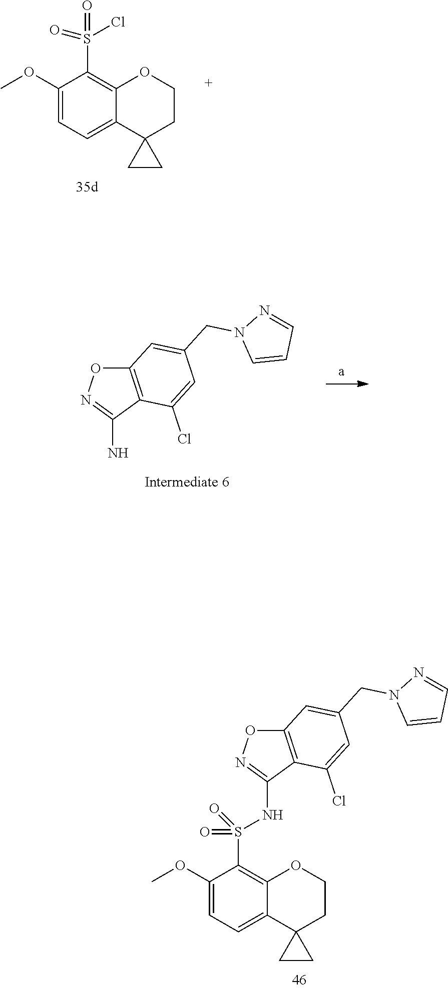 [SMILES: COc1ccc2c(c1S(=O)(=O)Cl)OCCC21CC1 ; Nc1noc2cc(Cn3cccn3)cc(Cl)c12 ; COc1ccc2c(c1S(=O)(=O)Nc1noc3cc(Cn4cccn4)cc(Cl)c13)OCCC21CC1]

### Step a:

### N-(6-((1H-Pyrazol-1-yl)methyl)-4-chlorobenzo[d]isoxazol-3-yl)-7-methoxyspiro[chromane-4,1′-cyclopropane]-8-sulfonamide

Sodium hydride (60%, 40 mg, 1.00 mmol) was added to a solution of intermediate 6 (50 mg, 0.20 mmol) in N,N-dimethylformamide (1 mL) at 0° C. The reaction system was stirred at 0° C. for 15 min. Then, compound 35d (76 mg, 0.26 mmol) was added to the system, and stirring was continued for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 46 (18 mg, yield: 17.9%).

¹HNMR (400 MHz, DMSO-d₆): δ10.11 (s, 1H), 7.90 (d, 1H), 7.51 (d, 1H), 7.46 (s, 1H), 7.26 (s, 1H), 6.91 (d, 1H), 6.61 (d, 1H), 6.32 (t, 1H), 5.51 (s, 2H), 4.17 (t, 2H), 3.69 (s, 3H), 1.73 (t, 2H), 0.99-0.97 (m, 2H), 0.84-0.81 (m, 2H).

MS m/z (ESI): 501.0 [M+H]⁺.

### Example 53

### Synthesis of Compound 47

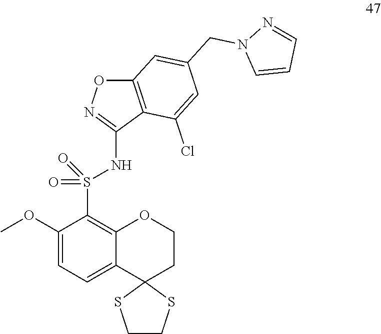 [SMILES: COc1ccc2c(c1S(=O)(=O)Nc1noc3cc(Cn4cccn4)cc(Cl)c13)OCCC21SCCS1]

### N-(6-((1H-Pyrazol-1-yl)methyl)-4-chlorobenzo[d]isoxazol-3-yl)-7-methoxyspiro[chromane-4,2′-[1,3]dithiolane]-8-sulfonamide

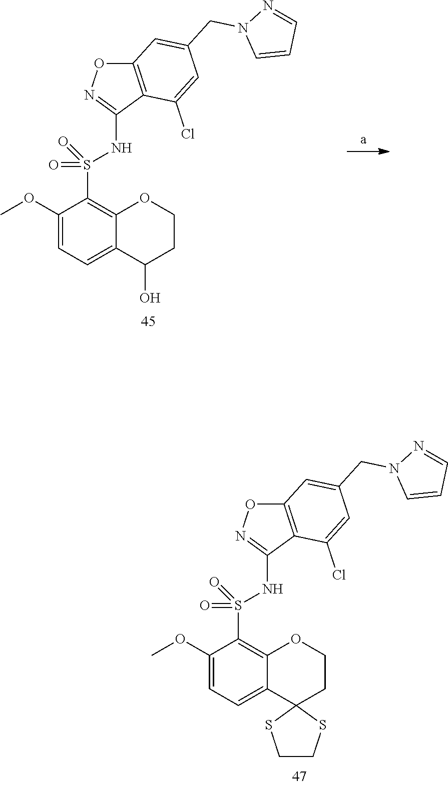 [SMILES: COc1ccc2c(c1S(=O)(=O)Nc1noc3cc(Cn4cccn4)cc(Cl)c13)OCCC2O ; COc1ccc2c(c1S(=O)(=O)Nc1noc3cc(Cn4cccn4)cc(Cl)c13)OCCC21SCCS1]

### Step a:

### N-(6-((1H-Pyrazol-1-yl)methyl)-4-chlorobenzo[d]isoxazol-3-yl)-7-methoxyspiro[chromane-4,2′-[1,3]dithiolane]-8-sulfonamide

1,2-Ethanedithiol (50 mg, 0.53 mmol) and boron trifluoride diethyl etherate (72 mg, 0.53 mmol) were added to a solution of compound 45 (65 mg, 0.13 mmol) in dichloromethane (5 mL) at 25° C. The reaction system was stirred at 25° C. for 16 h. After the reaction was completed, the reaction mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 47 (13.9 mg, yield: 18.5%).

¹HNMR (400 MHz, DMSO-d₆): δ10.30 (s, 1H), 7.96 (d, 1H), 7.91 (d, 1H), 7.51 (d, 1H), 7.46 (s, 1H), 7.25 (s, 1H), 6.78 (d, 1H), 6.31 (t, 1H), 5.50 (s, 2H), 4.21-4.19 (m, 2H), 3.73 (s, 3H), 3.62-3.59 (m, 2H), 3.52-3.46 (m, 2H), 2.41 (t, 2H).

MS m/z (ESI): 565.0 [M+H]⁺.

### Example 54

### Synthesis of Compound 49

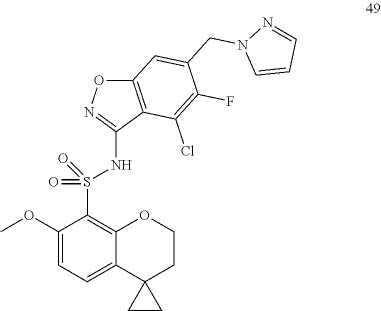 [SMILES: COc1ccc2c(c1S(=O)(=O)Nc1noc3cc(Cn4cccn4)c(F)c(Cl)c13)OCCC21CC1]

### N-(6-((1H-Pyrazol-1-yl)methyl)-4-chloro-5-fluorobenzo[d]isoxazol-3-yl)-7-methoxyspiro[chromane-4,1′-cyclopropane]-8-sulfonamide

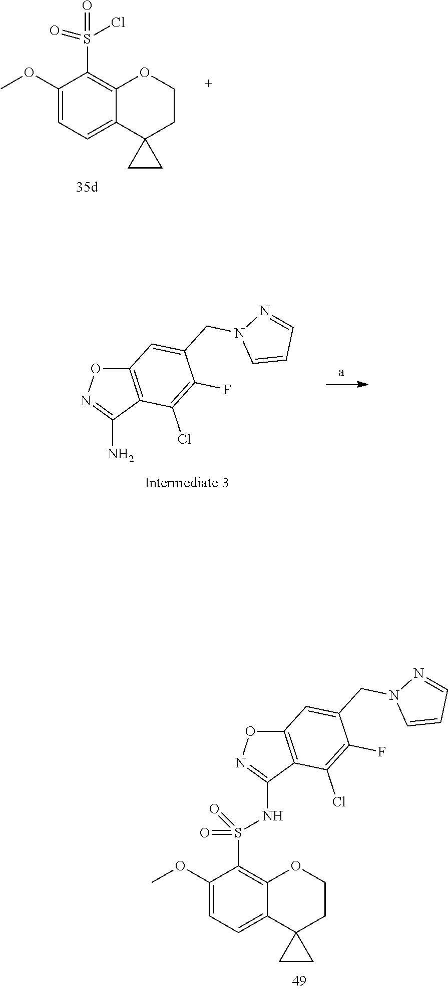 [SMILES: COc1ccc2c(c1S(=O)(=O)Cl)OCCC21CC1 ; Nc1noc2cc(Cn3cccn3)c(F)c(Cl)c12 ; COc1ccc2c(c1S(=O)(=O)Nc1noc3cc(Cn4cccn4)c(F)c(Cl)c13)OCCC21CC1]

### Step a:

### N-(6-((1H-Pyrazol-1-yl)methyl)-4-chloro-5-fluorobenzo[d]isoxazol-3-yl)-7-methoxyspiro[chromane-4,1′-cyclopropane]-8-sulfonamide

Sodium hydride (60%, 36 mg, 0.90 mmol) was added to a solution of intermediate 3 (50 mg, 0.19 mmol) in N,N-dimethylformamide (1 mL) at 0° C. The reaction system was stirred at 0° C. for 15 min. Then, compound 35d (68 mg, 0.24 mmol) was added to the system, and stirring was continued for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 49 (7.0 mg, yield: 7.2%).

¹HNMR (400 MHz, DMSO-d₆): δ10.33 (s, 1H), 7.88 (d, 1H), 7.51 (d, 1H), 7.42 (s, 1H), 6.90 (d, 1H), 6.61 (d, 1H), 6.31 (t, 1H), 5.56 (s, 2H), 4.19 (t, 2H), 3.70 (s, 3H), 1.74 (t, 2H), 0.99-0/97 (m, 2H), 0.84-0.82 (m, 2H).

MS m/z (ESI): 519.0 [M+H]⁺.

### Example 55

### Synthesis of Compound 51

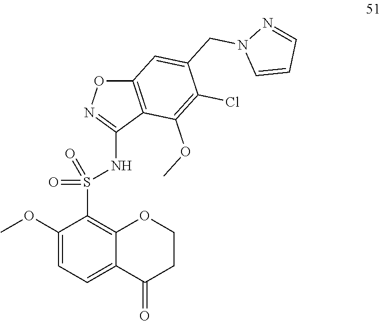 [SMILES: COc1ccc2c(c1S(=O)(=O)Nc1noc3cc(Cn4cccn4)c(Cl)c(OC)c13)OCCC2=O]

### N-(6-((1H-Pyrazol-1-yl)methyl)-5-chloro-4-methoxybenzo[d]isoxazol-3-yl)-7-methoxy-4-oxochromane-8-sulfonamide

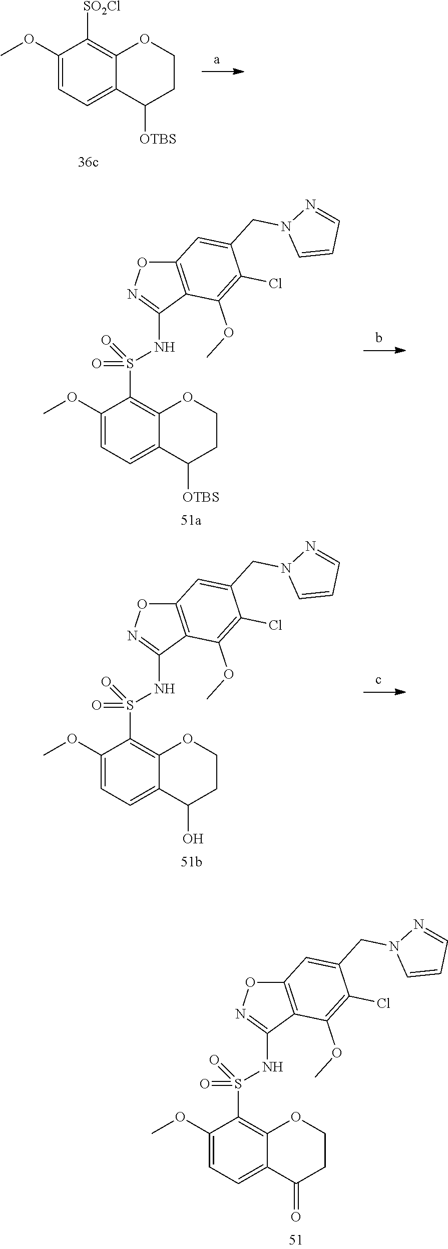 [SMILES: COc1ccc2c(c1S(=O)(=O)Cl)OCCC2O[Si](C)(C)C(C)(C)C ; COc1ccc2c(c1S(=O)(=O)Nc1noc3cc(Cn4cccn4)c(Cl)c(OC)c13)OCCC2O[Si](C)(C)C(C)(C)C ; COc1ccc2c(c1S(=O)(=O)Nc1noc3cc(Cn4cccn4)c(Cl)c(OC)c13)OCCC2O ; COc1ccc2c(c1S(=O)(=O)Nc1noc3cc(Cn4cccn4)c(Cl)c(OC)c13)OCCC2=O]

### Step a:

### N-(6-((1H-Pyrazol-1-yl)methyl)-5-chloro-4-methoxybenzo[d]isoxazol-3-yl)-4-((tert-butyldimethylsilyl)oxy)-7-methoxychromane-8-sulfonamide

Sodium hydride (60%, 86 mg, 2.15 mmol) was added to a solution of intermediate 5 (120 mg, 0.43 mmol) in N,N-dimethylformamide (2 mL) at 0° C. The reaction system was stirred at 0° C. for 15 min. Then, compound 36c (212 mg, 0.54 mmol) was added to the system, and stirring was continued for 15 min. After the reaction was completed, the reaction mixture was warmed to room temperature, quenched with a saturated aqueous ammonium chloride solution, and extracted with ethyl acetate (50 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=49:1) to give compound 51a (171 mg, yield: 62.5%).

### Step b:

### N-(6-((1H-Pyrazol-1-yl)methyl)-5-chloro-4-methoxybenzo[d]isoxazol-3-yl)-4-hydroxy-7-methoxychromane-8-sulfonamide

Tetrabutylammonium fluoride trihydrate (226 mg, 0.81 mmol) was added to a solution of compound 51a (171 mg, 0.27 mmol) in tetrahydrofuran (15 mL). The reaction system was stirred for 18 h. After the reaction was completed, the reaction mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=19:1) to give compound 51b (109 mg, yield: 77.7%).

¹ H NMR (400 MHz, CDCl₃) 38.17 (s, 1H), 7.61 (d, 1H), 7.51 (d, 1H), 7.42 (d, 1H), 6.56 (d, 1H), 6.54 (s, 1H), 6.36 (t, 1H), 5.51 (s, 2H), 4.71-4.69 (m, 1H), 4.44-4.40 (m, 1H), 4.33-4.27 (m, 1H), 4.13 (s, 3H), 3.89 (s, 3H), 1.82 (d, 1H), 1.28-1.24 (in, 1H).

### Step c:

### N-(6-((1H-Pyrazol-1-yl)methyl)-5-chloro-4-methoxybenzo[d]isoxazol-3-yl)-7-methoxy-4-oxochromane-8-sulfonamide

Manganese dioxide (260 mg, 2.99 mmol) was added to a solution of compound 51b (104 mg, 0.20 mmol) in chloroform (10 mL). The reaction system was stirred at 30° C. for 18 h. After the reaction was completed, the reaction mixture was cooled to room temperature and filtered under reduced pressure, and the filtrate was concentrated under reduced pressure. The resulting crude product was purified by high-performance liquid chromatography (Welchrom C18, 20×150 mm, m; mobile phase A: 5 mM aqueous ammonium bicarbonate solution, mobile phase B: acetonitrile; gradient ratio:phase B 5%-95%) to give compound 51 (9 mg, yield: 8.7%). ¹HNMR (400 MHz, DMSO-d₆): δ10.90 (s, 1H), 7.99 (d, 1H), 7.88 (d, 1H), 7.55 (d, 1H), 6.95 (d, 1H), 6.78 (s, 1H), 6.34 (t, 1H), 5.57 (s, 2H), 4.50 (t, 2H), 3.95 (s, 3H), 3.88 (s, 3H), 2.74 (t, 2H).

MS m/z (ESI): 519.0 [M+H]⁺.

### Example 56

### Synthesis of Compound 52

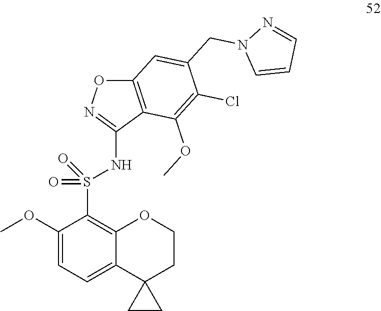 [SMILES: COc1ccc2c(c1S(=O)(=O)Nc1noc3cc(Cn4cccn4)c(Cl)c(OC)c13)OCCC21CC1]

### N-(6-((1H-Pyrazol-1-yl)methyl)-5-chloro-4-methoxybenzo[d]isoxazol-3-yl)-7-methoxyspiro[chromane-4,1′-cyclopropane]-8-sulfonamide

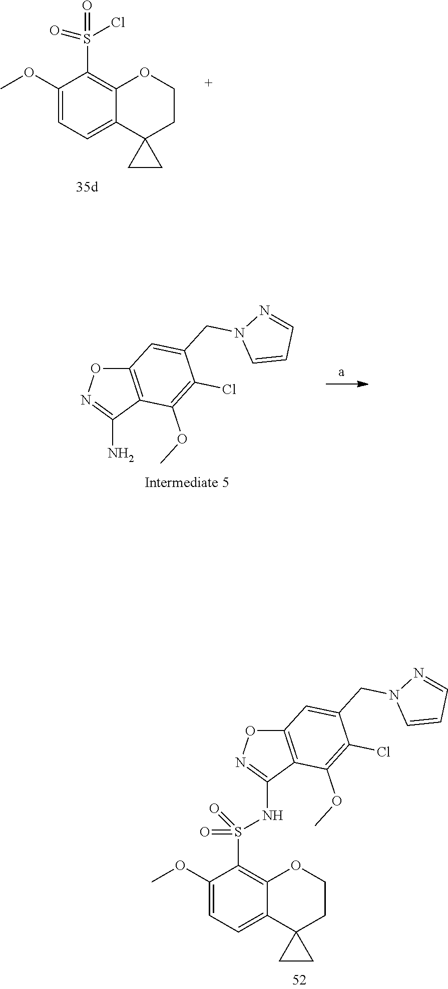 [SMILES: COc1ccc2c(c1S(=O)(=O)Cl)OCCC21CC1 ; COc1c(Cl)c(Cn2cccn2)cc2onc(N)c12 ; COc1ccc2c(c1S(=O)(=O)Nc1noc3cc(Cn4cccn4)c(Cl)c(OC)c13)OCCC21CC1]

### Step a:

### N-(6-((1H-Pyrazol-1-yl)methyl)-5-chloro-4-methoxybenzo[d]isoxazol-3-yl)-7-methoxyspiro[chromane-4,1′-cyclopropane]-8-sulfonamide

Sodium hydride (60%, 36 mg, 0.90 mmol) was added to a solution of intermediate 5 (50 mg, 0.18 mmol) in N,N-dimethylformamide (1 mL) at 0° C. The reaction system was stirred at 0° C. for 15 min. Then, compound 35d (68 mg, 0.24 mmol) was added to the system, and stirring was continued for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 52 (7 mg, yield: 7.3%).

¹HNMR (400 MHz, DMSO-d₆): δ10.33 (s, 1H), 7.88 (d, 1H), 7.55 (d, 1H), 6.90 (d, 1H), 6.76 (s, 1H), 6.61 (d, 1H), 6.35 (t, 1H), 5.56 (s, 2H), 4.19 (t, 2H), 3.97 (s, 3H), 3.70 (s, 3H), 1.74 (t, 2H), 0.99-0.97 (m, 2H), 0.84-0.82 (m, 2H).

MS m/z (ESI): 531.0 [M+H]⁺.

### Example 57

### Synthesis of Compound 53

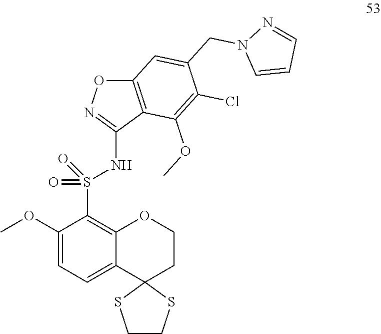 [SMILES: COc1ccc2c(c1S(=O)(=O)Nc1noc3cc(Cn4cccn4)c(Cl)c(OC)c13)OCCC21SCCS1]

### N-(6-((1H-Pyrazol-1-yl)methyl)-5-chloro-4-methoxybenzo[d]isoxazol-3-yl)-7-methoxyspiro[chromane-4,2′-[1,3]dithiolane]-8-sulfonamide

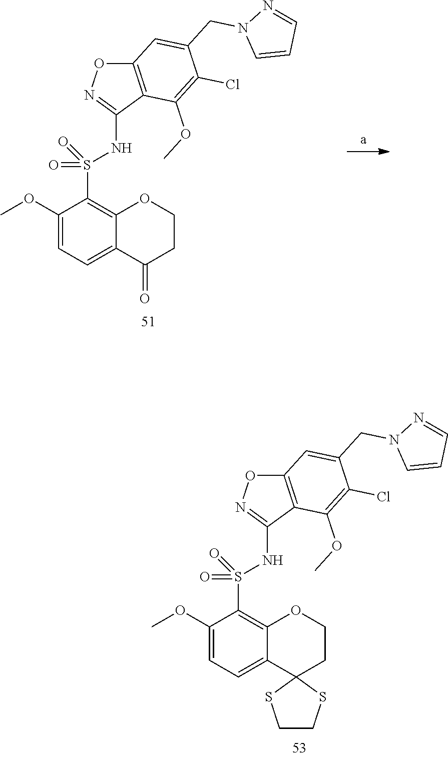 [SMILES: COc1ccc2c(c1S(=O)(=O)Nc1noc3cc(Cn4cccn4)c(Cl)c(OC)c13)OCCC2=O ; COc1ccc2c(c1S(=O)(=O)Nc1noc3cc(Cn4cccn4)c(Cl)c(OC)c13)OCCC21SCCS1]

### Step a:

### N-(6-((1H-Pyrazol-1-yl)methyl)-5-chloro-4-methoxybenzo[d]isoxazol-3-yl)-7-methoxyspiro[chromane-4,2′-[1,3]dithiolane]-8-sulfonamide

1,2-Ethanedithiol (76 mg, 0.81 mmol) and boron trifluoride diethyl etherate (115 mg, 0.81 mmol) were added to a solution of compound 51 (60 mg, 0.12 mmol) in dichloromethane (6 mL) at 25° C. The reaction system was stirred at 25° C. for 18 h. After the reaction was completed, the reaction mixture was quenched with a saturated aqueous ammonium chloride solution and extracted with dichloromethane (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 53 (25.3 mg, yield: 36.8%).

¹HNMR (400 MHz, DMSO-d₆): δ10.53 (s, 1H), 7.98 (d, 1H), 7.88 (d, 1H), 7.55 (d, 1H), 6.82-6.80 (m, 2H), 6.35 (t, 1H), 5.57 (s, 2H), 4.25 (t, 2H), 3.91 (s, 3H), 3.76 (s, 3H), 3.66-3.59 (m, 2H), 3.53-3.46 (m, 2H), 2.46-2.42 (m, 2H).

MS m/z (ESI): 595.0 [M+H]⁺.

### Example 58

### Synthesis of Compound 72

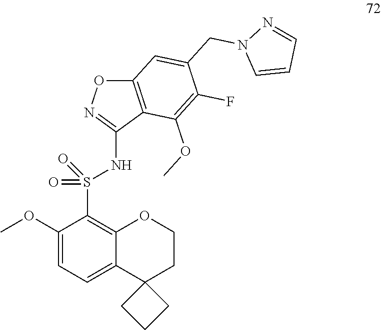 [SMILES: COc1ccc2c(c1S(=O)(=O)Nc1noc3cc(Cn4cccn4)c(F)c(OC)c13)OCCC21CCC1]

### N-(6-((1H-Pyrazol-1-yl)methyl)-5-fluoro-4-methoxybenzo[d]isoxazol-3-yl)-7-methoxyspiro[chromane-4,1′-cyclobutane]-8-sulfonamide

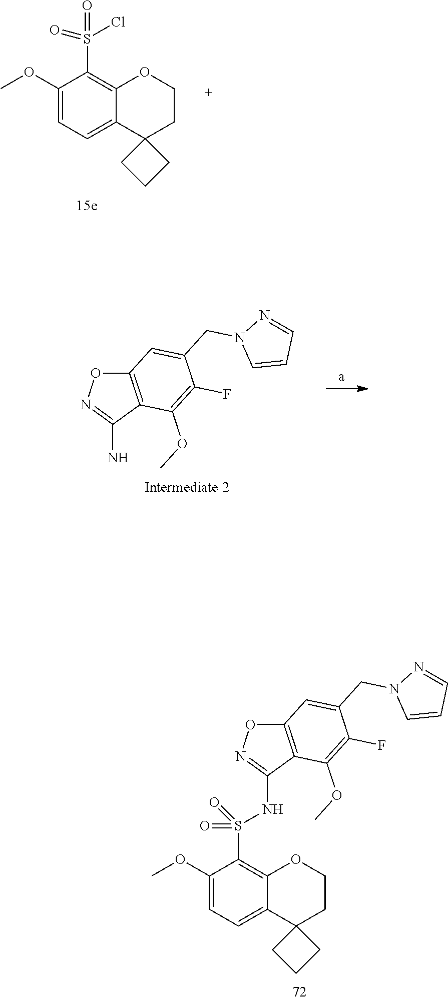 [SMILES: COc1ccc2c(c1S(=O)(=O)Cl)OCCC21CCC1 ; COc1c(F)c(Cn2cccn2)cc2onc(N)c12 ; COc1ccc2c(c1S(=O)(=O)Nc1noc3cc(Cn4cccn4)c(F)c(OC)c13)OCCC21CCC1]

### Step a:

### N-(6-((1H-Pyrazol-1-yl)methyl)-5-fluoro-4-methoxybenzo[d]isoxazol-3-yl)-7-methoxyspiro[chromane-4,1′-cyclobutane]-8-sulfonamide

Sodium hydride (60%, 46 mg, 1.15 mmol) was added to a solution of intermediate 2 (60 mg, 0.23 mmol) in N,N-dimethylformamide (1 mL) at 0° C. The reaction system was stirred at 0° C. for 15 min. Then, compound 15e (68 mg, 0.24 mmol) was added to the system, and stirring was continued for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 72 (45 mg, yield: 37.2%).

¹HNMR (400 MHz, DMSO-d₆): δ10.00 (s, 1H), 7.85 (d, 1H), 7.74 (d, 1H), 7.50 (d, 1H), 6.91 (d, 1H), 6.74 (d, 1H), 6.31 (t, 1H), 5.50 (s, 2H), 4.06-4.03 (m, 5H), 3.73 (s, 3H), 2.33-2.29 (m, 2H), 2.03-1.88 (m, 6H).

MS m/z (ESI): 529.1 [M+H]⁺.

### Example 59

### Synthesis of Compound 73

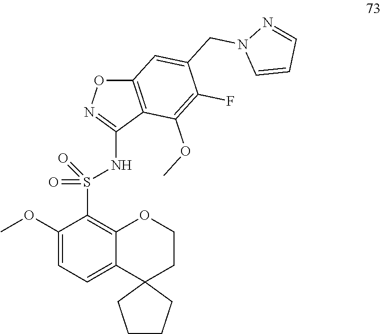 [SMILES: COc1ccc2c(c1S(=O)(=O)Nc1noc3cc(Cn4cccn4)c(F)c(OC)c13)OCCC21CCCC1]

### N-(6-((1H-Pyrazol-1-yl)methyl)-5-fluoro-4-methoxybenzo[d]isoxazol-3-yl)-7-methoxyspiro[chromane-4,1′-cyclopentane]-8-sulfonamide

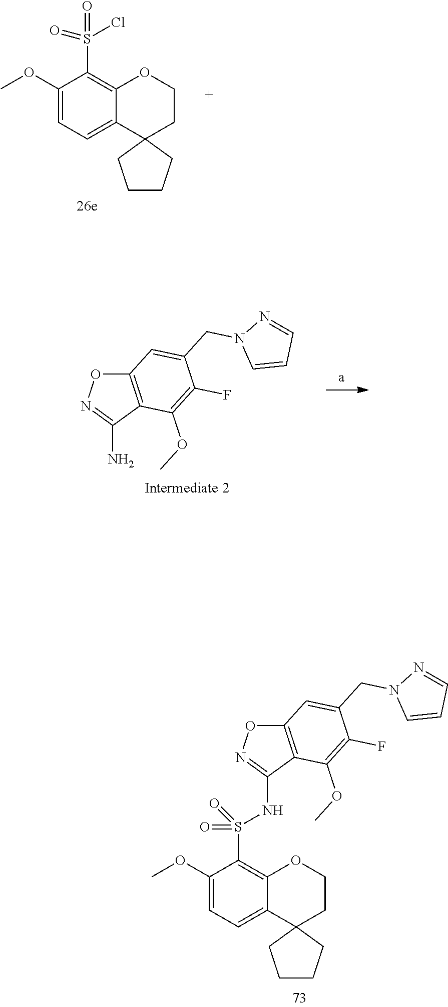 [SMILES: COc1ccc2c(c1S(=O)(=O)Cl)OCCC21CCCC1 ; COc1c(F)c(Cn2cccn2)cc2onc(N)c12 ; COc1ccc2c(c1S(=O)(=O)Nc1noc3cc(Cn4cccn4)c(F)c(OC)c13)OCCC21CCCC1]

### Step a:

### N-(6-((1H-Pyrazol-1-yl)methyl)-5-fluoro-4-methoxybenzo[d]isoxazol-3-yl)-7-methoxyspiro[chromane-4,1′-cyclopentane]-8-sulfonamide

Sodium hydride (60%, 53 mg, 1.33 mmol) was added to a solution of intermediate 2 (70 mg, 0.27 mmol) in N,N-dimethylformamide (1 mL) at 0° C. The reaction system was stirred at 0° C. for 15 min. Then, compound 26e (110 mg, 0.35 mmol) was added to the system, and stirring was continued for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 73 (44.7 mg, yield: 30.9%).

¹HNMR (400 MHz, DMSO-d₆): δ9.99 (s, 1H), 7.85 (d, 1H), 7.50 (d, 1H), 7.45 (d, 1H), 6.91 (d, 1H), 6.69 (d, 1H), 6.31 (t, 1H), 5.50 (s, 2H), 4.08 (t, 2H), 4.03 (d, 3H), 3.72 (s, 3H), 1.79-1.67 (m, 1OH).

MS m/z (ESI): 543.1 [M+H]⁺.

### Example 60

### Synthesis of Compound 101

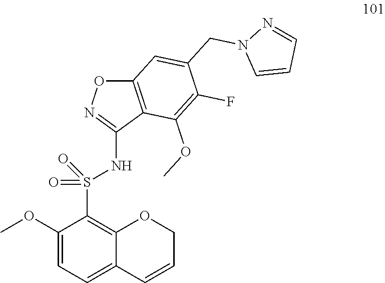 [SMILES: COc1ccc2c(c1S(=O)(=O)Nc1noc3cc(Cn4cccn4)c(F)c(OC)c13)OCC=C2]

### N-(6-((1H-Pyrazol-1-yl)methyl)-5-fluoro-4-methoxybenzo[d]isoxazol-3-yl)-7-methoxy-2H-chromene-8-sulfonamide

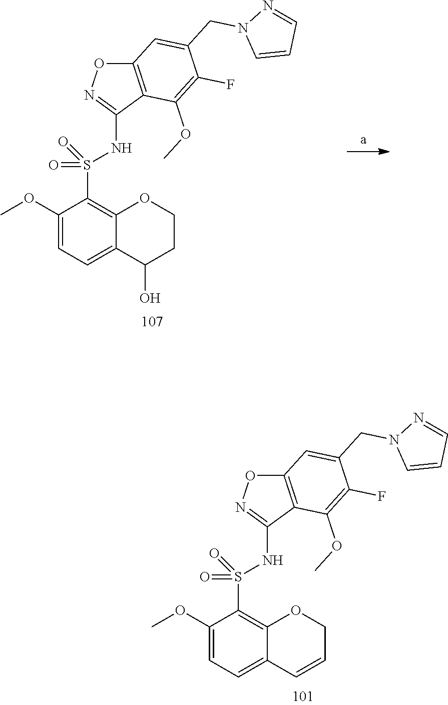 [SMILES: COc1ccc2c(c1S(=O)(=O)Nc1noc3cc(Cn4cccn4)c(F)c(OC)c13)OCCC2O ; COc1ccc2c(c1S(=O)(=O)Nc1noc3cc(Cn4cccn4)c(F)c(OC)c13)OCC=C2]

### Step a:

### N-(6-((1H-Pyrazol-1-yl)methyl)-5-fluoro-4-methoxybenzo[d]isoxazol-3-yl)-7-methoxy-2H-chromene-8-sulfonamide

p-Toluenesulfonyl chloride (121 mg, 0.63 mmol) was added to a solution of compound 107 (80 mg, 0.16 mmol) in pyridine (8 mL). The reaction system was heated to 110° C. and stirred for 4 h. After the reaction was completed, the mixture was quenched with a saturated aqueous ammonium chloride solution, adjusted to pH=5 with a 1 N aqueous hydrochloric acid solution, and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 101 (20.1 mg, yield: 26.1%). ¹HNMR (400 MHz, DMSO-d₆): δ10.29 (s, 1H), 7.86 (d, 1H), 7.50 (d, 1H), 7.23 (d, 1H), 6.92 (s, 1H), 6.68 (d, 1H), 6.46 (d, 1H), 6.31 (t, 1H), 5.81-5.76 (m, 1H), 5.50 (s, 2H), 4.69 (br, 2H), 4.04 (d, 3H), 3.75 (s, 3H).

MS m/z (ESI): 487.0 [M+H]⁺.

### Example 61

### Synthesis of Compound 106

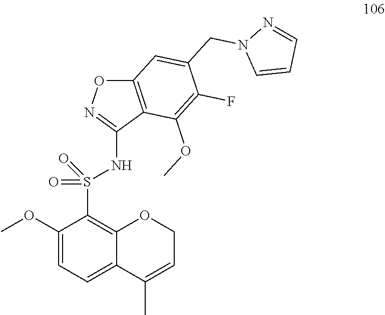 [SMILES: COc1ccc2c(c1S(=O)(=O)Nc1noc3cc(Cn4cccn4)c(F)c(OC)c13)OCC=C2C]

### N-(6-((1H-Pyrazol-1-yl)methyl)-5-fluoro-4-methoxybenzo[d]isoxazol-3-yl)-7-methoxy-4-methyl-2H-chromene-8-sulfonamide

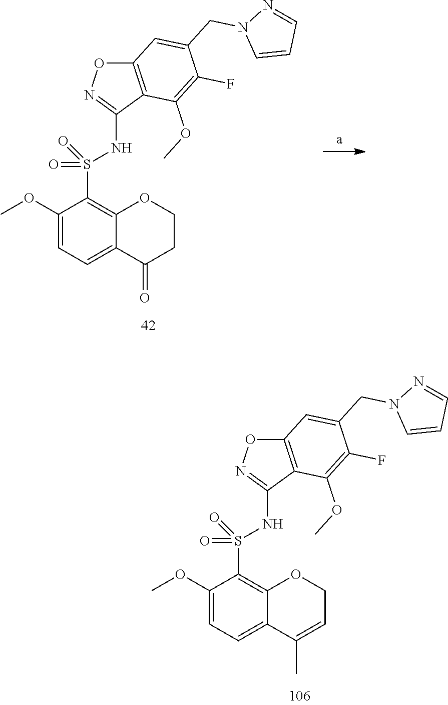 [SMILES: COc1ccc2c(c1S(=O)(=O)Nc1noc3cc(Cn4cccn4)c(F)c(OC)c13)OCCC2=O ; COc1ccc2c(c1S(=O)(=O)Nc1noc3cc(Cn4cccn4)c(F)c(OC)c13)OCC=C2C]

### Step a:

### N-(6-((1H-Pyrazol-1-yl)methyl)-5-fluoro-4-methoxybenzo[d]isoxazol-3-yl)-7-methoxy-4-methyl-2H-chromene-8-sulfonamide

Under a nitrogen atmosphere, methylmagnesium bromide (1.0 M in tetrahydrofuran, 2.6 mL, 2.60 mmol) was added to a solution of compound 42 (130 mg, 0.26 mmol) in tetrahydrofuran (5 mL) at 0° C., and the reaction system was stirred at 0° C. for 1 h. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 106 (7.6 mg, yield: 5.9%).

¹HNMR (400 MHz, DMSO-d₆): δ10.26 (s, 1H), 7.85 (d, 1H), 7.50 (d, 1H), 7.33-7.20 (m, 1H), 6.88-6.86 (m, 1H), 6.66-6.64 (m, 1H), 6.30 (t, 1H), 5.60-5.58 (m, 1H), 5.49 (s, 2H), 4.61-4.59 (m, 2H), 4.07 (s, 3H), 3.74 (s, 3H), 1.94 (s, 3H).

MS m/z (ESI): 501.1 [M+H]⁺.

### Example 62

### Synthesis of Compound 107

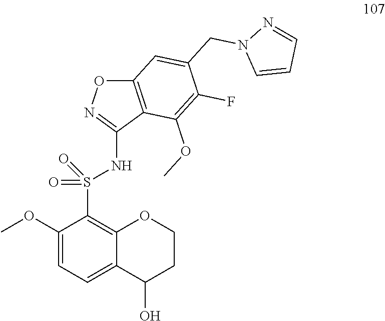 [SMILES: COc1ccc2c(c1S(=O)(=O)Nc1noc3cc(Cn4cccn4)c(F)c(OC)c13)OCCC2O]

### N-(6-((1H-Pyrazol-1-yl)methyl)-5-fluoro-4-methoxybenzo[d]isoxazol-3-yl)-4-hydroxy-7-methoxychromane-8-sulfonamide

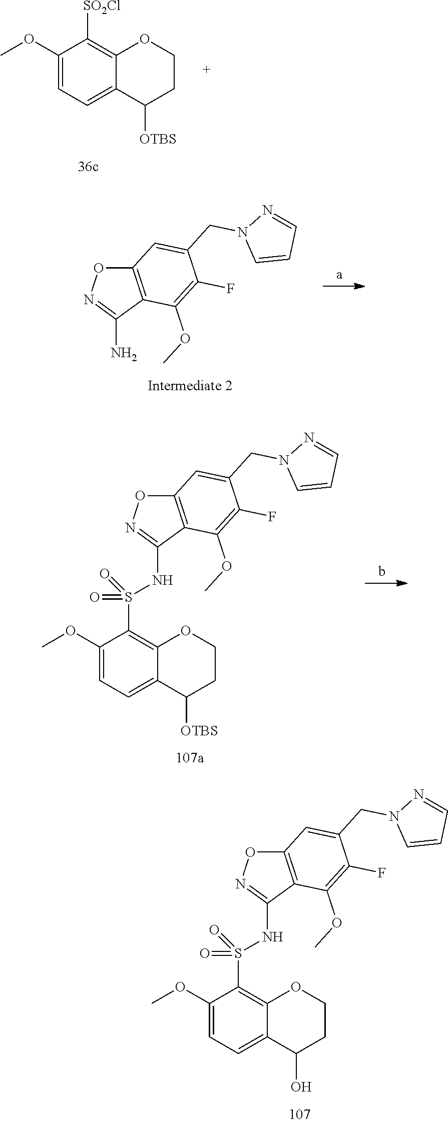 [SMILES: COc1ccc2c(c1S(=O)(=O)Cl)OCCC2O[Si](C)(C)C(C)(C)C ; COc1c(F)c(Cn2cccn2)cc2onc(N)c12 ; COc1ccc2c(c1S(=O)(=O)Nc1noc3cc(Cn4cccn4)c(F)c(OC)c13)OCCC2O[Si](C)(C)C(C)(C)C ; COc1ccc2c(c1S(=O)(=O)Nc1noc3cc(Cn4cccn4)c(F)c(OC)c13)OCCC2O]

### Step a:

### N-(6-((1H-Pyrazol-1-yl)methyl)-5-fluoro-4-methoxybenzo[d]isoxazol-3-yl)-4-((tert-butyldimethylsilyl)oxy)-7-methoxychromane-8-sulfonamide

Sodium hydride (60%, 61 mg, 1.53 mmol) was added to a solution of intermediate 2 (80 mg, 0.31 mmol) in N,N-dimethylformamide (1 mL) at 0° C. The reaction system was stirred at 0° C. for 15 min. Then, compound 36c (156 mg, 0.40 mmol) was added to the system, and stirring was continued for 15 min. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=2:1) to give compound 107a (135 mg, yield: 71.5%).

MS m/z (ESI): δ19.7 [M+H]⁺.

### Step b:

### N-(6-((1H-Pyrazol-1-yl)methyl)-5-fluoro-4-methoxybenzo[d]isoxazol-3-yl)-4-hydroxy-7-methoxychromane-8-sulfonamide

Tetrabutylammonium fluoride trihydrate (949 mg, 3.40 mmol) was added to a solution of compound 107a (700 mg, 1.13 mmol) in tetrahydrofuran (10 mL). The reaction system was stirred for 16 h. After the reaction was completed, the reaction mixture was quenched with an aqueous solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound 107 (300 mg, yield: 52.6%).

¹HNMR (400 MHz, DMSO-d₆): δ10.01 (s, 1H), 7.85 (d, 1H), 7.49 (d, 1H), 7.40-7.38 (m, 1H), 6.83-6.81 (m, 1H), 6.68-6.66 (m, 1H), 6.30 (t, 1H), 5.48 (s, 2H), 5.36-5.34 (m, 1H), 4.56-4.54 (m, 1H), 4.20-4.09 (m, 2H), 4.09 (s, 3H), 3.70 (s, 3H), 1.91-1.76 (m, 2H).

MS m/z (ESI): 505.1 [M+H]⁺.

### Example 63

### Synthesis of Compound 108

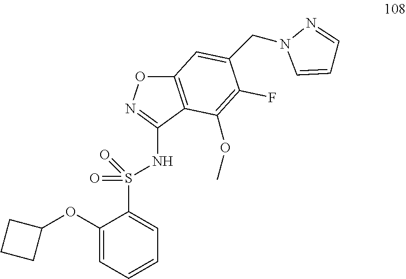 [SMILES: COc1c(F)c(Cn2cccn2)cc2onc(NS(=O)(=O)c3ccccc3OC3CCC3)c12]

### N-(6-((1H-Pyrazol-1-yl)methyl)-5-fluoro-4-methoxybenzo[d]isoxazol-3-yl)-2-cyclobutoxybenzenesulfonamide

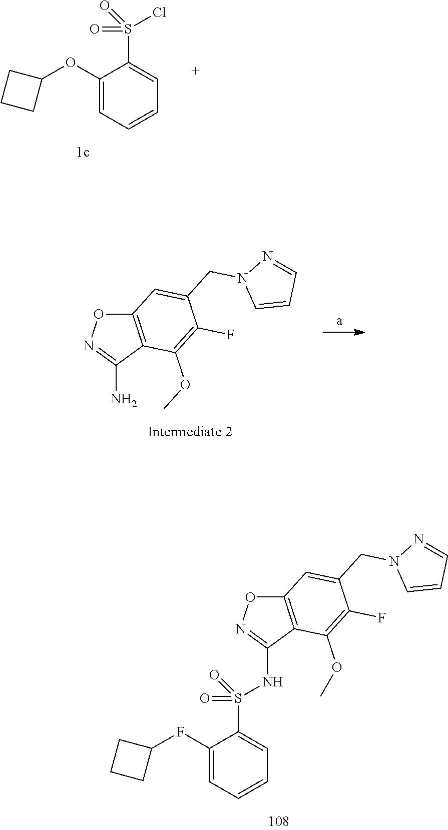

### Step a:

### N-(6-((1H-Pyrazol-1-yl)methyl)-5-fluoro-4-methoxybenzo[d]isoxazol-3-yl)-2-cyclobutoxybenzenesulfonamide

Sodium hydride (60%, 22.9 mg, 0.57 mmol) was added to a solution of intermediate 2 (50 mg, 0.19 mmol) in N,N-dimethylformamide (2 mL) at 0° C. The reaction system was stirred at 0° C. for 1 h. Then, compound 1c (94 mg, 0.38 mmol) was added to the system, and stirring was continued for 1 h. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 108 (35 mg, yield: 39.2%).

¹HNMR (400 MHz, DMSO-d₆): δ 10.37 (s, 1H), 7.85-7.82 (m, 2H), 7.59-7.55 (m, 1H), 7.49 (d, 1H), 7.09 (t, 1H), 6.98 (d, 1H), 6.93 (d, 1H), 6.30 (t, 1H), 5.50 (s, 2H), 4.74-4.67 (m, 1H), 3.98 (d, 3H), 2.26-2.19 (m, 2H), 1.83-1.73 (m, 2H), 1.62-1.47 (m, 2H).

MS m/z (ESI): 472.9 [M+H]⁺.

### Example 64

### Synthesis of Compound 103

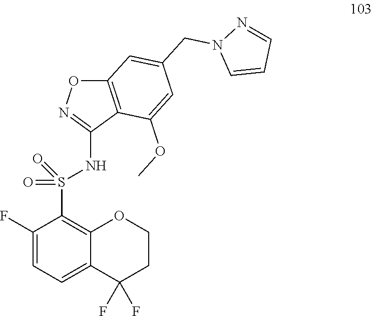 [SMILES: COc1cc(Cn2cccn2)cc2onc(NS(=O)(=O)c3c(F)ccc4c3OCCC4(F)F)c12]

### N-(6-((1H-Pyrazol-1-yl)methyl)-4-methoxybenzo[d]isoxazol-3-yl)-4,4,7-trifluorochromane-8-sulfonamide

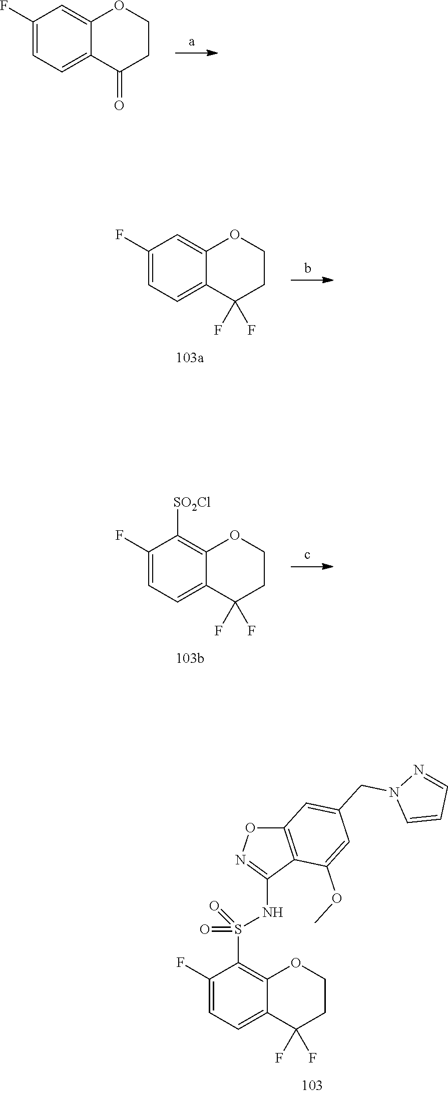 [SMILES: O=C1CCOc2cc(F)ccc21 ; Fc1ccc2c(c1)OCCC2(F)F ; O=S(=O)(Cl)c1c(F)ccc2c1OCCC2(F)F ; COc1cc(Cn2cccn2)cc2onc(NS(=O)(=O)c3c(F)ccc4c3OCCC4(F)F)c12]

### Step a:

### 4,4,7-Trifluorochromane

A solution system of 7-fluorochroman-4-one (2.50 g, 15.05 mmol) in bis(2-methoxyethyl)aminosulfur trifluoride (12 mL) was heated to 70° C. and stirred for 16 h. After the reaction was completed, the reaction mixture was quenched with ice water (40 mL) and extracted with dichloromethane (200 mL×2). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether) to give compound 103a (1.35 g, yield: 47.7%).

¹HNMR (400 MHz, CDCl₃): δ7.58-7.54 (m, 1H), 6.75-6.70 (m, 1H), 6.61-6.57 (m, 1H), 4.36 (t, 2H), 2.50-2.41 (m, 2H).

### Step b:

### 4,4,7-Trifluorochromane-8-sulfonyl chloride

Under a nitrogen atmosphere, n-butyllithium (2.5 M in n-hexane, 3.44 mL, 8.60 mmol) was added dropwise to a solution of compound 103a (1.35 g, 7.18 mmol) in tetrahydrofuran (15 mL) at −78° C., and the reaction system was stirred at −78° C. for 1 h. Then, sulfuryl chloride (1.16 g, 8.60 mmol) was added, and stirring was continued at −78° C. for 15 min. After the reaction was completed, the mixture was quenched with a saturated aqueous ammonium chloride solution and extracted with ethyl acetate (100 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=19:1) to give compound 103b (800 mg, yield: 38.9%).

¹HNMR (400 MHz, CDCl₃): δ7.95-7.91 (m, 1H), 6.96-6.91 (m, 1H), 4.64 (t, 2H), 2.63-2.53 (m, 2H).

### Step c:

### N-(6-((1H-Pyrazol-1-yl)methyl)-4-methoxybenzo[d]isoxazol-3-yl)-4,4,7-trifluorochromane-8-sulfonamide

Sodium hydride (60%, 98.26 mg, 2.46 mmol) was added to a solution of intermediate 1 (200 mg, 0.82 mmol) in N,N-dimethylformamide (2 mL) at 0° C. The reaction system was stirred at 0° C. for 15 min. Then, a solution of compound 103b (399.01 mg, 1.35 mmol) in N,N-dimethylformamide (1 mL) was added to the system, and stirring was continued for 15 min. After the reaction was completed, the mixture was quenched with a saturated aqueous ammonium chloride solution and extracted with dichloromethane (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=19:1) to give compound 103 (360 mg, yield: 88.9%).

¹HNMR (400 MHz, DMSO-d₆): δ 11.09 (brs, 1H), 7.91-7.87 (m, 2H), 7.50 (d, 1H), 7.08 (t, 1H), 6.87 (s, 1H), 6.74 (s, 1H), 6.30 (t, 1H), 5.45 (s, 2H), 4.30 (t, 2H), 3.77 (s, 3H), 2.55-2.46 (m, 2H).

MS m/z (ESI): 494.8 [M+H]⁺.

### Example 65

### Synthesis of Compound 102

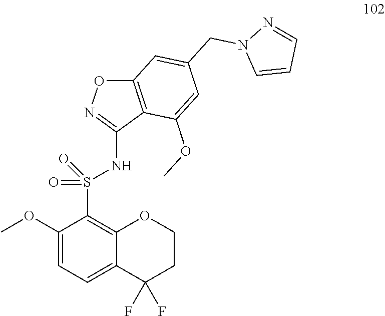 [SMILES: COc1ccc2c(c1S(=O)(=O)Nc1noc3cc(Cn4cccn4)cc(OC)c13)OCCC2(F)F]

### N-(6-((1H-Pyrazol-1-yl)methyl)-4-methoxybenzo[d]isoxazol-3-yl)-4,4-difluoro-7-methoxychromane-8-sulfonamide

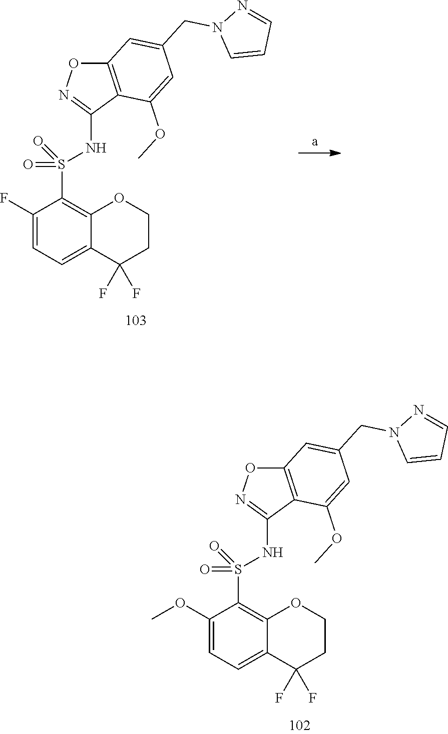 [SMILES: COc1cc(Cn2cccn2)cc2onc(NS(=O)(=O)c3c(F)ccc4c3OCCC4(F)F)c12 ; COc1ccc2c(c1S(=O)(=O)Nc1noc3cc(Cn4cccn4)cc(OC)c13)OCCC2(F)F]

### Step a:

### N-(6-((1H-Pyrazol-1-yl)methyl)-4-methoxybenzo[d]isoxazol-3-yl)-4,4-difluoro-7-methoxychromane-8-sulfonamide

Sodium methoxide (33% in methanol, 1 mL) was added to a solution of compound 103 (100 mg, 0.20 mmol) in methanol (2 mL). The reaction system was heated to 65° C. and stirred for 1 h. After the reaction was completed, the solution was quenched and adjusted to pH=5 with a 1 N aqueous hydrochloric acid solution, and extracted with dichloromethane (50 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 102 (23 mg, yield: 22.5%).

¹HNMR (400 MHz, DMSO-d₆): δ 10.13 (s, 1H), 7.87 (d, 1H), 7.76 (d, 1H), 7.50 (d, 1H), 6.91 (d, 1H), 6.85 (s, 1H), 6.75 (s, 1H), 6.30 (t, 1H), 5.45 (s, 2H), 4.29 (t, 2H), 3.82 (s, 3H), 3.81 (s, 3H), 2.50-2.43 (m, 2H).

MS m/z (ESI): 506.8 [M+H]⁺.

### Example 66

### Synthesis of Compound 105

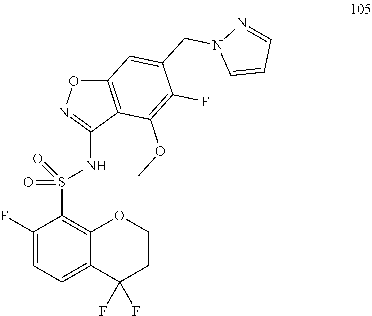 [SMILES: COc1c(F)c(Cn2cccn2)cc2onc(NS(=O)(=O)c3c(F)ccc4c3OCCC4(F)F)c12]

### N-(6-((1H-Pyrazol-1-yl)methyl)-5-fluoro-4-methoxybenzo[d]isoxazol-3-yl)-4,4,7-trifluorochromane-8-sulfonamide

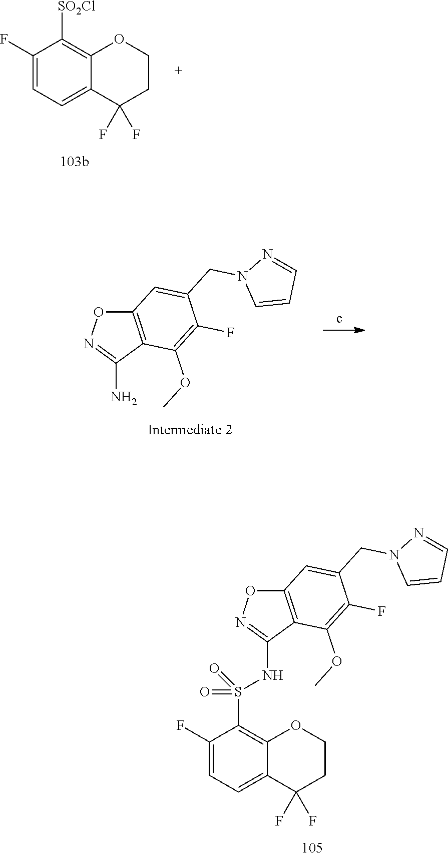 [SMILES: O=S(=O)(Cl)c1c(F)ccc2c1OCCC2(F)F ; COc1c(F)c(Cn2cccn2)cc2onc(N)c12 ; COc1c(F)c(Cn2cccn2)cc2onc(NS(=O)(=O)c3c(F)ccc4c3OCCC4(F)F)c12]

### Step a:

### N-(6-((1H-Pyrazol-1-yl)methyl)-5-fluoro-4-methoxybenzo[d]isoxazol-3-yl)-4,4,7-trifluorochromane-8-sulfonamide

Sodium hydride (60%, 76.30 mg, 1.91 mmol) was added to a solution of intermediate 2 (100 mg, 0.38 mmol) in N,N-dimethylformamide (1 mL) at 0° C. The reaction system was stirred at 0° C. for 15 min. Then, compound 103b (164.00 mg, 0.57 mmol) was added to the system, and stirring was continued for 15 min. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:tetrahydrofuran=85:15) to give compound 105 (120 mg, yield: 61.4%).

¹HNMR (400 MHz, DMSO-d₆): δ 11.34 (brs, 1H), 7.92-7.86 (m, 2H), 7.50-7.49 (m, 1H), 7.08 (t, 1H), 6.95 (d, 1H), 6.31 (t, 1H), 5.51 (s, 2H), 4.37-4.34 (m, 2H), 4.00 (d, 3H), 2.60-2.52 (m, 2H).

MS m/z (ESI): 512.8 [M+H]⁺.

### Example 67

### Synthesis of Compound 104

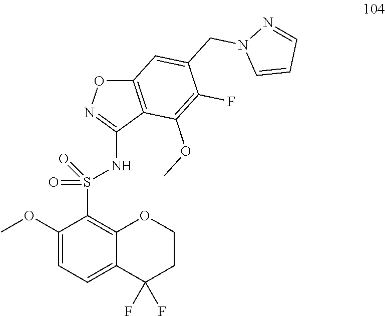 [SMILES: COc1ccc2c(c1S(=O)(=O)Nc1noc3cc(Cn4cccn4)c(F)c(OC)c13)OCCC2(F)F]

### N-(6-((1H-Pyrazol-1-yl)methyl)-5-fluoro-4-methoxybenzo[d]isoxazol-3-yl)-4,4-difluoro-7-methoxychromane-8-sulfonamide

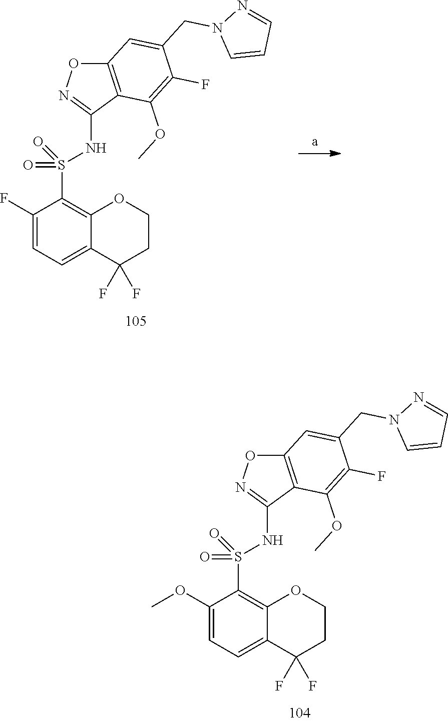 [SMILES: COc1c(F)c(Cn2cccn2)cc2onc(NS(=O)(=O)c3c(F)ccc4c3OCCC4(F)F)c12 ; COc1ccc2c(c1S(=O)(=O)Nc1noc3cc(Cn4cccn4)c(F)c(OC)c13)OCCC2(F)F]

### Step a:

### N-(6-((1H-Pyrazol-1-yl)methyl)-5-fluoro-4-methoxybenzo[d]isoxazol-3-yl)-4,4-difluoro-7-methoxychromane-8-sulfonamide

Sodium methoxide (33% in methanol, 2 mL) was added to a solution of compound 105 (80 mg, 0.16 mmol) in methanol (2 mL). The reaction system was heated to 65° C. and stirred for 2 h. After the reaction was completed, the solution was quenched and adjusted to pH=5 with a 1 N aqueous hydrochloric acid solution, and extracted with ethyl acetate (50 mL×2). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 104 (46 mg, yield: 56.2%).

¹HNMR (400 MHz, DMSO-d₆): δ 10.54 (s, 1H), 7.86 (d, 1H), 7.77 (d, 1H), 7.50-7.49 (m, 1H), 6.93-6.91 (m, 2H), 6.31 (t, 1H), 5.51 (s, 2H), 4.31 (t, 2H), 4.02 (d, 3H), 3.82 (s, 3H), 2.54-2.46 (m, 2H).

MS m/z (ESI): 524.9 [M+H]⁺.

### Example 68

### Synthesis of Compound 109

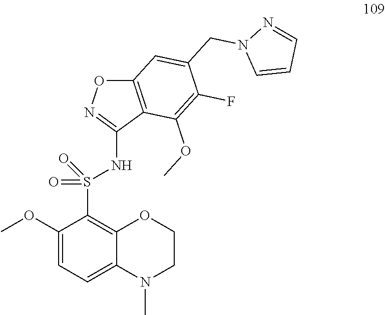 [SMILES: COc1ccc2c(c1S(=O)(=O)Nc1noc3cc(Cn4cccn4)c(F)c(OC)c13)OCCN2C]

### N-(6-((1H-Pyrazol-1-yl)methyl)-5-fluoro-4-methoxybenzo[d]isoxazol-3-yl)-7-methoxy-4-methyl-3,4-dihydro-2H-benzo[b][1,4]oxazine-8-sulfonamide

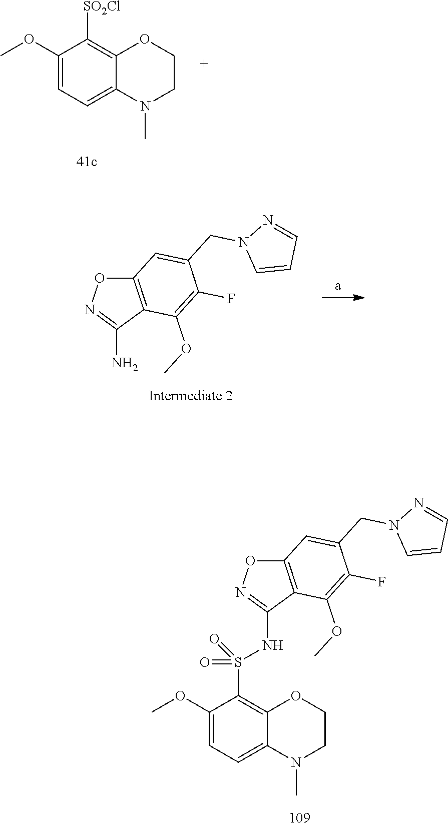 [SMILES: COc1ccc2c(c1S(=O)(=O)Cl)OCCN2C ; COc1c(F)c(Cn2cccn2)cc2onc(N)c12 ; COc1ccc2c(c1S(=O)(=O)Nc1noc3cc(Cn4cccn4)c(F)c(OC)c13)OCCN2C]

### Step a:

### N-(6-((1H-Pyrazol-1-yl)methyl)-5-fluoro-4-methoxybenzo[d]isoxazol-3-yl)-7-methoxy-4-methyl-3,4-dihydro-2H-benzo[b][1,4]oxazine-8-sulfonamide

Sodium hydride (60%, 72.00 mg, 1.80 mmol) was added to a solution of intermediate 2 (94 mg, 0.36 mmol) in N,N-dimethylformamide (3 mL) at 0° C. The reaction system was stirred at 0° C. for 10 min. Then, compound 41c (120.00 mg, 0.43 mmol) was added to the system, and stirring was continued for 15 min. After the reaction was completed, the system was quenched with a saturated aqueous ammonium chloride solution, adjusted to pH=4 with a 0.5 N diluted aqueous hydrochloric acid solution, and then extracted with ethyl acetate (15 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 109 (62 mg, yield: 34.4%). ¹HNMR (400 MHz, DMSO-d₆): δ 9.98 (brs, 1H), 7.86 (d, 1H), 7.50 (d, 1H), 6.90-6.87 (m, 2H), 6.58 (d, 1H), 6.31 (t, 1H), 5.50 (s, 2H), 4.20 (t, 2H), 4.06 (d, 3H), 3.66 (s, 3H), 3.12 (t, 2H), 2.77 (s, 3H).

MS m/z (ESI): 504.1 [M+H]⁺.

### Example 69

### Synthesis of Compound 112

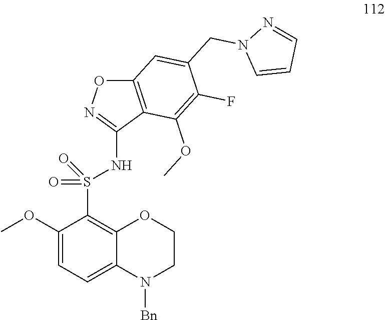 [SMILES: COc1ccc2c(c1S(=O)(=O)Nc1noc3cc(Cn4cccn4)c(F)c(OC)c13)OCCN2Cc1ccccc1]

### N-(6-((1H-Pyrazol-1-yl)methyl)-5-fluoro-4-methoxybenzo[d]isoxazol-3-yl)-4-benzyl-7-methoxy-3,4-dihydro-2H-benzo[b][1,4]oxazine-8-sulfonamide

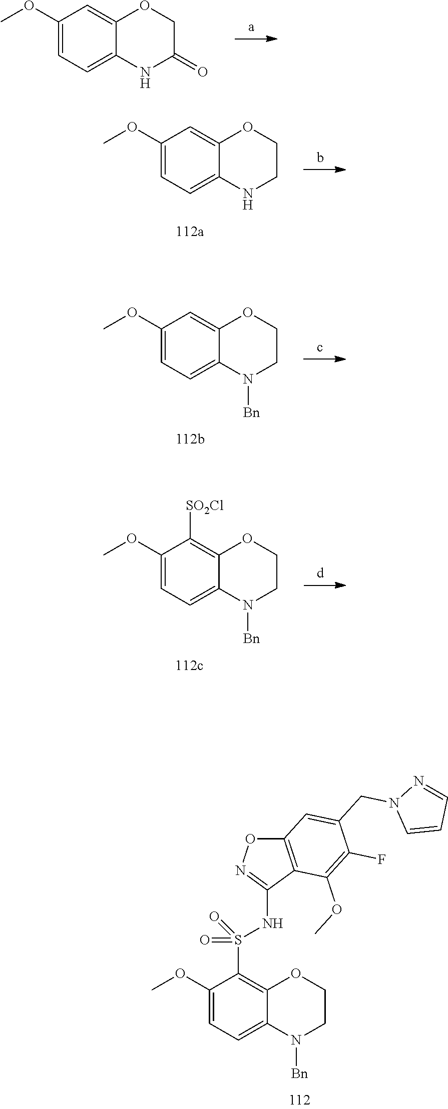 [SMILES: COc1ccc2c(c1)OCC(=O)N2 ; COc1ccc2c(c1)OCCN2 ; COc1ccc2c(c1)OCCN2Cc1ccccc1 ; COc1ccc2c(c1S(=O)(=O)Cl)OCCN2Cc1ccccc1 ; COc1ccc2c(c1S(=O)(=O)Nc1noc3cc(Cn4cccn4)c(F)c(OC)c13)OCCN2Cc1ccccc1]

### Step a:

### 7-Methoxy-3,4-dihydro-2H-benzo[b][1,4]oxazine

Under a nitrogen atmosphere, a borane solution (1 M in tetrahydrofuran, 13.40 mL, 13.40 mmol) was added to a solution of 7-methoxy-2H-benzo[b][1,4]oxazin-3(4H)-one (1.20 g, 6.70 mmol) in tetrahydrofuran (40 mL) at 0° C., and the reaction system was heated to 75° C. and stirred for 4 h. After the reaction was completed, the reaction mixture was concentrated under reduced pressure. The resulting crude product was purified by flash silica gel column chromatography (petroleum ether:ethyl acetate=2:1) to give compound 112a (0.82 g, yield: 74.1%).

MS m/z (ESI): 166.0 [M+H]⁺.

### Step b:

### 4-Benzyl-7-methoxy-3,4-dihydro-2H-benzo[b][1,4]oxazine

Under a nitrogen atmosphere, a solution of benzyl bromide (1.20 g, 7.02 mmol) in dichloromethane (30 mL) was added dropwise to a solution of compound 112a (900 mg, 5.45 mmol) and N,N-diisopropylethylamine (2.10 g, 16.25 mmol) in dichloromethane (50 mL), and the reaction system was stirred at room temperature for 2 h. After the reaction was completed, the reaction mixture was concentrated under reduced pressure. The resulting crude product was purified by flash silica gel column chromatography (petroleum ether:ethyl acetate=1:1) to give compound 112b (1.2 g, yield: 86.3%).

MS m/z (ESI): 256.2 [M+H]⁺.

### Step c:

### 4-Benzyl-7-methoxy-3,4-dihydro-2H-benzo[b][1,4]oxazine-8-sulfonyl chloride

Under a nitrogen atmosphere, n-butyllithium (2.5 M in n-hexane, 0.4 mL, 1.00 mmol) was added dropwise to a solution of compound 112b (200 mg, 0.78 mmol) in tetrahydrofuran (5 mL) at 0° C., and the reaction system was stirred at 0° C. for 1 h and then cooled to −78° C. Subsequently, sulfuryl chloride (127 mg, 0.94 mmol) was added, and stirring was continued at −78° C. for 15 min. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=1:1) to give compound 112c (23 mg, yield: 8.3%).

¹HNMR (400 MHz, CDCl₃): δ7.41-7.30 (m, 5H), 6.91 (d, 1H), 6.50 (d, 1H), 4.51 (t, 2H), 4.46 (s, 2H), 3.91 (s, 3H), 3.45 (t, 2H).

MS m/z (ESI): 354.3 [M+H]⁺.

### Step d:

### N-(6-((1H-Pyrazol-1-yl)methyl)-5-fluoro-4-methoxybenzo[d]isoxazol-3-yl)-4-benzyl-7-methoxy-3,4-dihydro-2H-benzo[b][1,4]oxazine-8-sulfonamide

Sodium hydride (60%, 72 mg, 1.80 mmol) was added to a solution of intermediate 2 (94 mg, 0.36 mmol) in N,N-dimethylformamide (3 mL) at 0° C. The reaction system was stirred at 0° C. for 10 min. Then, compound 112c (153 mg, 0.43 mmol) was added to the system, and stirring was continued for 15 min. After the reaction was completed, the system was quenched with a saturated aqueous ammonium chloride solution, adjusted to pH=4 with a 0.5 N aqueous hydrochloric acid solution, and extracted with ethyl acetate (20 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=4:1) to give compound 112 (110 mg, yield: 52.9%).

¹HNMR (400 MHz, DMSO-d₆): δ9.99 (s, 1H), 7.86 (d, 1H), 7.50 (d, 1H), 7.32-7.24 (m, 5H), 6.92 (d, 1H), 6.84 (d, 1H), 6.55 (d, 1H), 6.31 (t, 1H), 5.51 (s, 2H), 4.42 (s, 2H), 4.19 (t, 2H), 4.04 (d, 3H), 3.63 (s, 3H), 3.30-3.28 (m, 2H).

MS m/z (ESI): 580.2 [M+H]⁺.

### Example 70

### Synthesis of Compound 111

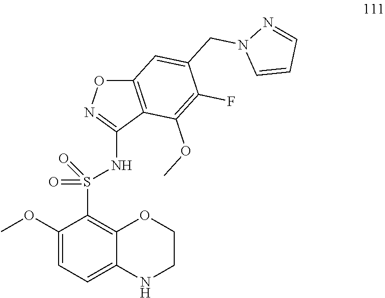 [SMILES: COc1ccc2c(c1S(=O)(=O)Nc1noc3cc(Cn4cccn4)c(F)c(OC)c13)OCCN2]

### N-(6-((1H-Pyrazol-1-yl)methyl)-5-fluoro-4-methoxybenzo[d]isoxazol-3-yl)-7-methoxy-3,4-dihydro-2H-benzo[b][1,4]oxazine-8-sulfonamide

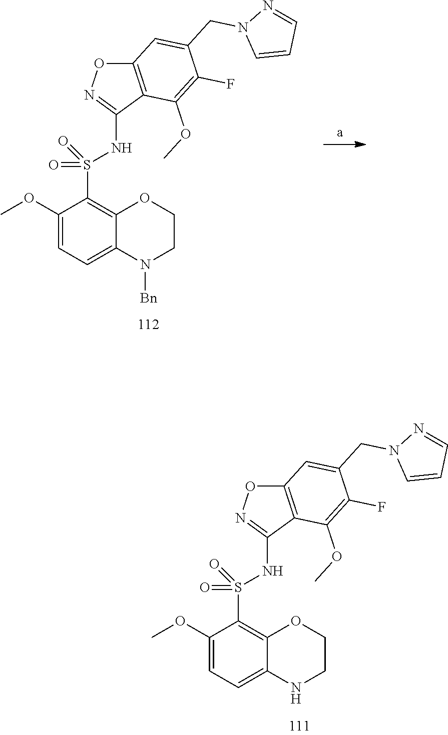 [SMILES: COc1ccc2c(c1S(=O)(=O)Nc1noc3cc(Cn4cccn4)c(F)c(OC)c13)OCCN2Cc1ccccc1 ; COc1ccc2c(c1S(=O)(=O)Nc1noc3cc(Cn4cccn4)c(F)c(OC)c13)OCCN2]

### Step a:

### N-(6-((1H-Pyrazol-1-yl)methyl)-5-fluoro-4-methoxybenzo[d]isoxazol-3-yl)-7-methoxy-3,4-dihydro-2H-benzo[b][1,4]oxazine-8-sulfonamide

Under a hydrogen atmosphere, palladium on carbon (138 mg, 1.30 mmol), palladium hydroxide (364 mg, 2.59 mmol), and 1,1,2-trichloroethane (575 mg, 2.59 mmol) were added to a solution of compound 112 (500 mg, 0.86 mmol) in methanol (20 mL), and the reaction system was stirred at 25° C. for 2 h. After the reaction was completed, the reaction system was filtered through celite, and the filtrate was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 111 (390 mg, yield: 92.4%).

¹HNMR (400 MHz, DMSO-d₆): δ 9.86 (brs, 1H), 7.85 (d, 1H), 7.50 (d, 1H), 6.89 (d, 1H), 6.72 (d, 1H), 6.53 (d, 1H), 6.31 (t, 1H), 5.66 (s, 1H), 5.55 (s, 2H), 4.08 (s, 3H), 4.08-4.05 (m, 2H), 3.63 (s, 3H), 3.19-3.17 (m, 2H).

MS m/z (ESI): 490.1 [M+H]⁺.

### Example 71

### Synthesis of Compound 110

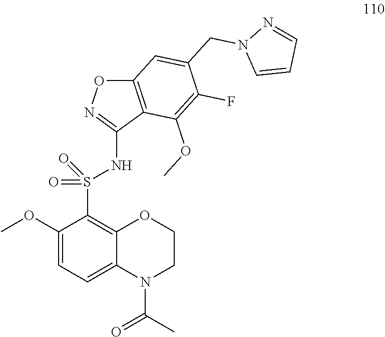 [SMILES: COc1ccc2c(c1S(=O)(=O)Nc1noc3cc(Cn4cccn4)c(F)c(OC)c13)OCCN2C(C)=O]

### N-(6-((1H-Pyrazol-1-yl)methyl)-5-fluoro-4-methoxybenzo[d]isoxazol-3-yl)-4-acetyl-7-methoxy-3,4-dihydro-2H-benzo[b][1,4]oxazine-8-sulfonamide

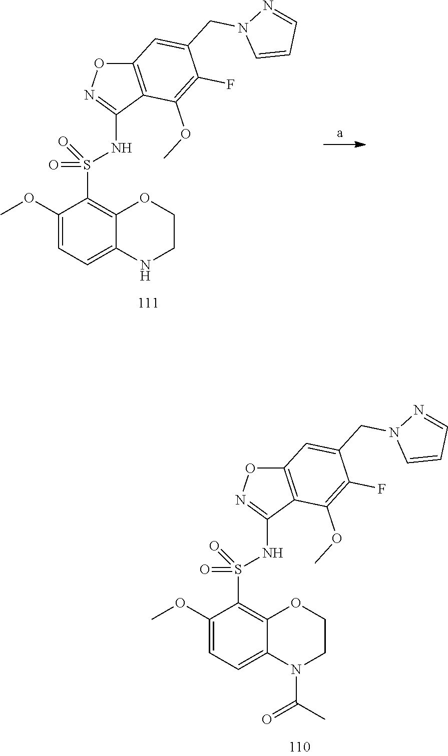 [SMILES: COc1ccc2c(c1S(=O)(=O)Nc1noc3cc(Cn4cccn4)c(F)c(OC)c13)OCCN2 ; COc1ccc2c(c1S(=O)(=O)Nc1noc3cc(Cn4cccn4)c(F)c(OC)c13)OCCN2C(C)=O]

### Step a:

### N-(6-((1H-Pyrazol-1-yl)methyl)-5-fluoro-4-methoxybenzo[d]isoxazol-3-yl)-4-acetyl-7-methoxy-3,4-dihydro-2H-benzo[b][1,4]oxazine-8-sulfonamide

N,N-Diisopropylethylamine (392 mg, 3.03 mmol) was added to a solution of compound 111 (99 mg, 0.20 mmol) and acetic anhydride (248 mg, 2.43 mmol) in dichloromethane (50 mL). The reaction system was stirred at 25° C. for 18 h. After the reaction was completed, the reaction system was diluted with water and extracted with dichloromethane (10 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 110 (17 mg, yield: 15.8%).

¹HNMR (400 MHz, DMSO-d₆): δ 10.28 (brs, 1H), 8.03 (brs, 1H), 7.85 (d, 1H), 7.50 (d, 1H), 6.90-6.71 (m, 2H), 6.31 (t, 1H), 5.50 (s, 2H), 4.27-4.25 (m, 2H), 4.04 (s, 3H), 3.78-3.74 (m, 5H), 2.19 (s, 3H).

MS m/z (ESI): 532.1 [M+H]⁺.

### Example 72

### Synthesis of Compound 114

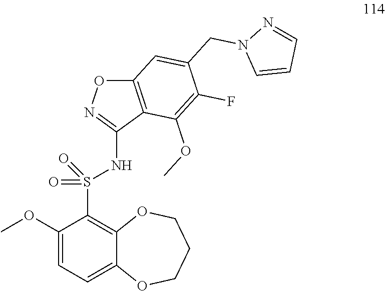 [SMILES: COc1ccc2c(c1S(=O)(=O)Nc1noc3cc(Cn4cccn4)c(F)c(OC)c13)OCCCO2]

### N-(6-((1H-Pyrazol-1-yl)methyl)-5-fluoro-4-methoxybenzo[d]isoxazol-3-yl)-7-methoxy-3,4-dihydro-2H-benzo[b][1,4]dioxepine-6-sulfonamide

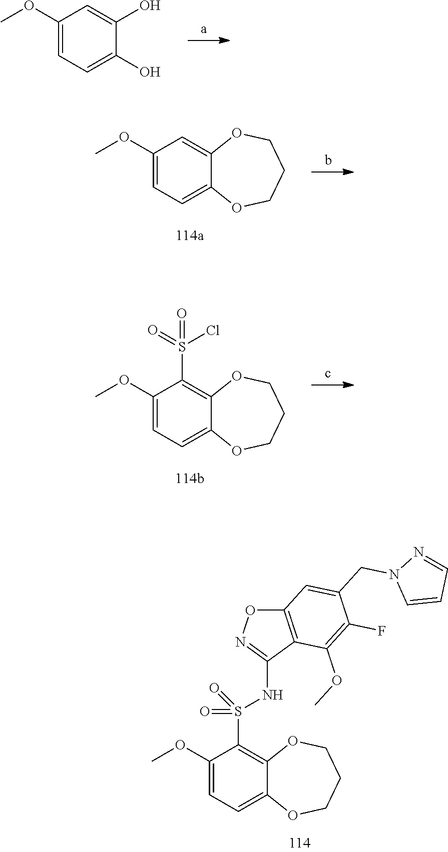

### Step a:

### 7-Methoxy-3,4-dihydro-2H-benzo[b][1,4]dioxepine

Under a nitrogen atmosphere, 1,3-dibromopropane (562 mg, 2.78 mmol) was added dropwise to a solution of 4-methoxybenzene-1,2-diol (300 mg, 2.14 mmol) and potassium carbonate (1.04 g, 7.52 mmol) in ethanol (6 mL) at 20° C., and the reaction system was heated to 85° C. and stirred for 16 h. After the reaction was completed, the mixture was diluted with an aqueous solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=50:1) to give compound 114a (184 mg, yield: 47.7%).

¹HNMR (400 MHz, CDCl₃): δ6.83 (d, 1H), 6.48 (d, 1H), 6.43-6.40 (m, 1H), 4.12 (t, 2H), 4.06 (t, 2H), 3.76 (s, 3H), 2.13-2.06 (m, 2H).

### Step b:

### 7-Methoxy-3,4-dihydro-2H-benzo[b][1,4]dioxepine-6-sulfonyl chloride

Under a nitrogen atmosphere, n-butyllithium (2.5 M in n-hexane, 0.49 mL, 1.23 mmol) was added dropwise to a solution of compound 114a (184 mg, 1.02 mmol) in tetrahydrofuran (5 mL) at 0° C., and the reaction system was stirred at 0° C. for 1 h and then cooled to −78° C. Subsequently, sulfuryl chloride (152 mg, 1.13 mmol) was added, and stirring was continued at −78° C. for 10 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=5:1) to give compound 114b (119 mg, yield: 41.8%).

¹HNMR (400 MHz, CDCl₃): δ7.22 (d, 1H), 6.59 (d, 1H), 4.29 (t, 2H), 4.11 (t, 2H), 3.86 (s, 3H), 2.22-2.17 (m, 2H).

### Step c:

### N-(6-((1H-Pyrazol-1-yl)methyl)-5-fluoro-4-methoxybenzo[d]isoxazol-3-yl)-7-methoxy-3,4-dihydro-2H-benzo[b][1,4]dioxepine-6-sulfonamide

Sodium hydride (60%, 53 mg, 1.33 mmol) was added to a solution of intermediate 2 (70 mg, 0.27 mmol) in N,N-dimethylformamide (1 mL) at 0° C. The reaction system was stirred at 0° C. for 1 h. Then, compound 114b (112 mg, 0.40 mmol) was added to the system, and stirring was continued for 1 h. After the reaction was completed, the mixture was quenched with a saturated aqueous ammonium chloride solution and extracted with ethyl acetate (10 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous ammonium bicarbonate solution, mobile phase B: acetonitrile; gradient ratio:phase B 5%-95%) to give compound 114 (17 mg, yield: 12.6%).

¹HNMR (400 MHz, DMSO-d₆): δ10.38 (s, 1H), 7.86 (d, 1H), 7.50 (d, 1H), 7.23 (d, 1H), 6.91 (d, 1H), 6.80 (d, 1H), 6.31 (t, 1H), 5.50 (s, 2H), 4.07-4.05 (m, 2H), 4.02-4.00 (m, 5H), 3.33 (s, 3H), 2.06-2.04 (m, 2H).

MS m/z (ESI): 505.0 [M+H]⁺.

### Example 73

### Synthesis of Compound 115

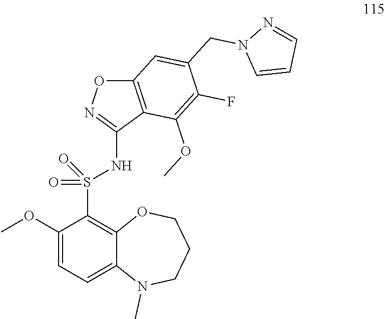 [SMILES: COc1ccc2c(c1S(=O)(=O)Nc1noc3cc(Cn4cccn4)c(F)c(OC)c13)OCCCN2C]

### N-(6-((1H-Pyrazol-1-yl)methyl)-5-fluoro-4-methoxybenzo[d]isoxazol-3-yl)-8-methoxy-5-methyl-2,3,4,5-tetrahydrobenzo[b][1,4]oxazepine-9-sulfonamide

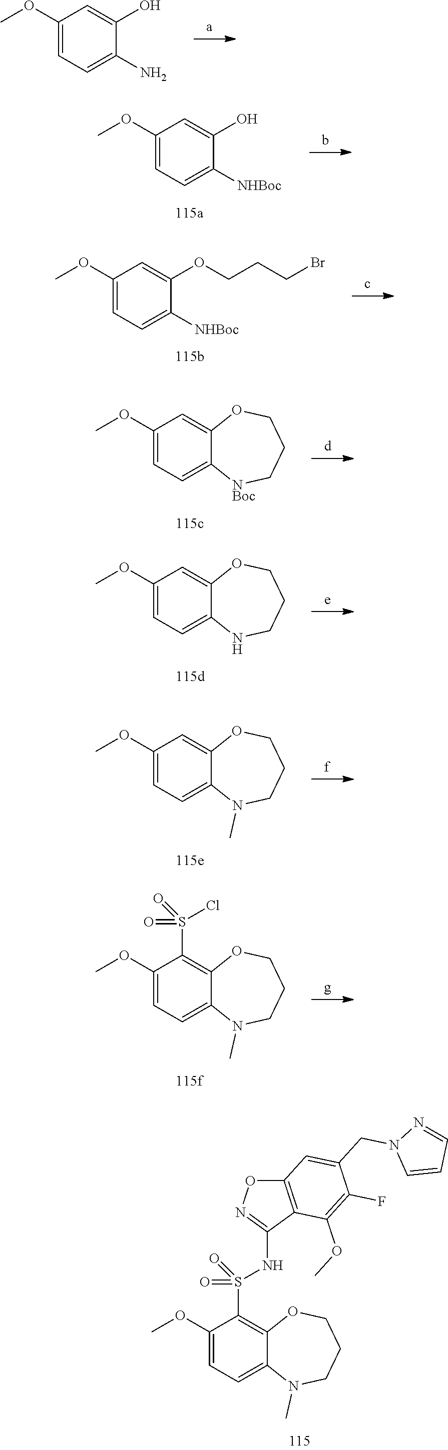

### Step a:

### tert-Butyl (2-hydroxy-4-methoxyphenyl)carbamate

Under a nitrogen atmosphere, a solution of di-tert-butyl dicarbonate (6.58 g, 30.15 mmol) in tetrahydrofuran (10 mL) was added dropwise to a solution of 2-amino-5-methoxyphenol (4.00 g, 28.75 mmol) in tetrahydrofuran (80 mL), and the reaction system was stirred at 25° C. for 5 h. After the reaction was completed, the mixture was diluted with an aqueous solution and extracted with ethyl acetate (70 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 115a (6.20 g, yield: 90.1%).

MS m/z (ESI): 184.0 [M+H−56]⁺.

### Step b:

### tert-Butyl (2-(3-bromopropoxy)-4-methoxyphenyl)carbamate

A solution of compound 115a (3.80 g, 15.88 mmol), 1,3-dibromopropane (14.45 g, 71.57 mmol), and potassium carbonate (17.58 g, 127.20 mmol) in acetonitrile (100 mL) was heated to 60° C. and stirred for 1 h. After the reaction was completed, the mixture was diluted with an aqueous solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=19:1) to give compound 115b (3.00 g, yield: 52.4%).

MS m/z (ESI): 260.0 [M+H-Boc]⁺.

### Step c:

### tert-Butyl 8-methoxy-3,4-dihydrobenzo[b][1,4]oxazepine-5(2H)-carboxylate

Under a nitrogen atmosphere, sodium hydride (60%, 0.56 g, 14.00 mmol) was added to a solution of compound 115b (2.00 g, 5.55 mmol) in tetrahydrofuran (50 mL) at 0° C., and the reaction system was stirred at 0° C. for 2 h. After the reaction was completed, the mixture was quenched with a saturated aqueous ammonium chloride solution and extracted with ethyl acetate (50 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=19:1) to give compound 115c (1.16 g, yield: 74.8%).

¹HNMR (400 MHz, DMSO-d₆): δ7.09 (d, 1H), 6.59 (dd, 1H), 6.55 (d, 1H), 4.02-3.99 (m, 2H), 3.72 (s, 3H), 3.55-3.53 (m, 2H), 1.91-1.89 (m, 2H), 1.44-1.32 (m, 9H).

MS m/z (ESI): 224.2 [M+H−56]⁺.

### Step d:

### 8-Methoxy-2,3,4,5-tetrahydrobenzo[b][1,4]oxazepine

Trifluoroacetic acid (5 mL) was added to a solution of compound 115c (1.16 g, 4.15 mmol) in dichloromethane (30 mL) at 0° C. The reaction system was stirred at 25° C. for 5 h. After the reaction was completed, the mixture was quenched with a saturated aqueous sodium bicarbonate solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=3:1) to give compound 115d (0.69 g, yield: 92.7%).

MS m/z (ESI): 180.3 [M+H]⁺.

### Step e:

### 8-Methoxy-5-methyl-2,3,4,5-tetrahydrobenzo[b][1,4]oxazepine

Under a nitrogen atmosphere, paraformaldehyde (0.83 g, 27.64 mmol) and acetic acid (20 mg) were added to a solution of compound 115d (0.99 g, 5.52 mmol) and sodium cyanoborohydride (1.74 g, 27.69 mmol) in tetrahydrofuran (20 mL), and the reaction system was heated to 50° C. and stirred for 3 h. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:ethyl acetate=9:1) to give compound 115e (0.88 g, yield: 82.4%).

¹HNMR (400 MHz, DMSO-d₆): δ6.79 (d, 1H), 6.54 (dd, 1H), 6.47 (d, 1H), 3.95 (t, 2H), 3.66 (s, 3H), 2.95 (t, 2H), 2.76 (s, 3H), 1.92-1.89 (m, 2H).

MS m/z (ESI): 194.2 [M+H]⁺.

### Step f:

### 8-Methoxy-5-methyl-2,3,4,5-tetrahydrobenzo[b][1,4]oxazepine-9-sulfonyl chloride

Under a nitrogen atmosphere, n-butyllithium (2.5 M in n-hexane, 1.99 mL, 4.98 mmol) was added dropwise to a solution of compound 115e (875 mg, 4.53 mmol) in tetrahydrofuran (9 mL) at 0° C., and the reaction system was stirred at 0° C. for 1 h and then cooled to −78° C. Subsequently, sulfuryl chloride (733 mg, 5.43 mmol) was added, and stirring was continued at −78° C. for 15 min. After the reaction was completed, the mixture was quenched with a saturated aqueous ammonium chloride solution and extracted with ethyl acetate (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (petroleum ether:tetrahydrofuran=2:1) to give compound 115f (410 mg, yield: 31.0%).

¹HNMR (400 MHz, CDCl₃): δ77.14 (d, 1H), 6.71 (d, 1H), 4.25 (t, 2H), 3.91 (s, 3H), 3.10 (t, 2H), 2.89 (s, 3H), 2.10-2.05 (m, 2H).

MS m/z (ESI): 292.0 [M+H]⁺.

### Step g:

### N-(6-((1H-Pyrazol-1-yl)methyl)-5-fluoro-4-methoxybenzo[d]isoxazol-3-yl)-8-methoxy-5-methyl-2,3,4,5-tetrahydrobenzo[b][1,4]oxazepine-9-sulfonamide

Sodium hydride (60%, 38 mg, 0.95 mmol) was added to a solution of intermediate 2 (50 mg, 0.19 mmol) in N,N-dimethylformamide (1 mL) at 0° C. The reaction system was stirred at 0° C. for 15 min. Then, compound 115f (72 mg, 0.25 mmol) was added to the system, and stirring was continued for 15 min. After the reaction was completed, the system was quenched with a saturated aqueous sodium chloride solution, then adjusted to pH=5 with acetic acid, and extracted with ethyl acetate (10 mL×3). The organic layers were combined, washed with a saturated aqueous NaCl solution, and dried over anhydrous sodium sulfate. After filtration, the mother liquor was concentrated under reduced pressure. The resulting crude product was purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 115 (28.7 mg, yield: 29.1%).

¹HNMR (400 MHz, DMSO-d₆): δ10.06 (brs, 1H), 7.85 (d, 1H), 7.49 (d, 1H), 7.09-7.07 (m, 1H), 6.88-6.75 (m, 2H), 6.30 (t, 1H), 5.49 (s, 2H), 4.03 (s, 3H), 3.96 (t, 2H), 3.68 (s, 3H), 2.93 (t, 2H), 2.76 (s, 3H), 1.89-1.87 (m, 2H).

MS m/z (ESI): 518.0 [M+H]⁺.

### Example 74

### Synthesis of Compound 120

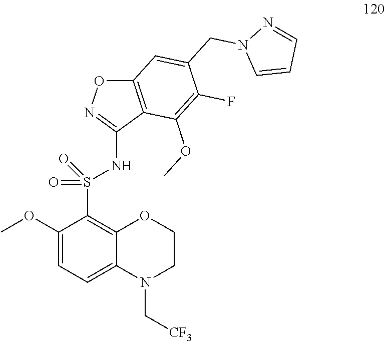 [SMILES: COc1ccc2c(c1S(=O)(=O)Nc1noc3cc(Cn4cccn4)c(F)c(OC)c13)OCCN2CC(F)(F)F]

### N-(6-((1H-Pyrazol-1-yl)methyl)-5-fluoro-4-methoxybenzo[d]isoxazol-3-yl)-7-methoxy-4-(2,2,2-trifluoroethyl)-3,4-dihydro-2H-benzo[b][1,4]oxazine-8-sulfonamide

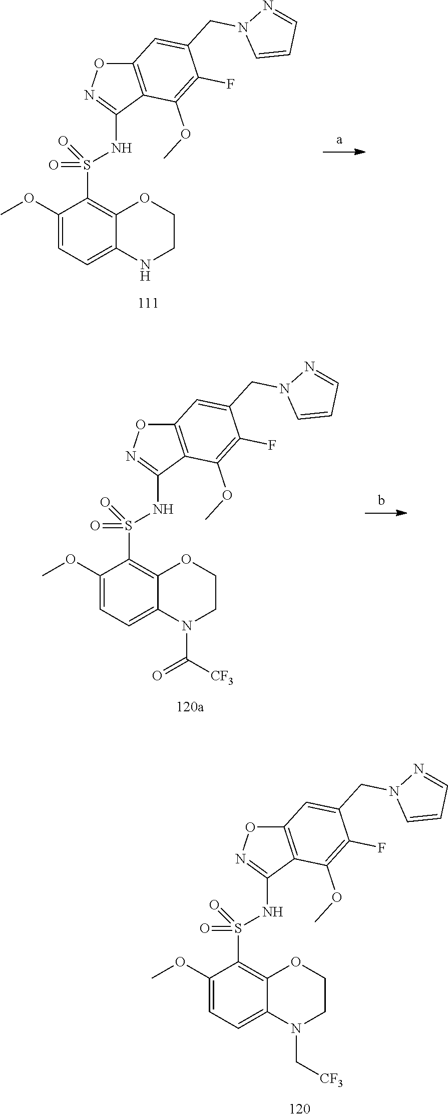 [SMILES: COc1ccc2c(c1S(=O)(=O)Nc1noc3cc(Cn4cccn4)c(F)c(OC)c13)OCCN2 ; COc1ccc2c(c1S(=O)(=O)Nc1noc3cc(Cn4cccn4)c(F)c(OC)c13)OCCN2C(=O)C(F)(F)F ; COc1ccc2c(c1S(=O)(=O)Nc1noc3cc(Cn4cccn4)c(F)c(OC)c13)OCCN2CC(F)(F)F]

### Step a:

### N-(6-((1H-Pyrazol-1-yl)methyl)-5-fluoro-4-methoxybenzo[d]isoxazol-3-yl)-7-methoxy-4-(2,2,2-trifluoroacetyl)-3,4-dihydro-2H-benzo[b][1,4]oxazine-8-sulfonamide

Under a nitrogen atmosphere, trifluoroacetic anhydride (103 mg, 0.49 mmol) was added to a solution of compound 111 (200 mg, 0.41 mmol) and triethylamine (62 mg, 0.61 mmol) in dichloromethane (3 mL) at 0° C., and the reaction system was stirred at 25° C. for 1 h. After the reaction was completed, the mixture was quenched with an ice aqueous solution and extracted with dichloromethane (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=19:1) to give compound 120a (190 mg, yield: 79.4%).

MS m/z (ESI): 586.0 [M+H]⁺.

### Step b:

### N-(6-((1H-Pyrazol-1-yl)methyl)-5-fluoro-4-methoxybenzo[d]isoxazol-3-yl)-7-methoxy-4-(2,2,2-trifluoroethyl)-3,4-dihydro-2H-benzo[b][1,4]oxazine-8-sulfonamide

Borane-tetrahydrofuran complex (1 M in tetrahydrofuran, 0.65 mL, 0.65 mmol) was added to a solution of compound 120a (190 mg, 0.32 mmol) in tetrahydrofuran (5 mL) at 0° C. The reaction system was heated to 60° C. and stirred for 6 h. After the reaction was completed, the mixture was quenched with a 3 M aqueous hydrochloric acid solution and extracted with dichloromethane (30 mL×3). The organic layers were combined, washed with a saturated aqueous NaCl solution, and dried over anhydrous sodium sulfate. After filtration, the mother liquor was concentrated under reduced pressure. The resulting crude product was purified by flash silica gel column chromatography (dichloromethane:methanol=19:1) to give a crude product, which was then purified by high-performance liquid chromatography (Welchrom C18, 20×150 mm, 5 μm; mobile phase A: 5 mM aqueous formic acid solution, mobile phase B: acetonitrile; gradient ratio:phase B 5%-95%) to give compound 120 (31 mg, yield: 16.7%).

¹HNMR (400 MHz, DMSO-d₆): δ10.09 (brs, 1H), 7.86 (d, 1H), 7.50 (d, 1H), 7.06 (d, 1H), 6.89 (d, 1H), 6.64 (d, 1H), 6.30 (t, 1H), 5.50 (s, 2H), 4.16-4.09 (m, 4H), 4.05 (d, 3H), 3.66 (s, 3H), 3.37 (t, 2H).

MS m/z (ESI): 572.1 [M+H]⁺.

### Example 75

### Synthesis of Compound 121

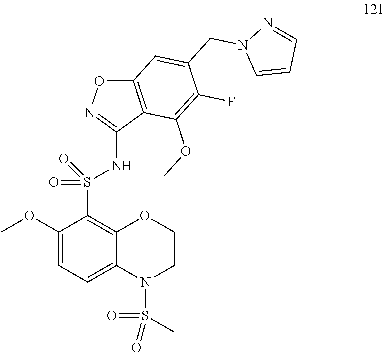 [SMILES: COc1ccc2c(c1S(=O)(=O)Nc1noc3cc(Cn4cccn4)c(F)c(OC)c13)OCCN2S(C)(=O)=O]

### N-(6-((1H-Pyrazol-1-yl)methyl)-5-fluoro-4-methoxybenzo[d]isoxazol-3-yl)-7-methoxy-4-(methylsulfonyl)-3,4-dihydro-2H-benzo[b][1,4]oxazine-8-sulfonamide

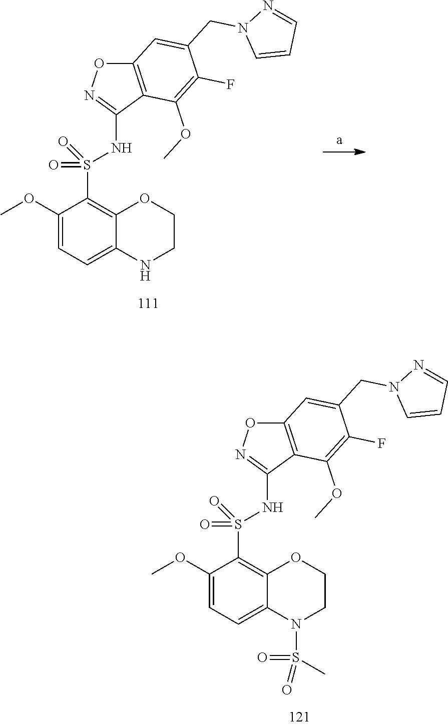 [SMILES: COc1ccc2c(c1S(=O)(=O)Nc1noc3cc(Cn4cccn4)c(F)c(OC)c13)OCCN2 ; COc1ccc2c(c1S(=O)(=O)Nc1noc3cc(Cn4cccn4)c(F)c(OC)c13)OCCN2S(C)(=O)=O]

### Step a:

### N-(6-((1H-Pyrazol-1-yl)methyl)-5-fluoro-4-methoxybenzo[d]isoxazol-3-yl)-7-methoxy-4-(methylsulfonyl)-3,4-dihydro-2H-benzo[b][1,4]oxazine-8-sulfonamide

Triethylamine (87 mg, 0.86 mmol) and methanesulfonyl chloride (98 mg, 0.86 mmol) were added to a solution of compound 111 (140 mg, 0.29 mmol) in dichloromethane (25 mL). The reaction system was stirred at room temperature for 16 h. After the reaction was completed, the mixture was diluted with an aqueous solution and extracted with dichloromethane (30 mL×3). The organic layers were combined, washed with a saturated aqueous NaCl solution, and dried over anhydrous sodium sulfate. After filtration, the mother liquor was concentrated under reduced pressure. The resulting crude product was recrystallized from ethanol to give compound 121 (32 mg, yield: 19.70%).

¹HNMR (400 MHz, DMSO-d₆): δ10.44 (s, 1H), 7.86 (d, 1H), 7.69 (d, 1H), 7.50 (d, 1H), 6.93 (d, 1H), 6.79 (d, 1H), 6.31 (t, 1H), 5.50 (s, 2H), 4.27 (t, 2H), 4.02 (d, 3H), 3.75-3.72 (m, 5H), 3.07 (s, 3H).

MS m/z (ESI): 568.0 [M+H]⁺.

TABLE 2
Typical compounds prepared in the present disclosure

| Compound | | | MS: |
|---|---|---|---|
| No. | Structural formula | Chemical name | (M + H)⁺ |
| 1 | 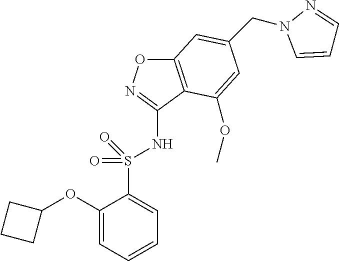 [SMILES: COc1cc(Cn2cccn2)cc2onc(NS(=O)(=O)c3ccccc3OC3CCC3)c12] | N-(6-((1H-Pyrazol-1-yl)methyl)-4- methoxybenzo[d]isoxazol-3-yl)-2- cyclobutoxybenzenesulfonamide | 455.0 |
| 2 | 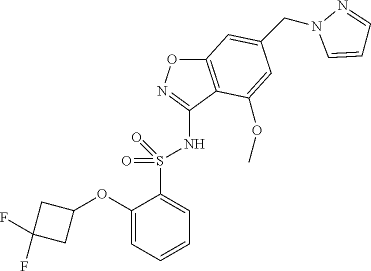 [SMILES: COc1cc(Cn2cccn2)cc2onc(NS(=O)(=O)c3ccccc3OC3CC(F)(F)C3)c12] | N-(6-((1H-Pyrazol-1-yl)methyl)-4- methoxybenzo[d]isoxazol-3-yl)-2-(3,3- difluorocyclobutoxy)benzenesulfonamide | 491.0 |
| 3 | 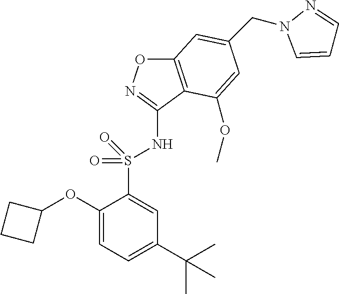 [SMILES: COc1cc(Cn2cccn2)cc2onc(NS(=O)(=O)c3cc(C(C)(C)C)ccc3OC3CCC3)c12] | N-(6-((1H-Pyrazol-1-yl)methyl)-4- methoxybenzo[d]isoxazol-3-yl)-5-(tert-butyl)-2- cyclobutoxybenzenesulfonamide | 511.1 |
| 4 | 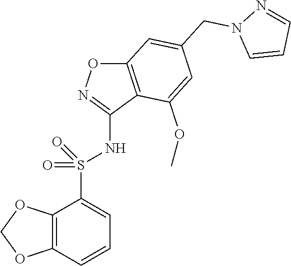 [SMILES: COc1cc(Cn2cccn2)cc2onc(NS(=O)(=O)c3cccc4c3OCO4)c12] | N-(6-((1H-Pyrazol-1-yl)methyl)-4- methoxybenzo[d]isoxazol-3- yl)benzo[d][1,3]dioxole-4-sulfonamide | 428.9 |
| 5 | 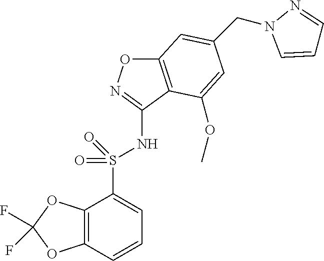 [SMILES: COc1cc(Cn2cccn2)cc2onc(NS(=O)(=O)c3cccc4c3OC(F)(F)O4)c12] | N-(6-((1H-Pyrazol-1-yl)methyl)-4- methoxybenzo[d]isoxazol-3-yl)-2,2- difluorobenzo[d][1,3]dioxole-4-sulfonamide | 464.9 |
| 6 | 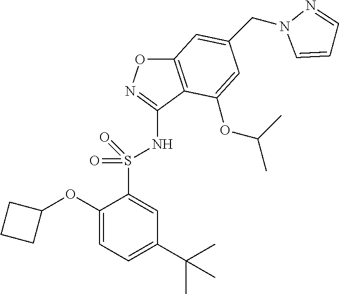 [SMILES: CC(C)Oc1cc(Cn2cccn2)cc2onc(NS(=O)(=O)c3cc(C(C)(C)C)ccc3OC3CCC3)c12] | N-(6-((1H-Pyrazol-1-yl)methyl)-4- isopropoxybenzo[d]isoxazol-3-yl)-5-(tert-butyl)-2- cyclobutoxybenzenesulfonamide | 539.2 |
| 7 | 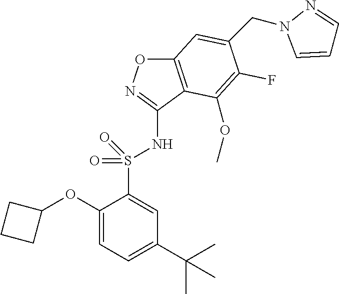 [SMILES: COc1c(F)c(Cn2cccn2)cc2onc(NS(=O)(=O)c3cc(C(C)(C)C)ccc3OC3CCC3)c12] | N-(6-((1H-Pyrazol-1-yl)methyl)-5-fluoro-4- methoxybenzo[d]isoxazol-3-yl)-5-(tert-butyl)-2- cyclobutoxybenzenesulfonamide | 529.0 |
| 8 | 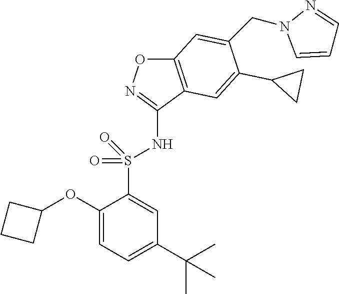 [SMILES: CC(C)(C)c1ccc(OC2CCC2)c(S(=O)(=O)Nc2noc3cc(Cn4cccn4)c(C4CC4)cc23)c1] | N-(6-((1H-Pyrazol-1-yl)methyl)-5- cyclopropylbenzo[d]isoxazol-3-yl)-5-(tert-butyl)- 2-cyclobutoxybenzenesulfonamide | 521.1 |
| 9 | 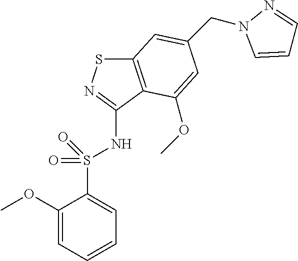 [SMILES: COc1ccccc1S(=O)(=O)Nc1nsc2cc(Cn3cccn3)cc(OC)c12] | N-(6-((1H-Pyrazol-1-yl)methyl)-4- methoxybenzo[d]isothiazol-3-yl)-2- methoxybenzenesulfonamide | 430.9 |
| 10 | 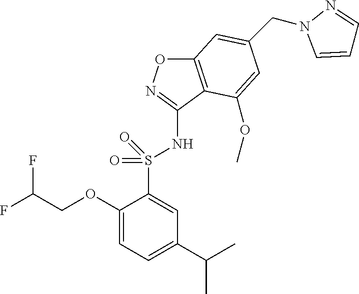 [SMILES: COc1cc(Cn2cccn2)cc2onc(NS(=O)(=O)c3cc(C(C)C)ccc3OCC(F)F)c12] | N-(6-((1H-Pyrazol-1-yl)methyl)-4- methoxybenzo[d]isoxazol-3-yl)-2-(2,2- difluoroethoxy)-5-isopropylbenzenesulfonamide | 507.0 |
| 11 | 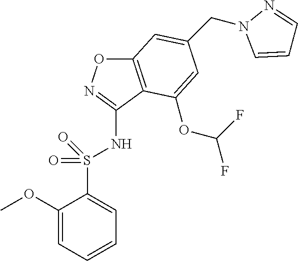 [SMILES: COc1ccccc1S(=O)(=O)Nc1noc2cc(Cn3cccn3)cc(OC(F)F)c12] | N-(6-((1H-Pyrazol-1-yl)methyl)-4- (difluoromethoxy)benzo[d]isoxazol-3-yl)-2- methoxybenzenesulfonamide | 450.9 |
| 12 | 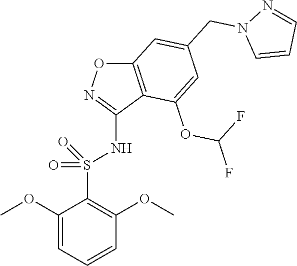 [SMILES: COc1cccc(OC)c1S(=O)(=O)Nc1noc2cc(Cn3cccn3)cc(OC(F)F)c12] | N-(6-((1H-Pyrazol-1-yl)methyl)-4- (difluoromethoxy)benzo[d]isoxazol-3-yl)-2,6- dimethoxybenzenesulfonamide | 481.0 |
| 13 | 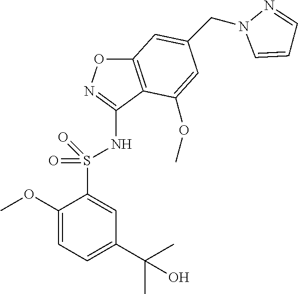 [SMILES: COc1ccc(C(C)(C)O)cc1S(=O)(=O)Nc1noc2cc(Cn3cccn3)cc(OC)c12] | N-(6-((1H-Pyrazol-1-yl)methyl)-4- methoxybenzo[d]isoxazol-3-yl)-5-(2- hydroxypropan-2-yl)-2- methoxybenzenesulfonamide | 473.1 |
| 14 | 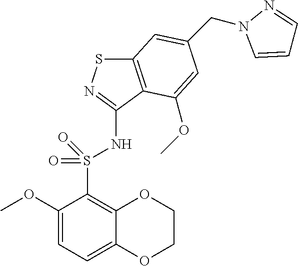 [SMILES: COc1ccc2c(c1S(=O)(=O)Nc1nsc3cc(Cn4cccn4)cc(OC)c13)OCCO2] | N-(6-((1H-Pyrazol-1-yl)methyl)-4- methoxybenzo[d]isoxazol-3-yl)-6-methoxy-2,3- dihydrobenzo[b][1,4]dioxine-5-sulfonamide | 473.0 |
| 15 | 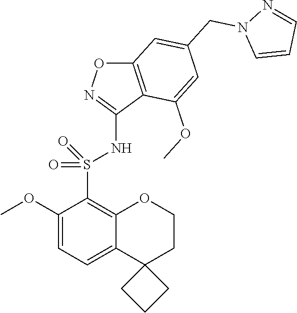 [SMILES: COc1ccc2c(c1S(=O)(=O)Nc1noc3cc(Cn4cccn4)cc(OC)c13)OCCC21CCC1] | N-(6-((1H-Pyrazol-1-yl)methyl)-4- methoxybenzo[d]isoxazol-3-yl)-7- methoxyspiro[chromane-4,1′-cyclobutane]-8- sulfonamide | 511.0 |
| 16 | 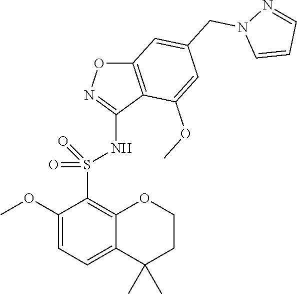 [SMILES: COc1ccc2c(c1S(=O)(=O)Nc1noc3cc(Cn4cccn4)cc(OC)c13)OCCC2(C)C] | N-(6-((1H-Pyrazol-1-yl)methyl)-4- methoxybenzo[d]isoxazol-3-yl)-7-methoxy-4,4- dimethylchromane-8-sulfonamide | 499.0 |
| 17 | 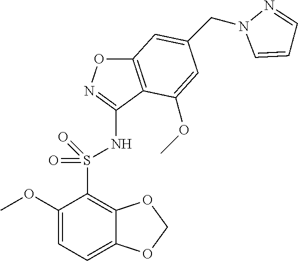 [SMILES: COc1ccc2c(c1S(=O)(=O)Nc1noc3cc(Cn4cccn4)cc(OC)c13)OCO2] | N-(6-((1H-Pyrazol-1-yl)methyl)-4- methoxybenzo[d]isoxazol-3-yl)-5- methoxybenzo[d][1,3]dioxole-4-sulfonamide | 459.0 |
| 18 | 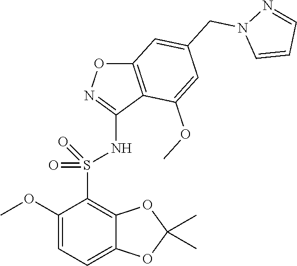 [SMILES: COc1ccc2c(c1S(=O)(=O)Nc1noc3cc(Cn4cccn4)cc(OC)c13)OC(C)(C)O2] | N-(6-((1H-Pyrazol-1-yl)methyl)-4- methoxybenzo[d]isoxazol-3-yl)-5-methoxy-2,2- dimethylbenzo[d][1,3]dioxole-4-sulfonamide | 487.0 |
| 19 | 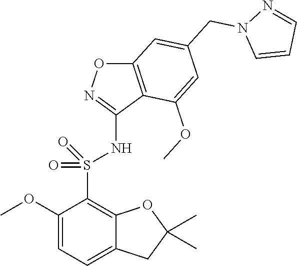 [SMILES: COc1ccc2c(c1S(=O)(=O)Nc1noc3cc(Cn4cccn4)cc(OC)c13)OC(C)(C)C2] | N-(6-((1H-Pyrazol-1-yl)methyl)-4- methoxybenzo[d]isoxazol-3-yl)-6-methoxy-2,2- dimethyl-2,3-dihydrobenzofuran-7-sulfonamide | 485.0 |
| 20 | 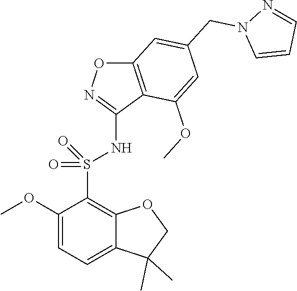 [SMILES: COc1ccc2c(c1S(=O)(=O)Nc1noc3cc(Cn4cccn4)cc(OC)c13)OCC2(C)C] | N-(6-((1H-Pyrazol-1-yl)methyl)-4- methoxybenzo[d]isoxazol-3-yl)-6-methoxy-3,3- dimethyl-2,3-dihydrobenzofuran-7-sulfonamide | 485.0 |
| 21 | 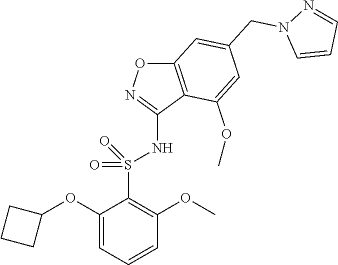 [SMILES: COc1cccc(OC2CCC2)c1S(=O)(=O)Nc1noc2cc(Cn3cccn3)cc(OC)c12] | N-(6-((1H-Pyrazol-1-yl)methyl)-4- methoxybenzo[d]isoxazol-3-yl)-2-cyclobutoxy-6- methoxybenzenesulfonamide | 485.0 |
| 22 | 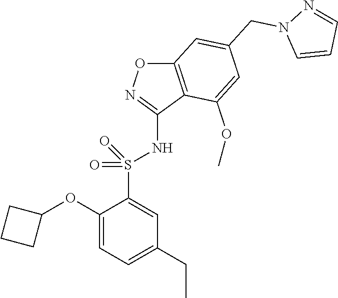 [SMILES: CCc1ccc(OC2CCC2)c(S(=O)(=O)Nc2noc3cc(Cn4cccn4)cc(OC)c23)c1] | N-(6-((1H-Pyrazol-1-yl)methyl)-4- methoxybenzo[d]isoxazol-3-yl)-2-cyclobutoxy-5- ethylbenzenesulfonamide | 483.0 |
| 23 | 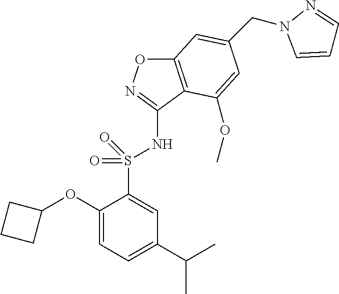 [SMILES: COc1cc(Cn2cccn2)cc2onc(NS(=O)(=O)c3cc(C(C)C)ccc3OC3CCC3)c12] | N-(6-((1H-Pyrazol-1-yl)methyl)-4- methoxybenzo[d]isoxazol-3-yl)-2-cyclobutoxy-5- isopropylbenzenesulfonamide | 497.0 |
| 24 | 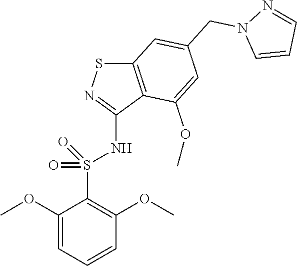 [SMILES: COc1cccc(OC)c1S(=O)(=O)Nc1nsc2cc(Cn3cccn3)cc(OC)c12] | N-(6-((1H-Pyrazol-1-yl)methyl)-4- methoxybenzo[d]isothiazol-3-yl)-2,6- dimethoxybenzenesulfonamide | 460.9 |
| 25 | 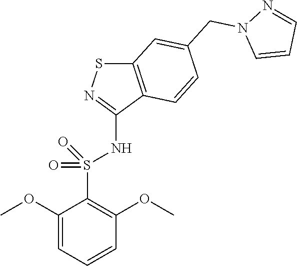 [SMILES: COc1cccc(OC)c1S(=O)(=O)Nc1nsc2cc(Cn3cccn3)ccc12] | N-(6-((1H-Pyrazol-1- yl)methyl)benzo[d]isothiazol-3-yl)-2,6- dimethoxybenzenesulfonamide | 430.9 |
| 26 | 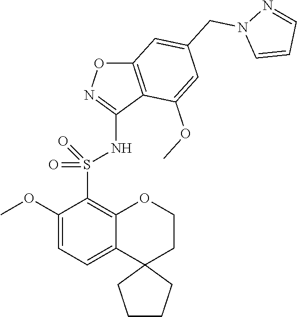 [SMILES: COc1ccc2c(c1S(=O)(=O)Nc1noc3cc(Cn4cccn4)cc(OC)c13)OCCC21CCCC1] | N-(6-((1H-Pyrazol-1-yl)methyl)-4- methoxybenzo[d]isoxazol-3-yl)-7- methoxyspiro[chromane-4,1′-cyclopentane]-8- sulfonamide | 525.0 |
| 27 | 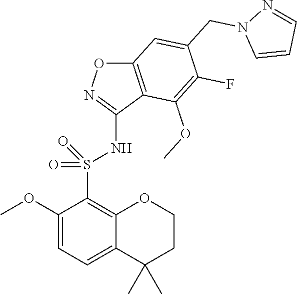 [SMILES: COc1ccc2c(c1S(=O)(=O)Nc1noc3cc(Cn4cccn4)c(F)c(OC)c13)OCCC2(C)C] | N-(6-((1H-Pyrazol-1-yl)methyl)-5-fluoro-4- methoxybenzo[d]isoxazol-3-yl)-7-methoxy-4,4- dimethylchromane-8-sulfonamide | 517.1 |
| 28 | 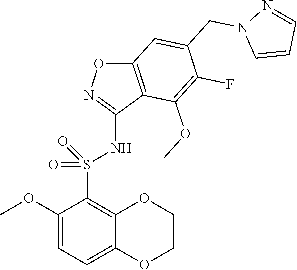 [SMILES: COc1ccc2c(c1S(=O)(=O)Nc1noc3cc(Cn4cccn4)c(F)c(OC)c13)OCCO2] | N-(6-((1H-Pyrazol-1-yl)methyl)-5-fluoro-4- methoxybenzo[d]isoxazol-3-yl)-6-methoxy-2,3- dihydrobenzo[b][1,4]dioxine-5-sulfonamide | 491.1 |
| 29 | 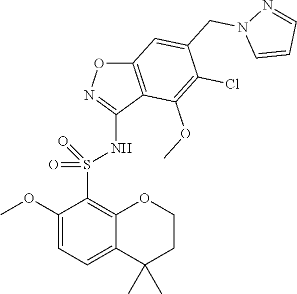 [SMILES: COc1ccc2c(c1S(=O)(=O)Nc1noc3cc(Cn4cccn4)c(Cl)c(OC)c13)OCCC2(C)C] | N-(6-((1H-Pyrazol-1-yl)methyl)-5-chloro-4- methoxybenzo[d]isoxazol-3-yl)-7-methoxy-4,4- dimethylchromane-8-sulfonamide | 533.1 |
| 30 | 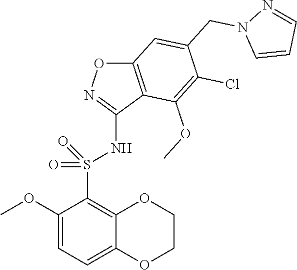 [SMILES: COc1ccc2c(c1S(=O)(=O)Nc1noc3cc(Cn4cccn4)c(Cl)c(OC)c13)OCCO2] | N-(6-((1H-Pyrazol-1-yl)methyl)-5-chloro-4- methoxybenzo[d]isoxazol-3-yl)-6-methoxy-2,3- dihydrobenzo[b][1,4]dioxine-5-sulfonamide | 507.0 |
| 31 | 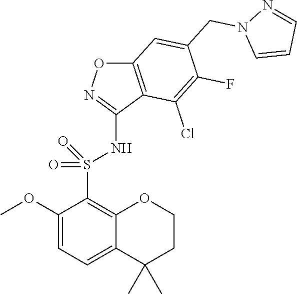 [SMILES: COc1ccc2c(c1S(=O)(=O)Nc1noc3cc(Cn4cccn4)c(F)c(Cl)c13)OCCC2(C)C] | N-(6-((1H-Pyrazol-1-yl)methyl)-4-chloro-5- fluorobenzo[d]isoxazol-3-yl)-7-methoxy-4,4- dimethylchromane-8-sulfonamide | 521.1 |
| 32 | 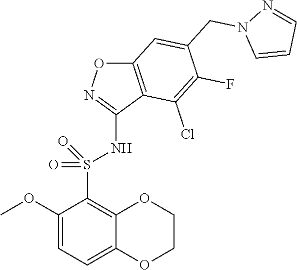 [SMILES: COc1ccc2c(c1S(=O)(=O)Nc1noc3cc(Cn4cccn4)c(F)c(Cl)c13)OCCO2] | N-(6-((1H-Pyrazol-1-yl)methyl)-4-chloro-5- fluorobenzo[d]isoxazol-3-yl)-6-methoxy-2,3- dihydrobenzo[b][1,4]dioxine-5-sulfonamide | 495.0 |
| 33 | 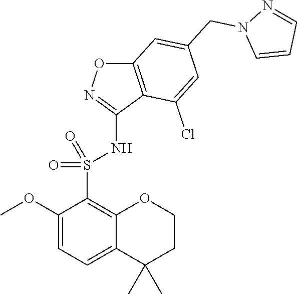 [SMILES: COc1ccc2c(c1S(=O)(=O)Nc1noc3cc(Cn4cccn4)cc(Cl)c13)OCCC2(C)C] | N-(6-((1H-Pyrazol-1-yl)methyl)-4- chlorobenzo[d]isoxazol-3-yl)-7-methoxy-4,4- dimethylchromane-8-sulfonamide | 503.1 |
| 34 | 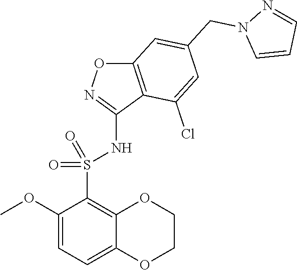 [SMILES: COc1ccc2c(c1S(=O)(=O)Nc1noc3cc(Cn4cccn4)cc(Cl)c13)OCCO2] | N-(6-((1H-Pyrazol-1-yl)methyl)-4- chlorobenzo[d]isoxazol-3-yl)-6-methoxy-2,3- dihydrobenzo[b][1,4]dioxine-5-sulfonamide | 477.0 |
| 35 | 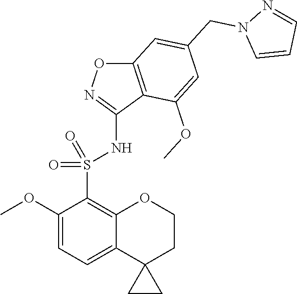 [SMILES: COc1ccc2c(c1S(=O)(=O)Nc1noc3cc(Cn4cccn4)cc(OC)c13)OCCC21CC1] | N-(6-((1H-Pyrazol-1-yl)methyl)-4- methoxybenzo[d]isoxazol-3-yl)-7- methoxyspiro[chromane-4,1′-cyclopropane]-8- sulfonamide | 497.1 |
| 36 | 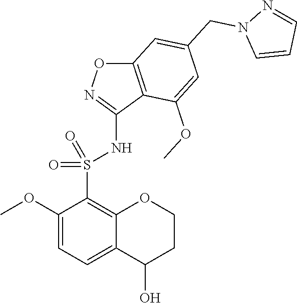 [SMILES: COc1ccc2c(c1S(=O)(=O)Nc1noc3cc(Cn4cccn4)cc(OC)c13)OCCC2O] | N-(6-((1H-Pyrazol-1-yl)methyl)-4- methoxybenzo[d]isoxazol-3-yl)-4-hydroxy-7- methoxychromane-8-sulfonamide | 487.0 |
| 37 | 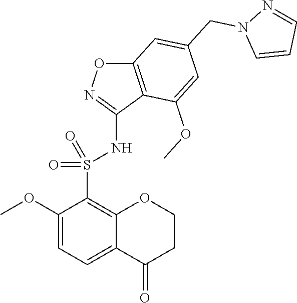 [SMILES: COc1ccc2c(c1S(=O)(=O)Nc1noc3cc(Cn4cccn4)cc(OC)c13)OCCC2=O] | N-(6-((1H-Pyrazol-1-yl)methyl)-4- methoxybenzo[d]isoxazol-3-yl)-7-methoxy-4- oxochromane-8-sulfonamide | 485.1 |
| 38 | 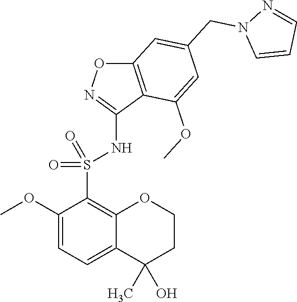 [SMILES: COc1ccc2c(c1S(=O)(=O)Nc1noc3cc(Cn4cccn4)cc(OC)c13)OCCC2(C)O] | N-(6-((1H-Pyrazol-1-yl)methyl)-4- methoxybenzo[d]isoxazol-3-yl)-4-hydroxy-7- methoxy-4-methylchromane-8-sulfonamide | 501.1 |
| 39 | 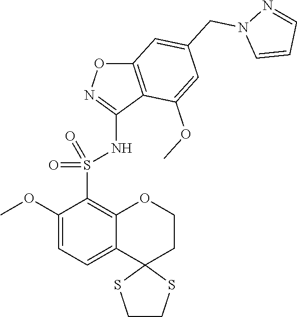 [SMILES: COc1ccc2c(c1S(=O)(=O)Nc1noc3cc(Cn4cccn4)cc(OC)c13)OCCC21SCCS1] | N-(6-((1H-Pyrazol-1-yl)methyl)-4- methoxybenzo[d]isoxazol-3-yl)-7- methoxyspiro[chromane-4,2′-[1,3]dithiolane]-8- sulfonamide | 561.1 |
| 40 | 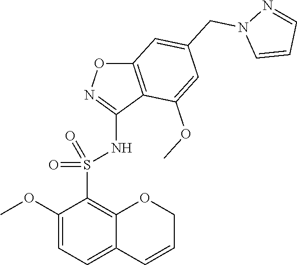 [SMILES: COc1ccc2c(c1S(=O)(=O)Nc1noc3cc(Cn4cccn4)cc(OC)c13)OCC=C2] | N-(6-((1H-Pyrazol-1-yl)methyl)-4- methoxybenzo[d]isoxazol-3-yl)-7-methoxy-2H- chromene-8-sulfonamide | 469.1 |
| 41 | 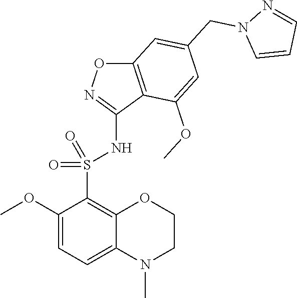 [SMILES: COc1ccc2c(c1S(=O)(=O)Nc1noc3cc(Cn4cccn4)cc(OC)c13)OCCN2C] | N-(6-((1H-Pyrazol-1-yl)methyl)-4- methoxybenzo[d]isoxazol-3-yl)-7-methoxy-4- methyl-3,4-dihydro-2H-benzo[b][1,4]oxazine-8- sulfonamide | 486.1 |
| 42 | 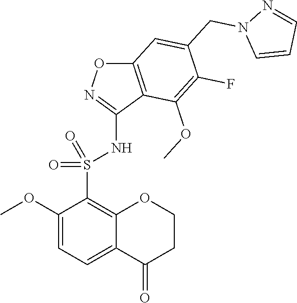 [SMILES: COc1ccc2c(c1S(=O)(=O)Nc1noc3cc(Cn4cccn4)c(F)c(OC)c13)OCCC2=O] | N-(6-((1H-Pyrazol-1-yl)methyl)-5-fluoro-4- methoxybenzo[d]isoxazol-3-yl)-7-methoxy-4- oxochromane-8-sulfonamide | 503.1 |
| 43 | 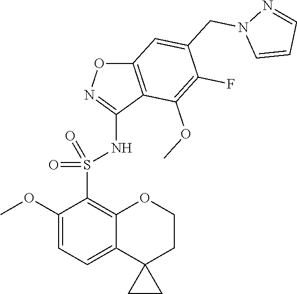 [SMILES: COc1ccc2c(c1S(=O)(=O)Nc1noc3cc(Cn4cccn4)c(F)c(OC)c13)OCCC21CC1] | N-(6-((1H-Pyrazol-1-yl)methyl)-5-fluoro-4- methoxybenzo[d]isoxazol-3-yl)-7- methoxyspiro[chromane-4,1′-cyclopropane]-8- sulfonamide | 515.1 |
| 44 | 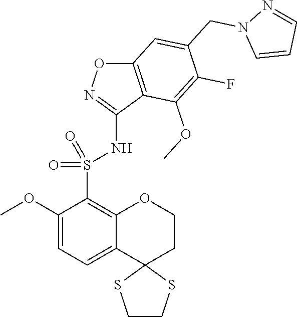 [SMILES: COc1ccc2c(c1S(=O)(=O)Nc1noc3cc(Cn4cccn4)c(F)c(OC)c13)OCCC21SCCS1] | N-(6-((1H-Pyrazol-1-yl)methyl)-5-fluoro-4- methoxybenzo[d]isoxazol-3-yl)-7- methoxyspiro[chromane-4,2′-[1,3]dithiolane]-8- sulfonamide | 579.1 |
| 45 | 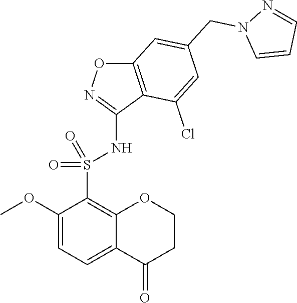 [SMILES: COc1ccc2c(c1S(=O)(=O)Nc1noc3cc(Cn4cccn4)cc(Cl)c13)OCCC2=O] | N-(6-((1H-Pyrazol-1-yl)methyl)-4- chlorobenzo[d]isoxazol-3-yl)-7-methoxy-4- oxochromane-8-sulfonamide | 489.0 |
| 46 | 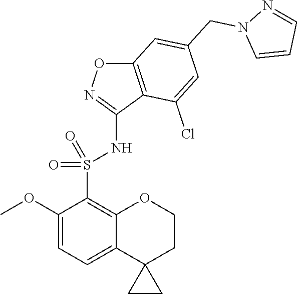 [SMILES: COc1ccc2c(c1S(=O)(=O)Nc1noc3cc(Cn4cccn4)cc(Cl)c13)OCCC21CC1] | N-(6-((1H-Pyrazol-1-yl)methyl)-4- chlorobenzo[d]isoxazol-3-yl)-7- methoxyspiro[chromane-4,1′-cyclopropane]-8- sulfonamide | 501.0 |
| 47 | 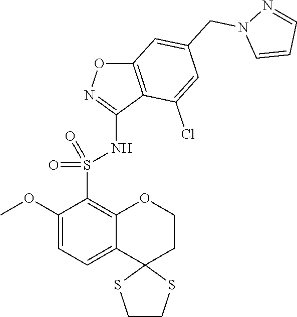 [SMILES: COc1ccc2c(c1S(=O)(=O)Nc1noc3cc(Cn4cccn4)cc(Cl)c13)OCCC21SCCS1] | N-(6-((1H-Pyrazol-1-yl)methyl)-4- chlorobenzo[d]isoxazol-3-yl)-7- methoxyspiro[chromane-4,2′-[1,3]dithiolane]-8- sulfonamide | 565.0 |
| 48 | 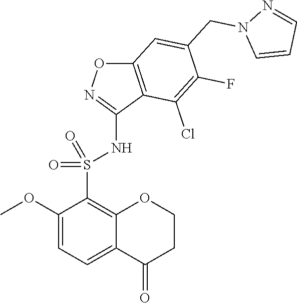 [SMILES: COc1ccc2c(c1S(=O)(=O)Nc1noc3cc(Cn4cccn4)c(F)c(Cl)c13)OCCC2=O] | N-(6-((1H-Pyrazol-1-yl)methyl)-4-chloro-5- fluorobenzo[d]isoxazol-3-yl)-7-methoxy-4- oxochromane-8-sulfonamide | 507.0 |
| 49 | 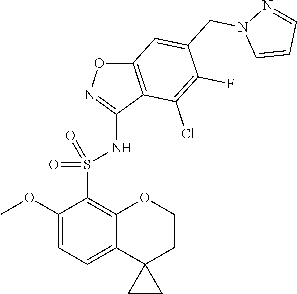 [SMILES: COc1ccc2c(c1S(=O)(=O)Nc1noc3cc(Cn4cccn4)c(F)c(Cl)c13)OCCC21CC1] | N-(6-((1H-Pyrazol-1-yl)methyl)-4-chloro-5- fluorobenzo[d]isoxazol-3-yl)-7- methoxyspiro[chromane-4,1′-cyclopropane]-8- sulfonamide | 519.0 |
| 50 | 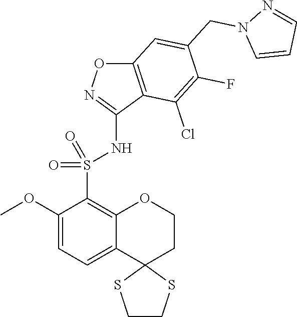 [SMILES: COc1ccc2c(c1S(=O)(=O)Nc1noc3cc(Cn4cccn4)c(F)c(Cl)c13)OCCC21SCCS1] | N-(6-((1H-Pyrazol-1-yl)methyl)-4-chloro-5- fluorobenzo[d]isoxazol-3-yl)-7- methoxyspiro[chromane-4,2′-[1,3]dithiolane]-8- sulfonamide | 583.0 |
| 51 | 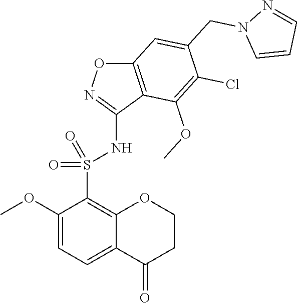 [SMILES: COc1ccc2c(c1S(=O)(=O)Nc1noc3cc(Cn4cccn4)c(Cl)c(OC)c13)OCCC2=O] | N-(6-((1H-Pyrazol-1-yl)methyl)-5-chloro-4- methoxybenzo[d]isoxazol-3-yl)-7-methoxy-4- oxochromane-8-sulfonamide | 519.0 |
| 52 | 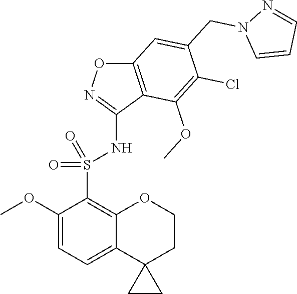 [SMILES: COc1ccc2c(c1S(=O)(=O)Nc1noc3cc(Cn4cccn4)c(Cl)c(OC)c13)OCCC21CC1] | N-(6-((1H-Pyrazol-1-yl)methyl)-5-chloro-4- methoxybenzo[d]isoxazol-3-yl)-7- methoxyspiro[chromane-4,1′-cyclopropane]-8- sulfonamide | 531.0 |
| 53 | 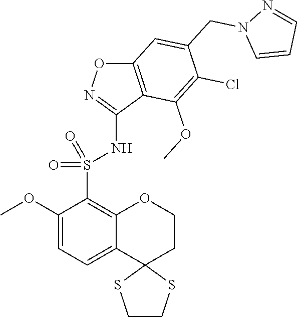 [SMILES: COc1ccc2c(c1S(=O)(=O)Nc1noc3cc(Cn4cccn4)c(Cl)c(OC)c13)OCCC21SCCS1] | N-(6-((1H-Pyrazol-1-yl)methyl)-5-chloro-4- methoxybenzo[d]isoxazol-3-yl)-7- methoxyspiro[chromane-4,2′-[1,3]dithiolane]-8- sulfonamide | 595.0 |
| 54 | 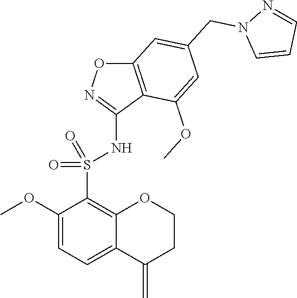 [SMILES: C=C1CCOc2c1ccc(OC)c2S(=O)(=O)Nc1noc2cc(Cn3cccn3)cc(OC)c12] | N-(6-((1H-Pyrazol-1-yl)methyl)-4-methoxy- benzo[d]isoxazol-3-yl)-7-methoxy-4- methylenechromane-8-sulfonamide | 483.1 |
| 55 | 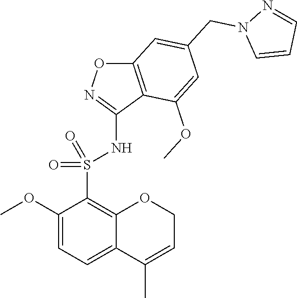 [SMILES: COc1ccc2c(c1S(=O)(=O)Nc1noc3cc(Cn4cccn4)cc(OC)c13)OCC=C2C] | N-(6-((1H-Pyrazol-1-yl)methyl)-4-methoxy-3a,7a- dihydrobenzo[d]isoxazol-3-yl)-7-methoxy-4- methyl-2H-chromene-8-sulfonamide | 483.1 |
| 56 | 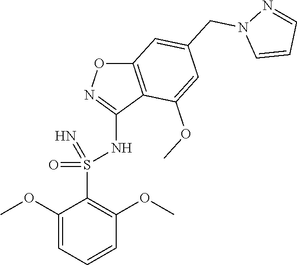 [SMILES: COc1cccc(OC)c1S(=N)(=O)Nc1noc2cc(Cn3cccn3)cc(OC)c12] | N-(6-((1H-Pyrazol-1-yl)methyl)-4- methoxybenzo[d]isoxazol-3-yl)-2,6- dimethoxybenzenesulfonimidamide | 444.1 |
| 57 | 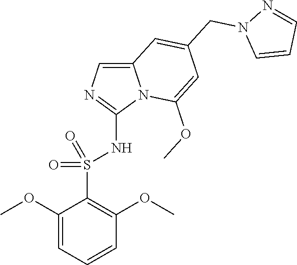 [SMILES: COc1cccc(OC)c1S(=O)(=O)Nc1ncc2cc(Cn3cccn3)cc(OC)n12] | N-(7-((1H-Pyrazol-1-yl)methyl)-5- methoxyimidazo[1,5-a]pyridin-3-yl)-2,6- dimethoxybenzenesulfonamide | 444.1 |
| 58 | 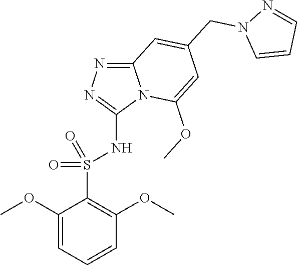 [SMILES: COc1cccc(OC)c1S(=O)(=O)Nc1nnc2cc(Cn3cccn3)cc(OC)n12] | N-(7-((1H-Pyrazol-1-yl)methyl)-5-methoxy- [1,2,4]triazolo[4,3-a]pyridin-3-yl)-2,6- dimethoxybenzenesulfonamide | 445.1 |
| 59 | 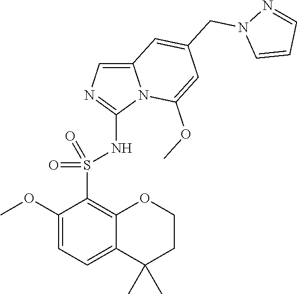 [SMILES: COc1ccc2c(c1S(=O)(=O)Nc1ncc3cc(Cn4cccn4)cc(OC)n13)OCCC2(C)C] | N-(7-((1H-Pyrazol-1-yl)methyl)-5- methoxyimidazo[1,5-a]pyridin-3-yl)-7-methoxy- 4,4-dimethylchromane-8-sulfonamide | 498.2 |
| 60 | 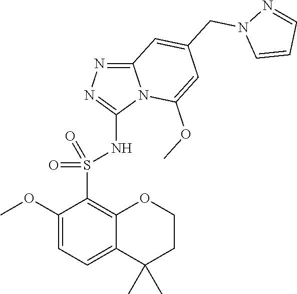 [SMILES: COc1ccc2c(c1S(=O)(=O)Nc1nnc3cc(Cn4cccn4)cc(OC)n13)OCCC2(C)C] | N-(7-((1H-Pyrazol-1-yl)methyl)-5-methoxy- [1,2,4]triazolo[4,3-a]pyridin-3-yl)-7-methoxy-4,4- dimethylchromane-8-sulfonamide | 499.2 |
| 61 | 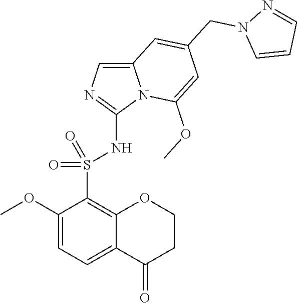 [SMILES: COc1ccc2c(c1S(=O)(=O)Nc1ncc3cc(Cn4cccn4)cc(OC)n13)OCCC2=O] | N-(7-((1H-Pyrazol-1-yl)methyl)-5- methoxyimidazo[1,5-a]pyridin-3-yl)-7-methoxy-4- oxochromane-8-sulfonamide | 484.1 |
| 62 | 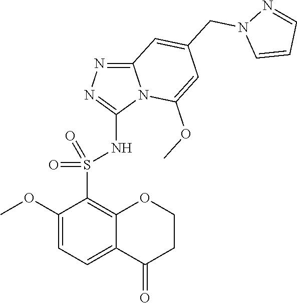 [SMILES: COc1ccc2c(c1S(=O)(=O)Nc1nnc3cc(Cn4cccn4)cc(OC)n13)OCCC2=O] | N-(7-((1H-Pyrazol-1-yl)methyl)-5-methoxy- [1,2,4]triazolo[4,3-a]pyridin-3-yl)-7-methoxy-4- oxochromane-8-sulfonamide | 485.1 |
| 63 | 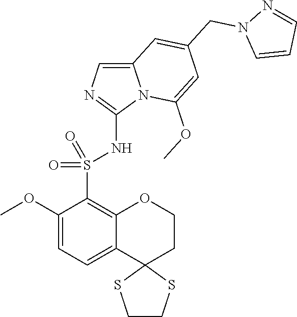 [SMILES: COc1ccc2c(c1S(=O)(=O)Nc1ncc3cc(Cn4cccn4)cc(OC)n13)OCCC21SCCS1] | N-(7-((1H-Pyrazol-1-yl)methyl)-5- methoxyimidazo[1,5-a]pyridin-3-yl)-7- methoxyspiro[chromane-4,2′-[1,3]dithiolane]-8- sulfonamide | 560.1 |
| 64 | 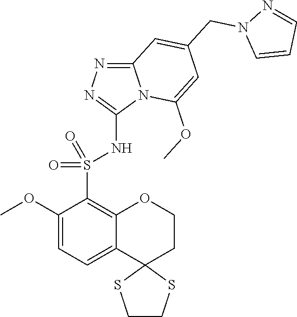 [SMILES: COc1ccc2c(c1S(=O)(=O)Nc1nnc3cc(Cn4cccn4)cc(OC)n13)OCCC21SCCS1] | N-(7-((1H-Pyrazol-1-yl)methyl)-5-methoxy- [1,2,4]triazolo[4,3-a]pyridin-3-yl)-7- methoxyspiro[chromane-4,2′-[1,3]dithiolane]-8- sulfonamide | 561.1 |
| 65 | 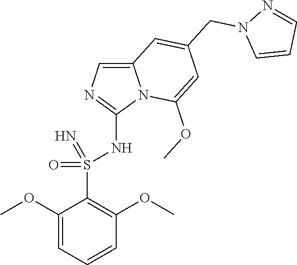 [SMILES: COc1cccc(OC)c1S(=N)(=O)Nc1ncc2cc(Cn3cccn3)cc(OC)n12] | N-(7-((1H-Pyrazol-1-yl)methyl)-5- methoxyimidazo[1,5-a]pyridin-3-yl)-2,6- dimethoxybenzenesulfonimidamide | 443.1 |
| 66 | 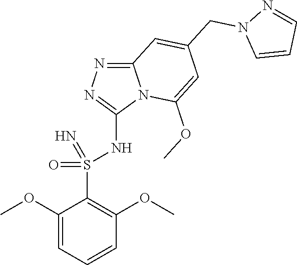 [SMILES: COc1cccc(OC)c1S(=N)(=O)Nc1nnc2cc(Cn3cccn3)cc(OC)n12] | N-(7-((1H-Pyrazol-1-yl)methyl)-5-methoxy- [1,2,4]triazolo[4,3-a]pyridin-3-yl)-2,6- dimethoxybenzenesulfonimidamide | 444.1 |
| 67 | 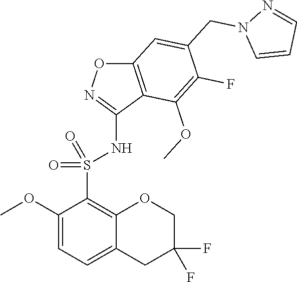 [SMILES: COc1ccc2c(c1S(=O)(=O)Nc1noc3cc(Cn4cccn4)c(F)c(OC)c13)OCC(F)(F)C2] | N-(6-((1H-Pyrazol-1-yl)methyl)-5-fluoro-4- methoxybenzo[d]isoxazol-3-yl)-3,3-difluoro-7- methoxychromane-8-sulfonamide | 425.1 |
| 68 | 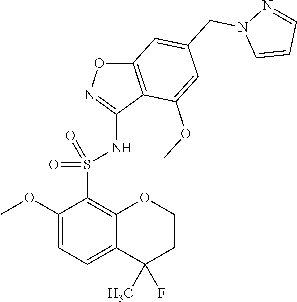 [SMILES: COc1ccc2c(c1S(=O)(=O)Nc1noc3cc(Cn4cccn4)cc(OC)c13)OCCC2(C)F] | N-(6-((1H-Pyrazol-1-yl)methyl)-4- methoxybenzo[d]isoxazol-3-yl)-4-fluoro-7- methoxy-4-methylchromane-8-sulfonamide | 503.1 |
| 69 | 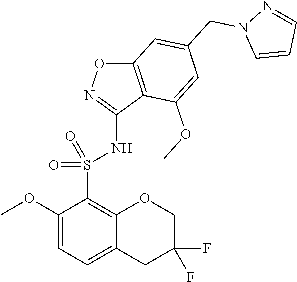 [SMILES: COc1ccc2c(c1S(=O)(=O)Nc1noc3cc(Cn4cccn4)cc(OC)c13)OCC(F)(F)C2] | N-(6-((1H-Pyrazol-1-yl)methyl)-4- methoxybenzo[d]isoxazol-3-yl)-3,3-difluoro-7- methoxychromane-8-sulfonamide | 507.1 |
| 70 | 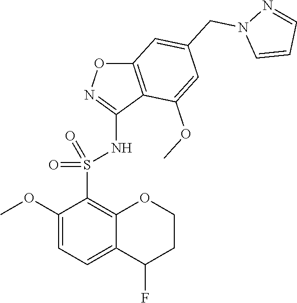 [SMILES: COc1ccc2c(c1S(=O)(=O)Nc1noc3cc(Cn4cccn4)cc(OC)c13)OCCC2F] | N-(6-((1H-Pyrazol-1-yl)methyl)-4- methoxybenzo[d]isoxazol-3-yl)-4-fluoro-7- methoxychromane-8-sulfonamide | 491.1 |
| 71 | 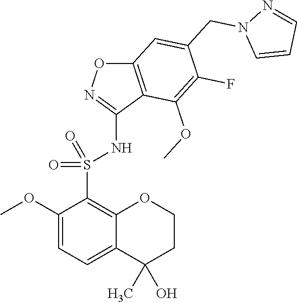 [SMILES: COc1ccc2c(c1S(=O)(=O)Nc1noc3cc(Cn4cccn4)c(F)c(OC)c13)OCCC2(C)O] | N-(6-((1H-Pyrazol-1-yl)methyl)-5-fluoro-4- methoxybenzo[d]isoxazol-3-yl)-4-hydroxy-7- methoxy-4-methylchromane-8-sulfonamide | 519.1 |
| 72 | 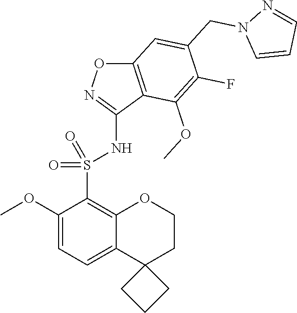 [SMILES: COc1ccc2c(c1S(=O)(=O)Nc1noc3cc(Cn4cccn4)c(F)c(OC)c13)OCCC21CCC1] | N-(6-((1H-Pyrazol-1-yl)methyl)-5-fluoro-4- methoxybenzo[d]isoxazol-3-yl)-7- methoxyspiro[chromane-4,1′-cyclobutane]-8- sulfonamide | 529.1 |
| 73 | 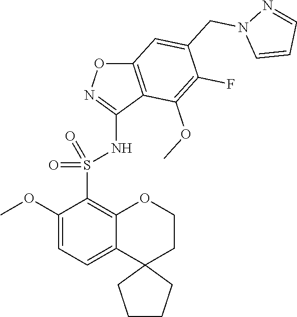 [SMILES: COc1ccc2c(c1S(=O)(=O)Nc1noc3cc(Cn4cccn4)c(F)c(OC)c13)OCCC21CCCC1] | N-(6-((1H-Pyrazol-1-yl)methyl)-5-fluoro-4- methoxybenzo[d]isoxazol-3-yl)-7- methoxyspiro[chromane-4,1′-cyclopentane]-8- sulfonamide | 543.1 |
| 74 | 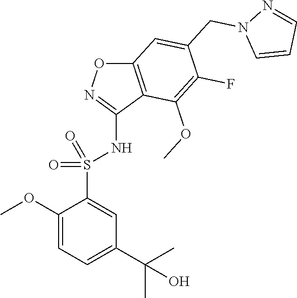 [SMILES: COc1ccc(C(C)(C)O)cc1S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-5-(2- hydroxypropan-2-yl)-2- methoxybenzenesulfonamide | 491.1 |
| 75 | 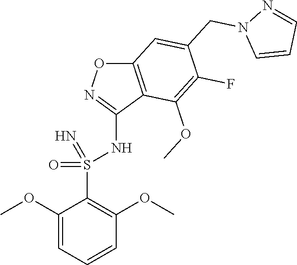 [SMILES: COc1cccc(OC)c1S(=N)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-2,6- dimethoxybenzenesulfonimidamide | 462.1 |
| 76 | 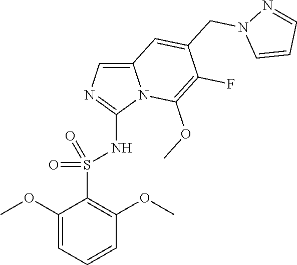 [SMILES: COc1cccc(OC)c1S(=O)(=O)Nc1ncc2cc(Cn3cccn3)c(F)c(OC)n12] | N-(7-((1H-Pyrazol-1-yl)methyl)-6-fluoro-5- methoxyimidazo[1,5-a]pyridin-3-yl)-2,6- dimethoxybenzenesulfonamide | 462.1 |
| 77 | 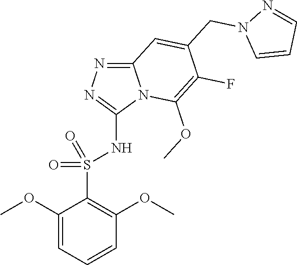 [SMILES: COc1cccc(OC)c1S(=O)(=O)Nc1nnc2cc(Cn3cccn3)c(F)c(OC)n12] | N-(7-((1H-Pyrazol-1-yl)methyl)-6-fluoro-5- methoxy-[1,2,4]triazolo[4,3-a]pyridin-3-yl)-2,6- dimethoxybenzenesulfonamide | 463.1 |
| 78 | 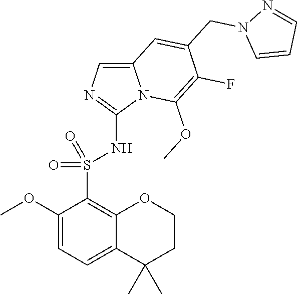 [SMILES: COc1ccc2c(c1S(=O)(=O)Nc1ncc3cc(Cn4cccn4)c(F)c(OC)n13)OCCC2(C)C] | N-(7-((1H-Pyrazol-1-yl)methyl)-6-fluoro-5- methoxyimidazo[1,5-a]pyridin-3-yl)-7-methoxy- 4,4-dimethylchromane-8-sulfonamide | 516.2 |
| 79 | 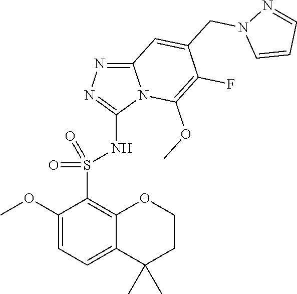 [SMILES: COc1ccc2c(c1S(=O)(=O)Nc1nnc3cc(Cn4cccn4)c(F)c(OC)n13)OCCC2(C)C] | N-(7-((1H-Pyrazol-1-yl)methyl)-6-fluoro-5- methoxy-[1,2,4]triazolo[4,3-a]pyridin-3-yl)-7- methoxy-4,4-dimethylchromane-8-sulfonamide | 517.2 |
| 80 | 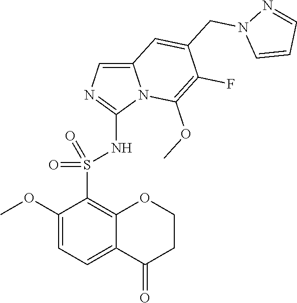 [SMILES: COc1ccc2c(c1S(=O)(=O)Nc1ncc3cc(Cn4cccn4)c(F)c(OC)n13)OCCC2=O] | N-(7-((1H-Pyrazol-1-yl)methyl)-6-fluoro-5- methoxyimidazo[1,5-a]pyridin-3-yl)-7-methoxy-4- oxochromane-8-sulfonamide | 502.1 |
| 81 | 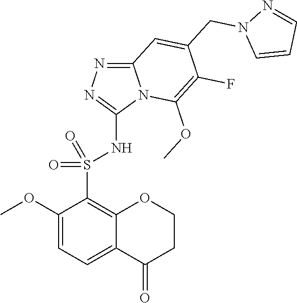 [SMILES: COc1ccc2c(c1S(=O)(=O)Nc1nnc3cc(Cn4cccn4)c(F)c(OC)n13)OCCC2=O] | N-(7-((1H-Pyrazol-1-yl)methyl)-6-fluoro-5- methoxy-[1,2,4]triazolo[4,3-a]pyridin-3-yl)-7- methoxy-4-oxochromane-8-sulfonamide | 503.1 |
| 82 | 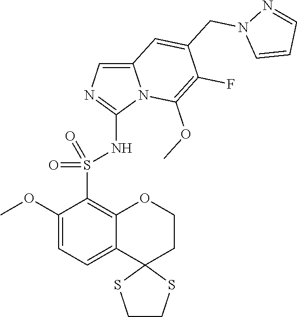 [SMILES: COc1ccc2c(c1S(=O)(=O)Nc1ncc3cc(Cn4cccn4)c(F)c(OC)n13)OCCC21SCCS1] | N-(7-((1H-Pyrazol-1-yl)methyl)-6-fluoro-5- methoxyimidazo[1,5-a]pyridin-3-yl)-7- methoxyspiro[chromane-4,2′-[1,3]dithiolane]-8- sulfonamide | 578.1 |
| 83 | 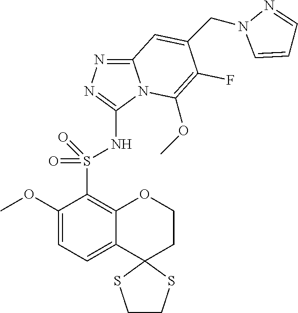 [SMILES: COc1ccc2c(c1S(=O)(=O)Nc1nnc3cc(Cn4cccn4)c(F)c(OC)n13)OCCC21SCCS1] | N-(7-((1H-Pyrazol-1-yl)methyl)-6-fluoro-5- methoxy-[1,2,4]triazolo[4,3-a]pyridin-3-yl)-7- methoxyspiro[chromane-4,2′-[1,3]dithiolane]-8- sulfonamide | 579.1 |
| 84 | 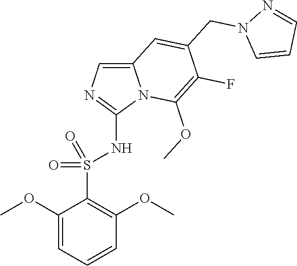 [SMILES: COc1cccc(OC)c1S(=O)(=O)Nc1ncc2cc(Cn3cccn3)c(F)c(OC)n12] | N-(7-((1H-Pyrazol-1-yl)methyl)-6-fluoro-5- methoxyimidazo[1,5-a]pyridin-3-yl)-2,6- dimethoxybenzenesulfonimidamide | 461.1 |
| 85 | 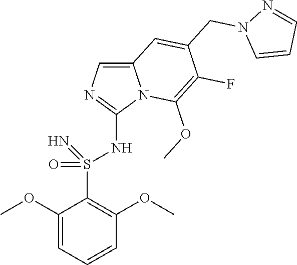 [SMILES: COc1cccc(OC)c1S(=N)(=O)Nc1ncc2cc(Cn3cccn3)c(F)c(OC)n12] | N-(7-((1H-Pyrazol-1-yl)methyl)-6-fluoro-5- methoxy-[1,2,4]triazolo[4,3-a]pyridin-3-yl)-2,6- dimethoxybenzenesulfonimidamide | 462.1 |
| 86 | 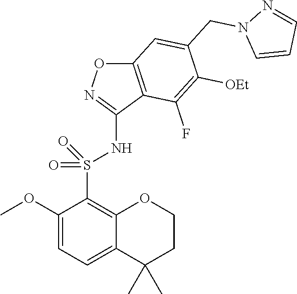 [SMILES: CCOc1c(Cn2cccn2)cc2onc(NS(=O)(=O)c3c(OC)ccc4c3OCCC4(C)C)c2c1F] | N-(6-((1H-Pyrazol-1-yl)methyl)-5-ethoxy-4- fluorobenzo[d]isoxazol-3-yl)-7-methoxy-4,4- dimethylchromane-8-sulfonamide | 531.2 |
| 87 | 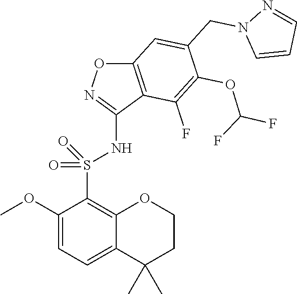 [SMILES: COc1ccc2c(c1S(=O)(=O)Nc1noc3cc(Cn4cccn4)c(OC(F)F)c(F)c13)OCCC2(C)C] | N-(6-((1H-Pyrazol-1-yl)methyl)-5- (difluoromethoxy)-4-fluorobenzo[d]isoxazol-3-yl)- 7-methoxy-4,4-dimethylchromane-8-sulfonamide | 553.1 |
| 88 | 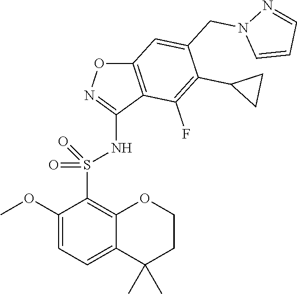 [SMILES: COc1ccc2c(c1S(=O)(=O)Nc1noc3cc(Cn4cccn4)c(C4CC4)c(F)c13)OCCC2(C)C] | N-(6-((1H-Pyrazol-1-yl)methyl)-5-cyclopropyl-4- fluorobenzo[d]isoxazol-3-yl)-7-methoxy-4,4- dimethylchromane-8-sulfonamide | 527.2 |
| 89 | 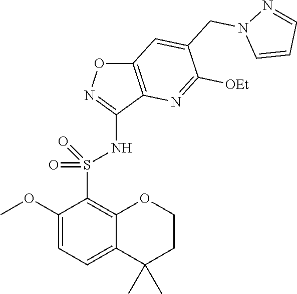 [SMILES: CCOc1nc2c(NS(=O)(=O)c3c(OC)ccc4c3OCCC4(C)C)noc2cc1Cn1cccn1] | N-(6-((1H-Pyrazol-1-yl)methyl)-5- ethoxyisoxazolo[4,5-b]pyridin-3-yl)-7-methoxy- 4,4-dimethylchromane-8-sulfonamide | 514.2 |
| 90 | 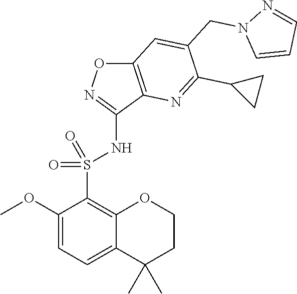 [SMILES: COc1ccc2c(c1S(=O)(=O)Nc1noc3cc(Cn4cccn4)c(C4CC4)nc13)OCCC2(C)C] | N-(6-((1H-Pyrazol-1-yl)methyl)-5- cyclopropylisoxazolo[4,5-b]pyridin-3-yl)-7- methoxy-4,4-dimethylchromane-8-sulfonamide | 510.2 |
| 91 | 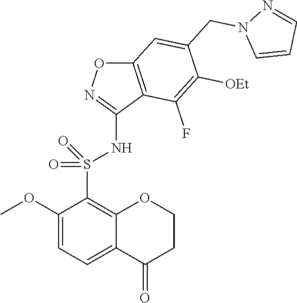 [SMILES: CCOc1c(Cn2cccn2)cc2onc(NS(=O)(=O)c3c(OC)ccc4c3OCCC4=O)c2c1F] | N-(6-((1H-Pyrazol-1-yl)methyl)-5-ethoxy-4- fluorobenzo[d]isoxazol-3-yl)-7-methoxy-4- oxochromane-8-sulfonamide | 517.1 |
| 92 | 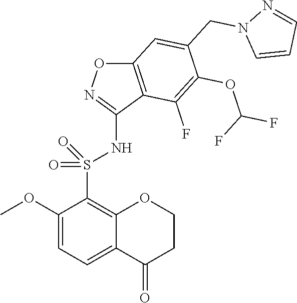 [SMILES: COc1ccc2c(c1S(=O)(=O)Nc1noc3cc(Cn4cccn4)c(OC(F)F)c(F)c13)OCCC2=O] | N-(6-((1H-Pyrazol-1-yl)methyl)-5- (difluoromethoxy)-4-fluorobenzo[d]isoxazol-3-yl)- 7-methoxy-4-oxochromane-8-sulfonamide | 539.1 |
| 93 | 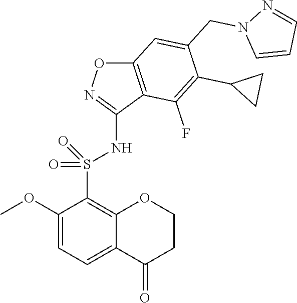 [SMILES: COc1ccc2c(c1S(=O)(=O)Nc1noc3cc(Cn4cccn4)c(C4CC4)c(F)c13)OCCC2=O] | N-(6-((1H-Pyrazol-1-yl)methyl)-5-cyclopropyl-4- fluorobenzo[d]isoxazol-3-yl)-7-methoxy-4- oxochromane-8-sulfonamide | 513.1 |
| 94 | 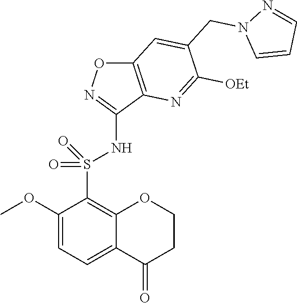 [SMILES: CCOc1nc2c(NS(=O)(=O)c3c(OC)ccc4c3OCCC4=O)noc2cc1Cn1cccn1] | N-(6-((1H-Pyrazol-1-yl)methyl)-5- ethoxyisoxazolo[4,5-b]pyridin-3-yl)-7-methoxy-4- oxochromane-8-sulfonamide | 500.1 |
| 95 | 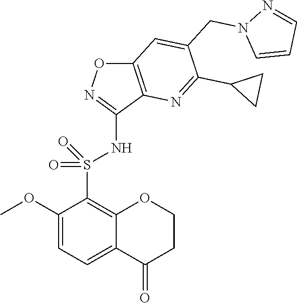 [SMILES: COc1ccc2c(c1S(=O)(=O)Nc1noc3cc(Cn4cccn4)c(C4CC4)nc13)OCCC2=O] | N-(6-((1H-Pyrazol-1-yl)methyl)-5- cyclopropylisoxazolo[4,5-b]pyridin-3-yl)-7- methoxy-4-oxochromane-8-sulfonamide | 496.1 |
| 96 | 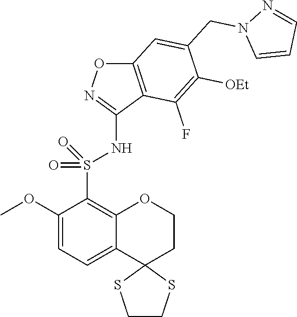 [SMILES: CCOc1c(Cn2cccn2)cc2onc(NS(=O)(=O)c3c(OC)ccc4c3OCCC43SCCS3)c2c1F] | N-(6-((1H-Pyrazol-1-yl)methyl)-5-ethoxy-4- fluorobenzo[d]isoxazol-3-yl)-7- methoxyspiro[chromane-4,2′-[1,3]dithiolane]-8- sulfonamide | 593.1 |
| 97 | 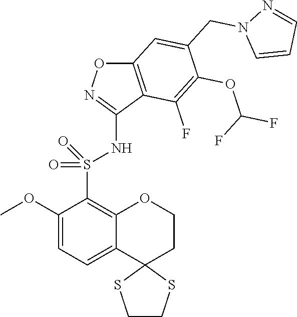 [SMILES: COc1ccc2c(c1S(=O)(=O)Nc1noc3cc(Cn4cccn4)c(OC(F)F)c(F)c13)OCCC21SCCS1] | N-(6-((1H-Pyrazol-1-yl)methyl)-5- (difluoromethoxy)-4-fluorobenzo[d]isoxazol-3-yl)- 7-methoxyspiro[chromane-4,2′-[1,3]dithiolane]-8- sulfonamide | 615.1 |
| 98 | 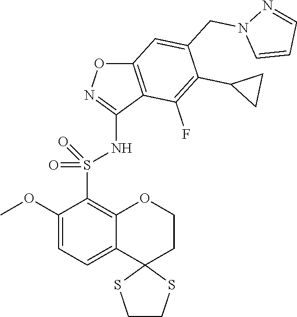 [SMILES: COc1ccc2c(c1S(=O)(=O)Nc1noc3cc(Cn4cccn4)c(C4CC4)c(F)c13)OCCC21SCCS1] | N-(6-((1H-Pyrazol-1-yl)methyl)-5-cyclopropyl-4- fluorobenzo[d]isoxazol-3-yl)-7- methoxyspiro[chromane-4,2′-[1,3]dithiolane]-8- sulfonamide | 589.1 |
| 99 | 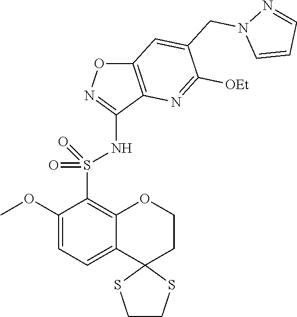 [SMILES: CCOc1nc2c(NS(=O)(=O)c3c(OC)ccc4c3OCCC43SCCS3)noc2cc1Cn1cccn1] | N-(6-((1H-Pyrazol-1-yl)methyl)-5- ethoxyisoxazolo[4,5-b]pyridin-3-yl)-7- methoxyspiro[chromane-4,2′-[1,3]dithiolane]-8- sulfonamide | 576.1 |
| 100 | 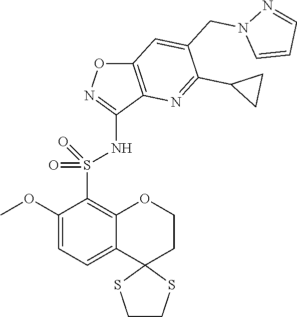 [SMILES: COc1ccc2c(c1S(=O)(=O)Nc1noc3cc(Cn4cccn4)c(C4CC4)nc13)OCCC21SCCS1] | N-(6-((1H-Pyrazol-1-yl)methyl)-5- cyclopropylisoxazolo[4,5-b]pyridin-3-yl)-7- methoxyspiro[chromane-4,2′-[1,3]dithiolane]-8- sulfonamide | 572.1 |
| 101 | 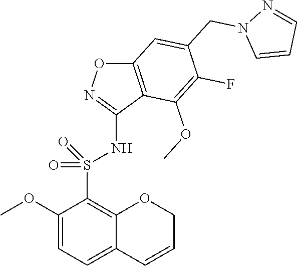 [SMILES: COc1ccc2c(c1S(=O)(=O)Nc1noc3cc(Cn4cccn4)c(F)c(OC)c13)OCC=C2] | N-(6-((1H-Pyrazol-1-yl)methyl)-5-fluoro-4- methoxybenzo[d]isoxazol-3-yl)-7-methoxy-2H- chromene-8-sulfonamide | 487.0 |
| 102 | 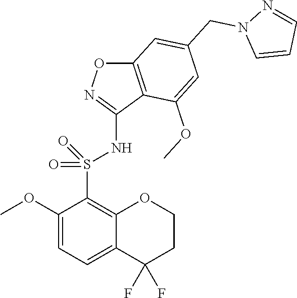 [SMILES: COc1ccc2c(c1S(=O)(=O)Nc1noc3cc(Cn4cccn4)cc(OC)c13)OCCC2(F)F] | N-(6-((1H-Pyrazol-1-yl)methyl)-4- methoxybenzo[d]isoxazol-3-yl)-4,4-difluoro-7- methoxychromane-8-sulfonamide | 506.8 |
| 103 | 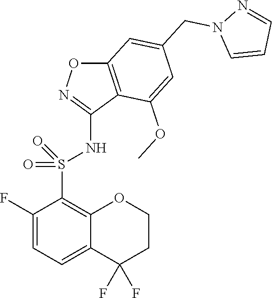 [SMILES: COc1cc(Cn2cccn2)cc2onc(NS(=O)(=O)c3c(F)ccc4c3OCCC4(F)F)c12] | N-(6-((1H-Pyrazol-1-yl)methyl)-4- methoxybenzo[d]isoxazol-3-yl)-4,4,7- trifluorochromane-8-sulfonamide | 494.8 |
| 104 | 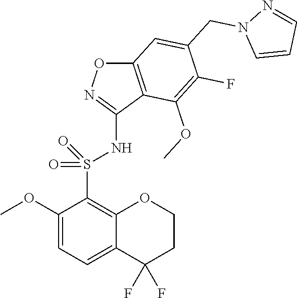 [SMILES: COc1ccc2c(c1S(=O)(=O)Nc1noc3cc(Cn4cccn4)c(F)c(OC)c13)OCCC2(F)F] | N-(6-((1H-Pyrazol-1-yl)methyl)-5-fluoro-4- methoxybenzo[d]isoxazol-3-yl)-4,4-difluoro-7- methoxychromane-8-sulfonamide | 524.9 |
| 105 | 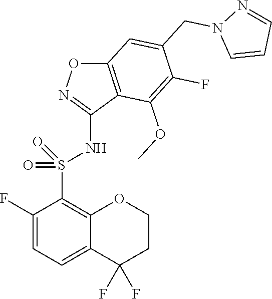 [SMILES: COc1c(F)c(Cn2cccn2)cc2onc(NS(=O)(=O)c3c(F)ccc4c3OCCC4(F)F)c12] | N-(6-((1H-Pyrazol-1-yl)methyl)-5-fluoro-4- methoxybenzo[d]isoxazol-3-yl)-4,4,7- trifluorochromane-8-sulfonamide | 512.8 |
| 106 | 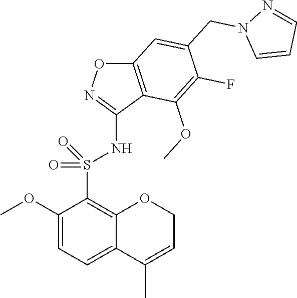 [SMILES: COc1ccc2c(c1S(=O)(=O)Nc1noc3cc(Cn4cccn4)c(F)c(OC)c13)OCC=C2C] | N-(6-((1H-Pyrazol-1-yl)methyl)-5-fluoro-4- methoxybenzo[d]isoxazol-3-yl)-7-methoxy-4- methyl-2H-chromene-8-sulfonamide | 501.1 |
| 107 | 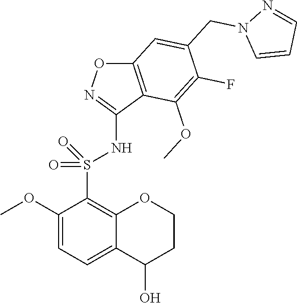 [SMILES: COc1ccc2c(c1S(=O)(=O)Nc1noc3cc(Cn4cccn4)c(F)c(OC)c13)OCCC2O] | N-(6-((1H-Pyrazol-1-yl)methyl)-5-fluoro-4- methoxybenzo[d]isoxazol-3-yl)-4-hydroxy-7- methoxychromane-8-sulfonamide | 505.1 |
| 108 | 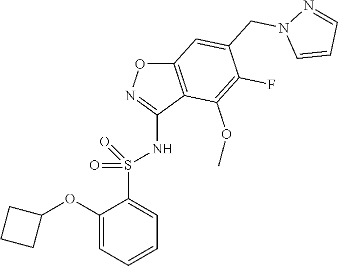 [SMILES: COc1c(F)c(Cn2cccn2)cc2onc(NS(=O)(=O)c3ccccc3OC3CCC3)c12] | N-(6-((1H-Pyrazol-1-yl)methyl)-5-fluoro-4- methoxybenzo[d]isoxazol-3-yl)-2- cyclobutoxybenzenesulfonamide | 472.9 |
| 109 | 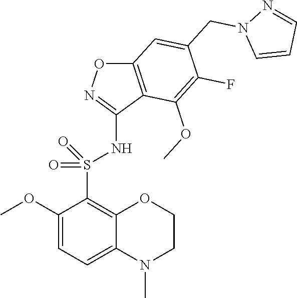 [SMILES: COc1ccc2c(c1S(=O)(=O)Nc1noc3cc(Cn4cccn4)c(F)c(OC)c13)OCCN2C] | N-(6-((1H-Pyrazol-1-yl)methyl)-5-fluoro-4- methoxybenzo[d]isoxazol-3-yl)-7-methoxy-4- methyl-3,4-dihydro-2H-benzo[b][1,4]oxazine-8- sulfonamide | 504.1 |
| 110 | 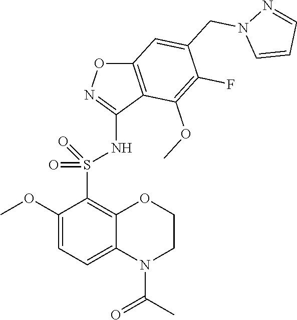 [SMILES: COc1ccc2c(c1S(=O)(=O)Nc1noc3cc(Cn4cccn4)c(F)c(OC)c13)OCCN2C(C)=O] | N-(6-((1H-Pyrazol-1-yl)methyl)-5-fluoro-4- methoxybenzo[d]isoxazol-3-yl)-4-acetyl-7- methoxy-3,4-dihydro-2H-benzo[b][1,4]oxazine-8- sulfonamide | 532.1 |
| 111 | 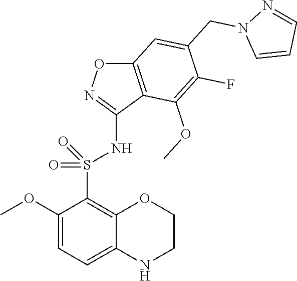 [SMILES: COc1ccc2c(c1S(=O)(=O)Nc1noc3cc(Cn4cccn4)c(F)c(OC)c13)OCCN2] | N-(6-((1H-Pyrazol-1-yl)methyl)-5-fluoro-4- methoxybenzo[d]isoxazol-3-yl)-7-methoxy-3,4- dihydro-2H-benzo[b][1,4]oxazine-8-sulfonamide | 490.1 |
| 112 | 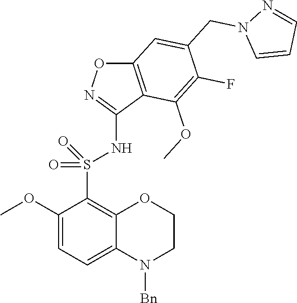 [SMILES: COc1ccc2c(c1S(=O)(=O)Nc1noc3cc(Cn4cccn4)c(F)c(OC)c13)OCCN2Cc1ccccc1] | N-(6-((1H-Pyrazol-1-yl)methyl)-5-fluoro-4- methoxybenzo[d]isoxazol-3-yl)-4-benzyl-7- methoxy-3,4-dihydro-2H-benzo[b][1,4]oxazine-8- sulfonamide | 580.2 |
| 113 | 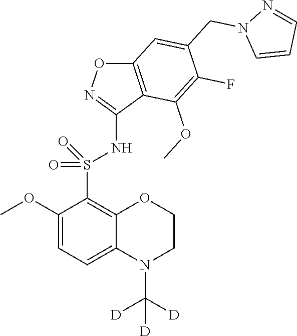 [SMILES: [2H]C([2H])([2H])N1CCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-7-methoxy-4- (methyl-d₃)-3,4-dihydro-2H-benzo[b][1,4]oxazine- 8-sulfonamide | 507.2 |
| 114 | 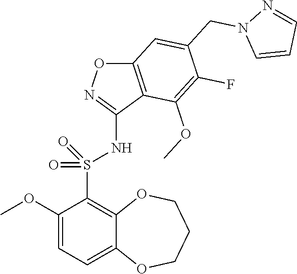 [SMILES: COc1ccc2c(c1S(=O)(=O)Nc1noc3cc(Cn4cccn4)c(F)c(OC)c13)OCCCO2] | N-(6-((1H-Pyrazol-1-yl)methyl)-5-fluoro-4- methoxybenzo[d]isoxazol-3-yl)-7-methoxy-3,4- dihydro-2H-benzo[b][1,4]dioxepine-6- sulfonamide | 505.0 |
| 115 | 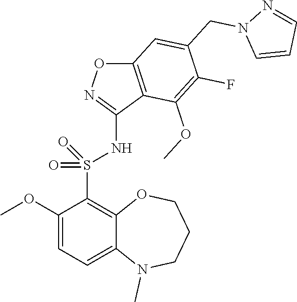 [SMILES: COc1ccc2c(c1S(=O)(=O)Nc1noc3cc(Cn4cccn4)c(F)c(OC)c13)OCCCN2C] | N-(6-((1H-Pyrazol-1-yl)methyl)-5-fluoro-4- methoxybenzo[d]isoxazol-3-yl)-8-methoxy-5- methyl-2,3,4,5-tetrahydrobenzo[b][1,4]oxazepine- 9-sulfonamide | 518.0 |
| 116 | 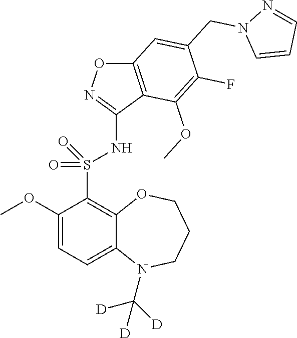 [SMILES: [2H]C([2H])([2H])N1CCCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-8-methoxy-5- (methyl-d₃)-2,3,4,5- tetrahydrobenzo[b][1,4]oxazepine-9-sulfonamide | 521.2 |
| 117 | 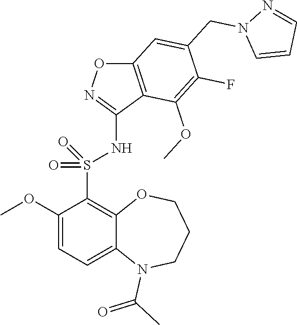 [SMILES: COc1ccc2c(c1S(=O)(=O)Nc1noc3cc(Cn4cccn4)c(F)c(OC)c13)OCCCN2C(C)=O] | N-(6-((1H-Pyrazol-1-yl)methyl)-5-fluoro-4- methoxybenzo[d]isoxazol-3-yl)-5-acetyl-8- methoxy-2,3,4,5- tetrahydrobenzo[b][1,4]oxazepine-9-sulfonamide | 546.1 |
| 118 | 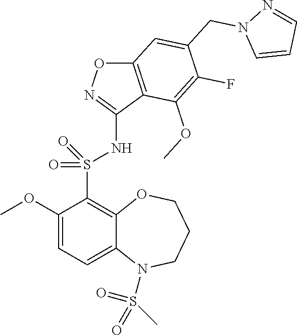 [SMILES: COc1ccc2c(c1S(=O)(=O)Nc1noc3cc(Cn4cccn4)c(F)c(OC)c13)OCCCN2S(C)(=O)=O] | N-(6-((1H-Pyrazol-1-yl)methyl)-5-fluoro-4- methoxybenzo[d]isoxazol-3-yl)-8-methoxy-5- (methylsulfonyl)-2,3,4,5- tetrahydrobenzo[b][1,4]oxazepine-9-sulfonamide | 582.1 |
| 119 | 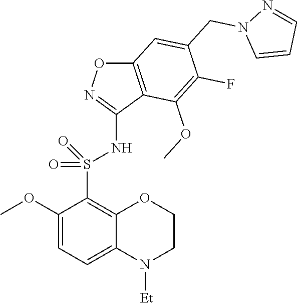 [SMILES: CCN1CCOc2c1ccc(OC)c2S(=O)(=O)Nc1noc2cc(Cn3cccn3)c(F)c(OC)c12] | N-(6-((1H-Pyrazol-1-yl)methyl)-5-fluoro-4- methoxybenzo[d]isoxazol-3-yl)-4-ethyl-7- methoxy-3,4-dihydro-2H-benzo[b][1,4]oxazine-8- sulfonamide | 518.1 |
| 120 | 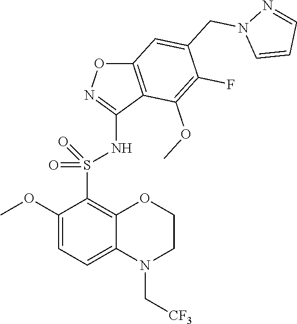 [SMILES: COc1ccc2c(c1S(=O)(=O)Nc1noc3cc(Cn4cccn4)c(F)c(OC)c13)OCCN2CC(F)(F)F] | N-(6-((1H-Pyrazol-1-yl)methyl)-5-fluoro-4- methoxybenzo[d]isoxazol-3-yl)-7-methoxy-4- (2,2,2-trifluoroethyl)-3,4-dihydro-2H- benzo[b][1,4]oxazine-8-sulfonamide | 572.1 |
| 121 | 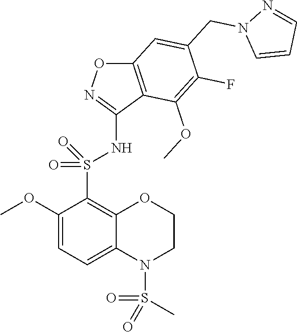 [SMILES: COc1ccc2c(c1S(=O)(=O)Nc1noc3cc(Cn4cccn4)c(F)c(OC)c13)OCCN2S(C)(=O)=O] | N-(6-((1H-Pyrazol-1-yl)methyl)-5-fluoro-4- methoxybenzo[d]isoxazol-3-yl)-7-methoxy-4- (methylsulfonyl)-3,4-dihydro-2H- benzo[b][1,4]oxazine-8-sulfonamide | 568.0 |
| 122 | 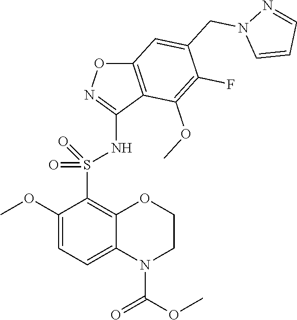 [SMILES: COC(=O)N1CCOc2c1ccc(OC)c2S(=O)(=O)Nc1noc2cc(Cn3cccn3)c(F)c(OC)c12] | Methyl 8-(N-(6-((1H-pyrazol-1-yl)methyl)-5- fluoro-4-methoxybenzo[d]isoxazol-3- yl)sulfamoyl)-7-methoxy-3,4-dihydro-4H- benzo[b][1,4]oxazine-4-carboxylate | 548.1 |
| 123 | 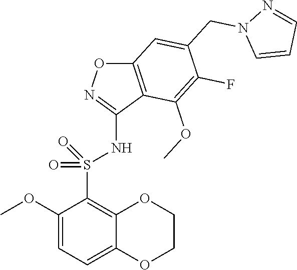 [SMILES: COc1ccc2c(c1S(=O)(=O)Nc1noc3cc(Cn4cccn4)c(F)c(OC)c13)OCCO2] | N-(6-((1H-Pyrazol-1-yl)methyl)-5-fluoro-4- methoxybenzo[d]isothiazol-3-yl)-6-methoxy-2,3- dihydrobenzo[b][1,4]dioxine-5-sulfonamide | 507.1 |
| 124 | 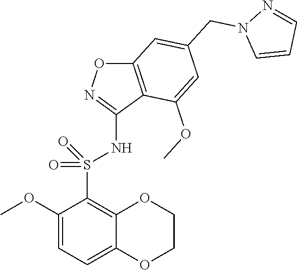 [SMILES: COc1ccc2c(c1S(=O)(=O)Nc1noc3cc(Cn4cccn4)cc(OC)c13)OCCO2] | N-(6-((1H-Pyrazol-1-yl)methyl)-4- methoxybenzo[d]isothiazol-3-yl)-6-methoxy-2,3- dihydrobenzo[b][1,4]dioxine-5-sulfonamide | 489.1 |
| 125 | 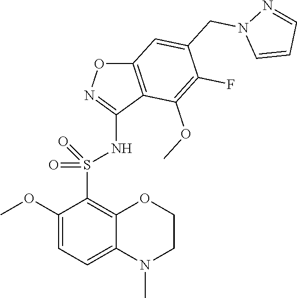 [SMILES: COc1ccc2c(c1S(=O)(=O)Nc1noc3cc(Cn4cccn4)c(F)c(OC)c13)OCCN2C] | N-(6-((1H-Pyrazol-1-yl)methyl)-5-fluoro-4- methoxybenzo[d]isothiazol-3-yl)-7-methoxy-4- methyl-3,4-dihydro-2H-benzo[b][1,4]oxazine-8- sulfonamide | 520.1 |
| 126 | 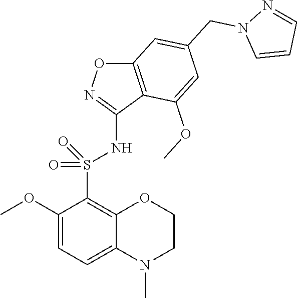 [SMILES: COc1ccc2c(c1S(=O)(=O)Nc1noc3cc(Cn4cccn4)cc(OC)c13)OCCN2C] | N-(6-((1H-Pyrazol-1-yl)methyl)-4- methoxybenzo[d]isothiazol-3-yl)-7-methoxy-4- methyl-3,4-dihydro-2H-benzo[b][1,4]oxazine-8- sulfonamide | 502.1 |
| 127 | 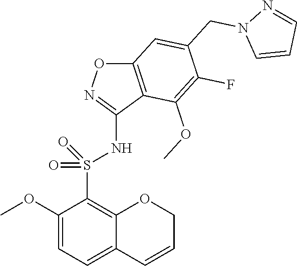 [SMILES: COc1ccc2c(c1S(=O)(=O)Nc1noc3cc(Cn4cccn4)c(F)c(OC)c13)OCC=C2] | N-(6-((1H-Pyrazol-1-yl)methyl)-5-fluoro-4- methoxybenzo[d]isothiazol-3-yl)-7-methoxy-2H- chromene-8-sulfonamide | 503.1 |
| 128 | 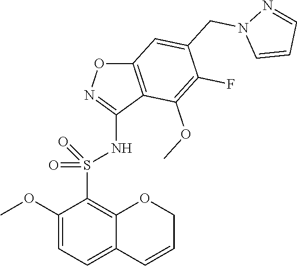 [SMILES: COc1ccc2c(c1S(=O)(=O)Nc1noc3cc(Cn4cccn4)c(F)c(OC)c13)OCC=C2] | N-(6-((1H-Pyrazol-1-yl)methyl)-4- methoxybenzo[d]isothiazol-3-yl)-7-methoxy-2H- chromene-8-sulfonamide | 485.1 |
| 129 | 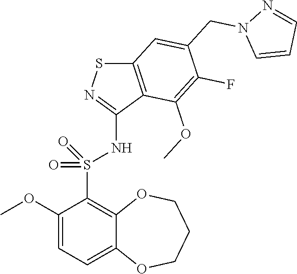 [SMILES: COc1ccc2c(c1S(=O)(=O)Nc1nsc3cc(Cn4cccn4)c(F)c(OC)c13)OCCCO2] | N-(6-((1H-Pyrazol-1-yl)methyl)-5-fluoro-4- methoxybenzo[d]isothiazol-3-yl)-7-methoxy-3,4- dihydro-2H-benzo[b][1,4]dioxepine-6- sulfonamide | 521.1 |
| 130 | 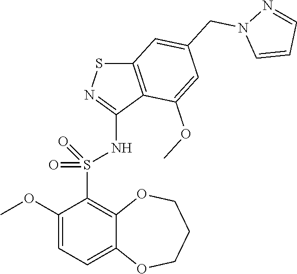 [SMILES: COc1ccc2c(c1S(=O)(=O)Nc1nsc3cc(Cn4cccn4)cc(OC)c13)OCCCO2] | N-(6-((1H-Pyrazol-1-yl)methyl)-4- methoxybenzo[d]isothiazol-3-yl)-7-methoxy-3,4- dihydro-2H-benzo[b][1,4]dioxepine-6- sulfonamide | 503.1 |
| 131 | 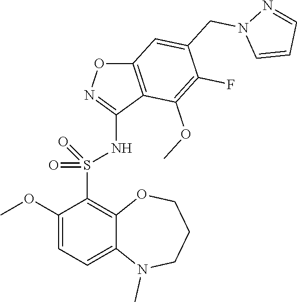 [SMILES: COc1ccc2c(c1S(=O)(=O)Nc1noc3cc(Cn4cccn4)c(F)c(OC)c13)OCCCN2C] | N-(6-((1H-Pyrazol-1-yl)methyl)-5-fluoro-4- methoxybenzo[d]isothiazol-3-yl)-8-methoxy-5- methyl-2,3,4,5-tetrahydrobenzo[b][1,4]oxazepine- 9-sulfonamide | 534.1 |
| 132 | 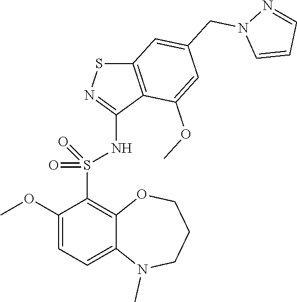 [SMILES: COc1ccc2c(c1S(=O)(=O)Nc1nsc3cc(Cn4cccn4)cc(OC)c13)OCCCN2C] | N-(6-((1H-Pyrazol-1-yl)methyl)-4- methoxybenzo[d]isothiazol-3-yl)-8-methoxy-5- methyl-2,3,4,5-tetrahydrobenzo[b][1,4]oxazepine- 9-sulfonamide | 516.1 |

Note:
Compounds other than Examples 1-75 included in Table 2 were prepared in a manner similar to that for Examples 1-75.

### Biological Evaluation

### Test Example 1: Experiment for Determining Inhibitory Effects of Compounds on Proliferation of Breast Cancer Cell Line ZR-75-1

### Experimental Principle

ATP is an indicator of the metabolism of living cells. The CellTiter-Glo® Luminescent Cell Viability Assay is a homogeneous assay method for determining the number of viable cells by quantifying ATP.

### Reagents and Instruments

1) ZR-75-1 (Cell Bank of Chinese Academy of Sciences, TCHul26)
2) RPMI-1640 medium (Gibco, 11875-093)
3) Fetal Bovine Serum, New Zealand (Gibco,10091-148)
4) Penicillin-streptomycin (10,000 U/mL) (Gibco, 15140-122)
5) Trypsin-EDTA (0.25%), containing phenol red (Gibco, 25200-072)
6) DPBS (Shanghai BasalMedia, B210KJ)
7) Corning® 96-well Clear Flat Bottom Ultra-Low Attachment Microplate (Corning, Cat. No. 3474)
8) 330 μL 96 Holes Deep Well Plate, transparents, 96 Round Holes, V-shaped Bottom, Low Retention (Stellar Scientific, DP33VR-9-NS)
9) Corning® 96-well solid white flat-bottom polystyrene microplate (Corning, Cat. No. 3917)
10) CellTiter-Glo® Luminescent Cell Viability Assay (Promega, G7573)
11) Countstar® BioMed automatic cell counter
12) Trypan blue solution, 0.4% (Gibco, Cat. No. 15250061)
13) 37° C. constant-temperature incubator (Thermo, FORMA STERI-CYCLE i160 LK)
14) Biosafety cabinet (Thermo, 1379)

### Experimental Procedures:

### Cell Plating

1) The formula of the complete medium for ZR-75-1 was as follows: 500 mL RPMI-1640+10% FBS+1% penicillin-streptomycin.
2) Thirty minutes before cell plating, the complete medium, serum, trypsin, and DPBS for use in ZR-75-1 cell culture were pre-warmed in a water bath at 37° C.
3) The ZR-75-1 cells were taken out of the incubator, and the medium was removed by pipetting. The cells were rinsed once with 5 mL of DPBS, and then the DPBS was discarded.
4) The cells in the T75 culture flask were digested with 1-3 mL of (0.25%) trypsin-EDTA. After the trypsin was added, the culture flask was incubated in an incubator at 37° C. for 2-5 min, and dispersion was confirmed by microscopic examination. Tapping or agitating the culture flask was avoided to prevent disturbing the cells, which might cause cell clumping.
5) 9 mL of the complete medium was added to terminate the digestion, thus forming a cell suspension, which was then mixed well by pipetting.
6) Cell suspension counting: 20 μL of the cell suspension was pipetted and diluted with 20 L of the 0.4% trypan blue solution (Gibco, Cat. No. 15250061). The total cell count and the percentage of cell viability were determined using the Countstar® BioMed automatic cell counter. Only cells with a cell viability of no less than 95% were used for the experiment.
7) The concentration of the cell suspension was adjusted to 2000 cells/well, and the cell suspension was added to a 96-well cell culture plate at 80 μL/well.
   Treatment of Cells with Compounds
1) Twenty-four hours after cell plating, the cell plate was taken out.
2) Each compound was prepared with a complete medium. The stock solution was a 10 mM solution in DMSO, which was first diluted with the medium to 100 μM, then diluted to 1 μM, and finally subjected to a 5-fold serial dilution across a total of 9 concentration gradients.
3) 20 μL of the compound diluted with the medium was added to each well of the cell culture plate, which already contained 80 μL of the medium. The final concentration started at 200 nM, followed by a 5-fold serial dilution across a total of 9 concentration gradients. Two replicate wells were set for each compound, and each plate could be used to test 3 compounds, with the DMSO content controlled within 0.5%.
4) The culture medium was replenished once every three days with a complete medium containing the same concentration of the test compound, with 20 μL/well added each time.

### Plate Reading Assay

After 10 days of incubation with the compound, the cell culture plate was taken out, and the CellTiter-Glo® reagent was added at 50 μL/well. The mixture was mixed well by shaking on a shaker for 20 min, then left to stand at room temperature for 20 min, and pipetted and transferred to a white plate at 100 μL/well. Then, readings were taken using a multi-mode microplate reader for assay.

TABLE 3
IC₅₀ ranges for inhibitory effects of compounds on proliferation of ZR-75-1 cells
A: IC₅₀ < 0.5 nM; B: IC₅₀ ≥ 0.5 nM to <2 nM; C: IC₅₀ ≥ 2 nM to <10 nM; D: IC₅₀ ≥ 10 nM to
<50 nM; E: IC₅₀ ≥ 50 nM.

| Compound No. | Structure | IC₅₀ (KAT6A) |
|---|---|---|
| PF-9363 | 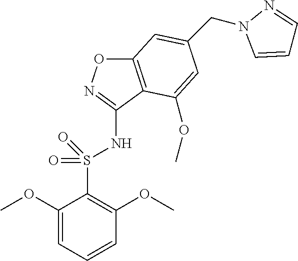 [SMILES: COc1cccc(OC)c1S(=O)(=O)Nc1noc2cc(Cn3cccn3)cc(OC)c12] | B |
| PF-8144 | 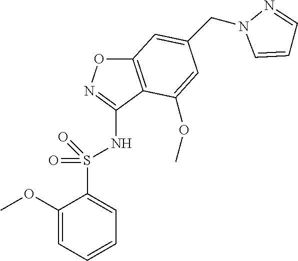 [SMILES: COc1ccccc1S(=O)(=O)Nc1noc2cc(Cn3cccn3)cc(OC)c12] | B |
| PF-0128 | 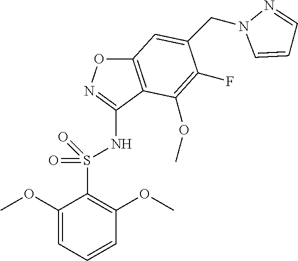 [SMILES: COc1cccc(OC)c1S(=O)(=O)Nc1noc2cc(Cn3cccn3)c(F)c(OC)c12] | B |
| 1 | 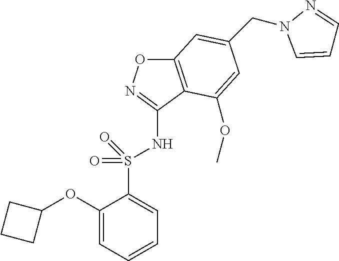 [SMILES: COc1cc(Cn2cccn2)cc2onc(NS(=O)(=O)c3ccccc3OC3CCC3)c12] | B |
| 2 | 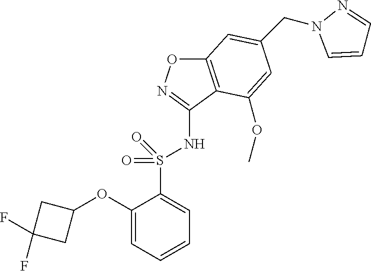 [SMILES: COc1cc(Cn2cccn2)cc2onc(NS(=O)(=O)c3ccccc3OC3CC(F)(F)C3)c12] | C |
| 3 | 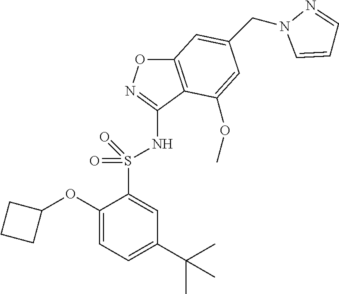 [SMILES: COc1cc(Cn2cccn2)cc2onc(NS(=O)(=O)c3cc(C(C)(C)C)ccc3OC3CCC3)c12] | C |
| 4 | 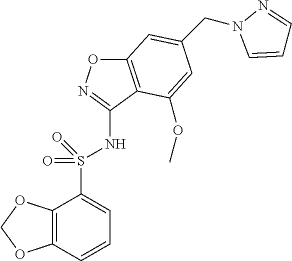 [SMILES: COc1cc(Cn2cccn2)cc2onc(NS(=O)(=O)c3cccc4c3OCO4)c12] | D |
| 5 | 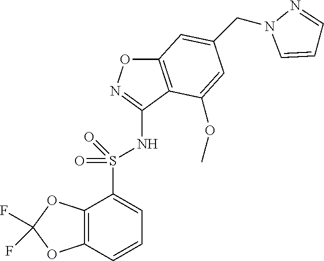 [SMILES: COc1cc(Cn2cccn2)cc2onc(NS(=O)(=O)c3cccc4c3OC(F)(F)O4)c12] | E |
| 6 | 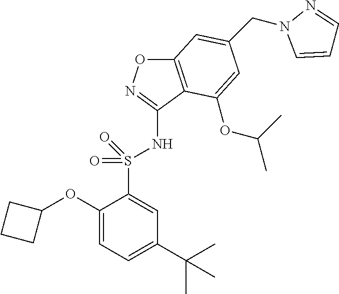 [SMILES: CC(C)Oc1cc(Cn2cccn2)cc2onc(NS(=O)(=O)c3cc(C(C)(C)C)ccc3OC3CCC3)c12] | C |
| 7 | 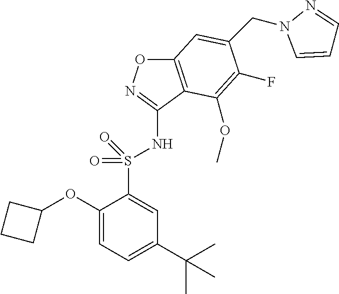 [SMILES: COc1c(F)c(Cn2cccn2)cc2onc(NS(=O)(=O)c3cc(C(C)(C)C)ccc3OC3CCC3)c12] | B |
| 8 | 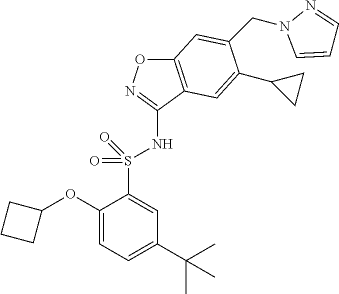 [SMILES: CC(C)(C)c1ccc(OC2CCC2)c(S(=O)(=O)Nc2noc3cc(Cn4cccn4)c(C4CC4)cc23)c1] | D |
| 9 | 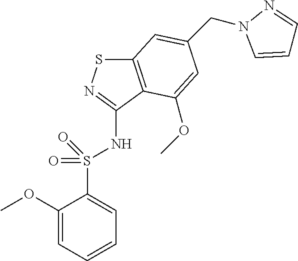 [SMILES: COc1ccccc1S(=O)(=O)Nc1nsc2cc(Cn3cccn3)cc(OC)c12] | E |
| 10 | 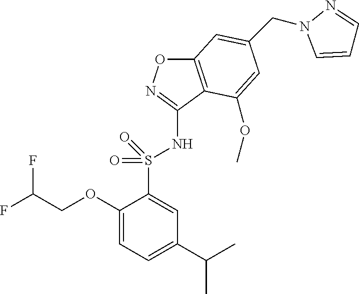 [SMILES: COc1cc(Cn2cccn2)cc2onc(NS(=O)(=O)c3cc(C(C)C)ccc3OCC(F)F)c12] | C |
| 11 | 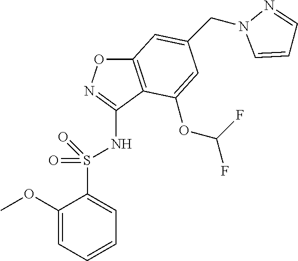 [SMILES: COc1ccccc1S(=O)(=O)Nc1noc2cc(Cn3cccn3)cc(OC(F)F)c12] | C |
| 12 | 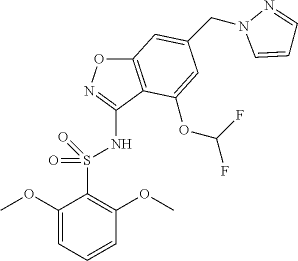 [SMILES: COc1cccc(OC)c1S(=O)(=O)Nc1noc2cc(Cn3cccn3)cc(OC(F)F)c12] | C |
| 13 | 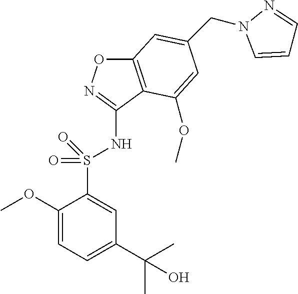 [SMILES: COc1ccc(C(C)(C)O)cc1S(=O)(=O)Nc1noc2cc(Cn3cccn3)cc(OC)c12] | B |
| 14 | 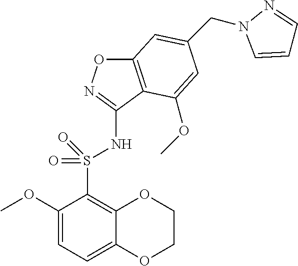 [SMILES: COc1ccc2c(c1S(=O)(=O)Nc1noc3cc(Cn4cccn4)cc(OC)c13)OCCO2] | B |
| 15 | 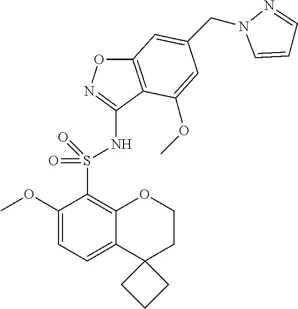 [SMILES: COc1ccc2c(c1S(=O)(=O)Nc1noc3cc(Cn4cccn4)cc(OC)c13)OCCC21CCC1] | A |
| 16 | 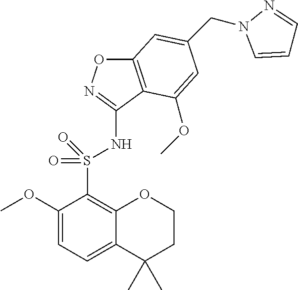 [SMILES: COc1ccc2c(c1S(=O)(=O)Nc1noc3cc(Cn4cccn4)cc(OC)c13)OCCC2(C)C] | A |
| 17 | 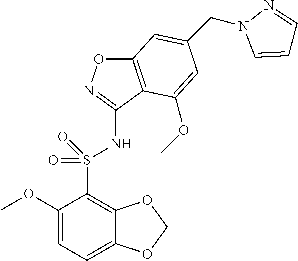 [SMILES: COc1ccc2c(c1S(=O)(=O)Nc1noc3cc(Cn4cccn4)cc(OC)c13)OCO2] | B |
| 18 | 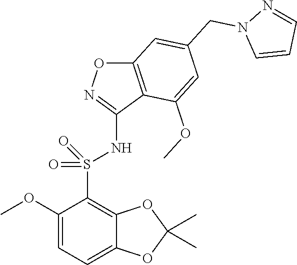 [SMILES: COc1ccc2c(c1S(=O)(=O)Nc1noc3cc(Cn4cccn4)cc(OC)c13)OC(C)(C)O2] | C |
| 19 | 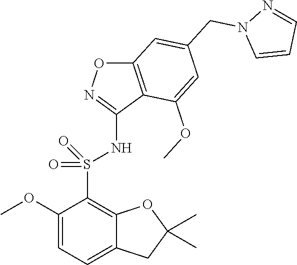 [SMILES: COc1ccc2c(c1S(=O)(=O)Nc1noc3cc(Cn4cccn4)cc(OC)c13)OC(C)(C)C2] | C |
| 20 | 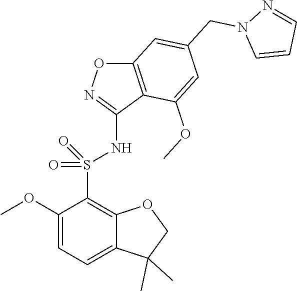 [SMILES: COc1ccc2c(c1S(=O)(=O)Nc1noc3cc(Cn4cccn4)cc(OC)c13)OCC2(C)C] | B |
| 21 | 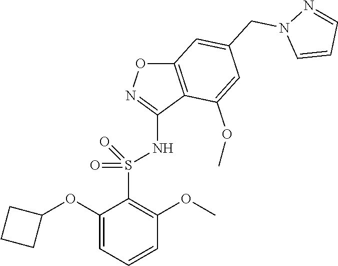 [SMILES: COc1cccc(OC2CCC2)c1S(=O)(=O)Nc1noc2cc(Cn3cccn3)cc(OC)c12] | C |
| 22 | 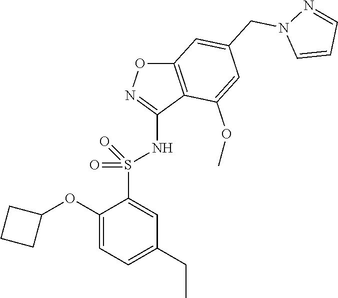 [SMILES: CCc1ccc(OC2CCC2)c(S(=O)(=O)Nc2noc3cc(Cn4cccn4)cc(OC)c23)c1] | C |
| 23 | 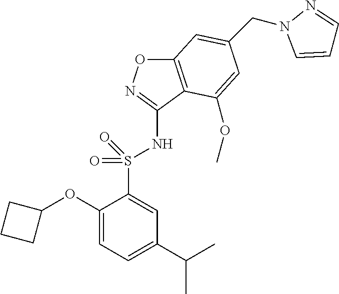 [SMILES: COc1cc(Cn2cccn2)cc2onc(NS(=O)(=O)c3cc(C(C)C)ccc3OC3CCC3)c12] | C |
| 24 | 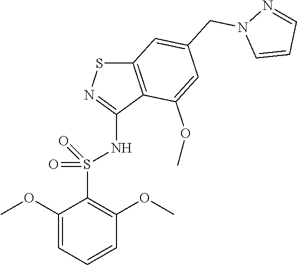 [SMILES: COc1cccc(OC)c1S(=O)(=O)Nc1nsc2cc(Cn3cccn3)cc(OC)c12] | D |
| 25 | 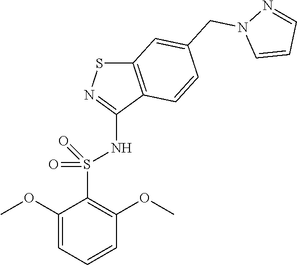 [SMILES: COc1cccc(OC)c1S(=O)(=O)Nc1nsc2cc(Cn3cccn3)ccc12] | E |
| 26 | 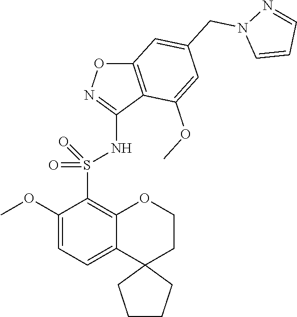 [SMILES: COc1ccc2c(c1S(=O)(=O)Nc1noc3cc(Cn4cccn4)cc(OC)c13)OCCC21CCCC1] | A |
| 27 | 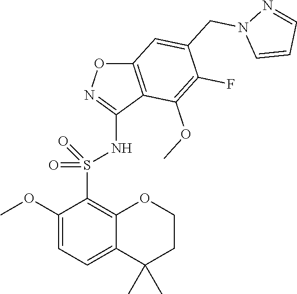 [SMILES: COc1ccc2c(c1S(=O)(=O)Nc1noc3cc(Cn4cccn4)c(F)c(OC)c13)OCCC2(C)C] | A |
| 28 | 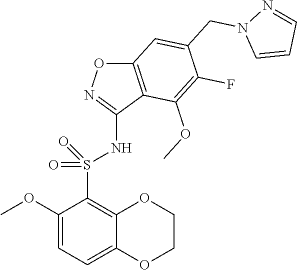 [SMILES: COc1ccc2c(c1S(=O)(=O)Nc1noc3cc(Cn4cccn4)c(F)c(OC)c13)OCCO2] | A |
| 29 | 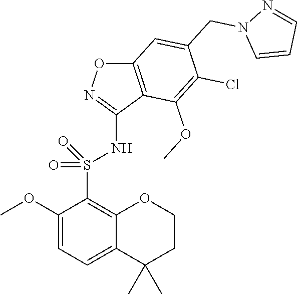 [SMILES: COc1ccc2c(c1S(=O)(=O)Nc1noc3cc(Cn4cccn4)c(Cl)c(OC)c13)OCCC2(C)C] | D |
| 30 | 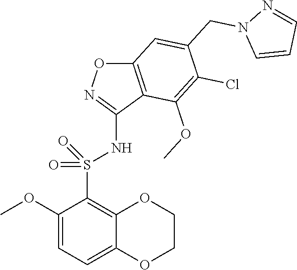 [SMILES: COc1ccc2c(c1S(=O)(=O)Nc1noc3cc(Cn4cccn4)c(Cl)c(OC)c13)OCCO2] | D |
| 31 | 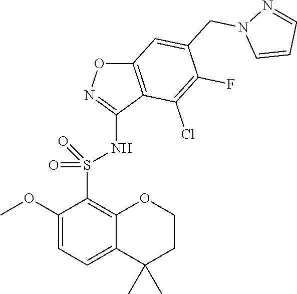 [SMILES: COc1ccc2c(c1S(=O)(=O)Nc1noc3cc(Cn4cccn4)c(F)c(Cl)c13)OCCC2(C)C] | B |
| 32 | 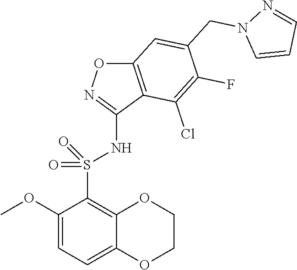 [SMILES: COc1ccc2c(c1S(=O)(=O)Nc1noc3cc(Cn4cccn4)c(F)c(Cl)c13)OCCO2] | C |
| 33 | 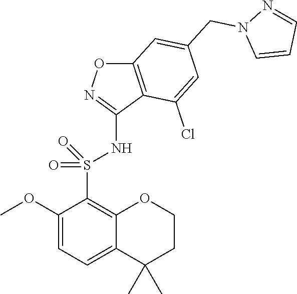 [SMILES: COc1ccc2c(c1S(=O)(=O)Nc1noc3cc(Cn4cccn4)cc(Cl)c13)OCCC2(C)C] | B |
| 34 | 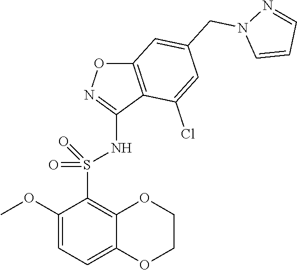 [SMILES: COc1ccc2c(c1S(=O)(=O)Nc1noc3cc(Cn4cccn4)cc(Cl)c13)OCCO2] | C |
| 35 | 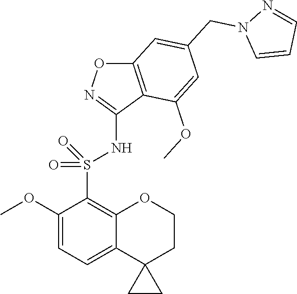 [SMILES: COc1ccc2c(c1S(=O)(=O)Nc1noc3cc(Cn4cccn4)cc(OC)c13)OCCC21CC1] | A |
| 36 | 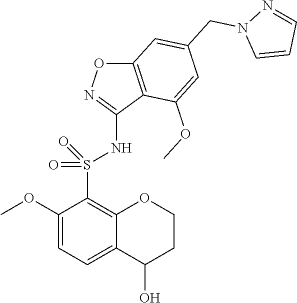 [SMILES: COc1ccc2c(c1S(=O)(=O)Nc1noc3cc(Cn4cccn4)cc(OC)c13)OCCC2O] | A |
| 37 | 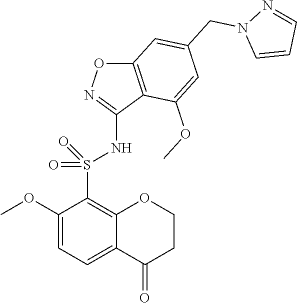 [SMILES: COc1ccc2c(c1S(=O)(=O)Nc1noc3cc(Cn4cccn4)cc(OC)c13)OCCC2=O] | A |
| 38 | 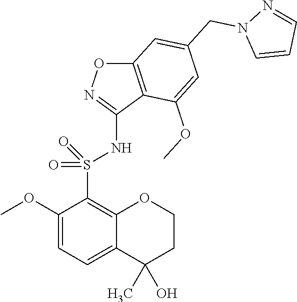 [SMILES: COc1ccc2c(c1S(=O)(=O)Nc1noc3cc(Cn4cccn4)cc(OC)c13)OCCC2(C)O] | A |
| 39 | 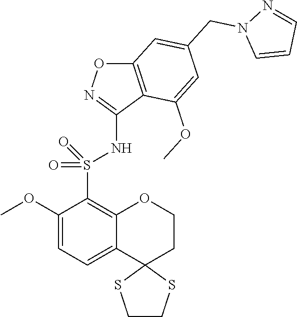 [SMILES: COc1ccc2c(c1S(=O)(=O)Nc1noc3cc(Cn4cccn4)cc(OC)c13)OCCC21SCCS1] | A |
| 40 | 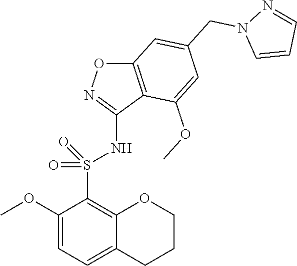 [SMILES: COc1ccc2c(c1S(=O)(=O)Nc1noc3cc(Cn4cccn4)cc(OC)c13)OCCC2] | A |
| 41 | 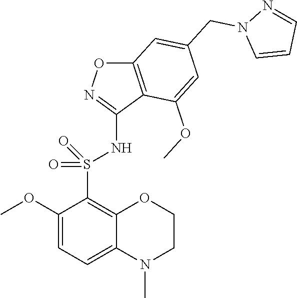 [SMILES: COc1ccc2c(c1S(=O)(=O)Nc1noc3cc(Cn4cccn4)cc(OC)c13)OCCN2C] | B |
| 42 | 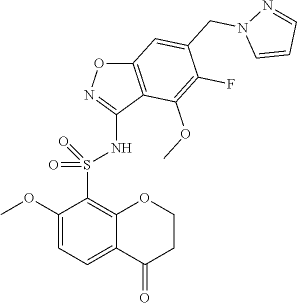 [SMILES: COc1ccc2c(c1S(=O)(=O)Nc1noc3cc(Cn4cccn4)c(F)c(OC)c13)OCCC2=O] | A |
| 43 | 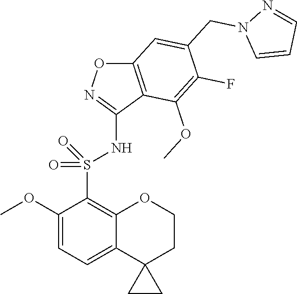 [SMILES: COc1ccc2c(c1S(=O)(=O)Nc1noc3cc(Cn4cccn4)c(F)c(OC)c13)OCCC21CC1] | A |
| 44 | 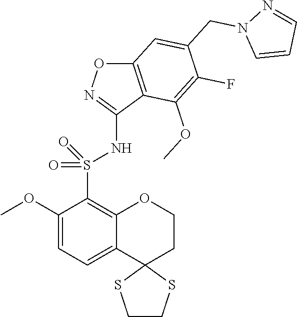 [SMILES: COc1ccc2c(c1S(=O)(=O)Nc1noc3cc(Cn4cccn4)c(F)c(OC)c13)OCCC21SCCS1] | A |
| 45 | 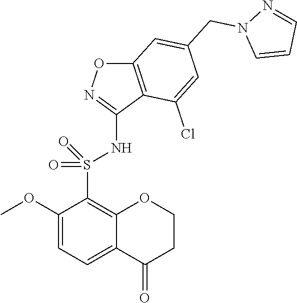 [SMILES: COc1ccc2c(c1S(=O)(=O)Nc1noc3cc(Cn4cccn4)cc(Cl)c13)OCCC2=O] | C |
| 46 | 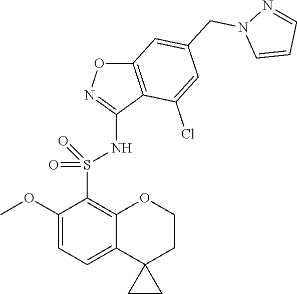 [SMILES: COc1ccc2c(c1S(=O)(=O)Nc1noc3cc(Cn4cccn4)cc(Cl)c13)OCCC21CC1] | C |
| 47 | 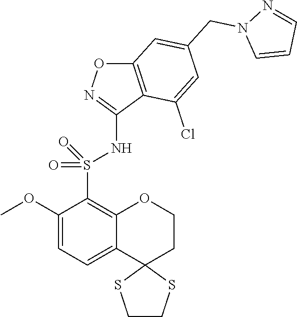 [SMILES: COc1ccc2c(c1S(=O)(=O)Nc1noc3cc(Cn4cccn4)cc(Cl)c13)OCCC21SCCS1] | B |
| 48 | 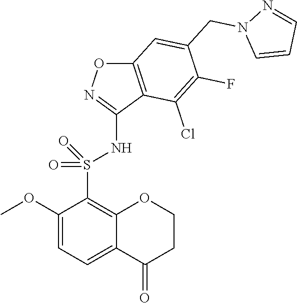 [SMILES: COc1ccc2c(c1S(=O)(=O)Nc1noc3cc(Cn4cccn4)c(F)c(Cl)c13)OCCC2=O] | B |
| 49 | 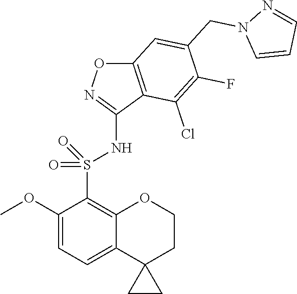 [SMILES: COc1ccc2c(c1S(=O)(=O)Nc1noc3cc(Cn4cccn4)c(F)c(Cl)c13)OCCC21CC1] | C |
| 52 | 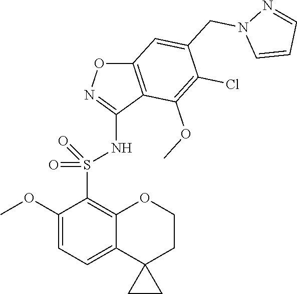 [SMILES: COc1ccc2c(c1S(=O)(=O)Nc1noc3cc(Cn4cccn4)c(Cl)c(OC)c13)OCCC21CC1] | E |
| 58 | 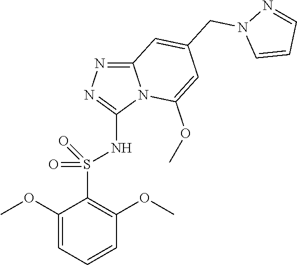 [SMILES: COc1cccc(OC)c1S(=O)(=O)Nc1nnc2cc(Cn3cccn3)cc(OC)n12] | E |
| 72 | 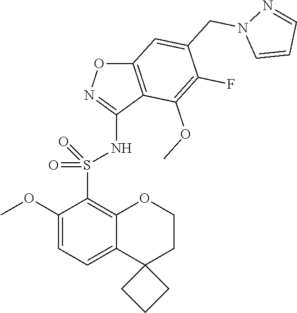 [SMILES: COc1ccc2c(c1S(=O)(=O)Nc1noc3cc(Cn4cccn4)c(F)c(OC)c13)OCCC21CCC1] | B |
| 73 | 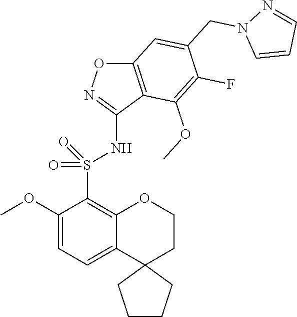 [SMILES: COc1ccc2c(c1S(=O)(=O)Nc1noc3cc(Cn4cccn4)c(F)c(OC)c13)OCCC21CCCC1] | B |
| 101 | 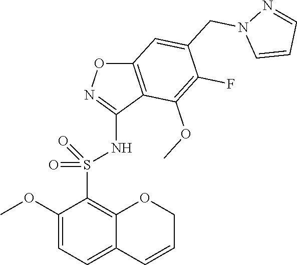 [SMILES: COc1ccc2c(c1S(=O)(=O)Nc1noc3cc(Cn4cccn4)c(F)c(OC)c13)OCC=C2] | A |
| 102 | 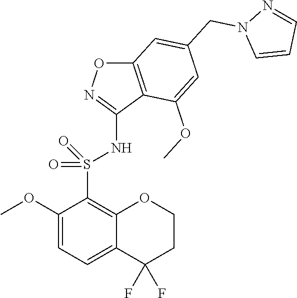 [SMILES: COc1ccc2c(c1S(=O)(=O)Nc1noc3cc(Cn4cccn4)cc(OC)c13)OCCC2(F)F] | B |
| 103 | 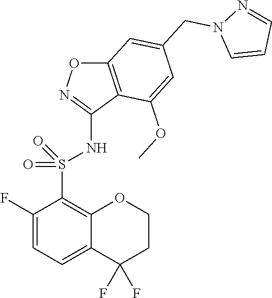 [SMILES: COc1cc(Cn2cccn2)cc2onc(NS(=O)(=O)c3c(F)ccc4c3OCCC4(F)F)c12] | D |
| 104 | 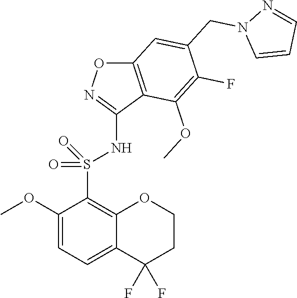 [SMILES: COc1ccc2c(c1S(=O)(=O)Nc1noc3cc(Cn4cccn4)c(F)c(OC)c13)OCCC2(F)F] | A |
| 105 | 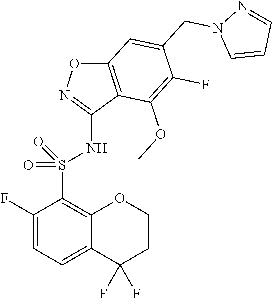 [SMILES: COc1c(F)c(Cn2cccn2)cc2onc(NS(=O)(=O)c3c(F)ccc4c3OCCC4(F)F)c12] | D |
| 106 | 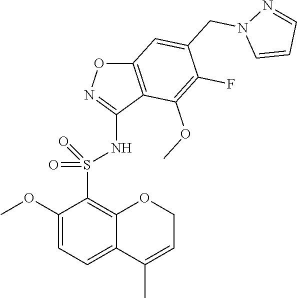 [SMILES: COc1ccc2c(c1S(=O)(=O)Nc1noc3cc(Cn4cccn4)c(F)c(OC)c13)OCC=C2C] | A |
| 107 | 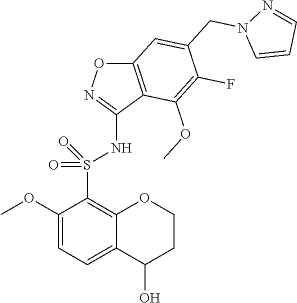 [SMILES: COc1ccc2c(c1S(=O)(=O)Nc1noc3cc(Cn4cccn4)c(F)c(OC)c13)OCCC2O] | B |
| 108 | 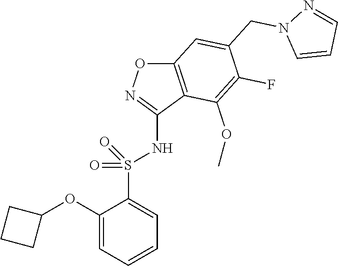 [SMILES: COc1c(F)c(Cn2cccn2)cc2onc(NS(=O)(=O)c3ccccc3OC3CCC3)c12] | B |
| 109 | 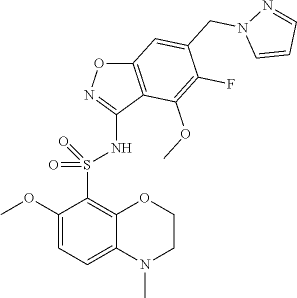 [SMILES: COc1ccc2c(c1S(=O)(=O)Nc1noc3cc(Cn4cccn4)c(F)c(OC)c13)OCCN2C] | A |
| 110 | 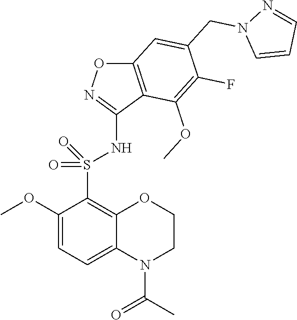 [SMILES: COc1ccc2c(c1S(=O)(=O)Nc1noc3cc(Cn4cccn4)c(F)c(OC)c13)OCCN2C(C)=O] | B |
| 111 | 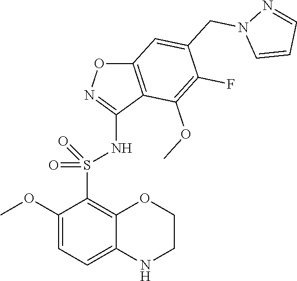 [SMILES: COc1ccc2c(c1S(=O)(=O)Nc1noc3cc(Cn4cccn4)c(F)c(OC)c13)OCCN2] | A |
| 112 | 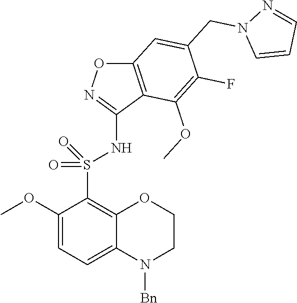 [SMILES: COc1ccc2c(c1S(=O)(=O)Nc1noc3cc(Cn4cccn4)c(F)c(OC)c13)OCCN2Cc1ccccc1] | C |
| 120 | 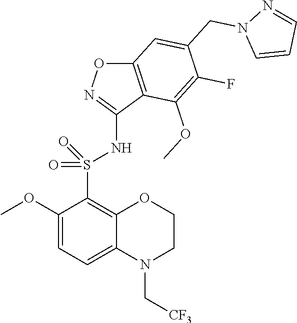 [SMILES: COc1ccc2c(c1S(=O)(=O)Nc1noc3cc(Cn4cccn4)c(F)c(OC)c13)OCCN2CC(F)(F)F] | C |
| 121 | 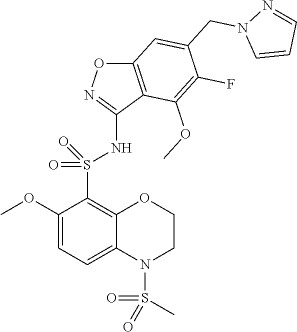 [SMILES: COc1ccc2c(c1S(=O)(=O)Nc1noc3cc(Cn4cccn4)c(F)c(OC)c13)OCCN2S(C)(=O)=O] | C |

Conclusion: Under the conditions in the inhibition experiment on the proliferation of the breast cancer cell line ZR-75-1 with high expression of KAT6A/B, some of the compounds of the present disclosure were potent inhibitors, among which compounds 15-16, 26-28, 35-40, 42-44, 101, 104, 106, 109, and 111 were more potent inhibitors against KAT6A/B compared to the reference compounds PF-9363, PF-8144, and PF-0128. The reference compounds PF-9363, PF-8144, and PF-0128 are disclosed in the patent WO2020/254946A1;
the reference compounds PF-9363 and PF-8144 are also disclosed in WO2022/013369A1.

### Test Example 2: Selective Inhibitory Effects of Test Compounds on Human KAT6 Enzymes (Radioisotope Method)

### Test Method

### 1) Experimental Reagents and Instruments

1. KAT6A (ACTIVE MOTIF, Cat. No. 81223)
2. KAT5 (BPS, Cat. No. 79422)
3. KAT7 (ACTIVE MOTIF, Cat. No. 31889)
4. KAT6B (ACTIVE MOTIF, Cat. No. 81224)
5. Acetyl coenzyme A, [Acetyl-³H, 250 μCi (9.25 MBq](PEIRKIN4 ELMER, Cat. No. NET290250UC)
6. Acetyl coenzyme A (CAYMVAN, Cat. No. 16160)
7. 384-well Flashplate (Perkin Elmer, Cat. No. SMP410A001PK)
8. Garcinol (MCE, Cat. No. HY-107569)
9. Automatic microplate pipetting system (Precision, PRC384U, BioTek)
10. Ultrasonic nanoliter liquid handling system (Echo 650 Liquid Handler, Echo 650, Labcyte)
11. Centrifuge (Avanti J-15R, Beckman Coulter)
12. Liquid scintillation counter (Microplate Counter, Microbeta2, Perkin Elmer)

### 2) Reagent Preparation

1. For KAT6A and KAT7, a 1× assay buffer was prepared as follows: 10 mM Tris-HCl pH 8.0, 0.01% Tween-20, and 1 mM DTT; for KAT6B and KAT5, a 1× assay buffer was prepared as follows: 50 mM Tris-HCl pH 8.0, 0.05% Tween-20, and 1 mM DTT.
2. Compound preparation: For KAT6A, KAT6B, and KAT7, the final concentration of the compounds started at 1 μM, followed by a 5-fold serial dilution across a total of 7 concentration gradients; for KAT5, the final concentration of the compounds started at 10 μM, followed by a 5-fold serial dilution across a total of 7 concentration gradients.
3. Enzyme solution preparation: The final concentrations were prepared using the respective 1× assay buffers, as shown in Table 4.
4. Substrate reaction solution preparation: The mixed substrates of H4 peptide and [3H]acetyl coenzyme A at final concentrations were prepared using the respective 1× assay buffers, as shown in Table 4.

### 3) Experimental Procedures

1. The enzyme solution was transferred to 384-well reaction plates at 10 μL/well. For each negative control well, 10 μL of the 1× assay buffer was transferred. The plates were then centrifuged at 1000 RPM for 1 min and incubated at room temperature for 15 min.
2. The substrate reaction solution was added at 10 μL/well, and the plates were centrifuged at 1000 RPM for 1 min to start the reaction.
3. The samples for KAT6A, KAT6B, KAT7, KAT8, and KAT5 were then incubated at room temperature for 60 min, 120 min, 45 min, 60 min, and 120 min, respectively.
4. Stop solution preparation: A stop solution was prepared by diluting 50 M cold Ac-CoA in 1× buffer.
5. The stop solution was added at 10 μL/well, and the plates were centrifuged at 100 RPM for 1 min to stop the reaction.
6. The mixtures in the reaction plates were transferred at 25 μL/well to new plates (Streptavidin FlashPlate HTS PLUS, High Capacity, 384-well).
7. The plates were incubated at room temperature for at least 60 min and then
8. read using MicroBeta2.

TABLE 4
Final concentrations of prepared enzyme solutions
and substrate reaction solutions

| | KAT5 | | | KAT7 |
|---|---|---|---|---|
| | (full- | KAT6A | KAT6B | (full- |
| Item | length) | (488-778) | (718-1008) | length) |
| [Enzyme] nM | 2.5 | 2.5 | 5 | 2 |
| [Biotin-H4(1-30)] nM | 400 | / | 400 | 600 |
| [Biotin-H3(1-21)] nM | / | 400 | / | / |
| [3H-Ac-CoA] nM | 800 | 250 | 560 | 550 |
| Stop reagent [Cold | 50 | 50 | 50 | 50 |
| Ac-CoA] μM | | | | |

### 4) Data Analysis

Raw data were obtained in EXCEL, and the inhibition rates were calculated:

$$Y = ( Max signal - Sample signal ) / ( Max signal - Min signal ) \times 100 \qquad Formula ( 1 )$$

$$Y : inhibition rate ;$$

Max signal: reaction signal from enzyme and substrate;
Sample signal: signal from sample;
Min signal: signal from substrate only (negative control).

The inhibition rate data were imported into XLFit Excel, and IC₅₀ values were obtained using formula (2):

$$Y = Bottom + ( Top - Bottom ) / ( 1 + ( ( {IC}_{50} / X ) \times HillSlope ) ) \qquad Formula ( 2 )$$

$$X : compound concentration ;$$

Top: value of the top plateau of the fitted regression curve;
Bottom: value of the bottom plateau of the fitted regression curve;
HillSlope: slope of the fitted regression curve.

The IC₅₀ value was substituted into formula (3) to obtain the enzymatic activity inhibition constant Ki:

$$Ki = {IC}_{50} / ( 1 + [ S ] / Km ) \qquad Formula ( 3 )$$

[S]: substrate concentration; Km: Michaelis constant.

### 5) Data Results

TABLE 5
Inhibitory activity of the compounds of the present
disclosure against KAT5/KAT6A/KAT6B/KAT7

| Compound | KAT6A/ | KAT6B/ | KAT5/ | KAT5/ | KAT5/ | KAT7/ | KAT7/ | KAT7/ |
|---|---|---|---|---|---|---|---|---|
| No. | Ki (nM) | Ki (nM) | Ki (nM) | KAT6A | KAT6B | Ki (nM) | KAT6A | KAT6B |
| PF-8144 | 0.58 | 5.18 | 672.32 | 1159 | 130 | 85.66 | 148 | 17 |
| 28 | 0.20 | 0.90 | 648.64 | 3243 | 721 | 40.59 | 203 | 45 |
| 101 | 0.21 | 0.65 | 79.61 | 379 | 122 | 7.39 | 35 | 11 |
| 109 | 0.47 | 0.90 | 286.20 | 609 | 318 | 54.68 | 116 | 61 |
| 110 | 0.29 | 1.60 | 1150.35 | 3967 | 719 | 64.53 | 223 | 40 |
| 114 | 0.38 | 2.49 | 1854.26 | 4880 | 745 | 173.88 | 458 | 70 |
| 115 | 0.63 | 6.48 | 5038.76 | 7998 | 778 | 492.57 | 782 | 76 |
| 121 | 0.28 | 0.90 | 576.94 | 2061 | 641 | 96.05 | 343 | 107 |

Conclusion: Some of the compounds of the present disclosure were potent KAT6A/KAT6B inhibitors, among which compounds 28, 101, 109, 110, 114, and 121 were more potent inhibitors against KAT6A/KAT6B compared to the reference compound PF-8144; compounds 28, 109, 110, 114, 115, and 121 exhibited better selective inhibitory effects on KAT6A/KAT6B compared to KAT5 or KAT7; some of the compounds possessed both stronger inhibitory activity and better selectivity.

### Test Example 3: Determination of Pharmacokinetic Data of Compounds in SD (Sprague Dawley) Rats

### Assay Objective

The pharmacokinetic characteristics of some of the compounds of the present disclosure were evaluated by a pharmacokinetic experiment in SD rats.

### Test Method

Female SD rats aged 6-8 weeks from Shanghai Jihui Laboratory Animal Care Co., Ltd. were selected. The test animals were fasted overnight before administration, and feeding was resumed 4 h after administration. The compounds were dissolved in 5% DMSO+5% Tween 80+90% normal saline. For a single intravenous administration, the administration dose was 1 mg/kg, and the administration volume was 2 mL/kg. For a single intragastric administration, the administration dose was 2 mg/kg, and the administration volume was 10 mL/kg. Blood was collected at 0.083 h (for intravenous administration only), 0.25 h, 0.5 h, 1 h, 2 h, 4 h, 8 h, and 24 h after administration. At each indicated time point, about 150 μL of blood sample was collected from each rat via the vein and placed into an EDTA-K₂ tube. Each sample was centrifuged (2000 g, 4° C., 5 min) within 15 min after sample collection to obtain plasma, which was then stored at −70° C. until analysis. The concentration of the corresponding test compound in the SD rat plasma was determined using UPLC-MS/MS.

### Pharmacokinetic Parameter Results

TABLE 6
Pharmacokinetic test results of representative
compounds of the present disclosure in SD rats

| | Route/dose of | | | | | |
|---|---|---|---|---|---|---|
| Compound | administration | CL | Vdss | AUC_{0-inf} | T_{1/2} | F |
| No. | (mg/kg) | (mL/min/Kg) | (L/Kg) | (hr*ng/mL) | (h) | (%) |
| PF-8144 | i.v./1 | 0.597 | 0.143 | 28318 | 3.16 | — |
| | p.o./2 | — | — | 55494 | 3.79 | 98 |
| 28 | i.v./1 | 0.761 | 0.295 | 22573 | 5.87 | — |
| | p.o./2 | — | — | 42761 | 6.73 | 94.7 |
| 42 | i.v./1 | 1.69 | 0.261 | 10249 | 3.79 | — |
| | p.o./2 | — | — | 11273 | 3.31 | 55 |
| 101 | i.v./1 | 0.35 | 0.237 | 48124 | 8.88 | — |
| | p.o./2 | — | — | 89071 | 9.21 | 92.5 |
| 104 | i.v./1 | 3.98 | 0.705 | 4200 | 2.83 | — |
| | p.o./2 | — | — | 6109 | 2.13 | 72.7 |
| 109 | i.v./1 | 0.185 | 0.119 | 90718 | 8.57 | — |
| | p.o./2 | — | — | 187103 | 9.73 | 96.0 |
| 114 | i.v./1 | 0.462 | 0.608 | 39220 | 17.2 | — |
| | p.o./2 | — | — | 97873 | 13.0 | 144 |
| 115 | i.v./1 | 0.363 | 0.408 | 47541 | 14.3 | — |
| | p.o./2 | — | — | 109392 | 15.9 | 105 |

Note:
CL represents clearance rate; Vdss represents apparent volume of distribution; AUC_{0-inf} represents area under the curve (_{0-inf}); T_{1/2} represents half-life; F represents bioavailability.
Conclusion: Under the conditions in this experiment, the representative compounds disclosed herein exhibited high bioavailability and exposure levels, with some of the representative compounds demonstrating lower in vivo clearance rates, longer half-lives, and higher exposure levels compared to the reference compound PF-8144.

### Test Example 4: Assay for Effects of Compounds on Histone Acetylation in Estrogen Receptor-Positive Breast Cancer Cells

### Assay Method: Western Blotting Method (Immunoblotting Assay)

### Materials and method

The effects of the test compounds on the acetylation level of H3K23 in ZR-75-1 cells (Nanjing Cobioer, CBP60402) were assayed.
1. On day 1, the cells were plated at 400,000 cells/well (2 mL/well) and cultured overnight in an incubator at 37° C. with 5% CO₂.
2. On day 2, compounds were added starting at 5 μM, with a 10-fold serial dilution across a total of 3 concentration gradients, and DMSO was added to the negative control wells.
3. On day 6, after 5 days of incubation with compounds, protein samples were collected: An ice box, pre-cooled PBS (4° C.), a cell lysis buffer RIPA (Beyotime, P0013B), and a protease inhibitor (100x) (Thermo Scientific™, 87786) were prepared. Then, 120 μL of the cell lysis buffer was added to each well. The mixture was placed on ice for about 15 min. Then, the cell lysate was taken out and centrifuged at 12,000 rpm for 20 min, and the supernatant was collected.
4. Protein quantification by BCA method (Beyotime, P0009)
Standards (8 concentrations) were prepared, and the samples and the standards were added to a 96-well plate. The samples were subjected to a 10-fold serial dilution (18 μL of PBS+2 μL of sample supernatant). A working solution was prepared and then added. The prepared working solution was a BCA working solution (solution A:solution B=50:1). After the addition of the working solution, the mixture was baked in an oven at 37° C. for 30 min. The absorbance was then measured using a microplate reader. Loading Buffer (Biofuraw™ LDS Sample Buffer, With Reducing, 180-8210D) was added to adjust the protein concentration to 1.4 g/L, followed by incubation in a metal bath at 100° C. for 10 min.

### 5. Electrophoresis and Development

The samples were taken out and thawed on ice. Before loading, they were vortexed and centrifuged. A pre-cast gel (Tanon™ 4-20% Bis-tris gradient pre-cast gel, 180-9115H) was installed, and the samples were loaded. The electrophoresis system was run at 120 V for 40 min.

Subsequently, the proteins were transferred to a PVDF membrane, and then the membrane was placed in 5% BSA for blocking for 1 h. Corresponding antibodies (Acetyl-Histone H3 (Lys23) (D6Y7M) Rabbit mAb (14932T, CST), Histone H4 (L64C1) Mouse mAb (2935T, CST), j-Actin Antibody (4967S, CST)) were added, and then the membrane was incubated overnight at 4° C.

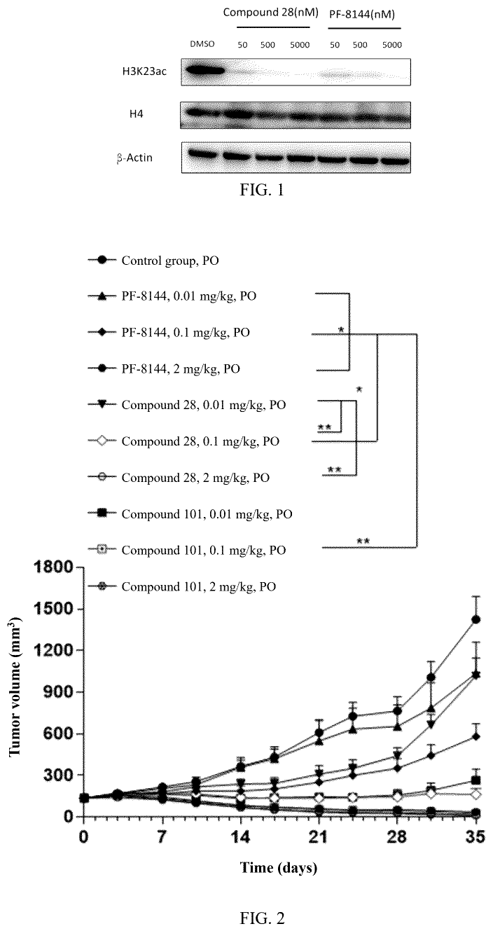

The next day, the PVDF membrane was taken out and washed (8 min×3) with TBST (with 0.5% Tween 20). Horseradish peroxidase-conjugated secondary antibodies (Anti-rabbit IgG, 7074P2 and Anti-mouse IgG, 7076P2, CST) were prepared and then incubated with the membrane on a shaker for 1 h. After the incubation was completed, the membrane was washed (8 min×3) with TBST. After the washing, a luminescence solution (Tanon, 180-506) was added for exposure. After the development was completed, the strips that had been incubated with Acetyl-Histone H3 (Lys23) (D6Y7M) Rabbit mAb were washed twice with TBST, followed by protein elution (Thermo Fisher, 46430). Subsequently, the above-mentioned blocking was performed again, followed by incubation with Histone H4 (L64C1) Mouse mAb and Anti-mouse IgG. Then, development was carried out again. The development results are shown in FIG. 1.

### 6. Results:

The test compound 28 and PF-8144 could both significantly reduce the acetylation level of H3K23.

### Test Example 5: Anti-Tumor Effects of Test Compounds in ER⁺KAT6^{high} Breast Cancer Cell-Xenograft Tumor Model

Assay method: ZR-75-1 cells were subcutaneously inoculated into the right back of each mouse to evaluate the efficacy of the compounds in the subcutaneous xenograft tumor model of ZR-75-1 cells.

### Materials and Method

1. Cell culture: ZR-75-1 cells were cultured as a monolayer in vitro under the following culture conditions: an RPMI 1640 medium supplemented with 10% fetal bovine serum and 1% Anti-anti (Antibiotic-Antimycotic), incubated in an incubator at 37° C. with 5% CO₂. The cells were digested with trypsin-EDTA twice a week for passaging as per conventional practice. When the cell saturation reached 80%-90% and the quantity met the requirement, the cells were harvested, counted, and inoculated.
2. Animals: Balb/c nude mice, female, 6-8 weeks old, weighing 18-22 g. A total of 128 mice were required (80 mice were enrolled). They were provided by Vital River.
3. Tumor inoculation: Two days before tumor inoculation, 17-beta estradiol tablets (0.18 mg) were subcutaneously administered, and 10 million ZR-75-1 tumor cells+matrigel (total 200 L) were subcutaneously inoculated into the right back of each mouse. When the mean tumor volume reached about 137 mm³, the mice were grouped for administration.
4. Experimental administration: The compounds were dissolved in 5% DMSO+5% Tween 80+90% Saline, and administered intragastrically (PO) daily for five weeks.
5. Experimental indicators: The experimental indicators were to investigate whether the tumor growth was inhibited or delayed or the tumor was cured. Tumor diameters were measured twice weekly using a vernier caliper. The tumor volume was calculated using the following formula: V=0.5a×b², where a and b represent the long diameter and short diameter of the tumor, respectively.
6. The anti-tumor efficacy of the compounds was evaluated by TGI (%) or relative tumor proliferation rate T/C (%). TGI (%) refers to the tumor growth inhibition rate. Calculation of TGI (%):TGI (%)=[(1−(mean tumor volume at the end of administration in a treatment group—mean tumor volume at the start of administration in the treatment group))/(mean tumor volume at the end of treatment in the solvent control group—mean tumor volume at the start of treatment in the solvent control group)]×100%. The calculation formula for the relative tumor proliferation rate T/C (%) was as follows: T/C %=TRTV/CRTV×100% (TRTV:RTV of the treatment group; CRTV:RTV of the negative control group). The relative tumor volume (RTV) was calculated based on the tumor measurement results using the following formula: RTV=Vt/V0, where V0 represents the mean tumor volume measured at the time of grouping for administration (i.e., PG-D0), and Vt represents the mean tumor volume at a certain measurement. TRTV and CRTV were derived from data measured on the same day.

### 7. Statistical Analysis

The mean and standard error of mean (SEM) of tumor volume at each time point for each group were included. The differences in tumor volume and tumor weight among the groups were statistically analyzed. For comparisons among three or more groups, one-way ANOVA was used for analysis. If the F-value showed a significant difference, the Games-Howell method was applied for testing. If the F-value showed no significant difference, the Dunnett's (2-sided) method was applied for analysis. The analysis was performed using SPSS 17.0, and p<0.05 was considered to indicate a significant difference.

For comparisons between different doses of the same drug or different drugs at the same dose, two-way ANOVA was used for analysis, and the Bonferroni method was applied for testing. The analysis was performed using GraphPad Prism 9.0, and p<0.05 was considered to indicate a significant difference. On day 35 after the start of administration, the data between different doses of the same drug or different drugs at the same dose were analyzed using one-way ANOVA. If the F-value showed a significant difference, the Games-Howell method was applied for testing. If the F-value showed no significant difference, the Dunnett's (2-sided) method was applied for analysis. The analysis was performed using SPSS 17.0, and p<0.05 was considered to indicate a significant difference.

TABLE 7
Animal experimental grouping and administration regimen

| | Number | | Dose | Preparation | Route of | |
|---|---|---|---|---|---|---|
| Group | (n) | Compound | (mg/kg) | volume (μL/g) | administration | Duration |
| 1 | 8 | Control group | — | 10 | PO | QD*5 W |
| | | (solvent) | | | | |
| 2 | 8 | PF-8144 | 0.01 | 10 | PO | QD*5 W |
| 3 | 8 | PF-8144 | 0.1 | 10 | PO | QD*5 W |
| 4 | 8 | PF-8144 | 2 | 10 | PO | QD*5 W |
| 5 | 8 | Compound 28 | 0.01 | 10 | PO | QD*5 W |
| 6 | 8 | Compound 28 | 0.1 | 10 | PO | QD*5 W |
| 7 | 8 | Compound 28 | 2 | 10 | PO | QD*5 W |
| 8 | 8 | Compound 101 | 0.01 | 10 | PO | QD*5 W |
| 9 | 8 | Compound 101 | 0.1 | 10 | PO | QD*5 W |
| 10 | 8 | Compound 101 | 2 | 10 | PO | QD*5 W |

### 8. Results

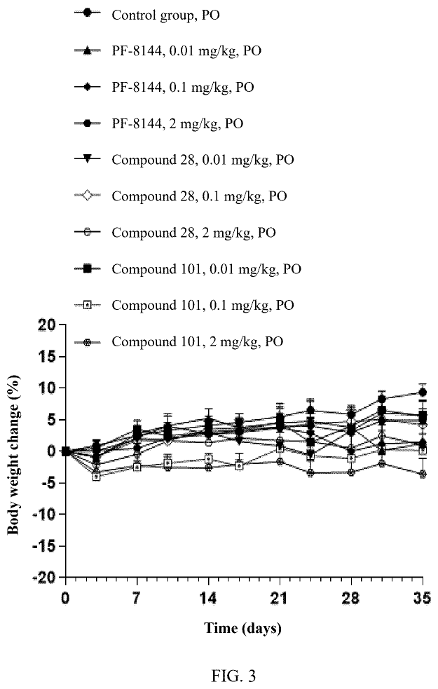

In this experiment, the in vivo efficacy of the compounds in the human breast cancer ZR-75-1 cell xenograft tumor model was evaluated. The tumor volumes and body weights of the mice in each group at different time points are shown in FIGS. 2 and 3.

On day 35 after the start of the administration, the tumor volume of the tumor-bearing mice in the control group reached 1,424 mm³. Compared to the control group (solvent), the mean tumor volumes for PF-8144 at 0.01 mg/kg, 0.1 mg/kg, and 2 mg/kg on day 35 after the grouping for administration were 1,035 mm³ (T/C=72.71%, TGI=30.19%, p=0.911), 580 mm³ (T/C=40.77%, TGI=65.53%, p=0.022), and 36 mm³ (T/C=2.51%, TGI=107.85%, p=0.001), respectively. The mean tumor volumes for compound 28 at 0.01 mg/kg, 0.1 mg/kg, and 2 mg/kg were 1,017 mm³ (T/C=76.14%, TGI=30.94%, p=0.654), 162 mm³ (T/C=11.36%, TGI=98.07%, p=0.002), and 12 mm³ (T/C=0.84%, TGI=109.69%, p=0.001), respectively. The mean tumor volumes for compound 101 at 0.01 mg/kg, 0.1 mg/kg, and 2 mg/kg were 264 mm³ (T/C=19.06%, TGI=89.81%, p=0.002), 25 mm³ (T/C=1.79%, TGI=108.64%, p=0.001), and 14 mm³ (T/C=0.96%, TGI=109.56%, p=0.001), respectively. Except for the 0.01 mg/kg dose groups of PF-8144 and compound 28, all administration groups exhibited significant anti-tumor effects. At the dose of 0.1 mg/kg, compound 28 and compound 101 were significantly superior in efficacy to PF-8144. Meanwhile, at all doses, the body weight changes in the compound 28 group and the compound 101 group showed no statistically significant difference compared to the PF-8144 group.

### Test Example 6: Anti-Tumor Effects of Test Compounds in ER⁺KAT6^{high} Breast Cancer Cell-Xenograft Tumor Model

Assay method: ZR-75-1 cells were subcutaneously inoculated into the right back of each mouse to evaluate the efficacy of the compounds in the subcutaneous xenograft tumor model of ZR-75-1 cells.

### Materials and Method

1. Cell culture: ZR-75-1 cells were cultured as a monolayer in vitro under the following culture conditions: an RPMI 1640 medium supplemented with 10% fetal bovine serum and 1% Anti-anti (Antibiotic-Antimycotic), incubated in an incubator at 37° C. with 5% CO₂. The cells were digested with trypsin-EDTA twice a week for passaging as per conventional practice. When the cell saturation reached 80%-90% and the quantity met the requirement, the cells were harvested, counted, and inoculated.
2. Animals: Balb/c nude mice, female, 6-8 weeks old, weighing 18-22 g. A total of 115 mice were required (72 mice were enrolled). They were provided by Vital River or other qualified suppliers.
3. Tumor inoculation: Two days before tumor inoculation, 17-beta estradiol tablets (0.18 mg) were subcutaneously administered, and 10 million ZR-75-1 tumor cells+matrigel (total 200 L) were subcutaneously inoculated into the right back of each mouse. When the mean tumor volume reached about 131 mm³, the mice were grouped for administration.
4. Experimental administration: The compounds were dissolved in 5% DMSO+5% Tween 80+90% Saline or 10% DMSO+10% Solutol+80% (10% HPP-CD in saline), and administered intragastrically (PO) daily for five weeks.
5. Experimental indicators: The experimental indicators were to investigate whether the tumor growth was inhibited or delayed or the tumor was cured. Tumor diameters were measured twice weekly using a vernier caliper. The tumor volume was calculated using the following formula: V=0.5a×b², where a and b represent the long diameter and short diameter of the tumor, respectively.
6. The anti-tumor efficacy of the compounds was evaluated by TGI (%) or relative tumor proliferation rate T/C (%). TGI (%) refers to the tumor growth inhibition rate. Calculation of TGI (%):TGI (%)=[(1−(mean tumor volume at the end of administration in a treatment group—mean tumor volume at the start of administration in the treatment group))/(mean tumor volume at the end of treatment in the solvent control group—mean tumor volume at the start of treatment in the solvent control group)]×100%. The calculation formula for the relative tumor proliferation rate T/C (%) was as follows: T/C %=TRTV/CRTV×100% (TRTV:RTV of the treatment group; CRTV:RTV of the negative control group). The relative tumor volume (RTV) was calculated based on the tumor measurement results using the following formula: RTV=Vt/V0, where V0 represents the mean tumor volume measured at the time of grouping for administration (i.e., PG-DO), and Vt represents the mean tumor volume at a certain measurement. TRTV and CRTV were derived from data measured on the same day.

### 7. Statistical Analysis

The mean and standard error of mean (SEM) of tumor volume at each time point for each group were included. The differences in tumor volume and tumor weight among the groups were statistically analyzed. For comparisons among three or more groups, one-way ANOVA was used for analysis. If the F-value showed a significant difference, the Games-Howell method was applied for testing. If the F-value showed no significant difference, the Dunnett's (2-sided) method was applied for analysis. The analysis was performed using SPSS 17.0, and p<0.05 was considered to indicate a significant difference.

For comparisons between different doses of the same drug or different drugs at the same dose, two-way ANOVA was used for analysis, and the Bonferroni method was applied for testing. The analysis was performed using GraphPad Prism 9.0, and p<0.05 was considered to indicate a significant difference. On day 34 after the start of administration, the data between different doses of the same drug or different drugs at the same dose were analyzed using one-way ANOVA. If the F-value showed a significant difference, the Games-Howell method was applied for testing. If the F-value showed no significant difference, the Dunnett's (2-sided) method was applied for analysis. The analysis was performed using SPSS 17.0, and p<0.05 was considered to indicate a significant difference.

TABLE 8
Animal experimental grouping and administration regimen

| | Number | | Dose | Preparation | Route of | |
|---|---|---|---|---|---|---|
| Group | (n) | Compound | (mg/kg) | volume (μL/g) | administration | Duration |
| 1 | 8 | Control group | — | 10 | PO | QD*5 W |
| | | (vehicle) | | | | |
| 2 | 8 | PF-8144 | 0.1 | 10 | PO | QD*5 W |
| 3 | 8 | PF-8144 | 0.3 | 10 | PO | QD*5 W |
| 4 | 8 | Compound 42 | 0.1 | 10 | PO | QD*5 W |
| 5 | 8 | Compound 42 | 0.3 | 10 | PO | QD*5 W |
| 6 | 8 | Compound 114 | 0.1 | 10 | PO | QD*5 W |
| 7 | 8 | Compound 114 | 0.3 | 10 | PO | QD*5 W |
| 8 | 8 | Compound 115 | 0.1 | 10 | PO | QD*5 W |
| 9 | 8 | Compound 115 | 0.3 | 10 | PO | QD*5 W |

### 8. Results

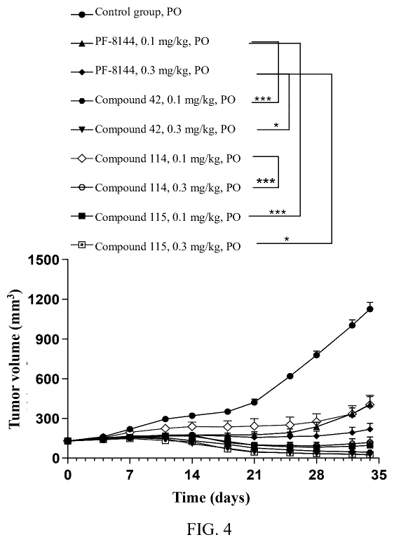

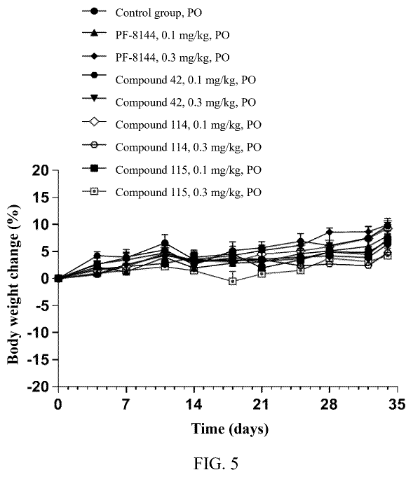

In this experiment, the in vivo efficacy of the compounds in the human breast cancer ZR-75-1 cell xenograft tumor model was evaluated. The tumor volumes and body weights of the mice in each group at different time points are shown in FIGS. 4 and 5.

On day 34 after the start of the administration, the tumor volume of the tumor-bearing mice in the control group reached 1,126 mm³. Compared to the control group (solvent), the mean tumor volumes for PF-8144 at 0.1 mg/kg and 0.3 mg/kg on day 34 after the grouping for administration were 407 mm³ (T/C=36.2%, TGI=72.2%, p<0.001) and 220 mm³ (T/C=19.5%, TGI=91.07%, p<0.001), respectively. The mean tumor volumes for compound 42 at 0.1 mg/kg and 0.3 mg/kg were 44 mm³ (T/C=3.9%, TGI=108.67%, p<0.001) and 25 mm³ (T/C=2.2%, TGI=110.58%, p<0.001), respectively. The mean tumor volumes for compound 114 at 0.1 mg/kg and 0.3 mg/kg were 403 mm³ (T/C=35.8%, TGI=72.61%, p<0.001) and 119 mm³ (T/C=10.5%, TGI=101.21%, p<0.001), respectively. The mean tumor volumes for compound 115 at 0.1 mg/kg and 0.3 mg/kg were 97 mm³ (T/C=8.6%, TGI=103.38%, p<0.001) and 27 mm³ (T/C=2.4%, TGI=110.47%, p<0.001), respectively. All administration groups exhibited significant anti-tumor effects. At the doses of 0.1 mg/kg and 0.3 mg/kg, compound 42 and compound 115 were significantly superior in efficacy to PF-8144. Meanwhile, at all doses, the body weight changes in the compound 42 group, the compound 114 group, and the compound 115 group showed no statistically significant difference compared to the PF-8144 group.

It will be appreciated that various modifications or changes may be made by those skilled in the art after reading the aforementioned content of the present disclosure, and such equivalent forms also fall within the scope defined by the appended claims of the present application.

1. A compound of formula (I) or a pharmaceutically acceptable salt, an ester, a stereoisomer, a tautomer, a polymorph, a hydrate, a solvate, an N-oxide, an isotopically labeled compound, a metabolite, or a prodrug thereof: 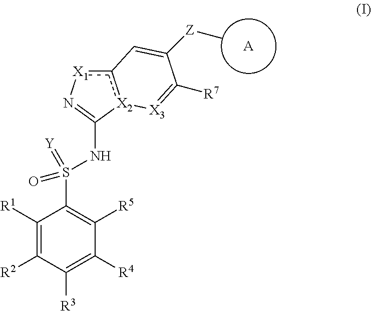
wherein:
the dashed line is an optional chemical bond;
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ halocycloalkyl, C₃₋₆ cycloalkoxy, C₃₋₆ halocycloalkoxy, 6- to 10-membered aryl, 5- to 10-membered heteroaryl, and C₁₋₆ hydroxyalkyl, or R⁴ and R⁵, together with the carbon atoms to which they are attached, form 4- to 10-membered heterocyclyl, 5- to 6-membered heteroaryl, 7- to 12-membered spiroheterocyclyl, or 8- to 15-membered fused heterocyclyl, wherein the 4- to 10-membered heterocyclyl, 5- to 6-membered heteroaryl, 7- to 12-membered spiroheterocyclyl, or 8- to 15-membered fused heterocyclyl is optionally substituted with one or more substituents selected from: hydroxyl, amino, halogen, C₁₋₆ alkyl, C₁₋₆ haloalkyl, C₁₋₆ hydroxyalkyl, C₁₋₆ alkoxy, C₁₋₆ haloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, oxo, ═CH₂, ═CHC₁₋₆ alkyl, ═C(C₁₋₆ alkyl)₂, ═CHC₁₋₆ haloalkyl, ═C(C₁₋₆ haloalkyl)₂, —NHC₁₋₆alkyl, —N(C₁₋₆ alkyl)₂, cyano, benzyl, C₃₋₆ cycloalkyl, C₃₋₆ heterocyclyl, C₃₋₆ halocycloalkyl, C₃₋₆ haloheterocyclyl, C₃₋₆ cycloalkoxy, C₃₋₆ halocycloalkoxy, 6- to 10-membered aryl, 5- to 10-membered heteroaryl, and C2-6 alkenyl; ring A is selected from 3- to 8-membered heterocyclyl, 6- to 10-membered aryl, and 5- to 10-membered heteroaryl and 6- to 12-membered fused heterocyclyl containing at least one nitrogen atom; ring A is optionally substituted with one or more substituents selected from: halogen, amino, hydroxyl, cyano, C₁₋₆ alkyl, C₁₋₆ haloalkyl, C₁₋₆ alkoxy, 3- to 8-membered cycloalkoxy, C₁₋₆ haloalkoxy, 3- to 8-membered halocycloalkoxy, oxo, and C₂₋₆ alkenyl;
X₁ is O, S, C, or N;
X₂ is N or C;
X₃ is N or CR₆;
R⁶ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy;
R⁷ is selected from a hydrogen atom, halogen, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, C₃₋₈ cycloalkoxy, and C₃₋₈ halocycloalkoxy; and
Y is O or NH;
Z is —CH₂—, —CF₂—, —C(O)—, O, —S(O)—, —S(O)₂—, or —NH—.

2. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the compound is a compound of formula (I1-1), (11-2), or (11-3), 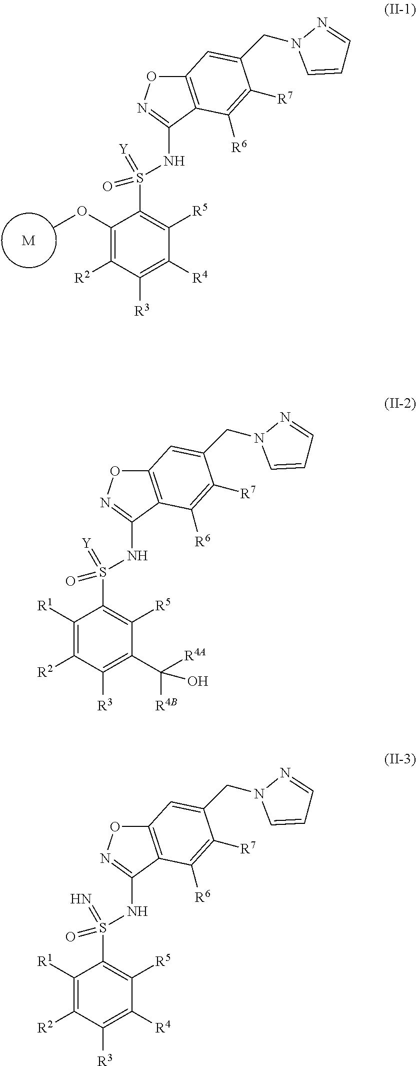
wherein:
R¹ is selected from C₁₋₄ alkoxy, C₁₋₄ haloalkoxy, C₃₋₆ cycloalkoxy, and C₃₋₆ halocycloalkoxy; preferably, R¹ is selected from methoxy, difluoromethoxy, trifluoromethoxy, —OCH₂CHF₂, cyclobutyloxy, and 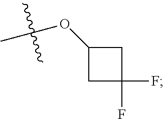 [SMILES: *OC1CC(F)(F)C1]
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy; preferably, R² and R³ are identical or different and are each independently selected from a hydrogen atom, halogen, C₁₋₄ alkyl, and C₁₋₄ alkoxy; more preferably, R² and R³ are identical or different and are each independently selected from a hydrogen atom and halogen; most preferably, both R² and R³ are hydrogen atoms;
R⁴ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ halocycloalkyl, C₃₋₆ cycloalkoxy, C₃₋₆ halocycloalkoxy, and C₁₋₆ hydroxyalkyl; preferably, R⁴ is selected from a hydrogen atom, C₁₋₆ alkyl, C₃₋₆ heterocyclyl, and C₁₋₆ hydroxyalkyl; more preferably, R⁴ is selected from a hydrogen atom, ethyl, isopropyl, and tert-butyl;
R^{4A} and R^{4B} are identical or different and are each independently selected from C₁₋₆ alkyl and C₁₋₆ haloalkyl; preferably, both R^{4A} and R^{4B} are methyl;
R⁵ is selected from a hydrogen atom, C₁₋₄ alkoxy, C₁₋₄ haloalkoxy, C₄₋₆ cycloalkoxy, and C₄₋₆ halocycloalkoxy; preferably, R⁵ is selected from a hydrogen atom, methoxy, difluoromethoxy, trifluoromethoxy, —OCH₂CHF₂, cyclobutyloxy, and 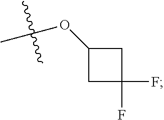 [SMILES: *OC1CC(F)(F)C1]
more preferably, R⁵ is a hydrogen atom or methoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; preferably, R⁶ and R⁷ are identical or different and are each independently selected from a hydrogen atom, a chlorine atom, a fluorine atom, methoxy, ethoxy, isopropoxy, difluoromethoxy, and cyclopropyl;
ring M is selected from 4- to 7-membered cycloalkyl and 4- to 7-membered halocycloalkyl;
preferably, ring M is selected from cyclobutyl and 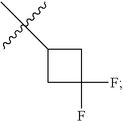 [SMILES: *C1CC(F)(F)C1]
Y is O or NH; preferably, Y is O.

3. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the compound is a compound of formula (III), 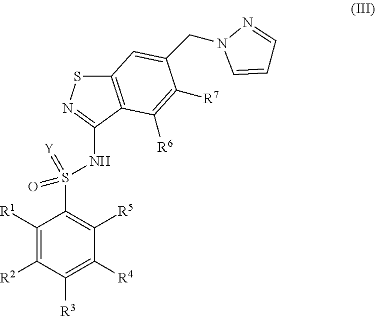
wherein:
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; preferably, R¹ is selected from C₁₋₆ alkoxy, C₁₋₆ haloalkoxy, C₃₋₆ cycloalkoxy, and C₃₋₆ halocycloalkoxy, and R² and R³ are identical or different and are each independently selected from a hydrogen atom, halogen, and C₁₋₆ alkyl; more preferably, R¹ is methoxy, and both R² and R³ are hydrogen atoms;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ cycloalkoxy, and C₁₋₆ hydroxyalkyl; preferably, R⁴ is selected from a hydrogen atom, C₁₋₆ alkyl, C₃₋₆ heterocyclyl, and C₁₋₆ hydroxyalkyl, and R⁵ is selected from a hydrogen atom, C₁₋₄ alkoxy, C₁₋₄ haloalkoxy, C₃₋₆ cycloalkoxy, and C₃₋₆ halocycloalkoxy; more preferably, R⁴ is selected from a hydrogen atom, ethyl, isopropyl, tert-butyl, and 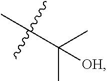 [SMILES: *C(C)(C)O]
and R⁵ is selected from a hydrogen atom, methoxy, difluoromethoxy, trifluoromethoxy, —OCH₂CHF₂, cyclobutyloxy, and 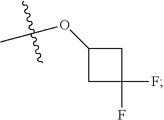 [SMILES: *OC1CC(F)(F)C1]
most preferably, R⁴ is a hydrogen atom, and R⁵ is a hydrogen atom or methoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; preferably, R⁶ and R⁷ are identical or different and are each independently selected from a hydrogen atom, a chlorine atom, a fluorine atom, methoxy, ethoxy, isopropoxy, difluoromethoxy, and cyclopropyl;
Y is O or NH; preferably, Y is O.

4. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the compound is a compound of formula (IV), 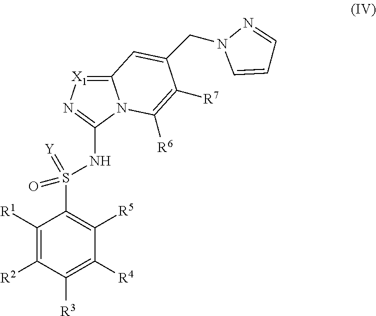
wherein:
R¹, R², and R³ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; preferably, R¹ is selected from C₁₋₆ alkoxy, C₁₋₆ haloalkoxy, C₃₋₆ cycloalkoxy, and C₃₋₆ halocycloalkoxy, and R² and R³ are identical or different and are each independently selected from a hydrogen atom, halogen, and C₁₋₆ alkyl; more preferably, R¹ is methoxy, and both R² and R³ are hydrogen atoms;
R⁴ and R⁵ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, C₃₋₆ cycloalkoxy, and C₁₋₆ hydroxyalkyl; preferably, R⁴ is selected from a hydrogen atom, C₁₋₆ alkyl, C₃₋₆ heterocyclyl, and C₁₋₆ hydroxyalkyl, and R⁵ is selected from a hydrogen atom, C₁₋₄ alkoxy, C₁₋₄ haloalkoxy, C₃₋₆ cycloalkoxy, and C₃₋₆ halocycloalkoxy; more preferably, R⁴ is selected from a hydrogen atom, ethyl, isopropyl, tert-butyl, and 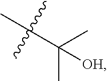 [SMILES: *C(C)(C)O]
and R⁵ is selected from a hydrogen atom, methoxy, difluoromethoxy, trifluoromethoxy, —OCH₂CHF₂, cyclobutyloxy, and 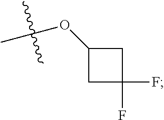 [SMILES: *OC1CC(F)(F)C1]
most preferably, R⁴ is a hydrogen atom, and R⁵ is a hydrogen atom or methoxy; R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy; preferably, R⁶ and R⁷ are identical or different and are each independently selected from a hydrogen atom, a chlorine atom, a fluorine atom, methoxy, ethoxy, isopropoxy, difluoromethoxy, and cyclopropyl;
X₁ is C or N;
Y is O or NH; preferably, Y is O.

5. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein
the compound is a compound of formula (V), 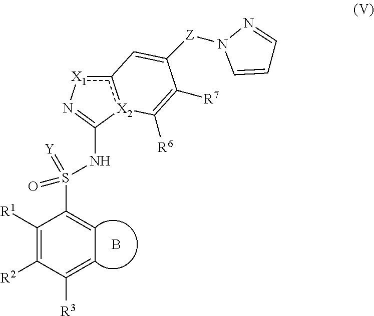
wherein:
the dashed line is an optional chemical bond;
R¹, R², R³, R⁶, R⁷, Y, X₁, X₂, and Z are as defined in claim 1;
ring B is selected from 4- to 10-membered heterocyclyl, 5- to 6-membered heteroaryl, and 7- to 12-membered spiroheterocyclyl; ring B is optionally substituted with one or more substituents selected from: hydroxyl, amino, halogen, C₁₋₆ alkyl, C₁₋₆ haloalkyl, C₁₋₆ alkoxy, C₁₋₆ haloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, oxo, ═CH₂, ═CHC₁₋₆ alkyl, ═C(C₁₋₆ alkyl)₂, ═CHC₁₋₆ haloalkyl, ═C(C₁₋₆ haloalkyl)₂, —NHC₁₋₆ alkyl, —N(C₁₋₆ alkyl)₂, cyano, benzyl, C₃₋₆ cycloalkyl, C₃₋₆ heterocyclyl, C₃₋₆ halocycloalkyl, C₃₋₆ cycloalkoxy, C₃₋₆ halocycloalkoxy, 6- to 10-membered aryl, 5- to 10-membered heteroaryl, and C₂₋₆ alkenyl.

6. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein in the compound of formula (V), ring B is 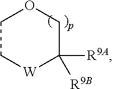
wherein the dashed line represents a chemical bond shared by ring B and the benzene ring; W is selected from —O—, —NR^{9C}—, —CR^{9D}R^{9E}—, 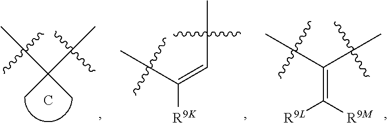
and —C(O)—, and W is connected to a carbon atom ortho to the carbon atom where R³ is located on the benzene ring;
p is 0, 1, or 2;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, benzyl, 6- to 10-membered aryl, 5- to 10-membered heteroaryl, and C₂₋₆ alkenyl;
R^{9D} and R^{9E} are identical or different and are each independently selected from a hydrogen atom, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, —NHC₁₋₆ alkyl, —N(C₁₋₆ alkyl)₂, C₃₋₆ cycloalkyl, C₃₋₆ halocycloalkyl, C₃₋₆ cycloalkoxy, C₃₋₆ halocycloalkoxy, and hydroxyl;
ring C is selected from 3- to 8-membered cycloalkyl, 3- to 8-membered heterocyclyl, 6- to 12-membered spirocyclyl, 6- to 12-membered spiroheterocyclyl, and 5- to 12-membered bridged heterocyclyl; ring C is optionally substituted with one or more substituents selected from halogen, hydroxyl, amino, C₁₋₆ alkyl, and C₁₋₆ haloalkyl;
R^{9K} is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, 6- to 10-membered aryl, 5- to 10-membered heteroaryl, and hydroxyl, wherein the C₃₋₆ cycloalkyl and C₃₋₆ heterocyclyl are optionally substituted with one or more substituents selected from: halogen, hydroxyl, methyl, and methoxy;
R^{9M} and R^{9L} are identical or different and are each independently selected from a hydrogen atom, C₁₋₆ alkyl, and C₁₋₆ haloalkyl.

7. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein in the compound of formula (V), W is selected from —O—, —NR^{9C}—, 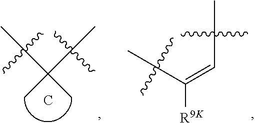
and —C(O)—; relatively preferably, W is selected from —O—, —NR^{9C}—, 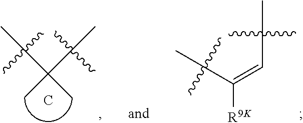
more preferably, W is selected from —O— and —NR^{9C}—.

8. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein the compound is a compound of formula (VI-1), (VI-2), (VI-3), or (VI-4), 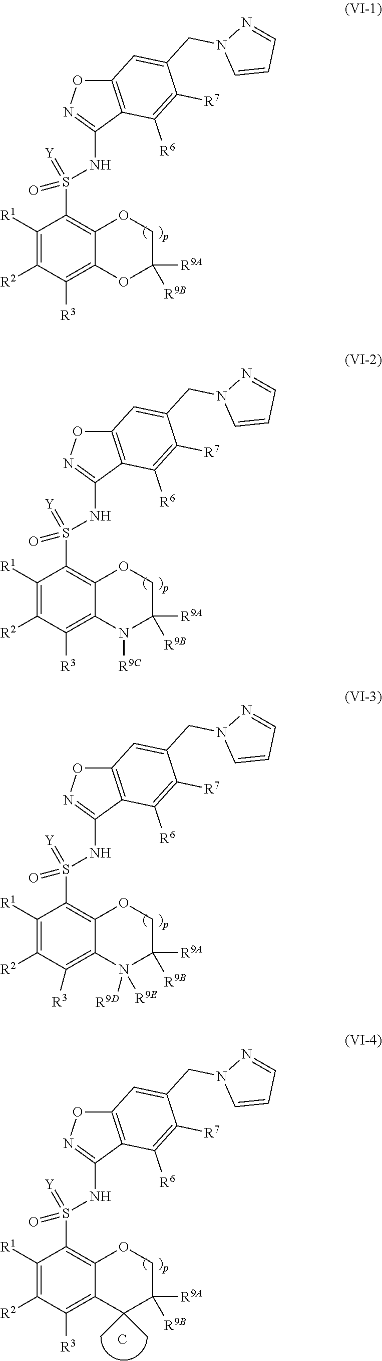
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, benzyl, 6- to 10-membered aryl, 5- to 10-membered heteroaryl, and C₂₋₆ alkenyl;
R^{9D} and R^{9E} are identical or different and are each independently selected from a hydrogen atom, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, —NHC₁₋₆ alkyl, —N(C₁₋₆ alkyl)₂, C₃₋₆ cycloalkyl, C₃₋₆ halocycloalkyl, C₃₋₆ cycloalkoxy, C₃₋₆ halocycloalkoxy, and hydroxyl;
ring C is selected from 3- to 8-membered cycloalkyl, 3- to 8-membered heterocyclyl, 6- to 12-membered spirocyclyl, 6- to 12-membered spiroheterocyclyl, and 5- to 12-membered bridged heterocyclyl; ring C is optionally substituted with one or more substituents selected from halogen, hydroxyl, amino, C₁₋₆ alkyl, and C₁₋₆ haloalkyl;
Y is O or NH;
p is 0, 1, or 2.

9. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R¹ is selected from a hydrogen atom, C₁₋₄ alkoxy, C₁₋₄ haloalkoxy, C₄₋₆ cycloalkoxy, and C₄₋₆ halocycloalkoxy.

10. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R¹ is selected from a hydrogen atom, methoxy, difluoromethoxy, trifluoromethoxy, —OCH₂CHF₂, cyclobutyloxy, and

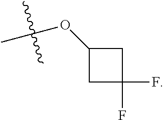 [SMILES: *OC1CC(F)(F)C1]

11. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R¹ is cyclobutyloxy or methoxy.

12. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R¹ is methoxy.

13. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R² and R³ are each independently selected from a hydrogen atom, halogen, C₁₋₄ alkyl, and C₁₋₄ alkoxy.

14. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein both R² and R³ are hydrogen atoms.

15. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R⁶ and R⁷ are identical or different and are each independently selected from a hydrogen atom, a chlorine atom, a fluorine atom, methoxy, ethoxy, isopropoxy, difluoromethoxy, and cyclopropyl.

16. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R⁶ and R⁷ are identical or different and are each independently selected from a hydrogen atom, a chlorine atom, a fluorine atom, and methoxy.

17. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R⁶ is methoxy.

18. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R⁷ is a hydrogen atom or a fluorine atom.

19. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein R⁷ is a fluorine atom.

20. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9A} and R^{9B} are identical or different and are each independently selected from a hydrogen atom, a fluorine atom, and methyl.

21. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein both R^{9A} and R^{9B} are hydrogen atoms.

22. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, heteroaryl, —C(O)C₁₋₆ alkyl, —C(O)C₃₋₆ cycloalkyl, —C(O)C₁₋₆ haloalkyl, —C(O)C₃₋₆ halocycloalkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, benzyl, and

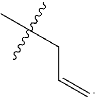 [SMILES: *CC=C]

23. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is a hydrogen atom.

24. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is methyl.

25. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is deuterated methyl.

26. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is ethyl.

27. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is trifluoroethyl.

28. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is —C(O)OCH₃.

29. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is benzyl.

30. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is —C(O)CH₃.

31. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9C} is —S(O)₂CH₃.

32. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9D} and R^{9E} are identical or different and are each independently selected from a hydrogen atom, a fluorine atom, methyl, and hydroxyl.

33. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein R^{9D} and R^{9E} are identical or different and are each independently selected from a fluorine atom and methyl.

34. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein ring C is selected from 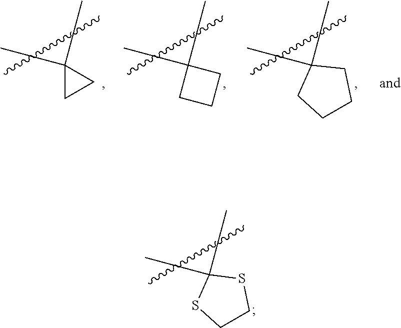
ring C is optionally substituted with one or more substituents selected from halogen, hydroxyl, amino, C₁₋₆ alkyl, and C₁₋₆ haloalkyl; preferably, ring C is selected from

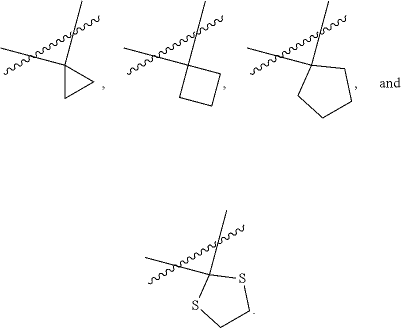

35. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein Y is O.

36. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein p is 1.

37. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 6, wherein p is 2.

38. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 5, wherein the compound is a compound of formula (VII-1), (VII-2), or (VII-3), 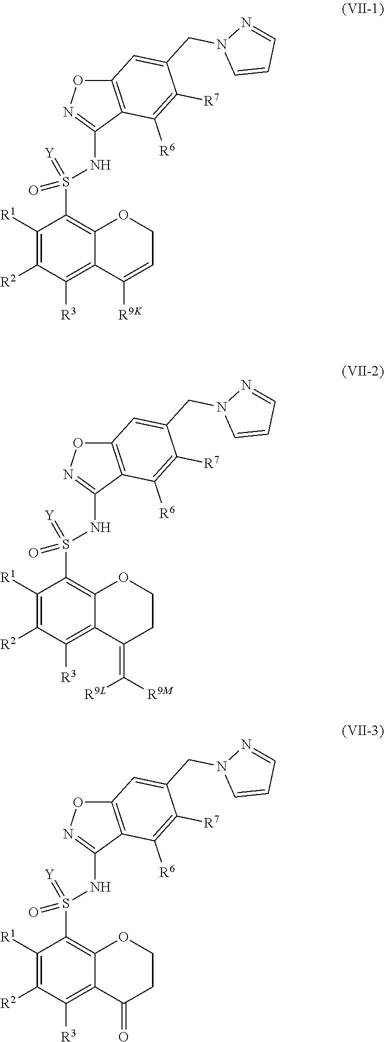
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9K} is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ heterocyclyl, 6- to 10-membered aryl, 5- to 10-membered heteroaryl, and hydroxyl, wherein the C₃₋₆ cycloalkyl and C₃₋₆ heterocyclyl are optionally substituted with one or more substituents selected from: halogen, hydroxyl, methyl, and methoxy;
R^{9M} and R^{9L} are identical or different and are each independently selected from a hydrogen atom, C₁₋₆ alkyl, and C₁₋₆ haloalkyl;
Y is O or NH.

39. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R¹ is selected from a hydrogen atom, C₁₋₄ alkoxy, C₁₋₄ haloalkoxy, C₄₋₆ cycloalkoxy, and C₄₋₆ halocycloalkoxy.

40. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R¹ is selected from a hydrogen atom, methoxy, difluoromethoxy, trifluoromethoxy, —OCH₂CHF₂, cyclobutyloxy, and

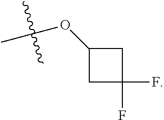 [SMILES: *OC1CC(F)(F)C1]

41. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R¹ is methoxy.

42. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R² and R³ are identical or different and are each independently selected from a hydrogen atom, halogen, C₁₋₄ alkyl, and C₁₋₄ alkoxy.

43. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R² and R³ are identical or different and are each independently selected from a hydrogen atom and halogen.

44. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein both R² and R³ are hydrogen atoms.

45. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R⁶ and R⁷ are identical or different and are each independently selected from a hydrogen atom, a chlorine atom, a fluorine atom, methoxy, ethoxy, isopropoxy, difluoromethoxy, and cyclopropyl.

46. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R⁶ is methoxy.

47. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R⁷ is selected from a hydrogen atom and a fluorine atom.

48. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R⁷ is a fluorine atom.

49. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R^{9K} is selected from a hydrogen atom, halogen, C₁₋₆ alkyl, C₁₋₆ haloalkyl, and hydroxyl.

50. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R^{9K} is selected from a hydrogen atom, methyl, and hydroxyl.

51. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R^{9K} is a hydrogen atom.

52. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R^{9M} and R^{9L} are identical or different and are each independently selected from a hydrogen atom and C₁₋₆ alkyl.

53. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein R^{9M} and R^{9L} are identical or different and are each independently selected from a hydrogen atom, a fluorine atom, and methyl.

54. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 38, wherein Y is O.

55. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the compound is isotopically substituted; preferably, the isotopic substitution is a substitution with a deuterium atom.

56. The compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the structural formula of the compound is selected from Table 1.

57. A method for preparing a compound or a pharmaceutically acceptable salt thereof, comprising: 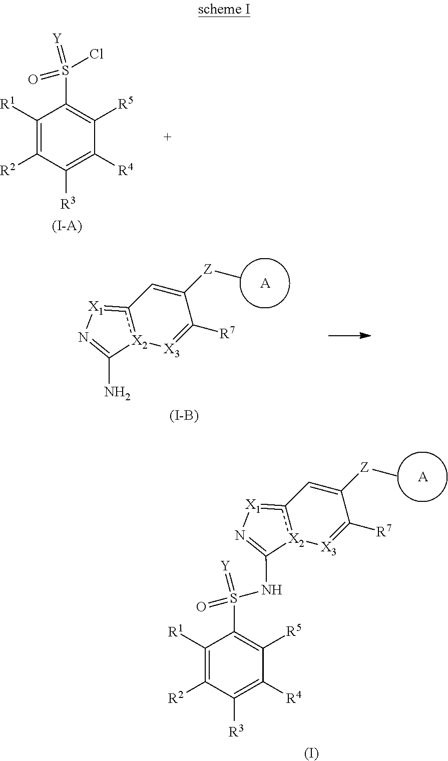
reacting a compound of formula (I-A) or a salt thereof with a compound of formula (I-B) or a salt thereof in the presence of a base to give a compound of formula (I) or a pharmaceutically acceptable salt thereof,
wherein:
the dashed line is an optional chemical bond, and
ring A, R¹, R², R³, R⁴, R⁵, R⁷, X₁, X₂, X₃, Y, and Z are as defined in claim 1;
scheme II: 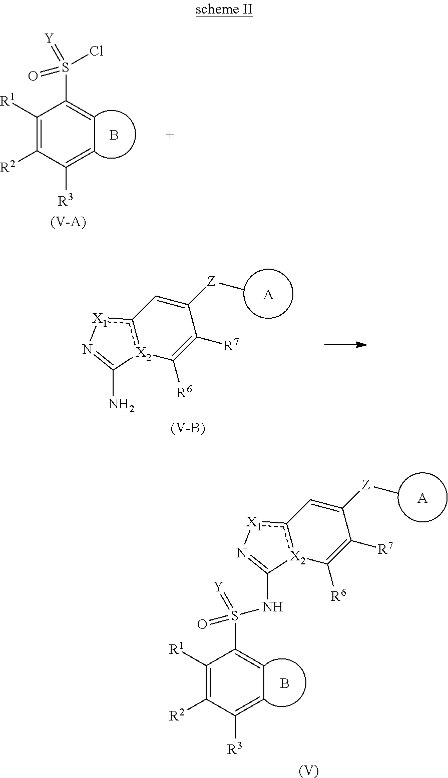
reacting a compound of formula (V-A) or a salt thereof with a compound of formula (V-B) or a salt thereof in the presence of a base to give a compound of formula (V) or a pharmaceutically acceptable salt thereof,
wherein:
the dashed line is an optional chemical bond;
ring B is selected from 4- to 10-membered heterocyclyl, 5- to 6-membered heteroaryl, and 7- to 12-membered spiroheterocyclyl; ring B is optionally substituted with one or more substituents selected from: hydroxyl, amino, halogen, C₁₋₆ alkyl, C₁₋₆ haloalkyl, C₁₋₆ alkoxy, C₁₋₆ haloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, oxo, ═CH₂, ═CHC₁₋₆ alkyl, ═C(C₁₋₆ alkyl)₂, ═CHC₁₋₆ haloalkyl, ═C(C₁₋₆ haloalkyl)₂, —NHC₁₋₆ alkyl, —N(C₁₋₆ alkyl)₂, cyano, benzyl, C₃₋₆ cycloalkyl, C₃₋₆ heterocyclyl, C₃₋₆ halocycloalkyl, C₃₋₆ cycloalkoxy, C₃₋₆ halocycloalkoxy, 6-to 10-membered aryl, 5- to 10-membered heteroaryl, and C₂₋₆ alkenyl; and
ring A, R¹, R², R³, R⁶, R⁷, X₁, X₂, Y, and Z are as defined in claim 1, scheme III: 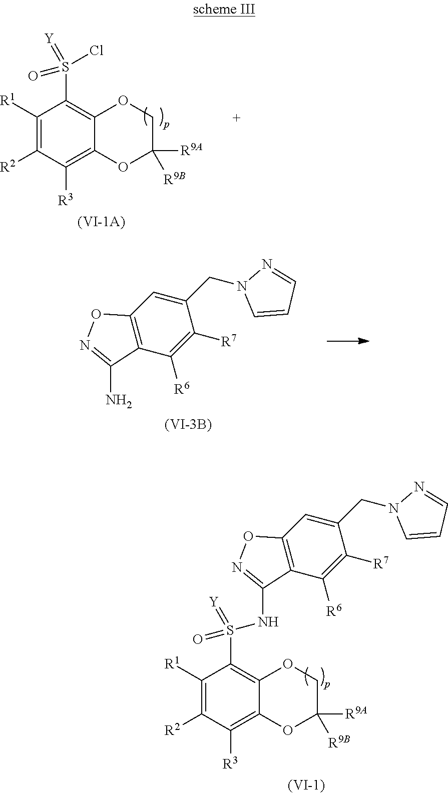
reacting a compound of formula (VI-1A) or a salt thereof with a compound of formula (VI-3B) or a salt thereof in the presence of a base to give a compound of formula (VI-1) or a pharmaceutically acceptable salt thereof,
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy:
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
Y is O or NH; and
p is 0, 1, or 2 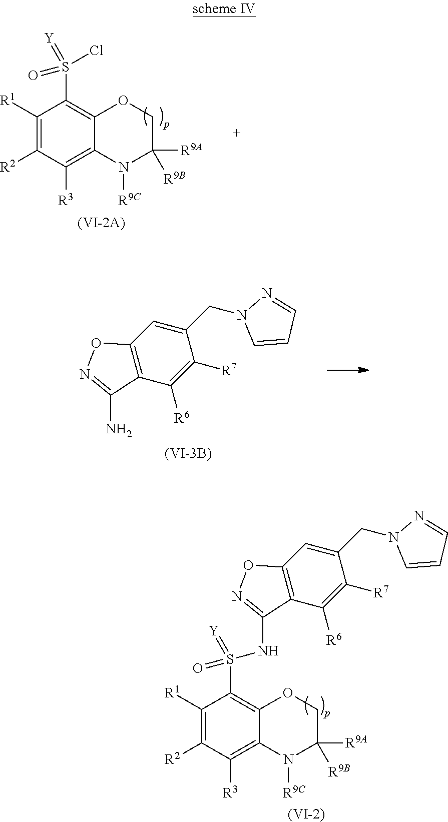
reacting a compound of formula (VI-2A) or a salt thereof with a compound of formula (VI-3B) or a salt thereof in the presence of a base to give a compound of formula (VI-2) or a pharmaceutically acceptable salt thereof,
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9C} is selected from a hydrogen atom, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, C₃₋₆ halocycloalkoxy, —C(O)C₁₋₆ alkyl, —C(O)C₃₋₆ cycloalkyl, —C(O)C₁₋₆ haloalkyl, —C(O)C₃₋₆ halocycloalkyl, —C(O)OC₁₋₆ alkyl, —S(O)₂C₁₋₆ alkyl, —S(O)₂C₃₋₆ cycloalkyl, —S(O)₂C₁₋₆ haloalkyl, —S(O)₂C₃₋₆ halocycloalkyl, —PO(C₁₋₆ alkyl)₂, —PO(C₃₋₆ cycloalkyl)₂, —PO(C₁₋₆ haloalkyl)₂, —PO(C₃₋₆ halocycloalkyl)₂, benzyl, 6- to 10-membered aryl, 5- to 10-membered heteroaryl, and C₂₋₆ alkenyl:
Y is O or NH; and
p is 0, 1, or 2
scheme V: 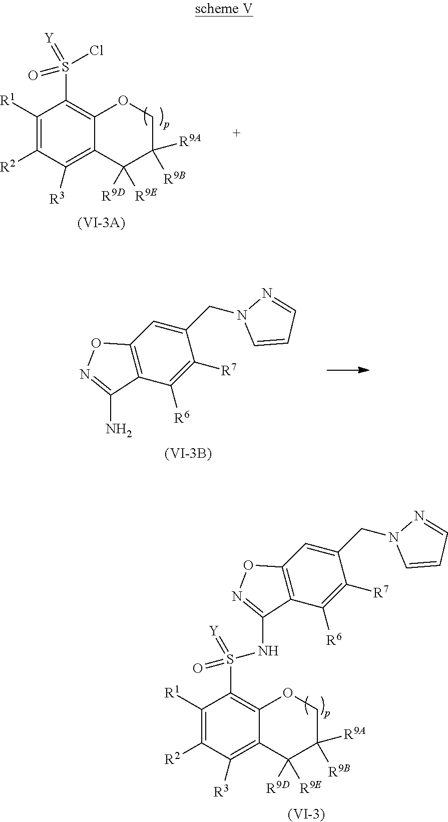
reacting a compound of formula (VI-3A) or a salt thereof with a compound of formula (VI-3B) or a salt thereof in the presence of a base to give a compound of formula (VI-3) or a pharmaceutically acceptable salt thereof,
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
R^{9D} and R^{9E} are identical or different and are each independently selected from a hydrogen atom, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, —NHC₁₋₆ alkyl, —N(C₁₋₆ alkyl)₂, C₃₋₆ cycloalkyl, C₃₋₆ halocycloalkyl, C₃₋₆ cycloalkoxy, C₃₋₆ halocycloalkoxy, and hydroxyl;
Y is O or NH; and
p is 0, 1, or 2
scheme VI: 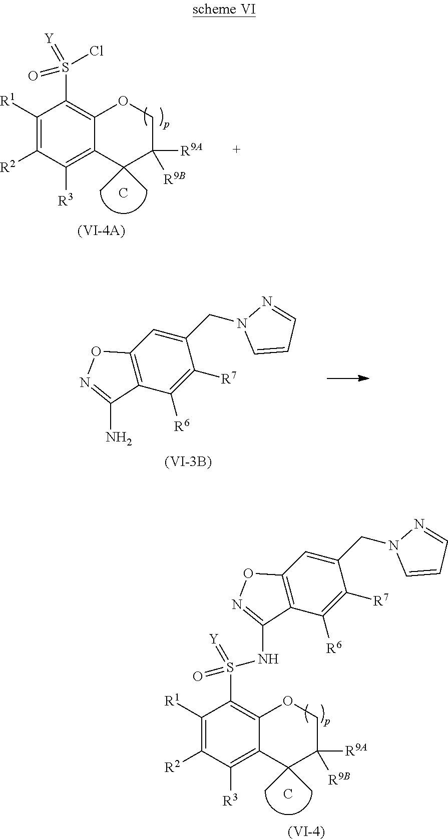
reacting a compound of formula (VI-4A) or a salt thereof with a compound of formula (VI-3B) or a salt thereof in the presence of a base to give a compound of formula (VI-4) or a pharmaceutically acceptable salt thereof,
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy;
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
R^{9A} and R^{9B} are identical or different and are each independently selected from a hydrogen atom, hydroxyl, oxo, halogen, C₁₋₆ alkyl, C₁₋₆ haloalkyl, C₁₋₆ alkoxy, C₁₋₆ haloalkoxy, —C(O)C₁₋₆ alkyl, —C(O)C₁₋₆ haloalkyl, cyano, C₃₋₆ cycloalkyl, C₃₋₆ halocycloalkyl, C₃₋₆ cycloalkoxy, and C₃₋₆ halocycloalkoxy;
Y is O or NH:
p is 0, 1, or 2; and
ring C is selected from 3- to 8-membered cycloalkyl, 3- to 8-membered heterocyclyl, 6-to 12-membered spirocyclyl, 6- to 12-membered spiroheterocyclyl, and 5- to 12-membered bridged heterocyclyl; ring C is optionally substituted with one or more substituents selected from halogen, hydroxyl, amino, C₁₋₆ alkyl, and C₁₋₆ haloalkyl; 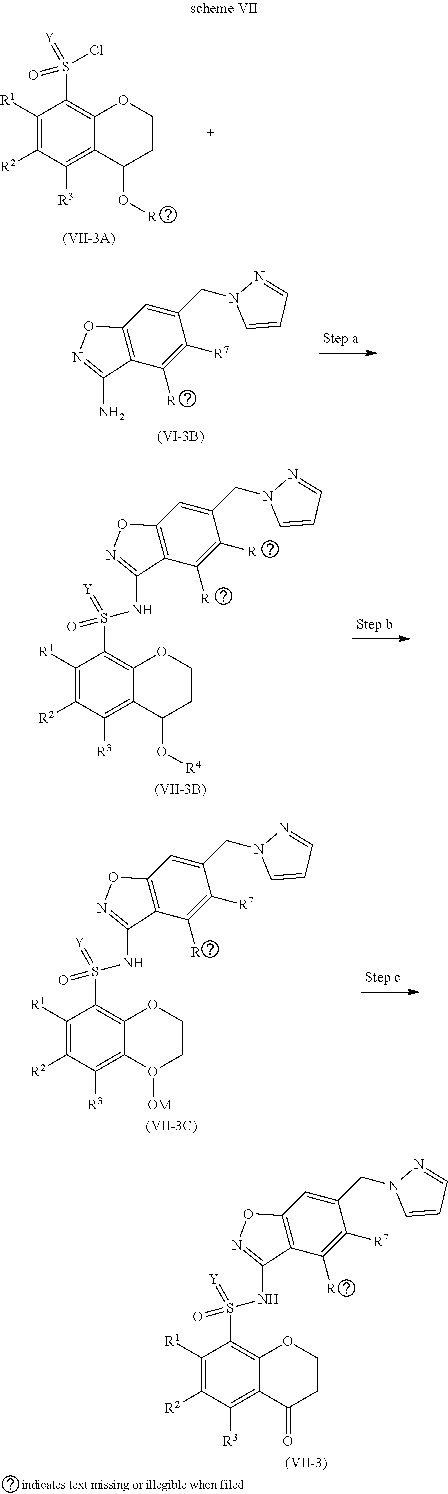
step a: reacting a compound of formula (VII-3A) or a salt thereof with a compound of formula (VI-3B) or a salt thereof in the presence of a base to give a compound of formula (VII-3B) or a pharmaceutically acceptable salt thereof;
step b: reacting the compound of formula (VII-3B) or the salt thereof with a deprotecting reagent to remove a hydroxyl protecting group to give a compound of formula (VII-3C) or a pharmaceutically acceptable salt thereof; and
step c: reacting the compound of formula (VII-3C) or the salt thereof with an oxidizing reagent for oxidation to give a compound of formula (VII-3) or a pharmaceutically acceptable salt thereof;
wherein:
R¹ is selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
R² and R³ are identical or different and are each independently selected from a hydrogen atom, halogen, C₁₋₄ alkyl, C₁₋₄ haloalkyl, C₁₋₄ alkoxy, and C₁₋₄ haloalkoxy;
R⁶ and R⁷ are identical or different and are each independently selected from a hydrogen atom, halogen, C₁₋₆ alkyl, C₁₋₆ haloalkyl, C₁₋₆ alkoxy, C₁₋₆ haloalkoxy, C₃₋₆ cycloalkyl, C₃₋₆ halocycloalkyl, C₃₋₆ cycloalkoxy, and C₃₋₆ halocycloalkoxy:
Y is O or NH:
R^{a} is the hydroxyl protecting group, preferably (trimethylsilyl)ethoxymethyl;
the deprotecting reagent in step b is an acid or a fluorine-containing reagent, preferably trifluoroacetic acid or tetrabutylammonium fluoride; and
the oxidizing reagent in step c is a reagent for oxidizing a hydroxyl group to a ketone, preferably manganese dioxide or Dess-Martin reagent;
wherein the base used in the reactions of the above schemes is selected from:
triethylamine, diisopropylethylamine, pyridine, 2,4-dimethylpyridine, 2,6-dimethylpyridine, n-butyllithium, lithium bis(trimethylsilyl)amide, sodium hydride, sodium hydroxide, cesium carbonate, and potassium carbonate; and
the reactions of the above schemes are preferably carried out in a solvent selected from:
ethylene glycol dimethyl ether, methanol, ethanol, acetonitrile, n-butanol, toluene, tetrahydrofuran, dichloromethane, dimethyl sulfoxide, 1,4-dioxane, N,N-dimethylformamide, N,N-dimethylacetamide, 1,2-dibromoethane, toluene, pyridine, and a mixture thereof.

58. A pharmaceutical composition, comprising the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, and one or more pharmaceutically acceptable carriers, diluents, or excipients.

59-65. (canceled)

66. A method for inhibiting KAT6A/B, comprising administering the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, or a pharmaceutical composition comprising the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1.

67. A method for treating or preventing a cancer in which KAT6A/B plays a role, comprising administering to a subject in need thereof the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, or a pharmaceutical composition comprising the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, wherein the cancer is selected from lung cancer, breast cancer, rectal cancer, colon cancer, esophageal cancer, gastric cancer, liver cancer, gallbladder cancer, cholangiocarcinoma, kidney cancer, bladder cancer, urothelial cancer, head and neck cancer, nasopharyngeal cancer, prostate cancer, cervical cancer, endometrial cancer, ovarian cancer, pancreatic cancer, melanoma, bone cancer, mesothelioma, gastrointestinal stromal tumor, sarcoma, brain glioma, thyroid cancer, salivary gland tumor, glioblastoma, neuroblastoma, gastric myxoma, lymphoma, leukemia, plasmacytoma, sinoatrial node tumor, and tenosynovial giant cell tumor.

68. The method according to claim 67, wherein the cancer is selected from breast cancer, prostate cancer, lung cancer, pancreatic cancer, ovarian cancer, cervical cancer, endometrial cancer, bladder cancer, brain glioma, malignant lymphoma, liver cancer, and leukemia.

69. The method according to claim 68, wherein the breast cancer is ER⁺ breast cancer or ER⁺/HER2⁻ breast cancer.

70. The method according to claim 68, wherein the lung cancer is non-small cell lung cancer.

71. The method according to claim 68, wherein the prostate cancer is castration-resistant prostate cancer.

72. A kit, comprising the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, or a pharmaceutical composition comprising the compound or the pharmaceutically acceptable salt, the ester, the stereoisomer, the tautomer, the polymorph, the hydrate, the solvate, the N-oxide, the isotopically labeled compound, the metabolite, or the prodrug thereof according to claim 1, and a package insert.
